(19) United States
(12) Patent Application Publication
(10) Pub. No.: US 2010/0305133 A1
(43) Pub. Date: Dec. 2, 2010

(54) Prolyl Hydroxylase Inhibitors

(75) Inventors: Mariela Colón, Collegeville, PA (US); Duke M. Fitch, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: SMITHKLINE BEECHAM CORPORATION

(21) Appl. No.: 12/744,704

(22) PCT Filed: Nov. 26, 2008
(86) PCT No.: PCT/US08/84791

§ 371 Date: May 26, 2010

Related U.S. Application Data
(60) Provisional application No. 60/991,279, filed on Nov. 30, 2007.
(60) Provisional application No. 61/023,913, filed on Jan. 28, 2008.
(60) Provisional application No. 61/023,937, filed on Jan. 28, 2008.

(51) Int. Cl. A61K 31/517; C07D 241/36; C07D 401/02; C07D 417/04; C07D 403/02; A61P 7/06

(52) U.S. Cl. 514/249; 544/353

(57) ABSTRACT

The invention described herein relates to certain quinoxaline-5-carboxamide derivatives of formula (I)

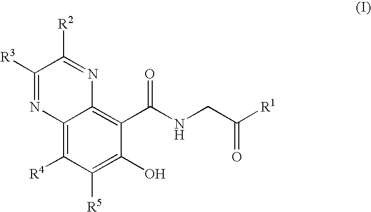

which are antagonists of HIF prolyl hydroxylases and are useful for treating diseases benefiting from the inhibition of this enzyme, anemia being one example.

### FIELD OF THE INVENTION

This invention relates to certain quinoxaline-5-carboxamide derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1alpha, HIF-2alpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

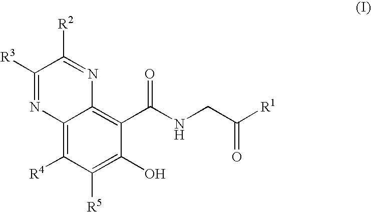

wherein:

R¹ is —NR⁶R⁷ or —OR⁸;

R², R³, R⁴, and R⁵ are each independently selected from the group consisting of hydrogen, nitro, cyano, halogen, —C(O)R¹¹, —C(O)OR¹¹, —OR¹¹, —SR¹¹, —S(O)R¹¹, —S(O)₂R¹¹, —NR⁹R¹⁰, —CONR⁹R¹⁰, —N(R⁹)C(O)R¹¹, —N(R⁹)C(O)OR¹¹, —OC(O)NR⁹R¹⁰, —N(R⁹)C(O)N⁹R¹⁰, —P(O)(OR¹¹)₂, —SO₂NR⁹R¹⁰, —N(R⁹)SO₂R¹¹, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R⁶ and R⁷ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R⁸ is hydrogen, or a cation, or C₁-C₄ alkyl;

R⁹ and R¹⁰ are each independently selected from the group consisting of hydrogen, C₁-Cₐ) alkyl, C₃-C₈cycloalkyl, C₁-C₁₀ alkyl-C₃-C₈cycloalkyl, C₃-C₈heterocycloalkyl, C₁-C₁₀ alkyl-C₃-C₈ heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁹ and R¹⁰ taken together with the nitrogen to which they are attached form a 5- or 6- or 7-membered saturated ring optionally containing one other heteroatom which is oxygen, nitrogen or sulphur;

each R¹¹ is independently selected from the group consisting of hydrogen, C₁-C₁₀ alkyl, C₂-C₁₀alkenyl, C₂-C₁₀ alkynyl, —CO(C₁-C₄ alkyl), —CO(aryl), —CO(heteroaryl), —CO(C₃-C₆ cycloalkyl), —CO(C₃-C₆heterocycloalkyl), —SO₂(C₁-C₄ alkyl), C₃-C₈ cycloalkyl, C₃-C₈ heterocycloalkyl, aryl, C₁-C₁₀alkyl-aryl, heteroaryl, and C₁-C₁₀alkyl-heteroaryl;

any carbon or heteroatom of R², R³, R⁴, R⁵, R⁶, R⁷, R⁸, R⁹, R¹⁰, or R¹¹ is unsubstituted or, where possible, is substituted with one or more substituents independently selected from C₁-C₆ alkyl, aryl, heteroaryl, halogen, —OR¹¹, —NR⁹R¹⁰, cyano, nitro, —C(O)R¹¹, —C(O)OR¹¹, —SR¹¹, —S(O)R¹¹, —S(O)₂R¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₁₀ alkenyl, C₂-C₁₀ alkynyl, C₃-C₈ cycloalkyl, C₃-C₈ heterocycloalkyl, C₅-C₈ cycloalkenyl, aryl or heteroaryl, wherein R⁹, R¹⁰, and R¹¹ are the same as defined above;

or a pharmaceutically acceptable salt or solvate thereof.

In a second aspect of the present invention, there is provided a compound of formula (I) or a salt or solvate thereof for use in mammalian therapy, e.g. treating amenia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁-C₄ alkyl” and “C₁-C₁₀ alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃-C₈ cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,4-dithiane, 1,3-dioxane, 1,3-dioxolane, piperidine, piperazine, 2,4-piperazinedione, pyrrolidine, 2-imidazoline, imidazolidine, pyrazolidine, pyrazoline, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, benzofuranyl, benzothiophenyl, benzimidazolyl, benzthiazolyl, indolizinyl, indolyl, isoindolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R¹ is —NR⁶R⁷ or —OR⁸;

R², R³, R⁴, and R⁵ are each independently selected from the group consisting of hydrogen, cyano, halogen, —C(O)R¹¹, —C(O)OR¹¹, —OR¹¹, —NR⁹R¹⁰, —CONR⁹R¹⁰, —N(R⁹)C(O)R¹¹, —N(R⁹)C(O)R¹¹, —N(R⁹)C(O)N⁹R¹⁰, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;

R⁶ and R⁷ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R⁸ is hydrogen, or a cation, or C₁-C₄ alkyl;

R⁹ and R¹⁰ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁹ and R¹⁰ taken together with the nitrogen to which they are attached form a 5- or 6- or 7-membered saturated ring optionally containing one other heteroatom which is oxygen, nitrogen or sulphur;

each R¹¹ is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;

any carbon or heteroatom of R², R³, R⁴, R⁵, R⁶, R⁷, R⁸, R⁹, R¹⁰, or R², is unsubstituted or, where possible, is substituted with one or more substituents independently selected from C₁-C₆ alkyl, aryl, heteroaryl, halogen, —OR¹¹, —NR⁹R¹⁰, cyano, —C(O)R¹¹, —C(O)OR¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R⁹, R¹⁰, and R¹¹ are the same as defined above;

Compounds of further interest are those wherein:

R¹ is —OR⁸;

R², R³, R⁴, and R⁵ are each independently selected from the group consisting of hydrogen, cyano, halogen, —OR¹¹, —NR⁹R¹⁰, —CONR⁹R¹⁰, C₁-C₆ alkyl, C₃-C₆ cycloalkyl, C₃-C₆ heterocycloalkyl, aryl, and heteroaryl;

R⁸ is hydrogen, or a cation;

R⁹ and R¹⁰ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁹ and R¹⁰ taken together with the nitrogen to which they are attached form a 5- or 6- or 7-membered saturated ring optionally containing one other heteroatom which is oxygen, nitrogen or sulphur;

each R¹¹ is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;

any carbon or heteroatom of R², R³, R⁴, R⁵, R⁸, R⁹, R¹⁰, or R¹¹ is unsubstituted or, where possible, is substituted with one or more substituents independently selected from C₁-C₆ alkyl, aryl, heteroaryl, halogen, —OR¹¹, —NR⁹R¹⁰, cyano, —C(O)R¹¹, —C(O)OR¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₆alkenyl, C₂-C₆ alkynyl, C₃-C₆ cycloalkyl, C₃-C₆ heterocycloalkyl, C₅-C₈ cycloalkenyl, aryl, or heteroaryl, wherein R⁹, R¹⁰, and R¹¹ are the same as defined above;

Of further interest are those compounds where:

R¹ is −OR⁸;

R⁴ is hydrogen;

R², R³, and R⁵ are each independently selected from the group consisting of hydrogen, cyano, halogen, —OR¹¹, —NR⁹R¹⁰, —CONR⁹R¹⁰, C₁-C₆ alkyl, C₃-C₆ cycloalkyl, C₃-C₆ heterocycloalkyl, aryl, and heteroaryl;

R⁸ is hydrogen, or a cation;

R⁹ and R¹⁰ are each independently selected from the group consisting of hydrogen, C₁-C₆ alkyl, C₃-C₆ cycloalkyl, C₃-C₆ heterocycloalkyl, aryl, and heteroaryl; or R⁹ and R¹⁰ taken together with the nitrogen to which they are attached form a 5- or 6- or 7-membered saturated ring optionally containing one other heteroatom which is oxygen, nitrogen or sulphur;

each R¹¹ is independently selected from the group consisting of hydrogen, C₁-C₆ alkyl, C₃-C₆ cycloalkyl, C₃-C₆ heterocycloalkyl, aryl, and heteroaryl;

any carbon or heteroatom of R², R³, R⁵, R⁸, R⁹, R¹⁰, or R¹¹ is unsubstituted or, where possible, is substituted with one or more substituents independently selected from C₁-C₆ alkyl, aryl, heteroaryl, halogen, —OR¹¹, —NR⁹R¹⁰, cyano, —C(O)R¹¹, —C(O)OR¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₆alkenyl, C₂-C₆ alkynyl, C₃-C₆ cycloalkyl, C₃-C₆ heterocycloalkyl, C₅-C₈ cycloalkenyl, aryl, or heteroaryl, wherein R⁹, R¹⁰, and R¹¹ are the same as defined above;

Specific compounds exemplified herein are:
1) N-[(6-hydroxy-3-phenyl-5-quinoxalinyl)carbonyl]glycine;
2) N-[(6-hydroxy-3-methyl-5-quinoxalinyl)carbonyl]glycine;
3) N-[(6-hydroxy-5-quinoxalinyl)carbonyl]glycine;
4) N-[(2-bromo-6-hydroxy-5-quinoxalinyl)carbonyl]glycine;
5) N-({6-hydroxy-2-[4-(trifluoromethyl)phenyl]-5-quinoxalinyl}carbonyl)glycine;
6) N-({6-hydroxy-2-[(phenylmethyl)amino]-5-quinoxalinyl}carbonyl)glycine;
7) N-{[6-hydroxy-2-(phenylamino)-5-quinoxalinyl]carbonyl}glycine;
8) N-{[6-hydroxy-2-(phenyloxy)-5-quinoxalinyl]carbonyl}glycine;
9) N-{[6-hydroxy-2-(1-piperidinyl)-5-quinoxalinyl]carbonyl}glycine;
10) N-{[7-(3,5-difluorophenyl)-6-hydroxy-5-quinoxalinyl]carbonyl}glycine;
11) N-[(7-bromo-6-hydroxy-5-quinoxalinyl)carbonyl]glycine;
12) N-{[7-(2-chlorophenyl)-6-hydroxy-5-quinoxalinyl]carbonyl}glycine;
13) N-{[6-hydroxy-7-(1-methylethyl)-5-quinoxalinyl]carbonyl}glycine;
14) N-[(6-hydroxy-2,3-dimethyl-5-quinoxalinyl)carbonyl]glycine;
15) N-[(7-bromo-6-hydroxy-3-phenyl-5-quinoxalinyl)carbonyl]glycine;
16) N-{[7-bromo-3-(3,4-difluorophenyl)-6-hydroxy-5-quinoxalinyl]carbonyl}glycine;
17) N-{[7-bromo-3-(2,4-difluorophenyl)-6-hydroxy-5-quinoxalinyl]carbonyl}glycine;
18) N-{[7-bromo-3-(1,1-dimethylethyl)-6-hydroxy-5-quinoxalinyl]carbonyl}glycine;
19) N-{[7-bromo-3-(4-cyclohexylphenyl)-6-hydroxy-5-quinoxalinyl]carbonyl}glycine;
20) N-{[7-bromo-3-(4-fluorophenyl)-6-hydroxy-5-quinoxalinyl]carbonyl}glycine;
21) N-{[6-hydroxy-7-(2-pyridinyl)-5-quinoxalinyl]carbonyl}glycine;
22) N-{[6-hydroxy-7-(1,3-thiazol-2-yl)-5-quinoxalinyl]carbonyl}glycine;
23) N-[(6-hydroxy-7-phenyl-5-quinoxalinyl)carbonyl]glycine;
24) N-{[6-hydroxy-7-(1-methyl-1H-imidazol-2-yl)-5-quinoxalinyl]carbonyl}glycine;
25) N-{[6-hydroxy-3-phenyl-7-(2-pyridinyl)-5-quinoxalinyl]carbonyl}glycine;
26) N-{[6-hydroxy-7-(3-pyridinyl)-5-quinoxalinyl]carbonyl}glycine;
27) N-{[3-(3,4-difluorophenyl)-6-hydroxy-7-(2-pyridinyl)-5-quinoxalinyl]carbonyl}glycine;
28) N-{[6-hydroxy-3-phenyl-7-(1,3-thiazol-2-yl)-5-quinoxalinyl]carbonyl}glycine;
29) N-{[3-(3,4-difluorophenyl)-6-hydroxy-7-(1,3-thiazol-2-yl)-5-quinoxalinyl]carbonyl}glycine;
30) N-[(7-butyl-6-hydroxy-5-quinoxalinyl)carbonyl]glycine;
31) N-{[6-hydroxy-7-(4-pyridinyl)-5-quinoxalinyl]carbonyl}glycine; and
32) N-{[6-hydroxy-7-(5-pyrimidinyl)-5-quinoxalinyl]carbonyl}glycine.
33) N-{[6-hydroxy-7-(1-methyl-1H-pyrazol-4-yl)-5-quinoxalinyl]carbonyl}glycine
34) N-{[6-hydroxy-7-(2-pyrazinyl)-5-quinoxalinyl]carbonyl}glycine
35) N-{[6-hydroxy-7-(4-methyl-1,3-thiazol-2-yl)-5-quinoxalinyl]carbonyl}glycine
36) N-{[7-(2-furanyl)-6-hydroxy-5-quinoxalinyl]carbonyl}glycine
37) N-{[6-hydroxy-7-(2-thienyl)-5-quinoxalinyl]carbonyl}glycine
38) N-{[6-hydroxy-7-(2-pyrimidinyl)-5-quinoxalinyl]carbonyl}glycine
39) N-{[6-hydroxy-7-(5-methyl-1,3-thiazol-2-yl)-5-quinoxalinyl]carbonyl}glycine
40) N-{[6-hydroxy-7-(1,3-oxazol-2-yl)-5-quinoxalinyl]carbonyl}glycine
41) N-[(6-hydroxy-8-phenyl-5-quinoxalinyl)carbonyl]glycine
42) N-{[6-hydroxy-7-(1H-indol-3-yl)-5-quinoxalinyl]carbonyl}glycine
43) N-{[6-hydroxy-7-(1H-pyrrol-3-yl)-5-quinoxalinyl]carbonyl}glycine
44) N-[(6-hydroxy-2-phenyl-5-quinoxalinyl)carbonyl]glycine
45) N-{[6-hydroxy-7-(1H-indol-2-yl)-5-quinoxalinyl]carbonyl}glycine
46) N-[(6-hydroxy-2-methyl-5-quinoxalinyl)carbonyl]glycine
47) N-{[3-(3,4-difluorophenyl)-6-hydroxy-7-(2-thienyl)-5-quinoxalinyl]carbonyl}glycine
48) N-{[6-hydroxy-2-phenyl-7-(2-pyridinyl)-5-quinoxalinyl]carbonyl}glycine
49) N-{[7-(1-cyclohexen-1-yl)-3-(3,4-difluorophenyl)-6-hydroxy-5-quinoxalinyl]carbonyl}glycine
50) N-{[7-(1,3-benzothiazol-2-yl)-6-hydroxy-5-quinoxalinyl]carbonyl}glycine
51) N-{[6-hydroxy-7-(1,3-thiazol-5-yl)-5-quinoxalinyl]carbonyl}glycine
52) N-[(7-fluoro-6-hydroxy-5-quinoxalinyl)carbonyl]glycine
53) N-{[7-cyclohexyl-3-(3,4-difluorophenyl)-6-hydroxy-5-quinoxalinyl]carbonyl}glycine
54) N-{[6-hydroxy-7-(3-thienyl)-5-quinoxalinyl]carbonyl}glycine
55) N-{[6-hydroxy-7-(1,3-thiazol-4-yl)-5-quinoxalinyl]carbonyl}glycine
56) N-{[7-(1-benzothien-2-yl)-6-hydroxy-5-quinoxalinyl]carbonyl}glycine
57) N-{[7-(1-benzothien-3-yl)-6-hydroxy-5-quinoxalinyl]carbonyl}glycine
58) N-[(6-hydroxy-3,7-diphenyl-5-quinoxalinyl)carbonyl]glycine
59) N-[(8-bromo-6-hydroxy-5-quinoxalinyl)carbonyl]glycine
60) N-({3-(3,4-difluorophenyl)-7-[4-(1,1-dimethylethyl)phenyl]-6-hydroxy-5-quinoxalinyl}carbonyl)glycine
61) N-{[3-(3,4-difluorophenyl)-6-hydroxy-7-phenyl-5-quinoxalinyl]carbonyl}glycine
62) N-{[3-(3,4-difluorophenyl)-7-(4-fluorophenyl)-6-hydroxy-5-quinoxalinyl]carbonyl}glycine
63) N-[(3-(3,4-difluorophenyl)-6-hydroxy-7-{3-[(1-methylethyl)oxy]phenyl}-5-quinoxalinyl)carbonyl]glycine
64) N-[(3-(3,4-difluorophenyl)-6-hydroxy-7-{4-[(1-methylethyl)oxy]phenyl}-5-quinoxalinyl)carbonyl]glycine
65) N-{[3-(3,4-difluorophenyl)-7-(3-fluorophenyl)-6-hydroxy-5-quinoxalinyl]carbonyl}glycine
66) N-[(6-hydroxy-2,3-diphenyl-5-quinoxalinyl)carbonyl]glycine
67) N-{[2-(3-fluorophenyl)-6-hydroxy-5-quinoxalinyl]carbonyl}glycine
68) N-{[6-hydroxy-8-(3-pyridinyl)-5-quinoxalinyl]carbonyl}glycine
69) N-[(6-hydroxy-2,7-diphenyl-5-quinoxalinyl)carbonyl]glycine
70) N-{[6-hydroxy-2-phenyl-7-(2-thienyl)-5-quinoxalinyl]carbonyl}glycine
71) N-{[6-hydroxy-8-(3-thienyl)-5-quinoxalinyl]carbonyl}glycine
72) N-[(6-hydroxy-2,7-di-2-thienyl-5-quinoxalinyl)carbonyl]glycine
73) N-{[6-hydroxy-8-(2-pyridinyl)-5-quinoxalinyl]carbonyl}glycine
74) N-{[6-hydroxy-8-(2-thienyl)-5-quinoxalinyl]carbonyl}glycine
75) N-[(6-hydroxy-2,7-di-1,3-thiazol-2-yl-5-quinoxalinyl)carbonyl]glycine
76) N-{[8-(2-furanyl)-6-hydroxy-5-quinoxalinyl]carbonyl}glycine
77) N-{[6-hydroxy-8-(1,3-thiazol-5-yl)-5-quinoxalinyl]carbonyl}glycine
78) N-{[6-hydroxy-8-(1,3-thiazol-4-yl)-5-quinoxalinyl]carbonyl}glycine
79) N-{[6-hydroxy-2-(3-pyridinyl)-5-quinoxalinyl]carbonyl}glycine
80) N-({6-hydroxy-2-[3-(methyloxy)phenyl]-5-quinoxalinyl}carbonyl)glycine
81) N-{[6-hydroxy-2-(2-hydroxyphenyl)-5-quinoxalinyl]carbonyl}glycine
82) N-({6-hydroxy-2-[4-(methyloxy)phenyl]-5-quinoxalinyl}carbonyl)glycine
83) N-[(6-hydroxy-2-{3-[(1-methylethyl)oxy]phenyl}-5-quinoxalinyl)carbonyl]glycine
84) N-{[8-(1-benzothien-2-yl)-6-hydroxy-5-quinoxalinyl]carbonyl}glycine
85) N-{[8-(1-cyclohexen-1-yl)-6-hydroxy-5-quinoxalinyl]carbonyl}glycine
86) N-({8-[2-fluoro-4-(trifluoromethyl)phenyl]-6-hydroxy-5-quinoxalinyl}carbonyl)glycine
87) N-{[8-(3-bromo-5-fluorophenyl)-6-hydroxy-5-quinoxalinyl]carbonyl}glycine
88) N-{[8-(4-bromo-2-fluorophenyl)-6-hydroxy-5-quinoxalinyl]carbonyl}glycine
89) N-{[2-(3,4-difluorophenyl)-6-hydroxy-7-(2-thienyl)-5-quinoxalinyl]carbonyl}glycine
90) N-{[8-(1-benzothien-3-yl)-6-hydroxy-5-quinoxalinyl]carbonyl}glycine
91) N-{[2-(3,5-difluorophenyl)-6-hydroxy-5-quinoxalinyl]carbonyl}glycine
92) N-{[6-hydroxy-2-(4-hydroxyphenyl)-5-quinoxalinyl]carbonyl}glycine
93) N-({2-[4-(dimethylamino)phenyl]-6-hydroxy-5-quinoxalinyl}carbonyl)glycine
94) N-({2-[2,4-bis(methyloxy)phenyl]-6-hydroxy-5-quinoxalinyl}carbonyl)glycine
95) N-{[2-(1-benzothien-2-yl)-6-hydroxy-5-quinoxalinyl]carbonyl}glycine
96) N-[(6-hydroxy-2-{4-[(1-methylethyl)oxy]phenyl}-5-quinoxalinyl)carbonyl]glycine
97) N-{[6-hydroxy-2-(4-pyridinyl)-5-quinoxalinyl]carbonyl}glycine
98) N-{[2-(4-fluorophenyl)-6-hydroxy-5-quinoxalinyl]carbonyl}glycine
99) N-{[2-(3,4-difluorophenyl)-6-hydroxy-5-quinoxalinyl]carbonyl}glycine
100) N-({6-hydroxy-2-[3-(trifluoromethyl)phenyl]-5-quinoxalinyl}carbonyl)glycine
101) N-({2-[3-(dimethylamino)phenyl]-6-hydroxy-5-quinoxalinyl}carbonyl)glycine
102) N-({6-hydroxy-2-[2-(methyloxy)phenyl]-5-quinoxalinyl}carbonyl)glycine
103) N-{[6-hydroxy-2-(2-thienyl)-5-quinoxalinyl]carbonyl}glycine
104) N-[(6-hydroxy-2-{2-[(1-methylethyl)oxy]phenyl}-5-quinoxalinyl)carbonyl]glycine
105) N-{[6-hydroxy-8-(1-methyl-1H-pyrazol-3-yl)-5-quinoxalinyl]carbonyl}glycine
106) N-{[8-(3,4-difluorophenyl)-6-hydroxy-5-quinoxalinyl]carbonyl}glycine
107) N-{[6-hydroxy-8-(1,3-thiazol-2-yl)-5-quinoxalinyl]carbonyl}glycine
108) N-{[6-hydroxy-2-(3-hydroxyphenyl)-5-quinoxalinyl]carbonyl}glycine
109) N-({2-[2,3-bis(methyloxy)phenyl]-6-hydroxy-5-quinoxalinyl}carbonyl)glycine
110) N-({2-[3,5-bis(methyloxy)phenyl]-6-hydroxy-5-quinoxalinyl}carbonyl)glycine
111) N-{[2-(1-benzothien-3-yl)-6-hydroxy-5-quinoxalinyl]carbonyl}glycine
112) N-({6-hydroxy-2-[2-(trifluoromethyl)phenyl]-5-quinoxalinyl}carbonyl)glycine
113) N-{[2-(2,4-difluorophenyl)-6-hydroxy-5-quinoxalinyl]carbonyl}glycine
114) N-{[8-(3-furanyl)-6-hydroxy-5-quinoxalinyl]carbonyl}glycine
115) N-[(6-hydroxy-2-oxo-3-phenyl-1,2-dihydro-5-quinoxalinyl)carbonyl]glycine
116) N-{[6-hydroxy-8-(3-nitrophenyl)-5-quinoxalinyl]carbonyl}glycine
117) N-{[6-hydroxy-8-(2-nitrophenyl)-5-quinoxalinyl]carbonyl}glycine
118) N-{[6-hydroxy-3-phenyl-2-(propylamino)-5-quinoxalinyl]carbonyl}glycine
119) N-({7-[2-fluoro-4-(trifluoromethyl)phenyl]-6-hydroxy-5-quinoxalinyl}carbonyl)glycine
120) N-{[6-hydroxy-2-(1-methyl-1H-pyrazol-4-yl)-5-quinoxalinyl]carbonyl}glycine
121) N-{[2-(2-fluorophenyl)-6-hydroxy-5-quinoxalinyl]carbonyl}glycine
122) N-({6-hydroxy-3-phenyl-2-[(phenylmethyl)amino]-5-quinoxalinyl}carbonyl)glycine
123) N-{[6-hydroxy-2-phenyl-3-(1,3-thiazol-2-yl)-5-quinoxalinyl]carbonyl}glycine
124) N-({2-[3,4-bis(methyloxy)phenyl]-6-hydroxy-5-quinoxalinyl}carbonyl)glycine
125)N-{[6-hydroxy-2-(3-thienyl)-5-quinoxalinyl]carbonyl}glycine
126) N-{[6-hydroxy-2-(1,3-thiazol-2-yl)-5-quinoxalinyl]carbonyl}glycine
127) N-{[2-(2,3-difluorophenyl)-6-hydroxy-5-quinoxalinyl]carbonyl}glycine
128) N-{[2-(1,3-benzothiazol-2-yl)-6-hydroxy-5-quinoxalinyl]carbonyl}glycine
129) N-({2-[3-(1,1-dimethylethyl)phenyl]-6-hydroxy-5-quinoxalinyl}carbonyl)glycine
130) N-({2-[4-(1,1-dimethylethyl)phenyl]-6-hydroxy-5-quinoxalinyl}carbonyl)glycine
131) N-{[7-(4-bromo-2-fluorophenyl)-6-hydroxy-5-quinoxalinyl]carbonyl}glycine
132) N-{[7-(3-bromo-5-fluorophenyl)-6-hydroxy-5-quinoxalinyl]carbonyl}glycine
133) N-{[6-hydroxy-3-phenyl-2-(1,3-thiazol-2-yl)-5-quinoxalinyl]carbonyl}glycine
134) N-[(7-chloro-6-hydroxy-5-quinoxalinyl)carbonyl]glycine
135) N-({2-[2-(dimethylamino)phenyl]-6-hydroxy-5-quinoxalinyl}carbonyl)glycine
136) N-{[7-(3,4-difluorophenyl)-6-hydroxy-2-(2-thienyl)-5-quinoxalinyl]carbonyl}glycine
137) N-({2-[2-(1,1-dimethylethyl)phenyl]-6-hydroxy-5-quinoxalinyl}carbonyl)glycine
138) N-({6-hydroxy-2-[4-(methylamino)phenyl]-5-quinoxalinyl}carbonyl)glycine
139) N-({6-hydroxy-2-[3-(methylamino)phenyl]-5-quinoxalinyl}carbonyl)glycine
140) N-[(7-ethenyl-6-hydroxy-5-quinoxalinyl)carbonyl]glycine
141) N-{[2-(3,4-difluorophenyl)-7-(3-fluorophenyl)-6-hydroxy-5-quinoxalinyl]carbonyl}glycine
142) N-({2-[3,5-bis(trifluoromethyl)phenyl]-6-hydroxy-5-quinoxalinyl}carbonyl)glycine
143) N-{[3,7-bis(3-fluorophenyl)-6-hydroxy-5-quinoxalinyl]carbonyl}glycine
144) N-{[6-hydroxy-2-(5-pyrimidinyl)-5-quinoxalinyl]carbonyl}glycine
145) N-{[7-bromo-6-hydroxy-2-(2-thienyl)-5-quinoxalinyl]carbonyl}glycine
146) N-{[2,7-bis(3,4-difluorophenyl)-6-hydroxy-5-quinoxalinyl]carbonyl}glycine
147) N-{[7-bromo-6-hydroxy-2-(1,3-thiazol-2-yl)-5-quinoxalinyl]carbonyl}glycine
148) N-{[7-(3-fluorophenyl)-6-hydroxy-2-(1,3-thiazol-2-yl)-5-quinoxalinyl]carbonyl}glycine
149) N-[(7-bromo-6-hydroxy-2-oxo-3-phenyl-1,2-dihydro-5-quinoxalinyl)carbonyl]glycine
150) N-{[7-(3-fluorophenyl)-3-(4-fluorophenyl)-6-hydroxy-5-quinoxalinyl]carbonyl}glycine
151) N-({6-hydroxy-2-[2-(methylamino)phenyl]-5-quinoxalinyl}carbonyl)glycine
152) N-{[2-(3,4-difluorophenyl)-6-hydroxy-7-(1,3-thiazol-2-yl)-5-quinoxalinyl]carbonyl}glycine and
153) N-{[6-hydroxy-2-(2-pyridinyl)-5-quinoxalinyl]carbonyl}glycine.

Processes for preparing the compound of formula (I) are also within the ambit of this invention. To illustrate, process for preparing a compound of formula (I)

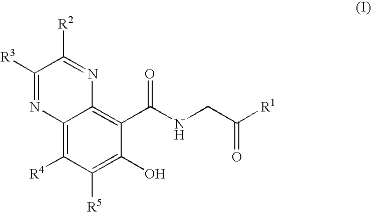

wherein R¹, R², R³, R⁴, and R⁵ are the same as defined above for formula (I), the process comprising treating a compound of formula A:

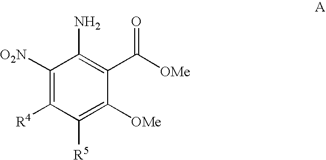

wherein R⁴ and R⁵ are the same as for those groups in formula (I), in a hydrogen atmosphere with an appropriate catalyst, such as palladium on charcoal, in an appropriate solvent, such as ethyl acetate or with an appropriate reducing agent, such as tin(II) chloride dihydrate, in an appropriate solvent, such as ethanol with or without acetonitrile, followed by addition of an appropriately substituted 1,2-dicarbonyl compound or a hydrate thereof, such as phenylglyoxal monohydrate, methyl glyoxal, glyoxal, glyoxylic acid ethyl ester, 2,3-butanedione, 3,4-difluorophenylglyoxal hydrate, 2,4-difluorophenylglyoxal hydrate, t-butylglyoxal, 4-cyclohexylphenylglyoxal hydrate, or 4-fluorophenylglyoxal hydrate, in an appropriate solvent, such as acetonitrile/water or methanol, with heating under either conventional thermal conditions or by microwave irradiation, to form a compound of formula B:

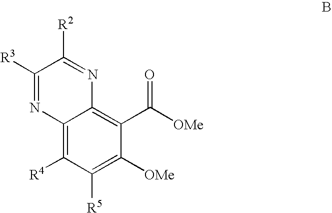

wherein R², R³, R⁴, and R⁵ are the same as for those groups in formula (I), which undergoes ether cleavage/ester hydrolysis with an appropriate reagent, such as boron tribromide, in an appropriate solvent, such as dichloromethane, and is then coupled with an appropriate glycine ester, such as glycine ethyl ester hydrochloride, and an appropriate base, such as triethylamine or diisopropylethylamine, and an appropriate coupling reagent, such as HATU or PyBOP, in an appropriate solvent, such as N,N-dimethylformamide or dichloromethane, followed by ester hydrolysis with an appropriate base, such as sodium hydroxide, in an appropriate solvent, such as ethanol or tetrahydrofuran/methanol, to form a compound of formula (I) where R¹ is —OH.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### DEFINITIONS

MgSO₄—Magnesium sulfate,
Na₂SO₄—Sodium sulfate,
Pd/C—Palladium on charcoal,
PyBOP—Benzotriazol-1-yl-oxytripyrrolidinophosphonium hexafluorophosphate,
HATU—2-(1H-7-azabenzotriazol-1-yl)-1,1,3,3-tetramethyluronium hexafluorophosphate.
HPLC—high performance liquid chromatography.

### CHEMICAL BACKGROUND

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

The compounds described herein may be made from commercially available starting materials or synthesized using known organic, inorganic and/or enzymatic processes.

### ILLUSTRATED METHODS OF PREPARATION

### Schemes

Included in the present invention is a process according to Schemes 1-5 for the synthesis of the compounds:

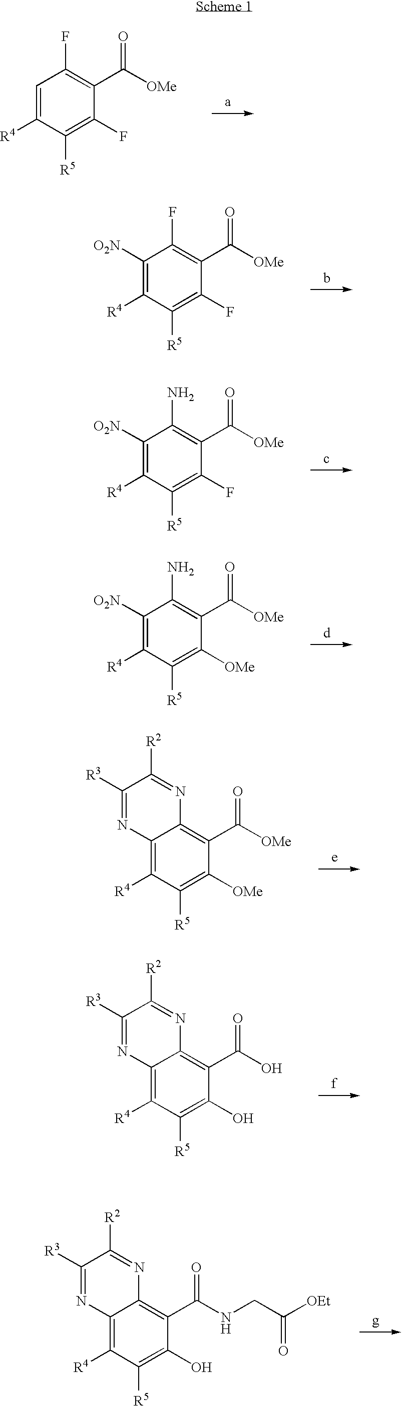

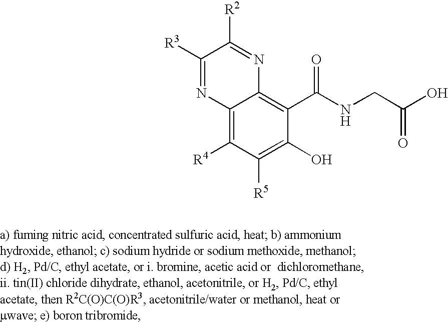

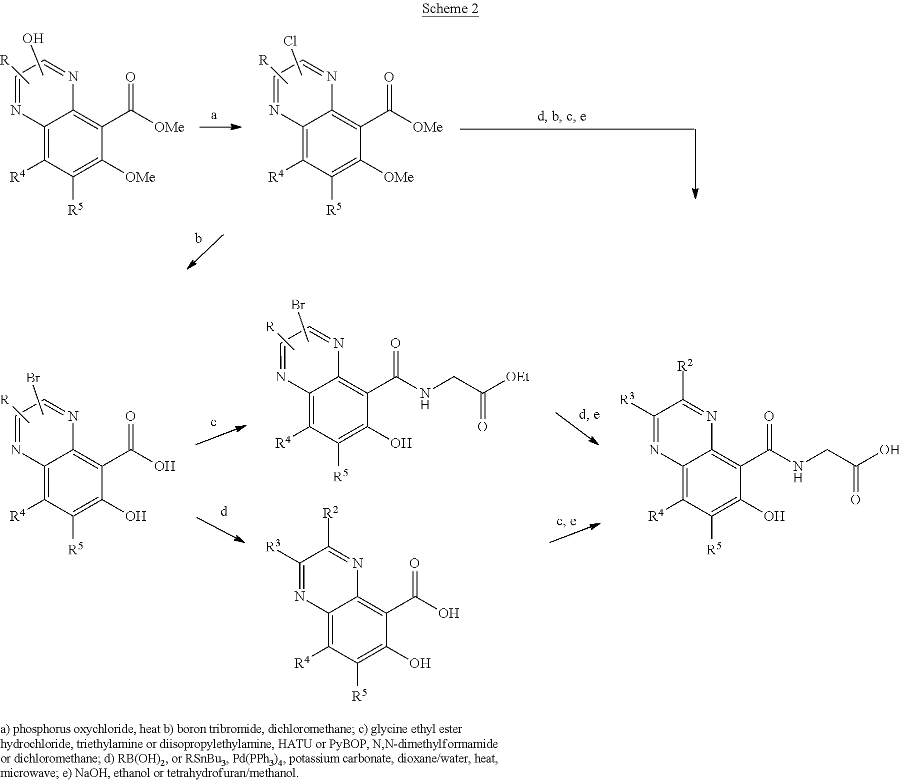

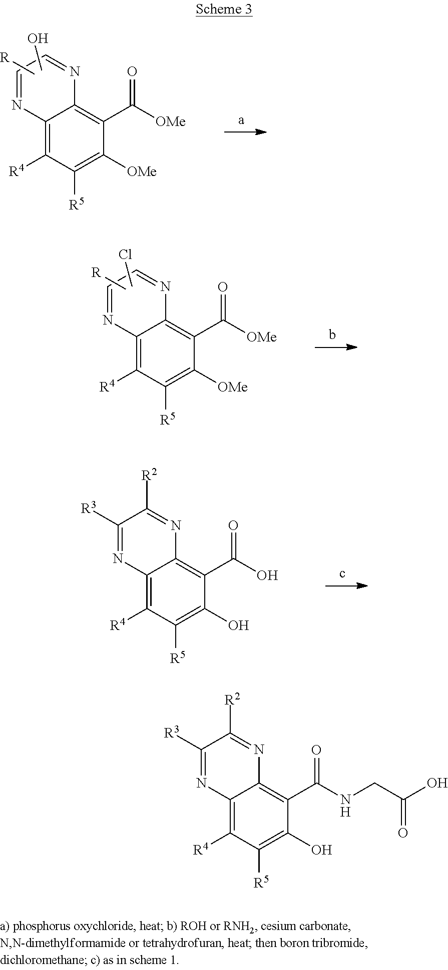

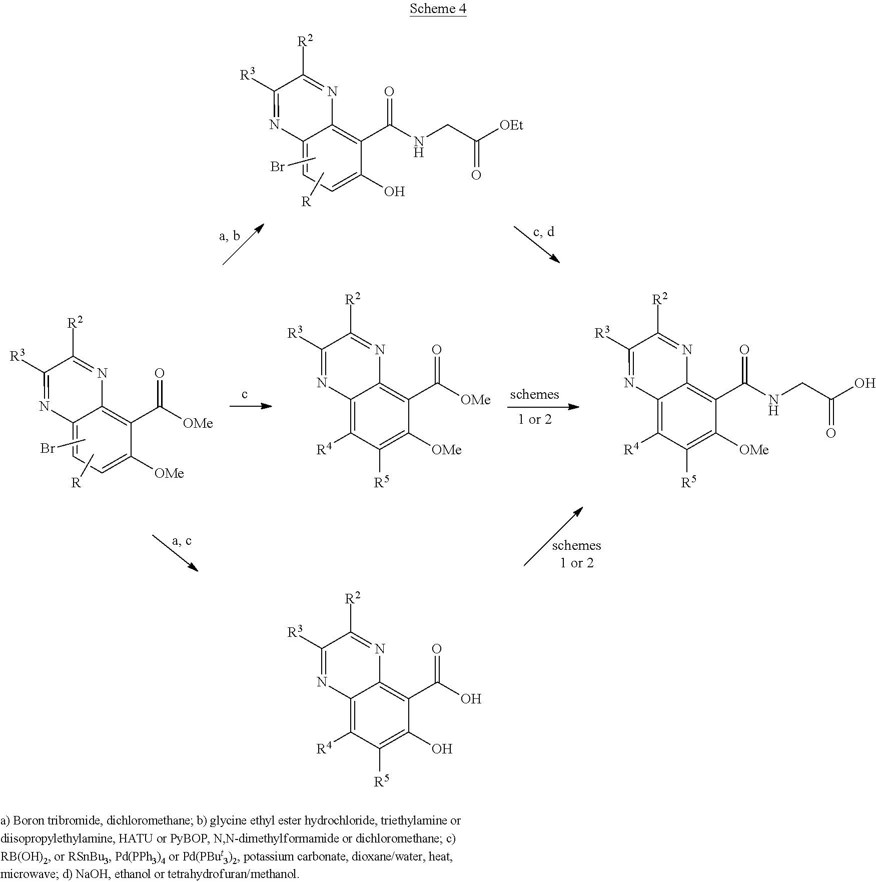

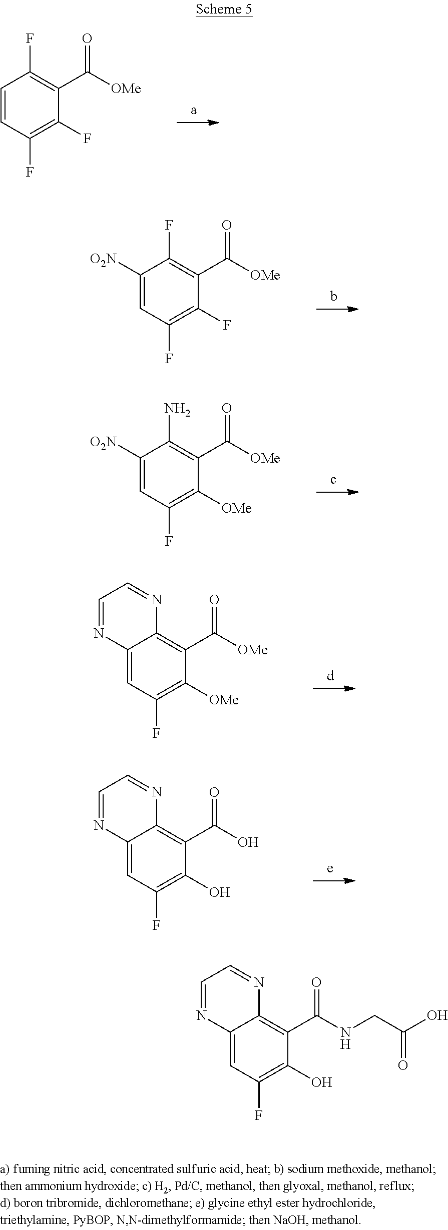

### EXAMPLES

### Example 1

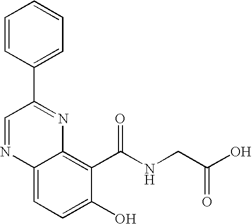

### N-[(6-hydroxy-3-phenyl-5-quinoxalinyl)carbonyl]glycine

### 1a) Methyl 2-amino-6-fluoro-3-nitrobenzoate

To fuming nitric acid (3.87 mL, 86.6 mmol) at 0° C. was slowly added concentrated sulfuric acid (7.27 mL, 136.4 mmol). After stirring for 5 min., methyl 2,6-difluorobenzoate (3.90 mL, 29.0 mmol) was added and the reaction mixture was allowed to warm to ambient temperature. After 30 min, the reaction mixture was poured into ice-water and extracted thrice with dichloromethane. The combined organic portions were washed with saturated aqueous sodium bicarbonate, dried over MgSO₄, filtered, and concentrated in vacuo to afford a colorless oil. MS (ES+) m/e 218 [M+H]⁺. Upon standing, the oil solidified to a white solid, which was dissolved in ethanol (50.0 mL) and treated with ammonium hydroxide (1.0 mL, 29% aqueous solution) at ambient temperature. After 4 h, additional ammonium hydroxide (0.8 mL, 29% aqueous solution) was added and the reaction mixture was stirred overnight. The solution was concentrated and the residual solid was washed with isopropanol, filtered, washed with water, and dried in vacuo to afford the title compound (5.69 g, 92%) as a yellow solid. ¹H NMR (400 MHz, DMSO-d6) δ ppm 8.33 (dd, J=9.5, 5.7 Hz, 1H) 8.11 (br. s., 2H) 6.62 (t, J=9.9 Hz, 1H) 3.89 (s, 3H). MS (ES+) m/e 215 [M+H]⁺.

### 1b) Methyl 2-amino-6-(methyloxy)-3-nitrobenzoate

To methanol (20.0 mL) at 0° C. was added sodium hydride (1.31 g, 32.7 mmol, 60% dispersion in mineral oil) followed by the compound from Example 1a) (5.69 g, 26.6 mmol). The mixture was allowed to warm to ambient temperature over 1 h and quenched with 1N aqueous hydrochloric acid (25.0 mL). The resulting precipitate was filtered, washed with water, and dried in vacuo to afford the title compound (3.20 g, 53%) as a bright yellow solid. ¹H NMR (400 MHz, CHLOROFORM-d) δ ppm 8.32 (d, J=9.6 Hz, 1H), 7.77 (s, 2H), 6.33 (d, J=9.6 Hz, 1H), 3.94 (s, 3H), 3.93 (s, 3H). MS (ES+) m/e 227 [M+H]⁺.

### 1c) Methyl 6-(methyloxy)-3-phenyl-5-quinoxalinecarboxylate

To a solution of the compound from Example 1b) (0.330 g, 1.46 mmol) in ethyl acetate (25.0 mL) was added 10% palladium on charcoal (0.078 g, 0.073 mmol), followed by evacuation of the reaction vessel and purging with nitrogen. The reduction was carried out under 50 psi of hydrogen gas with a Parr Shaker overnight. The reaction mixture was filtered through Celite®, washed through with ethyl acetate, and concentrated in vacuo. A suspension of the resulting yellow oil in water (15.0 mL) and acetonitrile (2.5 mL) was treated with phenylglyoxal monohydrate (0.220 g, 1.46 mmol) and heated to 60° C. for 1 h. Upon cooling, the reaction mixture was diluted with brine and extracted thrice with ethyl acetate. The combined organic layers were dried over MgSO₄, filtered, concentrated in vacuo, and purified via flash column chromatography (20-60% ethyl acetate in hexanes) to afford the title compound (0.280 g, 65%) as a yellow solid. ¹H NMR (400 MHz, CHLOROFORM-d) δ ppm 9.21 (s, 1H) 8.14-8.21 (m, 2H) 8.11 (d, J=9.3 Hz, 1H) 7.42-7.56 (m, 4H) 4.09 (s, 3H) 4.00 (s, 3H). MS (ES+) m/e 295 [M+H]⁺.

### 1d) 6-(Methyloxy)-3-phenyl-5-quinoxalinecarboxylic acid

To a solution of the compound from Example 1c) (0.280 g, 0.951 mmol) in methanol (5.0 mL) was added 6N aqueous sodium hydroxide (1.0 mL). The reaction mixture was heated to 60° C. for 2 h. Upon cooling, a white solid was filtered, the filtrate was acidified with 6N aqueous hydrochloric acid, and then extracted twice with ethyl acetate. The combined organic layers were dried over MgSO₄, filtered, and concentrated in vacuo. The aqueous layer was concentrated in vacuo, treated with methanol, filtered, and concentrated in vacuo. The material afforded from both phases was combined to afford the title compound (0.200 g, 75%) as a white solid. ¹H NMR (400 MHz, CHLOROFORM-d) δ ppm 9.38 (s, 1H), 8.37 (d, J=9.6 Hz, 1H), 8.10-8.19 (m, 2H), 7.75 (d, J=9.6 Hz, 1H), 7.61-7.68 (m, 3H), 4.24 (s, 3H). MS (ES+) m/e 281 [M+H]⁺.

### 1e) 6-Hydroxy-3-phenyl-5-quinoxalinecarboxylic acid

To a solution of the compound from Example 1d) (0.200 g, 0.714 mmol) in dichloromethane (20.0 mL) was added boron tribromide (1M solution in dichloromethane) (2.86 mL, 2.86 mmol). The reaction mixture was stirred overnight at ambient temperature, quenched by water, diluted with brine, and extracted twice with dichloromethane. The combined organic layers were dried over MgSO₄, filtered, and concentrated in vacuo to afford the title compound (0.120 g, 63%) as a yellow solid. ¹H NMR (400 MHz, CHLOROFORM-d) δ ppm 16.3 (s, 1H), 13.4 (s, 1H), 9.32 (s, 1H), 8.25 (d, J=9.3 Hz, 1H), 8.02-8.14 (m, 2H), 7.62-7.69 (m, 3H), 7.57 (d, J=9.3 Hz, 1H). MS (ES+) m/e 267 [M+H]⁺.

### 1 f) Ethyl N-[(6-hydroxy-3-phenyl-5-quinoxalinyl)carbonyl]glycinate

To a solution of the compound from Example 1e) (0.120 g, 0.451 mmol) and glycine ethyl ester hydrochloride (0.252 g, 1.80 mmol) in N,N-dimethylformamide (10.0 mL) were added triethylamine (0.376 mL, 2.71 mmol) and HATU (0.376 g, 0.992 mmol). The reaction mixture was stirred overnight at ambient temperature, quenched by water, diluted with brine, and extracted twice with ethyl acetate. The combined organic layers were dried over MgSO₄, filtered, and concentrated in vacuo. The residue was slurried in diethyl ether, filtered, and dried in vacuo to afford the title compound (0.043 g, 27%) as an off-white solid. ¹H NMR (400 MHz, CHLOROFORM-d) δ ppm 9.19 (s, 1H), 8.25 (d, J=9.3 Hz, 1H), 8.16-8.23 (m, 2H), 7.59-7.65 (m, 2H), 7.49-7.58 (m, 2H), 4.42 (d, J=5.1 Hz, 2H), 4.31 (q, J=7.2 Hz, 2H), 1.33 (t, J=7.2 Hz, 3H). MS (ES+) m/e 352 [M+H]⁺.

### 1g) N-[(6-hydroxy-3-phenyl-5-quinoxalinyl)carbonyl]glycine

To a solution of the compound from Example 1f) (0.043 g, 0.123 mmol) in ethanol (2.0 mL) was added 1N aqueous sodium hydroxide (1.0 mL). After stirring 20 min. at ambient temperature, the ethanol was removed by rotary evaporation and the solution was acidified with 1N aqueous hydrochloric acid. The resulting precipitate was filtered, washed with water, and dried in vacuo to afford the title compound (0.024 g, 60%) as a yellow solid. ¹H NMR (400 MHz, DMSO-d6) δ ppm 15.4 (br. s., 1H), 11.5 (t, J=5.3 Hz, 1H), 9.50 (s, 1H), 8.37 (m, 2H), 8.21 (d, J=9.3 Hz, 1H), 7.59-7.70 (m, 3H), 7.53 (d, J=9.1 Hz, 1H), 4.36 (d, J=5.3 Hz, 2H). MS (ES+) m/e 324 [M+H]⁺.

### Example 2

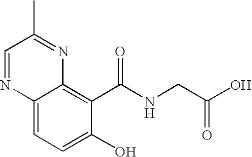

### N-[(6-hydroxy-3-methyl-5-quinoxalinyl)carbonyl]glycine

### 2a) Methyl 3-methyl-6-(methyloxy)-5-quinoxalinecarboxylate

To a solution of the compound from Example 1b) (0.307 g, 1.36 mmol) in ethyl acetate (25.0 mL) was added 10% palladium on charcoal (0.072 g, 0.068 mmol), followed by evacuation of the reaction vessel and purging with nitrogen. The reduction was carried out under 50 psi of hydrogen gas with a Parr Shaker overnight. The reaction mixture was filtered through Celite®, washed through with ethyl acetate, and concentrated in vacuo. A suspension of the resulting yellow oil in water (15.0 mL) and acetonitrile (5.0 mL) was treated with methyl glyoxal (40 wt % solution in water) (0.245 g, 1.36 mmol) and heated to 60° C. for 1 h. Upon cooling, the reaction mixture was diluted with brine and extracted thrice with ethyl acetate. The combined organic layers were dried over MgSO₄, filtered, and concentrated in vacuo to afford the title compound (0.273 g, 87%) as an orange solid. ¹H NMR (400 MHz, CHLOROFORM-d) δ ppm 8.62 (s, 1H), 8.17 (d, J=9.1 Hz, 1H), 7.51 (d, J=9.3 Hz, 1H) 4.08 (s, 3H), 4.04 (s, 3H), 2.76 (s, 3H). MS (ES+) m/e 233 [M+H]⁺.

### 2b) 6-Hydroxy-3-methyl-5-quinoxalinecarboxylic acid

To a solution of the compound from Example 2a) (0.273 g, 1.18 mmol) in dichloromethane (5.0 mL) was added boron tribromide (1M solution in dichloromethane) (3.62 mL, 3.62 mmol). The reaction mixture was stirred overnight at ambient temperature, quenched by water, diluted with brine, and extracted twice with dichloromethane. The combined organic layers were dried over MgSO₄, filtered, and concentrated in vacuo to afford the title compound (0.200 g, 83%) as a yellow solid. ¹H NMR (400 MHz, DMSO-d6) δ ppm 8.94 (s, 1H), 8.25 (d, J=9.3 Hz, 1H), 7.58 (d, J=9.1 Hz, 1H) 2.80 (s, 3H). MS (ES+) m/e 205 [M+H]⁺.

### 2c) N-[(6-hydroxy-3-methyl-5-quinoxalinyl)carbonyl]glycine

To a solution of the compound from Example 2b) (0.200 g, 0.980 mmol) and glycine ethyl ester hydrochloride (0.547 g, 3.92 mmol) in N,N-dimethylformamide (10.0 mL) were added triethylamine (0.819 mL, 5.88 mmol) and HATU (0.821 g, 2.16 mmol). The reaction mixture was stirred overnight at ambient temperature, quenched by water, diluted with brine, and extracted twice with ethyl acetate. The combined organic layers were dried over MgSO₄, filtered, and concentrated in vacuo. The residue was washed with diethyl ether, filtered, and dried in vacuo. A solution of the resulting solid in ethanol (3.0 mL) was treated with 1N aqueous sodium hydroxide (2.0 mL). After stirring 20 min. at ambient temperature, the ethanol was removed by rotary evaporation and the solution was acidified with 1N aqueous hydrochloric acid. The resulting precipitate was filtered, washed with water, and dried in vacuo to afford the title compound (0.031 g, 12%) as a dark red solid. ¹H NMR (400 MHz, DMSO-d6) δ ppm 15.2 (s, 1H) 11.7 (t, J=5.1 Hz, 1H) 8.81 (s, 1H), 8.12 (d, J=9.3 Hz, 1H), 7.45 (d, J=9.1 Hz, 1H) 4.25 (d, J=5.1 Hz, 2H) 2.80 (s, 3H). MS (ES+) m/e 262 [M+H]⁺.

### Example 3

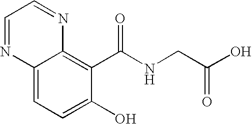

### N-[(6-hydroxy-5-quinoxalinyl)carbonyl]glycine

### 3a) Methyl 6-(methyloxy)-5-quinoxalinecarboxylate

To a solution of the compound from Example 1b) (0.315 g, 1.40 mmol) in ethyl acetate (20.0 mL) was added 10% palladium on charcoal (0.074 g, 0.070 mmol), followed by evacuation of the reaction vessel and purging with nitrogen. The reduction was carried out under 50 psi of hydrogen gas with a Parr Shaker overnight. The reaction mixture was filtered through Celite®, washed through with ethyl acetate, and concentrated in vacuo. A suspension of the resulting yellow oil in water (10.0 mL) and acetonitrile (2.0 mL) was treated with glyoxal (40 wt % solution in water) (0.200 g, 1.40 mmol) and heated to 60° C. for 1 h. Upon cooling, the reaction mixture was diluted with brine and extracted thrice with ethyl acetate. The combined organic layers were dried over MgSO₄, filtered, concentrated in vacuo, and purified via flash column chromatography (40-60% ethyl acetate in hexanes) to afford the title compound (0.097 g, 32%) as a yellow oil. ¹H NMR (400 MHz, CHLOROFORM-d) δ ppm 8.84 (d, J=1.8 Hz, 1H), 8.75 (d, J=1.8 Hz, 1H), 8.18 (d, J=9.3 Hz, 1H) 7.59 (d, J=9.3 Hz, 1H), 4.08 (s, 3H), 4.05 (s, 3H). MS (ES+) m/e 219 [M+H]⁺.

### 3b) 6-Hydroxy-5-quinoxalinecarboxylic acid

To a solution of the compound from Example 3a) (0.097 g, 0.445 mmol) in dichloromethane (3.0 mL) was added boron tribromide (1M solution in dichloromethane) (1.78 mL, 1.78 mmol). The reaction mixture was stirred overnight at ambient temperature, quenched by water, diluted with brine, and extracted twice with dichloromethane. The combined organic layers were dried over MgSO₄, filtered, and concentrated in vacuo to afford the title compound (0.067 g, 80%) as an orange solid. ¹H NMR (400 MHz, DMSO-d6) δ ppm 9.01 (d, J=2.3 Hz, 1H), 8.98 (d, J=2.0 Hz, 1H), 8.27 (d, J=9.3 Hz, 1H), 7.66 (d, J=9.6 Hz, 1H). MS (ES+) m/e 191 [M+H]⁺.

### 3c) N-[(6-hydroxy-5-quinoxalinyl)carbonyl]glycine

To a solution of the compound from Example 3b) (0.067 g, 0.352 mmol) and glycine ethyl ester hydrochloride (0.196 g, 1.41 mmol) in N,N-dimethylformamide (2.0 mL) were added triethylamine (0.294 mL, 2.11 mmol) and HATU (0.290 g, 0.774 mmol). The reaction mixture was stirred overnight at ambient temperature, quenched by water, diluted with brine, and extracted twice with ethyl acetate. The combined organic layers were dried over MgSO₄, filtered, and concentrated in vacuo. The residue was washed with diethyl ether, filtered, and dried in vacuo. A solution of the resulting solid in ethanol (2.0 mL) was treated with 1N aqueous sodium hydroxide (1.0 mL). After stirring 20 min. at ambient temperature, the ethanol was removed by rotary evaporation and the solution was acidified with 1N aqueous hydrochloric acid (2.0 mL). The resulting precipitate was filtered, washed with water, and dried in vacuo. The filtrate was further extracted twice with ethyl acetate, dried over MgSO₄, filtered, concentrated in vacuo, washed with water, dried in vacuo, and combined with the above material to afford the title compound (0.025 g, 29%) as a beige solid. ¹H NMR (400 MHz, DMSO-d6) δ ppm 11.4 (t, J=5.6 Hz, 1H) 8.95 (d, J=2.0 Hz, 1H), 8.91 (d, J=2.0 Hz, 1H), 8.19 (d, J=9.3 Hz, 1H), 7.56 (d, J=9.3 Hz, 1H), 4.23 (d, J=5.6 Hz, 2H). MS (ES+) m/e 248 [M+H]⁺.

### Example 4

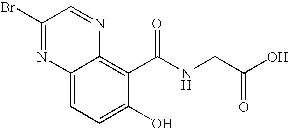

### N-[(2-bromo-6-hydroxy-5-quinoxalinyl)carbonyl]glycine

### 4a) Methyl 2,3-diamino-6-(methyloxy)benzoate

To a solution of the compound from Example 1b) (0.530 g, 2.34 mmol) in ethyl acetate (50.0 mL) was added 10% palladium on charcoal (0.125 g, 0.117 mmol), followed by evacuation of the reaction vessel and purging with nitrogen. The reduction was carried out under 50 psi of hydrogen gas with a Parr Shaker overnight. The reaction mixture was filtered through Celite®, washed through with ethyl acetate, and concentrated in vacuo to afford the desired compound as a dark, viscous oil (0.460 g, 100%). The material was used without further purification. ¹H NMR (400 MHz, CHLOROFORM-d) ppm 6.76 (d, J=8.3 Hz, 1H) 6.19 (d, J=8.6 Hz, 1H) 3.91 (s, 3H) 3.78 (s, 3H). MS (ES+) m/e 197 [M+H]⁺.

### 4b) Methyl 6-(methyloxy)-2-oxo-1,2-dihydro-5-quinoxalinecarboxylate

To a suspension of the compound from Example 4a) (0.460 g, 2.34 mmol) in acetonitrile (15.0 mL) was added glyoxylic acid ethyl ester (50% solution in toluene) (0.465 mL, 2.35 mmol), followed by stirring at ambient temperature for 15 min. The resulting precipitate was filtered, washed with acetonitrile and diethyl ether, and dried in vacuo to afford the title compound (0.280 g, 51%) as a light cream solid. ¹H NMR (400 MHz, DMSO-d6) ppm 12.4 (br. s., 1H), 8.19 (s, 1H), 7.46 (d, J=9.1 Hz, 1H), 7.35 (d, J=9.1 Hz, 1H), 3.84 (s, 3H), 3.83 (s, 3H). MS (ES+) m/e 235 [M+H]⁺.

### 4c) Methyl 2-chloro-6-(methyloxy)-5-quinoxalinecarboxylate

To a solution of the compound from Example 4b) (0.280 g, 1.20 mmol) was added phosphorus oxychloride (2.41 mL, 25.53 mmol). The reaction mixture was heated to reflux overnight, quenched by pouring into ice-water, and extracted twice with ethyl acetate. The combined organic layers were dried over Na₂SO₄, filtered, and concentrated in vacuo to afford the title compound (0.262 g, 86%) as a dark yellow solid. ¹H NMR (400 MHz, CHLOROFORM-d) δ ppm 8.76 (s, 1H), 8.09 (d, J=9.3 Hz, 1H), 7.60 (d, J=9.3 Hz, 1H), 4.06 (s, 3H), 4.05 (s, 3H). MS (ES+) m/e 253 [M+H]⁺.

### 4d) 2-Bromo-6-hydroxy-5-quinoxalinecarboxylic acid

To a solution of the compound from Example 4c) (0.262 g, 1.04 mmol) in dichloromethane (5.0 mL) was added boron tribromide (1M solution in dichloromethane) (4.16 mL, 4.16 mmol). The reaction mixture was stirred overnight at ambient temperature, quenched by water, diluted with brine, and extracted twice with dichloromethane. The combined organic layers were dried over MgSO₄, filtered, and concentrated in vacuo to afford the title compound (0.215 g, 77%) as a brown solid. ¹H NMR (400 MHz, DMSO-d6) δ ppm 11.8 (br. s., 1H), 9.04 (s, 1H), 8.09 (d, J=9.3 Hz, 1H), 7.62 (d, J=9.3 Hz, 1H). MS (ES+) m/e 269/271 [M+H]⁺.

### 4e) N-[(2-bromo-6-hydroxy-5-quinoxalinyl)carbonyl]glycine

To a solution of the compound from Example 4d) (0.215 g, 0.799 mmol) and glycine ethyl ester hydrochloride (0.448 g, 3.20 mmol) in N,N-dimethylformamide (10.0 mL) were added triethylamine (0.668 mL, 4.79 mmol) and HATU (0.669 g, 1.76 mmol). The reaction mixture was stirred overnight at ambient temperature, quenched by water, and filtered. A solution of the resulting solid in ethanol (5.0 mL) was treated with 6N aqueous sodium hydroxide (2.0 mL). After stirring 20 min. at ambient temperature, the ethanol was removed by rotary evaporation and the solution was acidified with 1N aqueous hydrochloric acid (3.0 mL). The mixture was concentrated in vacuo and water was added. The resulting precipitate was filtered, washed with water, and dried in vacuo to afford the title compound (0.008 g, 3%) as a purple solid. ¹H NMR (400 MHz, DMSO-d6) δ ppm 15.2 (br. s., 1H), 10.9 (t, J=5.6 Hz, 1H), 9.03 (s, 1H), 8.15 (d, J=9.3 Hz, 1H), 7.60 (d, J=9.3 Hz, 1H), 4.22 (d, J=5.6 Hz, 2H). MS (ES+) m/e 326/328 [M+H]⁺.

### Example 5

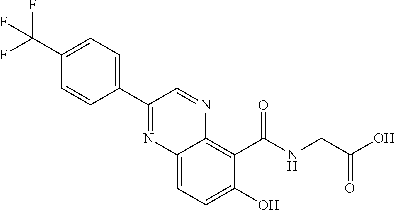

### N-(6-hydroxy-2-[4-(trifluoromethyl)phenyl]-5-quinoxalinyl 1 carbonyl)glycine

### 5a) Ethyl N-[(2-bromo-6-hydroxy-5-quinoxalinyl)carbonyl]glycinate

To a solution of 2-bromo-6-hydroxy-5-quinoxalinecarboxylic acid (prepared as in Example 4d) (0.181 g, 0.673 mmol) and glycine ethyl ester hydrochloride (0.380 g, 2.69 mmol) in N,N-dimethylformamide (10.0 mL) were added triethylamine (0.560 mL, 4.04 mmol) and HATU (0.560 g, 1.48 mmol). The reaction mixture was stirred overnight, concentrated in vacuo, and purified via flash column chromatography (60% ethyl acetate in hexanes) to afford the title compound (0.070 g, 29%) as a white solid. ¹H NMR (400 MHz, DMSO-d6) δ ppm 15.1 (s, 1H), 11.0 (t, J=5.6 Hz, 1H), 9.03 (s, 1H), 8.16 (d, J=9.3 Hz, 1H), 7.61 (d, J=9.3 Hz, 1H), 4.30 (d, J=5.6 Hz, 2H), 4.17 (q, J=7.1 Hz, 2H), 1.23 (t, J=7.2 Hz, 3H). MS (ES+) m/e 354/356 [M+H]⁺.

### 5b) N-({6-hydroxy-2-[4-(trifluoromethyl)phenyl]-5-quinoxalinyl}carbonyl)glycine

A solution of the compound from Example 5a) (0.051 g, 0.144 mmol), 4-trifluoromethylbenzeneboronic acid (0.027 mL, 0.143 mmol), potassium carbonate (0.059 g, 0.429 mmol), and tetrakis(triphenylphosphine)palladium(0) (0.004 g, 0.004 mmol) in 1,4-dioxane/water (3/1 solution) (1.0 mL) was heated to 100° C. for 20 min. in a Biotage Initiator® microwave synthesizer. Upon cooling, the reaction mixture was diluted with water and extracted twice with ethyl acetate. The combined organic layers were dried over MgSO₄, filtered, and concentrated in vacuo. A solution of the residue in ethanol was treated with 1N aqueous sodium hydroxide (1.0 mL). Following stirring at ambient temperature for 20 min., the reaction mixture was acidified with 1N aqueous hydrochloric acid (2.0 mL), concentrated in vacuo, washed with water, filtered, and dried in vacuo to afford the title compound (0.048 g, 86%) as a yellow solid. ¹H NMR (400 MHz, DMSO-d6) δ ppm 15.4 (s, 1H), 12.9 (br. s., 1H), 11.4 (t, J=5.6 Hz, 1H), 9.60 (s, 1H), 8.52 (d, J=8.1 Hz, 2H), 8.26 (d, J=9.1 Hz, 1H), 7.96 (d, J=8.3 Hz, 2H), 7.60 (d, J=9.3 Hz, 1H), 4.27 (d, J=5.6 Hz, 2H). MS (ES+) m/e 392 [M+H]⁺.

### Example 6

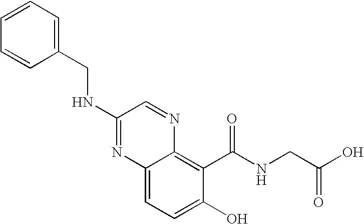

### N-({6-hydroxy-2-[(phenylmethyl)amino]-5-quinoxalinyl}carbonyl)glycine

### 6a) Methyl 6-(methyloxy)-2-[(phenylmethyl)amino]-5-quinoxalinecarboxylate

A solution of methyl 2-chloro-6-(methyloxy)-5-quinoxalinecarboxylate (prepared as in Example 4c) (0.115 g, 0.455 mmol) and benzylamine (0.186 mL, 1.70 mmol) in tetrahydrofuran (3.0 mL) was heated to 180° C. for 45 min. in a Biotage Initiator® microwave synthesizer. Upon cooling the reaction mixture was treated with saturated aqueous sodium bicarbonate and extracted thrice with ethyl acetate. The combined organic portions were dried over MgSO₄, filtered, concentrated in vacuo, and purified via flash column chromatography (60-80% ethyl acetate in hexanes) to afford the title compound (0.111 g, 76%) as an amber oil. ¹H NMR (400 MHz, CHLOROFORM-d) δ ppm 8.22 (s, 1H), 7.75 (d, J=9.3 Hz, 1H), 7.30-7.40 (m, 5H), 7.24-7.31 (m, 1H), 5.40 (br. s., 1H), 4.69 (d, J=5.6 Hz, 2H), 4.02 (s, 3H), 3.94 (s, 3H). MS (ES+) m/e 324 [M+H]⁺.

### 6b) 6-Hydroxy-2-[(phenylmethyl)amino]-5-quinoxalinecarboxylic acid

To a solution of the compound from Example 6a) (0.111 g, 0.343 mmol) in dichloromethane (2.0 mL) was added boron tribromide (1M solution in dichloromethane) (1.37 mL, 1.37 mmol). The reaction mixture was stirred overnight at ambient temperature, quenched by water, and extracted twice with dichloromethane. The combined organic layers were dried over MgSO₄, filtered, and concentrated in vacuo. The residue was washed with diethyl ether, filtered, and dried in vacuo to afford the title compound (0.067 g, 66%) as a brown solid. ¹H NMR (400 MHz, DMSO-d6) δ ppm 12.2 (br. s, 1H), 8.45 (s, 1H), 8.27 (t, J=5.7 Hz, 1H), 7.80 (d, J=9.3 Hz, 1H), 7.40-7.45 (m, 5H), 7.23-7.30 (m, 1H), 4.62 (d, J=5.3 Hz, 2H). MS (ES+) m/e 296 [M+H]⁺.

### 6c) N-({6-hydroxy-2-[(phenylmethyl)amino]-5-quinoxalinyl}carbonyl)glycine

To a solution of the compound from Example 6b) (0.067 g, 0.227 mmol) and glycine ethyl ester hydrochloride (0.126 g, 0.908 mmol) in N,N-dimethylformamide (5.0 mL) were added triethylamine (0.189 mL, 1.36 mmol) and HATU (0.189 g, 0.499 mmol). The reaction mixture was stirred overnight at ambient temperature, quenched by water, and concentrated in vacuo. The residue was washed with dichloromethane and filtered. The filtrate was purified via flash column chromatography (60-80% ethyl acetate in hexanes) to afford an orange solid which was dissolved in ethanol (1.0 mL) and treated with 1N aqueous sodium hydroxide (2.0 mL) at ambient temperature for 20 min. The ethanol was removed by rotary evaporation and the solution was acidified with 1N aqueous hydrochloric acid (3.0 mL). The mixture was concentrated in vacuo and water was added. The resulting precipitate was filtered, washed with water, and dried in vacuo to afford the title compound (0.052 g, 65%) as a bright yellow solid. ¹H NMR (400 MHz, DMSO-d6) δ ppm 14.3 (br. s, 1H), 11.3 (t, J=5.6 Hz, 1H), 8.43 (s, 1H), 8.07 (br. s, 1H), 7.71 (d, J=9.1 Hz, 1H), 7.39-7.45 (m, 2H), 7.31-7.39 (m, 2H), 7.21-7.30 (m, 2H), 4.61 (s, 2H), 4.18 (d, J=5.6 Hz, 2H). MS (ES+) m/e 353 [M+H]⁺.

### Example 7

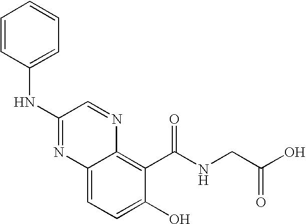

### N-{[6-hydroxy-2-(phenylamino)-5-quinoxalinyl]carbonyl}glycine

### 7a) Methyl 6-(methyloxy)-2-(phenylamino)-5-quinoxalinecarboxylate

A solution of methyl 2-chloro-6-(methyloxy)-5-quinoxalinecarboxylate (prepared as in Example 4c) (0.143 g, 0.566 mmol) and aniline (0.207 mL, 2.26 mmol) in tetrahydrofuran (3.0 mL) was heated to 180° C. for 45 min. in a Biotage Initiator® microwave synthesizer. Upon cooling the reaction mixture was treated with saturated aqueous sodium bicarbonate and extracted thrice with ethyl acetate. The combined organic portions were dried over MgSO₄, filtered, concentrated in vacuo, and purified via flash column chromatography (60-80% ethyl acetate in hexanes) to afford the title compound (0.094 g, 54%) as an orange solid. ¹H NMR (400 MHz, METHANOL-d4) δ ppm 8.42 (s, 1H), 7.92 (t, J=1.5 Hz, 1H), 7.88-7.91 (m, 1H), 7.84 (d, J=9.1 Hz, 1H), 7.55 (d, J=9.1 Hz, 1H), 7.29-7.43 (m, 2H), 6.97-7.09 (m, 1H), 3.96 (s, 3H), 3.95 (s, 3H). MS (ES+) m/e 310 [M+H]⁺.

### 7b) 6-Hydroxy-2-(phenylamino)-5-quinoxalinecarboxylic acid

To a solution of the compound from Example 7a) (0.094 g, 0.304 mmol) in dichloromethane (5.0 mL) was added boron tribromide (1M solution in dichloromethane) (1.21 mL, 1.21 mmol). The reaction mixture was stirred overnight at ambient temperature, quenched by water, and extracted twice with dichloromethane. The combined organic layers were dried over MgSO₄, filtered, and concentrated in vacuo. The residue was washed with diethyl ether, filtered, and dried in vacuo to afford the title compound (0.041 g, 48%) as a red solid. ¹H NMR (400 MHz, DMSO-d6) δ ppm 15.4 (br. s, 1H), 12.7 (s, 1H), 8.44 (s, 1H), 7.96 (d, J=9.3 Hz, 1H), 7.63 (d, J=1.0 Hz, 1H), 7.61 (d, J=0.8 Hz, 1H), 7.49 (d, J=9.3 Hz, 1H), 7.40-7.48 (m, 2H). MS (ES+) m/e 282 [M+H]⁺.

### 7c) N-{[6-hydroxy-2-(phenylamino)-5-quinoxalinyl]carbonyl}glycine

To a solution of the compound from Example 7b) (0.041 g, 0.146 mmol) and glycine ethyl ester hydrochloride (0.081 g, 0.584 mmol) in N,N-dimethylformamide (2.0 mL) were added triethylamine (0.122 mL, 0.876 mmol) and HATU (0.122 g, 0.321 mmol). The reaction mixture was stirred overnight at ambient temperature, quenched by water, diluted with brine, and extracted thrice with ethyl acetate. The combined organic portions were dried over MgSO₄, filtered, and concentrated in vacuo. A solution of the resulting residue in ethanol (2.0 mL) was treated with 1N aqueous sodium hydroxide (1.0 mL) at ambient temperature for 15 min. The mixture was concentrated in vacuo, dissolved in water, and acidified with 1N aqueous hydrochloric acid (2.0 mL). The resulting precipitate was filtered, washed with water, and dried in vacuo to afford the title compound (0.015 g, 30%) as a dark orange solid. ¹H NMR (400 MHz, DMSO-d6) δ ppm 14.5 (s, 1H), 12.9 (br. s, 1H), 11.3 (t, J=5.6 Hz, 1H), 9.92 (s, 1H), 8.60 (s, 1H), 7.93 (d, J=7.6 Hz, 2 H), 7.88 (d, J=9.1 Hz, 1H), 7.36-7.40 (m, 2H), 7.35 (d, J=2.5 Hz, 1H), 7.02 (t, J=7.3 Hz, 1H), 4.21 (d, J=5.6 Hz, 2H). MS (ES+) m/e 339 [M+H]⁺.

### Example 8

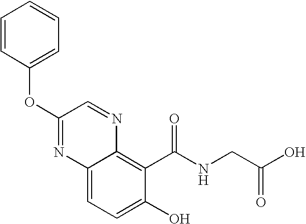

### N-{[6-hydroxy-2-(phenyloxy)-5-quinoxalinyl]carbonyl}glycine

### 8a) Methyl 6-(methyloxy)-2-(phenyloxy)-5-quinoxalinecarboxylate

A solution of methyl 2-chloro-6-(methyloxy)-5-quinoxalinecarboxylate (prepared as in Example 4c) (0.100 g, 0.396 mmol), phenol (0.038 g, 0.416 mmol), and cesium carbonate (0.463 g, 1.42 mmol) in N,N-dimethylformamide (3.0 mL) was heated to 150° C. for 30 min. in a Biotage Initiator® microwave synthesizer. Upon cooling the reaction mixture was treated with saturated aqueous sodium bicarbonate and extracted thrice with ethyl acetate. The combined organic portions were dried over MgSO₄, filtered, concentrated in vacuo, and purified via flash column chromatography (80-90% ethyl acetate in hexanes) to afford the title compound (0.101 g, 82%) as a yellow oil. ¹H NMR (400 MHz, CHLOROFORM-d) δ ppm 8.70 (s, 1H), 7.82 (d, J=9.1 Hz, 1H), 7.38-7.56 (m, 3H), 7.28-7.32 (m, 1H), 7.22-7.27 (m, 2H), 4.07 (s, 3H), 3.99 (s, 3H). MS (ES+) m/e 311 [M+H]⁺.

### 8b) 6-Hydroxy-2-(phenyloxy)-5-quinoxalinecarboxylic acid

To a solution of the compound from Example 8a) (0.101 g, 0.326 mmol) in dichloromethane (15.0 mL) was added boron tribromide (1M solution in dichloromethane) (1.30 mL, 1.30 mmol). The reaction mixture was stirred overnight at ambient temperature, quenched by water, and extracted thrice with dichloromethane. The combined organic layers were dried over MgSO₄, filtered, and concentrated in vacuo to afford the title compound (0.082 g, 90%). ¹H NMR (400 MHz, CHLOROFORM-d) δ ppm 15.4 (s, 1H), 12.9 (s, 1H), 8.63 (s, 1H), 7.90 (d, J=9.6 Hz, 1H), 7.44-7.58 (m, 3H), 7.30-7.36 (m, 1H), 7.28-7.29 (m, 1H), 7.26 (d, J=1.0 Hz, 1H). MS (ES+) m/e 283 [M+H]⁺.

### 8c) N-{[6-hydroxy-2-(phenyloxy)-5-quinoxalinyl]carbonyl}glycine

To a solution of the compound from Example 8b) (0.082 g, 0.292 mmol) and glycine ethyl ester hydrochloride (0.163 g, 1.17 mmol) in N,N-dimethylformamide (3.0 mL) were added triethylamine (0.244 mL, 1.75 mmol) and HATU (0.244 g, 0.642 mmol). The reaction mixture was stirred overnight at ambient temperature, quenched by water, diluted with brine, and extracted twice with ethyl acetate. The combined organic portions were dried over MgSO₄, filtered, and concentrated in vacuo. The residue was washed with dichloromethane and filtered. The filtrate was concentrated in vacuo, dissolved in ethanol (1.0 mL), and treated with 1N aqueous sodium hydroxide (1.0 mL) at ambient temperature for 15 min. The solution was concentrated in vacuo, dissolved in water, and acidified with 1N aqueous hydrochloric acid (2.0 mL). The resulting precipitate was filtered, washed with methanol, and dried in vacuo to afford the title compound (0.027 g, 27%) as a brown solid. ¹H NMR (400 MHz, DMSO-d6) δ ppm 14.8 (s, 1H), 12.9 (s, 1H), 11.2 (t, J=5.6 Hz, 1H), 8.87 (s, 1H), 7.84 (d, J=9.3 Hz, 1H), 7.47-7.54 (m, 2H), 7.44 (d, J=9.3 Hz, 1H), 7.27-7.37 (m, 3H), 4.24 (d, J=5.6 Hz, 2H). MS (ES+) m/e 340 [M+H]⁺.

### Example 9

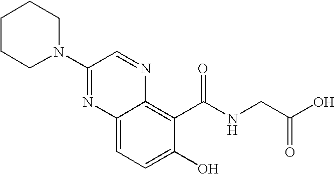

### N-{[6-hydroxy-2-(1-piperidinyl)-5-quinoxalinyl]carbonyl}glycine

### 9a) Methyl 6-(methyloxy)-2-(1-piperidinyl)-5-quinoxalinecarboxylate

A solution of methyl 2-chloro-6-(methyloxy)-5-quinoxalinecarboxylate (prepared as in Example 4c) (0.088 g, 0.349 mmol) and piperidine (0.138 mL, 1.40 mmol) in tetrahydrofuran (2.0 mL) was heated to 180° C. for 45 min. in a Biotage Initiator® microwave synthesizer. Upon cooling, the reaction mixture was treated with saturated aqueous sodium bicarbonate and extracted thrice with ethyl acetate. The combined organic portions were dried over MgSO₄, filtered, and concentrated in vacuo to afford the title compound (0.083 g, 79%) as an orange oil. ¹H NMR (400 MHz, CHLOROFORM-d) δ ppm 8.60 (s, 1H), 7.71 (d, J=9.1 Hz, 1H), 7.34 (d, J=9.3 Hz, 1H), 4.04 (s, 3H), 3.95 (s, 3H), 3.64-3.77 (m, 4H), 1.58-1.78 (m, 6H). MS (ES+) m/e 302 [M+H]⁺.

### 9b) 6-Hydroxy-2-(1-piperidinyl)-5-quinoxalinecarboxylic acid

To a solution of the compound from Example 9a) (0.083 g, 0.275 mmol) in dichloromethane (3.0 mL) was added boron tribromide (1M solution in dichloromethane) (1.10 mL, 1.10 mmol). The reaction mixture was stirred overnight at ambient temperature, quenched by water, and extracted twice with dichloromethane. The combined organic layers were dried over MgSO₄, filtered, and concentrated in vacuo to afford the title compound (0.058 g, 77%) as a red solid. ¹H NMR (400 MHz, CHLOROFORM-d) δ ppm 8.44 (s, 1H), 7.88 (d, J=7.6 Hz, 1H), 7.40 (d, J=9.3 Hz, 1H), 3.74-3.77 (m, 4H), 1.69-1.82 (m, 6H). MS (ES+) m/e 273 [M+H]⁺.

### 9c) N-{[6-hydroxy-2-(1-piperidinyl)-5-quinoxalinyl]carbonyl}glycine

To a solution of the compound from Example 9b) (0.058 g, 0.213 mmol) and glycine ethyl ester hydrochloride (0.119 g, 0.852 mmol) in N,N-dimethylformamide (2.0 mL) were added triethylamine (0.178 mL, 1.28 mmol) and HATU (0.178 g, 0.469 mmol). The reaction mixture was stirred overnight at ambient temperature, quenched by water, diluted with brine, and extracted thrice with ethyl acetate. The combined organic portions were dried over MgSO₄, filtered, concentrated in vacuo, and purified via flash column chromatography (0-10% methanol in dichloromethane). The resulting orange solid was dissolved in ethanol (1.0 mL) and treated with 1N aqueous sodium hydroxide (1.0 mL) at ambient temperature for 15 min. The solution was concentrated in vacuo, dissolved in water, acidified with 1N aqueous hydrochloric acid (2.0 mL), and extracted twice with ethyl acetate. The combined organic portions were dried over MgSO₄, filtered, concentrated in vacuo, washed with diethyl ether, and dried in vacuo to afford the title compound (0.007 g, 10%) as an orange solid. ¹H NMR (400 MHz, DMSO-d6) δ ppm 14.4 (s, 1H), 11.3 (t, J=5.6 Hz, 1H), 8.79 (s, 1H), 7.75 (d, J=9.3 Hz, 1H), 7.31 (d, J=9.3 Hz, 1H), 4.21 (d, J=5.6 Hz, 2H), 3.73-3.75 (m, 5H), 1.50-1.74 (m, 5H). MS (ES+) m/e 331 [M+H]⁺.

### Example 10

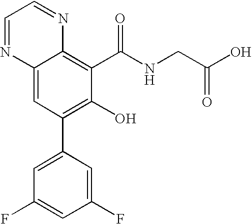

### N-{[7-(3,5-difluorophenyl)-6-hydroxy-5-quinoxalinyl]carbonyl}glycine

### 10a) Methyl 2-amino-5-bromo-6-(methyloxy)-3-nitrobenzoate

To a solution of the compound from Example 1b) (1.07 g, 4.73 mmol) in acetic acid (20.0 mL) was added bromine (0.316 mL, 6.15 mmol). After stirring 2 h at ambient temperature, the reaction mixture was cooled to 0° C., filtered, washed with water, and dried in vacuo to afford the title compound (1.01 g, 70%) as a bright yellow solid. ¹H NMR (400 MHz, DMSO-d6) δ ppm 8.35 (s, 1H), 7.52 (br. s., 2H), 3.90 (s, 3H), 3.83 (s, 3H). MS (ES+) m/e 305/307 [M+H]⁺.

### 10b) Methyl 7-bromo-6-(methyloxy)-5-quinoxalinecarboxylate

To a solution of the compound from Example 10a) (1.01 g, 3.31 mmol) in ethanol (54.0 mL) and acetonitrile (54.0 mL) was added tin(II) chloride dihydrate (8.01 g, 35.7 mmol). After stirring at reflux for 3 h, the reaction mixture was allowed to cool to ambient temperature, poured into water, basified with 6N aqueous sodium hydroxide, and extracted thrice with ethyl acetate. The combined organic layers were dried over MgSO₄, filtered, and concentrated in vacuo. The resulting orange oil was diluted in a mixture of acetonitrile (5.0 mL) and water (20.0 mL). The solution was treated with glyoxal (40 wt % solution in water) (0.370 g, 2.55 mmol) and heated to 60° C. for 1 h. Upon cooling, the reaction mixture was diluted with brine and extracted twice with ethyl acetate. The combined organic layers were dried over MgSO₄, filtered, concentrated in vacuo, and purified via flash column chromatography (0-10% methanol in dichloromethane) to afford the title compound (0.334 g, 48%) as a dark oil. ¹H NMR (400 MHz, CHLOROFORM-d) δ ppm 8.87 (d, J=1.8 Hz, 1H), 8.82 (d, J=1.8 Hz, 1H), 8.46 (s, 1H), 4.11 (s, 3H), 4.09 (s, 3H). MS (ES+) m/e 297/299 [M+H]⁺.

### 10c) 7-Bromo-6-hydroxy-5-quinoxalinecarboxylic acid

To a solution of the compound from Example 10b) (0.334 g, 1.12 mmol) in dichloromethane (20.0 mL) was added boron tribromide (1M solution in dichloromethane) (4.48 mL, 4.48 mmol). The reaction mixture was stirred overnight at ambient temperature, quenched by water, and extracted twice with ethyl acetate. The combined organic layers were dried over MgSO₄, filtered, and concentrated in vacuo to afford the title compound (0.271 g, 90%) as a dark yellow solid. ¹H NMR (400 MHz, DMSO-d6) δ ppm 15.4 (br. s., 1H), 9.00 (d, J=2.8 Hz, 1H), 8.92 (d, J=2.8 Hz, 1H), 8.72 (s, 1H). MS (ES+) m/e 269/271 [M+H]⁺.

### 10d) 7-(3,5-Difluorophenyl)-6-hydroxy-5-quinoxalinecarboxylic acid

A solution of the compound from Example 10c) (0.074 g, 0.275 mmol), 3,5-difluorobenzeneboronic acid (0.043 g, 0.275 mmol), potassium carbonate (0.114 g, 0.825 mmol), and tetrakis(triphenylphosphine)palladium(0) (0.009 g, 0.008 mmol) in 1,4-dioxane/water (3/1 solution) (2.0 mL) was heated to 100° C. for 20 min. in a Biotage Initiator® microwave synthesizer. Upon cooling, the reaction mixture was treated with water, acidified with 1N aqueous hydrochloric acid (˜2.0 mL), and extracted twice with ethyl acetate. The combined organic layers were dried over MgSO₄, filtered, concentrated in vacuo, and purified via flash column chromatography (0-10% methanol in dichloromethane) to afford the title compound (0.079 g, 95%) as a dark oil. ¹H NMR (400 MHz, DMSO-d6) δ ppm 15.2 (br. s., 1H), 9.05 (d, J=2.5 Hz, 1H), 8.97 (d, J=2.5 Hz, 1H), 8.41 (s, 1H), 7.47-7.60 (m, 2H), 7.39 (tt, J=9.4, 2.5, 2.4 Hz, 1H). MS (ES+) m/e 303 [M+H]⁺.

### 10e) N-{[7-(3,5-difluorophenyl)-6-hydroxy-5-quinoxalinyl]carbonyl}glycine

To a solution of the compound from Example 10d) (0.079 g, 0.261 mmol) and glycine ethyl ester hydrochloride (0.146 g, 1.04 mmol) in N,N-dimethylformamide (1.5 mL) were added diisopropylethylamine (0.202 mL, 1.56 mmol) and PyBOP (0.297 g, 0.570 mmol). The reaction mixture was stirred overnight at ambient temperature, quenched by water, acidified with 1N aqueous hydrochloric acid (2.0 mL), and extracted twice with ethyl acetate. The combined organic portions were dried over MgSO₄, filtered, concentrated in vacuo, and purified via flash column chromatography (0-10% methanol in dichloromethane). The resulting white solid was dissolved in ethanol (1.0 mL) and treated with 1N aqueous sodium hydroxide (1.0 mL) at ambient temperature for 15 min. The solution was concentrated in vacuo, dissolved in water, acidified with 1N aqueous hydrochloric acid (2.0 mL), filtered, and dried in vacuo to afford the title compound (0.004 g, 4%) as a pale yellow solid. ¹H NMR (400 MHz, DMSO-d6) δ ppm 11.6 (t, J=5.6 Hz, 1H), 8.97 (dd, J=9.6, 2.0 Hz, 2H), 8.31 (s, 1H), 7.44-7.54 (m, 2H), 7.36 (tt, J=9.4, 2.4 Hz, 1H), 4.27 (d, J=5.6 Hz, 2H). MS (ES+) m/e 360 [M+H]⁺.

### Example 11

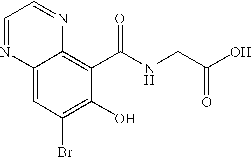

### N-[(7-bromo-6-hydroxy-5-quinoxalinyl)carbonyl]glycine

To a solution of 7-bromo-6-hydroxy-5-quinoxalinecarboxylic acid (prepared as in Example 10c) (0.100 g, 0.372 mmol) and glycine ethyl ester hydrochloride (0.208 g, 1.487 mmol) in dichloromethane (5.0 mL) were added triethylamine (0.311 ml, 2.230 mmol) and PyBOP (0.387 g, 0.743 mmol). The reaction mixture was stirred overnight at ambient temperature, quenched by water, and extracted twice with ethyl acetate. The combined organic portions were dried over MgSO₄, filtered, and concentrated in vacuo. The resulting yellow solid was dissolved in ethanol (5.0 mL) and treated with 1N aqueous sodium hydroxide (2.0 mL) at ambient temperature for 1 h. The solution was concentrated in vacuo, dissolved in water, acidified with 1N aqueous hydrochloric acid (4.0 mL), filtered, washed with water, and dried in vacuo to afford the title compound (0.072 g, 59%) as a brown solid. ¹H NMR (400 MHz, DMSO-d6) δ ppm 16.5 (s, 1H), 11.5 (t, J=5.6 Hz, 1H), 8.98 (d, J=1.5 Hz, 1H), 8.93 (d, J=1.5 Hz, 1H), 8.65 (s, 1H), 4.26 (d, J=5.6 Hz, 2H). MS (ES+) m/e 326/328 [M+H]⁺.

### Example 12

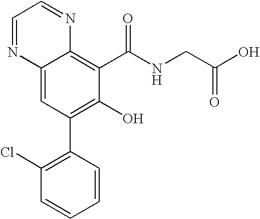

### N-{[7-(2-chlorophenyl)-6-hydroxy-5-quinoxalinyl]carbonyl}glycine

### 12a) 7-(2-Chlorophenyl)-6-hydroxy-5-quinoxalinecarboxylic acid

A solution of 7-bromo-6-hydroxy-5-quinoxalinecarboxylic acid (prepared as in Example 10c) (0.068 g, 0.253 mmol), 2-chlorobenzeneboronic acid (0.040 g, 0.253 mmol), potassium carbonate (0.104 g, 0.750 mmol), and tetrakis(triphenylphosphine)palladium(0) (0.009 g, 0.008 mmol) in 1,4-dioxane/water (3/1 solution) (1.0 mL) was heated to 100° C. for 20 min. in a Biotage Initiator® microwave synthesizer. Upon cooling, the reaction mixture was treated with water, acidified with 1N aqueous hydrochloric acid (˜2.0 mL), and extracted twice with ethyl acetate. The combined organic layers were dried over MgSO₄, filtered, concentrated in vacuo, and purified via flash column chromatography (0-10% methanol in dichloromethane) to afford the title compound (0.043 g, 57%) as a yellow solid. ¹H NMR (400 MHz, DMSO-d6) δ ppm 9.08 (d, J=2.3 Hz, 1H), 9.02 (d, J=2.5 Hz, 1H), 8.25 (s, 1H), 7.60-7.68 (m, 1H), 7.52-7.56 (m, 1H), 7.48-7.52 (m, 2H). MS (ES+) m/e 301 [M+H]⁺.

### 12b) N-{[7-(2-chlorophenyl)-6-hydroxy-5-quinoxalinyl]carbonyl}glycine

To a solution of the compound from Example 12a) (0.043 g, 0.143 mmol) and glycine ethyl ester hydrochloride (0.080 g, 0.573 mmol) in N,N-dimethylformamide (2.0 mL) were added diisopropylethylamine (0.149 mL, 0.858 mmol) and PyBOP (0.163 g, 0.315 mmol). The reaction mixture was stirred overnight at ambient temperature, quenched by water, and extracted thrice with ethyl acetate. The combined organic portions were dried over MgSO₄, filtered, concentrated in vacuo, and purified via flash column chromatography (0-10% methanol in dichloromethane). The resulting yellow oil was dissolved in ethanol (2.0 mL) and treated with 1N aqueous sodium hydroxide (1.0 mL) at ambient temperature for 20 min. The solution was concentrated in vacuo, dissolved in water, acidified with 1N aqueous hydrochloric acid (2.0 mL), filtered, washed with methanol, and dried in vacuo to afford the title compound (0.017 g, 33%) as a yellow solid. ¹H NMR (400 MHz, DMSO-d6) δ ppm 15.9 (br. s., 1H), 11.5 (t, J=5.6 Hz, 1H), 8.99 (d, J=2.0 Hz, 1H), 8.95 (d, J=2.0 Hz, 1H), 8.11 (s, 1H), 7.56-7.67 (m, 1H), 7.41-7.55 (m, 3H), 4.24 (d, J=5.6 Hz, 2H). MS (ES+) m/e 358 [M+H]⁺.

### Example 13

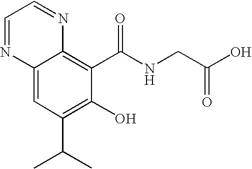

### N-{[6-hydroxy-7-(1-methylethyl)-5-quinoxalinyl]carbonyl}glycine

### 13a) Methyl 2-amino-5-bromo-6-(methyloxy)-3-nitrobenzoate

To a solution of methyl 2-amino-6-(methyloxy)-3-nitrobenzoate (prepared as in Example 1b) (0.420 g, 1.857 mmol) in dichloromethane (5.0 mL) was added bromine (0.100 mL, 1.940 mmol). After stirring 30 min. at ambient temperature, the reaction mixture was concentrated in vacuo, washed with hexanes, filtered, and dried in vacuo to afford the title compound (0.520 g, 92%) as a bright yellow solid. ¹H NMR (400 MHz, DMSO-d6) δ ppm 8.35 (s, 1H), 7.52 (br. s., 2H), 3.90 (s, 3H), 3.83 (s, 3H). MS (ES+) m/e 305/307 [M+H]⁺.

### 13b) Methyl 2-amino-5-(1-methylethenyl)-6-(methyloxy)-3-nitrobenzoate

A solution of the compound from Example 13a) (0.515 g, 1.688 mmol), isopropenylboronic acid pinacol ester (0.476 mL, 2.53 mmol), potassium carbonate (0.467 g, 3.38 mmol), and tetrakis(triphenylphosphine)palladium(0) (0.098 g, 0.084 mmol) in 1,4-dioxane (1.5 mL) and water (0.5 mL) was heated to 120° C. for 1 h in a Biotage Initiator® microwave synthesizer. Upon cooling, the reaction mixture was treated with water, diluted with brine, and extracted twice with ethyl acetate. The combined organic layers were dried over MgSO₄, filtered, concentrated in vacuo, and purified via flash column chromatography (10-30% ethyl acetate in hexanes) to afford the title compound (0.398 g, 89%) as a yellow oil. ¹H NMR (400 MHz, DMSO-d₆) δ ppm 7.97 (s, 1H), 7.38 (br. s., 2H), 5.20 (t, J=1.8 Hz, 1H), 5.17 (dd, J=1.9, 0.9 Hz, 1H), 3.88 (s, 3H), 3.70 (s, 3H), 2.04 (d, J=0.5 Hz, 3H). MS (ES+) m/e 267 [M+H]⁺.

### 13c) Methyl 7-(1-methylethyl)-6-(methyloxy)-5-quinoxalinecarboxylate

To a solution of the compound from Example 13b) (0.391 g, 1.47 mmol) in ethyl acetate (5.0 mL) was added 10% palladium on charcoal (0.078 g, 0.073 mmol), followed by evacuation of the reaction vessel and purging with 1 atmosphere of hydrogen. Following stirring at ambient temperature for 4 h, the reaction mixture was filtered through Celite®, washed through with ethyl acetate, and concentrated in vacuo. The resulting residue was dissolved in acetonitrile (1.0 mL) and water (5.0 mL), treated with glyoxal (40% aqueous solution) (0.185 mL, 1.615 mmol), and heated to 60° C. for 2 h. Upon cooling, the reaction mixture was diluted with brine and extracted twice with ethyl acetate. The combined organic layers were dried over MgSO₄, filtered, concentrated in vacuo, and purified via flash column chromatography (10-40% ethyl acetate in hexanes) to afford the title compound (0.210 g, 55%) as a clear, yellow oil. ¹H NMR (400 MHz, DMSO-d6) δ ppm 8.90 (d, J=2.0 Hz, 1H), 8.88 (d, J=2.0 Hz, 1H), 8.04 (s, 1H), 3.95 (s, 3H), 3.94 (s, 3H), 3.38 (qq, J=6.8 Hz, 1H), 1.32 (d, J=6.8 Hz, 6H). MS (ES+) m/e 261 [M+H]⁺.

### 13d) 6-Hydroxy-7-(1-methylethyl)-5-quinoxalinecarboxylic acid

To a solution of the compound from Example 13c) (0.201 g, 0.772 mmol) in dichloromethane (2.0 mL) was added boron tribromide (1M solution in dichloromethane) (3.10 mL, 3.10 mmol). The reaction mixture was stirred overnight at ambient temperature, quenched by water, diluted with brine, and extracted thrice with dichloromethane. The combined organic layers were dried over MgSO₄, filtered, concentrated in vacuo, and purified via flash column chromatography (30-100% ethyl acetate in hexanes) to afford the title compound (0.105 g, 59%) as a yellow solid. ¹H NMR (400 MHz, DMSO-d6) δ ppm 16.3 (s, 1H), 14.7 (s, 1H), 9.01 (d, J=2.3 Hz, 1H), 8.91 (d, J=2.3 Hz, 1H), 8.12 (s, 1H), 3.44 (qq, J=7.1, 6.9 Hz, 1H), 1.32 (d, J=6.8 Hz, 6H). MS (ES+) m/e 233 [M+H]⁺.

### 13e) Ethyl N-{[6-hydroxy-7-(1-methylethyl)-5-quinoxalinyl]carbonyl}glycinate

To a solution of the compound from Example 13d) (0.100 g, 0.431 mmol) and glycine ethyl ester hydrochloride (0.240 g, 1.722 mmol) in N,N-dimethylformamide (5.0 mL) were added triethylamine (0.360 mL, 2.58 mmol) and PyBOP (0.493 g, 0.947 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (10-40% ethyl acetate in hexanes) to afford the title compound (0.125 g, 91%) as a white solid. ¹H NMR (400 MHz, DMSO-d6) δ ppm 16.0 (s, 1H), 11.6 (t, J=5.6 Hz, 1H), 8.88 (s, 2H), 8.01 (s, 1H), 4.32 (d, J=5.6 Hz, 2H), 4.18 (q, J=7.1 Hz, 2H), 3.42 (qq, J=6.8 Hz, 1H), 1.31 (d, J=6.8 Hz, 6H), 1.23 (t, J=7.1 Hz, 3H). MS (ES+) m/e 318 [M+H]⁺.

### 13 f) N-{[6-hydroxy-7-(1-methylethyl)-5-quinoxalinyl]carbonyl}glycine

To a solution of the compound from Example 13e) (0.121 g, 0.381 mmol) in tetrahydrofuran (1.0 mL) and methanol (1.0 mL) was added 1N aqueous sodium hydroxide (1.0 mL, 1.00 mmol). After stirring 15 min. at ambient temperature, the reaction was quenched with 1N aqueous hydrochloric acid and the resulting precipitate was filtered, washed with water, and dried in vacuo to afford the title compound (0.096 g, 87%) as an off-white solid. ¹H NMR (400 MHz, DMSO-d6) δ ppm 16.1 (s, 1H), 12.9 (br. s., 1H), 11.5 (t, J=5.6 Hz, 1H), 8.88 (d, J=2.0 Hz, 1H), 8.87 (d, J=2.0 Hz, 1H), 8.01 (s, 1H), 4.24 (d, J=5.6 Hz, 2H), 3.43 (qq, J=6.8 Hz, 1H), 1.32 (d, J=6.8 Hz, 6H). MS (ES+) m/e 290 [M+H]⁺.

### Example 14

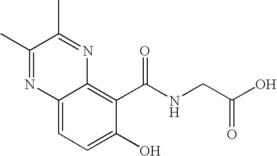

### N-[(6-hydroxy-2,3-dimethyl-5-quinoxalinyl)carbonyl]glycine

### 14a) Methyl 2,3-dimethyl-6-(methyloxy)-5-quinoxalinecarboxylate

To a solution of methyl 2-amino-6-(methyloxy)-3-nitrobenzoate (prepared as in Example 1b) (0.500 g, 2.211 mmol) in ethyl acetate (10.0 mL) was added 10% palladium on charcoal (0.118 g, 0.111 mmol), followed by evacuation of the reaction vessel and purging with 1 atmosphere of hydrogen. Following stirring at ambient temperature for 4 h, the reaction mixture was filtered through Celite®, washed through with ethyl acetate, and concentrated in vacuo. The resulting residue was dissolved in methanol (2.0 mL), treated with 2,3-butanedione (0.210 mL, 2.403 mmol), and heated to 100° C. for 20 min. in a Biotage Initiator® microwave synthesizer. Upon cooling, the reaction mixture was concentrated in vacuo and purified via flash column chromatography (20-60% ethyl acetate in hexanes) to afford the title compound (0.403 g, 74%) as an off-white solid. ¹H NMR (400 MHz, CHLOROFORM-d) δ ppm 8.00 (d, J=9.1 Hz, 1H), 7.43 (d, J=9.1 Hz, 1H), 4.05 (s, 3H), 3.99 (s, 3H), 2.68 (s, 3H), 2.67 (s, 3H). MS (ES+) m/e 247 [M+H]⁺.

### 14b) 6-Hydroxy-2,3-dimethyl-5-quinoxalinecarboxylic acid

To a solution of the compound from Example 14a) (0.403 g, 1.636 mmol) in dichloromethane (5.0 mL) was added boron tribromide (1M solution in dichloromethane) (6.50 mL, 6.50 mmol). The reaction mixture was stirred overnight at ambient temperature, quenched by water, diluted with brine, and extracted thrice with dichloromethane. The combined organic layers were dried over MgSO₄, filtered, concentrated in vacuo, and purified via flash column chromatography (0-10% methanol in dichloromethane) to afford the title compound (0.286 g, 80%) as a yellow solid. ¹H NMR (400 MHz, CHLOROFORM-d) δ ppm 16.2 (s, 1H), 13.1 (s, 1H), 8.10 (d, J=9.3 Hz, 1H), 7.47 (d, J=9.3 Hz, 1H), 2.81 (s, 3H), 2.77 (s, 3H). MS (ES+) m/e 219 [M+H]⁺.

### 14c) Ethyl N-[(6-hydr oxy-2,3-dimethyl-5-quinoxalinyl)carbonyl]glycinate

To a solution of the compound from Example 14b) (0.286 g, 1.31 mmol) and glycine ethyl ester hydrochloride (0.366 g, 2.62 mmol) in N,N-dimethylformamide (5.0 mL) were added triethylamine (0.550 mL, 3.95 mmol) and PyBOP (0.750 g, 1.44 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (10-60% ethyl acetate in hexanes) to afford the title compound (0.372 g, 94%) as a white solid. ¹H NMR (400 MHz, CHLOROFORM-d) δ ppm 14.8 (s, 1H), 12.0 (t, J=4.5 Hz, 1H), 8.03 (d, J=9.3 Hz, 1H), 7.38 (d, J=9.3 Hz, 1H), 4.36 (d, J=4.5 Hz, 2H), 4.32 (q, J=7.1 Hz, 2H), 2.83 (s, 3H), 2.74 (s, 3H), 1.36 (t, J=7.1 Hz, 3H). MS (ES+) m/e 304 [M+H]⁺.

### 14d) N-[(6-hydroxy-2,3-dimethyl-5-quinoxalinyl)carbonyl]glycine

To a solution of the compound from Example 14c) (0.372 g, 1.23 mmol) in methanol (2.0 mL) and tetrahydrofuran (2.0 mL) was added 1N aqueous sodium hydroxide (2.0 mL, 2.00 mmol). After stirring 15 min. at ambient temperature, the reaction was quenched with 1N aqueous hydrochloric acid and the resulting precipitate was filtered, washed with methanol, and dried in vacuo to afford the title compound (0.306 g, 91%) as an off-white solid. ¹H NMR (400 MHz, DMSO-d6) δ ppm 15.0 (s, 1H), 13.1 (br. s., 1H), 11.7 (t, J=4.8 Hz, 1H), 8.02 (d, J=9.3 Hz, 1H), 7.37 (d, J=9.3 Hz, 1H), 4.24 (d, J=4.8 Hz, 2H), 2.77 (s, 3H), 2.64 (s, 3H). MS (ES+) m/e 276 [M+H]⁺.

### Example 15

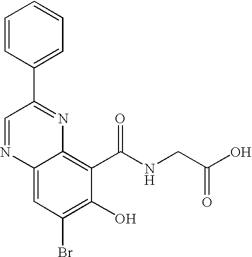

### N-[(7-bromo-6-hydroxy-3-phenyl-5-quinoxalinyl)carbonyl]glycine

### 15a) Methyl 7-bromo-6-(methyloxy)-3-phenyl-5-quinoxalinecarboxylate

To a solution of methyl 2-amino-5-bromo-6-(methyloxy)-3-nitrobenzoate (prepared as in Example 13a) (1.33 g, 4.36 mmol) in ethanol (60.0 mL), acetonitrile (60.0 mL), and water (2.0 mL) was added tin(II) chloride dihydrate (10.62 g, 47.1 mmol). After stirring at reflux for 3 h, the reaction mixture was allowed to cool to ambient temperature, poured into water, basified with 6N aqueous sodium hydroxide, and extracted thrice with ethyl acetate. The combined organic layers were dried over MgSO₄, filtered, and concentrated in vacuo. A portion of the resulting residue (0.132 g, 0.480 mmol) was dissolved in a mixture of acetonitrile (5.0 mL) and water (2.0 mL). The solution was treated with phenylglyoxal monohydrate (0.073 g, 0.480 mmol) and heated to 80° C. for 3 h. Upon cooling, the reaction mixture was poured into water, diluted with brine, and extracted thrice with ethyl acetate. The combined organic layers were dried over MgSO₄, filtered, concentrated in vacuo, and purified via flash column chromatography (20% ethyl acetate in hexanes). The product was then washed with diethyl ether, filtered, and dried in vacuo to afford the title compound (0.166 g, 93%) as an orange solid. ¹H NMR (400 MHz, CHLOROFORM-d) δ ppm 9.30 (s, 1H), 8.43 (s, 1H), 8.15-8.23 (m, 2H), 7.44-7.60 (m, 3H), 4.12 (s, 3H), 4.10 (s, 3H). MS (ES+) m/e 373/375 [M+H]⁺.

### 15b) 7-Bromo-6-hydroxy-3-phenyl-5-quinoxalinecarboxylic acid

To a solution of the compound from Example 15a) (0.152 g, 0.408 mmol) in dichloromethane (2.0 mL) was added boron tribromide (1M solution in dichloromethane) (1.22 mL, 1.22 mmol). The reaction mixture was stirred overnight at ambient temperature, quenched by water, diluted with brine, and extracted thrice with ethyl acetate. The combined organic layers were dried over MgSO₄, filtered, and concentrated in vacuo to afford the title compound (0.113 g, 80%) as a yellow solid. ¹H NMR (400 MHz, CHLOROFORM-d) δ ppm 16.4 (s, 1H), 14.2 (s, 1H), 9.33 (s, 1H), 8.61 (s, 1H), 8.04-8.11 (m, 2H), 7.50-7.77 (m, 3H). MS (ES+) m/e 345/347 [M+H]⁺.

### 15c) Ethyl N-[(7-bromo-6-hydroxy-3-phenyl-5-quinoxalinyl)carbonyl]glycinate

To a solution of the compound from Example 15b) (0.113 g, 0.327 mmol) and glycine ethyl ester hydrochloride (0.183 g, 1.31 mmol) in N,N-dimethylformamide (2.0 mL) were added diisopropylethylamine (0.342 mL, 1.96 mmol) and PyBOP (0.374 g, 0.719 mmol). The reaction mixture was stirred overnight at ambient temperature, quenched by water, diluted with brine, and extracted twice with ethyl acetate. The combined organic layers were dried over MgSO₄, filtered, concentrated in vacuo, and washed with methanol to afford the title compound (0.060 g, 43%) as a pale yellow solid. ¹H NMR (400 MHz, DMSO-d6) δ ppm 16.3 (s, 1H), 11.6 (t, J=5.6 Hz, 1H), 9.54 (s, 1H), 8.68 (s, 1H), 8.44 (m, 2H), 7.53-7.70 (m, 3H), 4.48 (d, J=5.6 Hz, 2H), 4.21 (q, J=7.1 Hz, 2H), 1.23 (t, J=7.1 Hz, 3H). MS (ES+) m/e 430/432 [M+H]⁺.

### 15d) N-[(7-bromo-6-hydroxy-3-phenyl-5-quinoxalinyl)carbonyl]glycine

To a solution of the compound from Example 15c) (0.060 g, 0.140 mmol) in ethanol (1.0 mL) was added 1N aqueous sodium hydroxide (1.0 mL). After stilling 15 min. at ambient temperature, a precipitate was collected by filtration and washed with ethanol. The solid was dissolved in water and acidified with 1N aqueous hydrochloric acid (2.0 mL). The resulting precipitate was filtered, washed with water, and dried in vacuo to afford the title compound (0.022 g, 39%) as a yellow solid. ¹H NMR (400 MHz, DMSO-d6) δ ppm 16.4 (s, 1H), 13.2 (br. s., 1H), 11.5 (t, J=5.3 Hz, 1H), 9.49 (s, 1H), 8.61 (s, 1H), 8.17-8.50 (m, 2H), 7.45-7.77 (m, 3H), 4.37 (d, J=5.3 Hz, 2H). MS (ES+) m/e 402/404 [M+H]⁺.

### Example 16

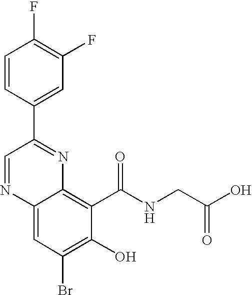

### N-{[7-bromo-3-(3,4-difluorophenyl)-6-hydroxy-5-quinoxalinyl]carbonyl}glycine

### 16a) Methyl 7-bromo-3-(3,4-difluorophenyl)-6-(methyloxy)-5-quinoxalinecarboxylate

To a solution of methyl 2-amino-5-bromo-6-(methyloxy)-3-nitrobenzoate (prepared as in Example 13a) (0.280 g, 0.918 mmol) in ethanol (15.0 mL) was added tin(II) chloride dihydrate (0.758 g, 3.36 mmol). After stilling at reflux for 2 h, the reaction mixture was allowed to cool to ambient temperature and poured into water, adjusted to pH˜8 with 5% aqueous sodium bicarbonate, and extracted thrice with ethyl acetate. The combined organic layers were dried over MgSO₄, filtered, and concentrated in vacuo. The resulting amber oil was diluted in methanol (2.0 mL), treated with 3,4-difluorophenylglyoxal hydrate (0.173 g, 0.918 mmol), and heated to 100° C. for 20 min. in a Biotage Initiator® microwave synthesizer. Upon cooling, a precipitate was collected by filtration, washed with methanol and hexanes, and dried in vacuo to afford the title compound (0.181 g, 48%) as a white solid. ¹H NMR (400 MHz, CHLOROFORM-d) δ ppm 9.24 (s, 1H), 8.44 (s, 1H), 7.98-8.11 (m, 1H), 7.89-7.96 (m, 1H), 7.30-7.41 (m, 1H), 4.13 (s, 3H), 4.10 (s, 3H). MS (ES+) m/e 409/411 [M+H]⁺.

### 16b) 7-Bromo-3-(3,4-difluorophenyl)-6-hydroxy-5-quinoxalinecarboxylic acid

To a solution of the compound from Example 16a) (0.181 g, 0.442 mmol) in dichloromethane (10.0 mL) was added boron tribromide (1M solution in dichloromethane) (1.33 mL, 1.33 mmol). The reaction mixture was stirred overnight at ambient temperature, quenched by water, diluted with brine, and extracted thrice with ethyl acetate. The combined organic layers were dried over MgSO₄, filtered and concentrated in vacuo. The residue was washed with dichloromethane, filtered, and dried in vacuo to afford the title compound (0.050 g, 30%) as a yellow solid. ¹H NMR (400 MHz, CHLOROFORM-d) δ ppm 9.61 (s, 1H), 8.75 (s, 1H), 8.31-8.42 (m, 1H), 8.04-8.15 (m, 1H), 7.67-7.90 (m, 1H). MS (ES+) m/e 381/383 [M+H]⁺.

### 16c) N-{[7-bromo-3-(3,4-difluorophenyl)-6-hydroxy-5-quinoxalinyl]carbonyl}glycine

To a solution of the compound from Example 16b) (0.050 g, 0.131 mmol) and glycine ethyl ester hydrochloride (0.027 g, 0.197 mmol) in N,N-dimethylformamide (2.0 mL) were added triethylamine (0.045 mL, 0.327 mmol) and PyBOP (0.075 g, 0.144 mmol). The reaction mixture was stirred overnight at ambient temperature, quenched by water, filtered, and washed with water and diethyl ether. The resulting white solid was dissolved in ethanol (2.0 mL) and treated with 1N aqueous sodium hydroxide (1.0 mL). After stirring 30 min. at ambient temperature, the reaction mixture was concentrated in vacuo, dissolved in water, and acidified with 1N aqueous hydrochloric acid (2.0 mL). The resulting precipitate was filtered, washed with water, and dried in vacuo to afford the title compound (0.006 g, 10%) as a yellow solid. ¹H NMR (400 MHz, DMSO-d6) δ ppm 16.3 (s, 1H), 11.6 (t, J=5.6 Hz, 1H), 9.54 (s, 1H), 8.68 (s, 1H), 8.44 (m, 2H), 7.53-7.70 (m, 3H), 4.48 (d, J=5.6 Hz, 2H), 4.21 (q, J=7.1 Hz, 2H), 1.23 (t, J=7.1 Hz, 3H). MS (ES+) m/e 438/440 [M+H]⁺.

### Example 17

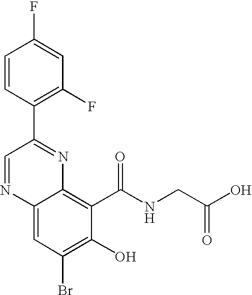

### N-{[7-bromo-3-(2,4-difluorophenyl)-6-hydroxy-5-quinoxalinyl]carbonyl}glycine

### 17a) Methyl 7-bromo-3-(2,4-difluorophenyl)-6-(methyloxy)-5-quinoxalinecarboxylate

To a solution of methyl 2-amino-5-bromo-6-(methyloxy)-3-nitrobenzoate (prepared as in Example 13a) (0.241 g, 0.789 mmol) in ethanol (10.0 mL) was added tin(II) chloride dihydrate (0.650 g, 2.87 mmol). After stirring at reflux for 2 h, the reaction mixture was allowed to cool to ambient temperature and poured into water, adjusted to pH˜8 with 5% aqueous sodium bicarbonate, and extracted thrice with ethyl acetate. The combined organic layers were dried over MgSO₄, filtered, and concentrated in vacuo. The resulting amber oil was diluted in methanol (2.0 mL), treated with 2,4-difluorophenylglyoxal hydrate (0.134 g, 0.789 mmol) and heated to 100° C. for 20 min. in a Biotage Initiator® microwave synthesizer. Upon cooling, a precipitate was collected by filtration, washed with methanol and hexanes, and dried in vacuo to afford the title compound (0.201 g, 62%) as a pink solid. ¹H NMR (400 MHz, CHLOROFORM-d) δ ppm 9.28 (d, J=2.8 Hz, 1H), 8.45 (s, 1H), 8.01-8.21 (m, 1H), 7.05-7.14 (m, 1H), 6.96-7.04 (m, 1H), 4.10 (s, 6H). MS (ES+) m/e 409/411 [M+H]⁺.

### 17b) 7-Bromo-3-(2,4-difluorophenyl)-6-hydroxy-5-quinoxalinecarboxylic acid

To a solution of the compound from Example 17a) (0.201 g, 0.491 mmol) in dichloromethane (10.0 mL) was added boron tribromide (1M solution in dichloromethane) (1.47 mL, 1.47 mmol). The reaction mixture was stirred overnight at ambient temperature, quenched by water, and extracted thrice with ethyl acetate. The title compound was collected by filtration of the combined organic layers and dried in vacuo. The filtrate was dried over MgSO₄, filtered, concentrated in vacuo, and combined with the above material to afford the title compound (0.064 g, 35%) as a white solid. ¹H NMR (400 MHz, CHLOROFORM-d) δ ppm 9.43 (d, J=1.5 Hz, 1H), 8.80 (s, 1H), 8.22-8.27 (m, 1H), 7.62-7.67 (m, 1H), 7.45-7.51 (m, 1H). MS (ES+) m/e 381/383 [M+H]⁺.

### 17c) N-{[7-bromo-3-(2,4-difluorophenyl)-6-hydroxy-5-quinoxalinyl]carbonyl}glycine

To a solution of the compound from Example 17b) (0.064 g, 0.168 mmol) and glycine ethyl ester hydrochloride (0.094 g, 0.672 mmol) in N,N-dimethylformamide (1.0 mL) were added triethylamine (0.140 mL, 1.01 mmol) and HATU (0.141 g, 0.370 mmol). The reaction mixture was stirred overnight at ambient temperature, quenched by water, filtered, and washed with water. The resulting white solid was diluted in ethanol (2.0 mL) and treated with 1N aqueous sodium hydroxide (2.0 mL). After stirring 1 h at ambient temperature, the reaction mixture was concentrated in vacuo, dissolved in water, and acidified with 1N aqueous hydrochloric acid (2.0 mL). The resulting precipitate was filtered, washed with water, and dried in vacuo to afford the title compound (0.013 g, 18%) as a yellow solid. ¹H NMR (400 MHz, DMSO-d6) δ ppm 11.4 (t, J=5.1 Hz, 1H), 9.27 (d, J=2.5 Hz, 1H), 8.67 (s, 1H), 8.13-8.32 (m, 1H), 7.48-7.62 (m, 1H), 7.24-7.43 (m, 1H), 4.27 (d, J=5.1 Hz, 2H). MS (ES+) m/e 438/440 [M+H]⁺.

### Example 18

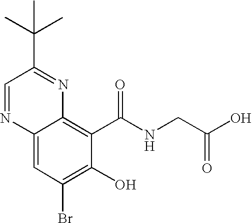

### N-{[7-bromo-3-(1,1-dimethylethyl)-6-hydroxy-5-quinoxalinyl]carbonyl}glycine

### 18a) Methyl 2,3-diamino-5-bromo-6-(methyloxy)benzoate

To a solution of methyl 2-amino-5-bromo-6-(methyloxy)-3-nitrobenzoate (prepared as in Example 13a) (1.13 g, 3.70 mmol) in ethanol (25.0 mL) was added tin(II) chloride dihydrate (3.06 g, 13.56 mmol). After stirring at reflux for 2 h, the reaction mixture was allowed to cool to ambient temperature and poured into water, adjusted to pH˜8 with 5% aqueous sodium bicarbonate, and extracted thrice with ethyl acetate. The combined organic layers were dried over MgSO₄, filtered, and concentrated in vacuo to afford title compound (0.99 g, 97%) as an amber oil. ¹H NMR (400 MHz, CHLOROFORM-d) δ ppm 7.53 (s, 1H) 3.97 (s, 3H) 3.86 (s, 3H) MS (ES+) m/e 275/277 [M+H]⁺.

### 18b) Methyl 7-bromo-3-(1,1-dimethylethyl)-6-(methyloxy)-5-quinoxalinecarboxylate

A mixture of methanol (0.200 mL), water (0.010 mL), and selenium dioxide (0.22 g, 1.983 mmol) was stirred under reflux until all the selenium dioxide was dissolved. Pinacolone (0.251 mL, 1.818 mmol) was added rapidly and the solution was vigorously stirred under reflux for 6 h. After cooling, the black residue was filtered and the yellow filtrate was diluted with acetonitrile (5.0 ml) and treated with the compound from Example 18a) (0.500 g, 1.818 mmol). The solution was stirred overnight at ambient temperature and then filtered. The dark red filtrate was diluted with brine and extracted thrice with ethyl acetate. The combined organic portions were dried over MgSO₄, filtered, concentrated in vacuo, and purified via flash column chromatography (10-40% ethyl acetate in hexanes) to afford the title compound (0.082 g, 13%) as an amber oil. ¹H NMR (400 MHz, CHLOROFORM-d) δ ppm 8.91 (s, 1H) 8.38 (s, 1H) 4.06 (s, 3H) 4.06 (s, 3H) 1.46 (s, 9H). MS (ES+) m/e 353/355 [M+H]⁺.

### 18c) 7-Bromo-3-(1,1-dimethylethyl)-6-hydroxy-5-quinoxalinecarboxylic acid

To a solution of the compound from Example 18b) (0.082 g, 0.232 mmol) in dichloromethane (4.0 mL) was added boron tribromide (1M solution in dichloromethane) (0.722 mL, 0.722 mmol). The reaction mixture was stirred overnight at ambient temperature, quenched by water, and extracted thrice with ethyl acetate. The combined organic portions were dried over MgSO₄, filtered, concentrated in vacuo, and purified via flash column chromatography (10-40% ethyl acetate in hexanes) to afford the title compound (0.060 g, 77%) as a bright yellow solid. ¹H NMR (400 MHz, CHLOROFORM-d) δ ppm 14.1 (s, 1H) 9.01 (s, 1H) 8.55 (s, 1H) 1.57 (s, 9H). MS (ES+) m/e 325/327 [M+H]⁺.

### 18d) N-{[7-bromo-3-(1,1-dimethylethyl)-6-hydroxy-5-quinoxalinyl]carbonyl}glycine

To a solution of the compound from Example 18c) (0.060 g, 0.185 mmol) and glycine ethyl ester hydrochloride (0.103 g, 0.738 mmol) in N,N-dimethylformamide (3.0 mL) were added triethylamine (0.154 mL, 1.107 mmol) and PyBOP (0.211 g, 0.406 mmol). The reaction mixture was stirred overnight at ambient temperature, quenched by water, and extracted thrice with ethyl acetate. The combined organic portions were dried over MgSO₄, filtered, and concentrated in vacuo. The resulting yellow oil was diluted with ethanol (3.0 mL) and treated with 1N aqueous sodium hydroxide (5.0 mL). Following stirring at ambient temperature for 1 h, the reaction mixture was concentrated in vacuo, and the resulting residue was dissolved in water and extracted with ethyl acetate. The aqueous layer was acidified with 1N aqueous hydrochloric acid, extracted with ethyl acetate, dried over MgSO₄, filtered, concentrated in vacuo, and purified via flash column chromatography (0-100% ethyl acetate in hexanes) to afford the title compound (0.0085 g, 12%) as a yellow solid. ¹H NMR (400 MHz, CHLOROFORM-d) δ ppm 15.9 (s, 1H) 11.9 (t, J=5.6 Hz, 1H) 8.91 (s, 1H) 8.48 (s, 1H) 4.49 (d, J=5.6 Hz, 2H) 1.55 (s, 9H). MS (ES+) m/e 382/384 [M+H]⁺.

### Example 19

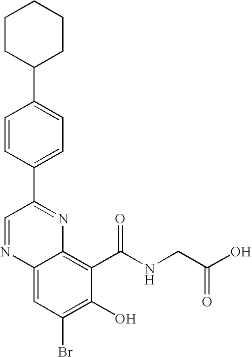

### N-{[7-bromo-3-(4-cyclohexylphenyl)-6-hydroxy-5-quinoxalinyl]carbonyl}glycine

### 19a) Methyl 7-bromo-3-(4-cyclohexylphenyl)-6-(methyloxy)-5-quinoxalinecarboxylate

A solution of methyl 2,3-diamino-5-bromo-6-(methyloxy)benzoate (prepared as in Example 18a) (0.261 g, 0.949 mmol) and 4-cyclohexylphenylglyoxal hydrate (0.222 g, 0.949 mmol) in methanol (3.0 mL) was heated to 100° C. for 20 min. in a Biotage Initiator® microwave synthesizer. Upon cooling, the reaction mixture was concentrated in vacuo and purified via flash column chromatography (0-10% methanol in dichloromethane) to afford the title compound (0.146 g, 34%) as a yellow solid. ¹H NMR (400 MHz, CHLOROFORM-d) δ ppm 9.28 (s, 1H), 8.42 (s, 1H), 8.12 (d, J=8.3 Hz, 2H), 7.41 (d, J=8.1 Hz, 2H), 4.13 (s, 3H), 4.10 (s, 3H), 2.47-2.71 (m, 1H), 1.86-2.00 (m, 4H), 1.75-1.85 (m, 1H), 1.40-1.58 (m, 4H), 1.27-1.39 (m, 1H). MS (ES+) m/e 455/457 [M+H]⁺.

### 19b) 7-Bromo-3-(4-cyclohexylphenyl)-6-hydroxy-5-quinoxalinecarboxylic acid

To a solution of the compound from Example 19a) (0.146 g, 0.321 mmol) in dichloromethane (10.0 mL) was added boron tribromide (1M solution in dichloromethane) (0.963 mL, 0.963 mmol). The reaction mixture was stirred overnight at ambient temperature, quenched by water, and extracted thrice with ethyl acetate. The combined organic portions were dried over MgSO₄, filtered, and concentrated in vacuo to afford the title compound (0.082 g, 60%) as a yellow solid. ¹H NMR (400 MHz, CHLOROFORM-d) δ ppm 14.2 (s, 1H) 9.31 (s, 1H) 8.58 (s, 1H) 8.01-8.04 (m, 1H) 7.99-8.01 (m, 1H) 7.49-7.51 (m, 1H) 7.46-7.48 (m, 1H) 2.52-2.72 (m, 1H) 1.85-2.01 (m, 4H) 1.81 (dd, J=13.8, 3.2 Hz, 1H) 1.37-1.53 (m, 5H). MS (ES+) m/e 427/429 [M+H]⁺.

### 19c) N-{[7-bromo-3-(4-cyclohexylphenyl)-6-hydroxy-5-quinoxalinyl]carbonyl}glycine

To a solution of the compound from Example 19b) (0.082 g, 0.192 mmol) and glycine ethyl ester hydrochloride (0.107 g, 0.768 mmol) in N,N-dimethylformamide (10.0 mL) were added triethylamine (0.160 mL, 1.151 mmol) and PyBOP (0.220 g, 0.422 mmol). The reaction mixture was stirred overnight at ambient temperature, quenched by water, filtered, and washed with water. The derived solid was diluted with ethanol (10.0 mL) and treated with 1N aqueous sodium hydroxide (2.0 mL). Following stirring at ambient temperature for 1 h, the reaction mixture was concentrated in vacuo, and the resulting residue was dissolved in water and treated with 1N aqueous hydrochloric acid. The solution was filtered and the resulting solid was washed with water and dried in vacuo to afford the title compound (0.066 g, 71%) as a yellow solid. ¹H NMR (400 MHz, DMSO-d₆) δ ppm 11.5 (t, J=5.3 Hz, 1H) 9.49 (s, 1H) 8.63 (s, 1H) 8.31 (d, J=8.3 Hz, 1H) 7.46 (d, J=8.3 Hz, 1H) 4.38 (d, J=5.3 Hz, 2H) 2.57-2.75 (m, 1H) 1.78-1.92 (m, 4H) 1.65-1.78 (m, 1H) 1.34-1.56 (m, 4H) 1.14-1.35 (m, 1H). MS (ES+) m/e 484/486 [M+H]⁺.

### Example 20

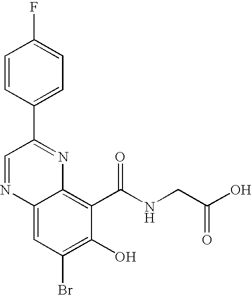

### N-{[7-bromo-3-(4-fluorophenyl)-6-hydroxy-5-quinoxalinyl]carbonyl}glycine

### 20a) Methyl 7-bromo-3-(4-fluorophenyl)-6-(methyloxy)-5-quinoxalinecarboxylate

A solution of methyl 2,3-diamino-5-bromo-6-(methyloxy)benzoate (prepared as in Example 18a) (0.414 g, 1.50 mmol) and 4-fluorophenylglyoxal hydrate (0.228 g, 1.50 mmol) in methanol (3.0 mL) was heated to 100° C. for 20 min. in a Biotage Initiator® microwave synthesizer. Upon cooling, the reaction mixture was concentrated in vacuo and purified via flash column chromatography (40-60% ethyl acetate in hexanes) to afford the title compound (0.105 g, 18%) as a yellow solid. ¹H NMR (400 MHz, CHLOROFORM-d) δ ppm 9.26 (s, 1H), 8.42 (s, 1H), 8.16-8.22 (m, 2H), 7.21-7.27 (m, 2H), 4.12 (s, 3H), 4.09 (s, 3H). MS (ES+) m/e 391/393 [M+H]⁺.

### 20b) 7-Bromo-3-(4-fluorophenyl)-6-hydroxy-5-quinoxalinecarboxylic acid

To a solution of the compound from Example 20a) (0.105 g, 0.268 mmol) in dichloromethane (10.0 mL) was added boron tribromide (1M solution in dichloromethane) (0.805 mL, 0.805 mmol). The reaction mixture was stirred overnight at ambient temperature, quenched by water, and extracted twice with ethyl acetate. The combined organic portions were dried over MgSO₄, filtered, and concentrated in vacuo to afford the title compound (0.038 g, 39%) as a yellow solid. ¹H NMR (400 MHz, CHLOROFORM-d) δ ppm 9.31 (s, 1H) 8.62 (s, 1H) 7.99-8.18 (m, 2H) 7.31-7.44 (m, 2H). MS (ES+) m/e 363/365 [M+H]⁺.

### 20c) N-{[7-bromo-3-(4-fluorophenyl)-6-hydroxy-5-quinoxalinyl]carbonyl}glycine

To a solution of the compound from Example 20b) (0.038 g, 0.105 mmol) and glycine ethyl ester hydrochloride (0.029 g, 0.209 mmol) in N,N-dimethylformamide (5.0 mL) were added triethylamine (0.088 mL, 0.628 mmol) and PyBOP (0.218 g, 0.419 mmol). The reaction mixture was stirred overnight at ambient temperature, quenched by water, filtered, and washed with water. The derived solid was diluted with ethanol (5.0 mL) and treated with 1N aqueous sodium hydroxide (1.0 mL). Following stirring at ambient temperature for 1 h, the reaction mixture was concentrated in vacuo, and the resulting residue was dissolved in water and treated with 1N aqueous hydrochloric acid. The solution was filtered and the resulting solid was washed with water and dried in vacuo to afford the title compound (0.010 g, 23%) as a yellow solid. ¹H NMR (400 MHz, DMSO-d₆) δ ppm 9.47 (s, 1H) 8.61 (s, 1H) 8.43 (dd, J=9.0, 5.4 Hz, 2H) 7.40 (t, J=8.8 Hz, 2H) 4.34 (s, 2H). MS (ES+) m/e 420/422 [M+H]⁺.

### Example 21

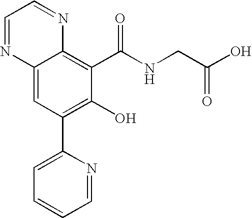

### N-{[6-hydroxy-7-(2-pyridinyl)-5-quinoxalinyl]carbonyl}glycine

### 21a) 6-Hydroxy-7-(2-pyridinyl)-5-quinoxalinecarboxylic acid

A solution of 7-bromo-6-hydroxy-5-quinoxalinecarboxylic acid (prepared as in Example 10c) (0.036 g, 0.134 mmol), copper(I) bromide-dimethyl sulfide complex (0.0028 g, 0.013 mmol), tetrakis(triphenylphosphine)palladium(0) (0.0077 g, 0.0067 mmol), and 2-(tributylstannyl)pyridine (0.044 mL, 0.134 mmol) in 1,4-dioxane (3.0 mL) was heated to 100° C. for 2 h in a sealed tube. Upon cooling, the reaction mixture was diluted with ethyl acetate, filtered through Celite®, washed through with ethyl acetate, and concentrated in vacuo. The derived residue was washed with diethyl ether, filtered, and dried in vacuo to afford the title compound (0.015 g, 42%) as an orange solid. ¹H NMR (400 MHz, DMSO-d6) δ ppm 15.5 (br. s., 1H), 14.2 (br. s., 1H), 8.81-9.04 (m, 2H), 8.78 (s, 1H), 8.39-8.65 (m, 1H), 7.99-8.22 (m, 1H), 7.38-7.78 (m, 2H). MS (ES+) m/e 268 [M+H]⁺.

### 21b) N-{[6-hydroxy-7-(2-pyridinyl)-5-quinoxalinyl]carbonyl}glycine

To a solution of the compound from Example 21a) (0.015 g, 0.056 mmol) and glycine ethyl ester hydrochloride (0.031 g, 0.225 mmol) in dichloromethane (2.0 mL) were added triethylamine (0.047 mL, 0.337 mmol) and PyBOP (0.058 g, 0.112 mmol). The reaction mixture was stirred overnight at ambient temperature, quenched by water, and extracted twice with ethyl acetate. The combined organic portions were dried over MgSO₄, filtered, and concentrated in vacuo. The residue was dissolved in ethanol (2.0 mL) and treated with 1N aqueous sodium hydroxide (0.056 mL). Following stirring at ambient temperature for 1 h, the reaction mixture was concentrated in vacuo, and the residue was dissolved in water and acidified with 1N aqueous hydrochloric acid. The resulting precipitate was filtered, washed with water and diethyl ether, and dried in vacuo to afford the title compound (0.009 g, 49%) as a beige solid. ¹H NMR (400 MHz, DMSO-d₆) δ ppm 12.9 (br. s., 1H), 11.5 (t, J=5.6 Hz, 1H), 8.97 (d, J=2.0 Hz, 1H), 8.95 (d, J=2.0 Hz, 1H), 8.78 (d, J=4.0 Hz, 1H), 8.59 (s, 1H), 8.15 (d, J=7.8 Hz, 1H), 7.90-8.01 (m, 1H), 7.42-7.54 (m, 1H), 4.27 (d, J=5.6 Hz, 2H). MS (ES+) m/e 325 [M+H]⁺.

### Example 22

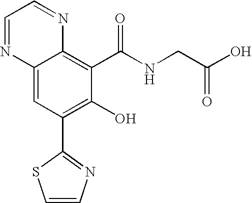

### N-{[6-hydroxy-7-(1,3-thiazol-2-yl)-5-quinoxalinyl]carbonyl}glycine

### 22a) Ethyl N-[(7-bromo-6-hydroxy-5-quinoxalinyl)carbonyl]glycinate

To a solution of 7-bromo-6-hydroxy-5-quinoxalinecarboxylic acid (prepared as in Example 10c) (0.306 g, 1.137 mmol) and glycine ethyl ester hydrochloride (0.635 g, 4.55 mmol) in dichloromethane (5.0 mL) were added triethylamine (0.951 ml, 6.82 mmol) and PyBOP (1.184 g, 2.275 mmol). The reaction mixture was stirred overnight at ambient temperature, quenched by water, and extracted twice with ethyl acetate. The combined organic portions were dried over MgSO₄, filtered, concentrated in vacuo, and purified via flash column chromatography (0-10% methanol in dichloromethane) to afford the title compound (0.220 g, 55%) as an off-white solid. ¹H NMR (400 MHz, METHANOL-d₄) δ ppm 8.91 (s, 1H), 8.82 (s, 1H), 8.52 (s, 1H), 4.36 (s, 2H), 4.27 (q, J=7.2 Hz, 2H), 1.32 (t, J=7.2 Hz, 3H). MS (ES+) m/e 354/356 [M+H]⁺.

### 22b) N-{[6-hydroxy-7-(1,3-thiazol-2-yl)-5-quinoxalinyl]carbonyl}glycine

To a solution of ethyl N-[(7-bromo-6-hydroxy-5-quinoxalinyl)carbonyl]glycinate (prepared as in Example 22a) (0.030 g, 0.085 mmol) in 1,4-dioxane (1.0 mL) were added 2-tributylstannanylthiazole (0.027 mL, 0.085 mmol) and tetrakis(triphenylphosphine)palladium(0) (0.0049 g, 0.0042 mmol), followed by heating to 100° C. overnight in a sealed tube. Upon cooling, the reaction mixture was diluted with ethyl acetate, filtered through Celite®, washed through with ethyl acetate, and concentrated in vacuo. The residue was washed with methanol, filtered, and then dissolved in ethanol (1.0 mL) and treated with 1N aqueous sodium hydroxide (1.0 mL). Following stirring at ambient temperature for 1 h, the reaction mixture was concentrated in vacuo, and the residue was dissolved in water and neutralized with 1N aqueous hydrochloric acid (1.0 mL). The resulting precipitate was filtered, washed with water and diethyl ether, and dried in vacuo to afford the title compound (0.0065 g, 23%) as a yellow solid. ¹H NMR (400 MHz, DMSO-d₆) δ ppm 13.0 (br. s., 1H), 11.6 (t, J=5.8 Hz, 1H), 9.07 (s, 1H), 9.00 (s, 2H), 8.16 (d, J=3.3 Hz, 1H), 8.04 (d, J=3.3 Hz, 1H), 4.31 (d, J=5.8 Hz, 2H). MS (ES+) m/e 331 [M+H]⁺.

### Example 23

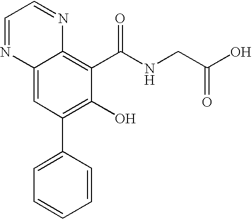

### N-[(6-hydroxy-7-phenyl-5-quinoxalinyl)carbonyl]glycine

A solution of N-[(6-hydroxy-7-bromo-5-quinoxalinyl)carbonyl]glycine (prepared as in Example 11) (0.020 g, 0.061 mmol), phenylboronic acid (0.0075 g, 0.061 mmol), potassium carbonate (0.025 g, 0.184 mmol), and tetrakis(triphenylphosphine)palladium(0) (0.0021 g, 0.0018 mmol) in 1,4-dioxane (1.0 mL) and water (0.330 mL) was heated to 100° C. for 20 min. in a Biotage Initiator® microwave synthesizer. Upon cooling, the reaction mixture was filtered through Celite®, washed through with ethyl acetate, and concentrated in vacuo. The residue was dissolved in water and acidified with 1N aqueous hydrochloric acid. The resulting precipitate was filtered, washed with water and methanol, and dried in vacuo to afford the title compound (0.017 g, 86%) as a dark yellow solid. ¹H NMR (400 MHz, DMSO-d6) δ ppm 16.2 (br. s., 1H), 11.6 (t, J=5.6 Hz, 1H), 8.95 (d, J=2.0 Hz, 1H), 8.93 (d, J=2.0 Hz, 1H), 8.17 (s, 1H), 7.71 (t, J=1.8 Hz, 1H), 7.66-7.70 (m, 1H), 7.49-7.55 (m, 2H), 7.41-7.49 (m, 1H), 4.27 (d, J=5.6 Hz, 2H). MS (ES+) m/e 324 [M+H]⁺.

### Example 24

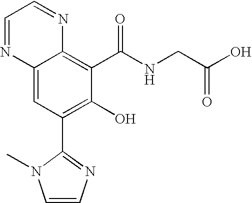

### N-{[6-hydroxy-7-(1-methyl-1H-imidazol-2-yl)-5-quinoxalinyl]carbonyl}glycine

To a solution of the compound from Example 22a) (0.044 g, 0.124 mmol) and tetrakis(triphenylphosphine)palladium(0) (0.00072 g, 0.0062 mmol) in 1,4-dioxane (2.0 mL) was added 1-methyl-2-(tributylstannanyl)-1H-imidazole (0.046 g, 0.124 mmol), followed by heating to 100° C. for 2 h in a sealed tube. Upon cooling, the reaction mixture was diluted with ethyl acetate, filtered through Celite®, washed through with ethyl acetate, and concentrated in vacuo. The residue was dissolved in ethanol (1.0 mL) and treated with 1N aqueous sodium hydroxide (1.242 mL). Following stirring at ambient temperature for 1 h, the reaction mixture was concentrated in vacuo, and the residue was dissolved in water and acidified with 1N aqueous hydrochloric acid. The reaction mixture was concentrated in vacuo and purified via C-18 reverse phase flash column chromatography (0-100% acetonitrile in water) to afford the title compound (0.0084 g, 21%) as a yellow solid. ¹H NMR (400 MHz, DMSO-d₆) δ ppm 11.5 (t, J=5.6 Hz, 1H), 8.97 (br. s., 1H), 8.94 (br. s., 1H), 8.18 (s, 1H), 7.82 (d, J=1.0 Hz, 1H), 7.19 (s, 1H), 4.20 (d, J=5.6 Hz, 2H), 3.64 (s, 3H). MS (ES+) m/e 328 [M+H]⁺.

### Example 25

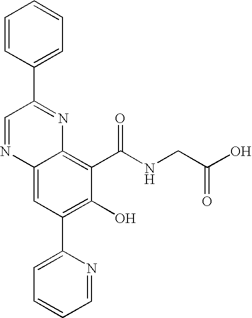

### N-{[6-hydroxy-3-phenyl-7-(2-pyridinyl)-5-quinoxalinyl]carbonyl}glycine

### 25a) Methyl 7-bromo-6-(methyloxy)-3-phenyl-5-quinoxalinecarboxylate

To a solution of methyl 2-amino-5-bromo-6-(methyloxy)-3-nitrobenzoate (prepared as in Example 13a) (0.675 g, 2.213 mmol) in ethyl acetate (10.0 mL) was added 10% palladium on charcoal (0.165 g, 0.155 mmol), followed by evacuation of the reaction vessel and purging with 1 atmosphere of hydrogen. Following stirring at ambient temperature for 2 h, the reaction mixture was filtered through Celite®, washed through with ethyl acetate, and concentrated in vacuo. The resulting residue was dissolved in methanol (2.0 mL), treated with phenylglyoxal hydrate (0.370 g, 2.434 mmol), and heated to 100° C. for 20 min. in a Biotage Initiator® microwave synthesizer. Upon cooling, the reaction mixture was filtered, washed with methanol, and dried in vacuo to afford the title compound (0.511 g, 62%) as a beige solid. ¹H NMR (400 MHz, CHLOROFORM-d) δ ppm 9.30 (s, 1H), 8.43 (s, 1H), 8.15-8.23 (m, 2H), 7.51-7.59 (m, 3H), 4.12 (s, 3H), 4.10 (s, 3H). MS (ES+) m/e 373/375 [M+H]⁺.

### 25b) 7-Bromo-6-hydroxy-3-phenyl-5-quinoxalinecarboxylic acid

To a solution of the compound from Example 25a) (0.506 g, 1.356 mmol) in dichloromethane (5.0 mL) was added boron tribromide (1M solution in dichloromethane) (5.0 mL, 5.00 mmol). The reaction mixture was stirred overnight at ambient temperature, quenched by water, filtered, washed with diethyl ether, and dried in vacuo to afford the title compound (0.442 g, 94%) as a pale yellow solid. ¹H NMR (400 MHz, DMSO-d₆) δ ppm 13.8 (br. s., 1H), 9.65 (s, 1H), 8.79 (s, 1H), 8.20-8.28 (m, 2H), 7.64-7.76 (m, 3H). MS (ES+) m/e 345/347 [M+H]⁺.

### 25c) Ethyl N-[(7-bromo-6-hydr oxy-3-phenyl-5-quinoxalinyl)carbonyl]glycinate

To a solution of the compound from Example 25b) (0.436 g, 1.263 mmol) and glycine ethyl ester hydrochloride (0.353 g, 2.53 mmol) in N,N-dimethylformamide (5.0 mL) were added triethylamine (0.530 mL, 3.80 mmol) and PyBOP (0.723 g, 1.390 mmol). The reaction mixture was stirred overnight at ambient temperature, quenched by water, filtered, washed with water and ethyl acetate, and dried in vacuo to afford the title compound (0.422 g, 78%) as a pale yellow solid. ¹H NMR (400 MHz, DMSO-d₆) δ ppm 16.3 (s, 1H), 11.6 (t, J=5.6 Hz, 1H), 9.54 (s, 1H), 8.67 (s, 1H), 8.31-8.38 (m, 2H), 7.62-7.66 (m, 3H), 4.48 (d, J=5.6 Hz, 2H), 4.21 (q, J=7.2 Hz, 2H), 1.23 (t, J=7.2 Hz, 3H). MS (ES+) m/e 430/432 [M+H]⁺.

### 25d) Ethyl N-{[6-hydroxy-3-phenyl-7-(2-pyridinyl)-5-quinoxalinyl]carbonyl}glycinate

To a solution of the compound from Example 25c) (0.160 g, 0.372 mmol) in 1,4-dioxane (2.0 mL) was added 2-(tributylstannyl)pyridine (0.140 mL, 0.426 mmol) and tetrakis(triphenylphosphine)palladium(0) (0.020 g, 0.017 mmol) followed by heating to 150° C. for 20 min. in a Biotage Initiator® microwave synthesizer. Upon cooling, the reaction mixture was concentrated in vacuo and purified via flash column chromatography (20-80% ethyl acetate in hexanes) to afford the title compound (0.143 g, 90%) as a light yellow solid. ¹H NMR (400 MHz, DMSO-d₆) δ ppm 16.2 (s, 1H), 11.4 (t, J=5.6 Hz, 1H), 9.55 (s, 1H), 8.79 (ddd, J=4.8, 1.8, 1.0 Hz, 1H), 8.65 (s, 1H), 8.37 (dd, J=6.7, 3.2 Hz, 2H), 8.21 (d, J=8.1 Hz, 1H), 7.98 (dt, J=7.8, 1.9 Hz, 1H), 7.60-7.67 (m, 3H), 7.50 (ddd, J=7.6, 4.8, 1.0 Hz, 1H), 4.45 (d, J=5.6 Hz, 2H), 4.22 (q, J=7.1 Hz, 2H), 1.24 (t, J=7.1 Hz, 3H). MS (ES+) m/e 429 [M+H]⁺.

### 25e) N-{[6-hydroxy-3-phenyl-7-(2-pyridinyl)-5-quinoxalinyl]carbonyl}glycine

To a suspension of the compound from Example 25d) (0.140 g, 0.327 mmol) in methanol (2.0 mL) and tetrahydrofuran (2.0 mL) was added 1N aqueous sodium hydroxide (1.00 mL, 1.00 mmol). After stirring 30 min. at ambient temperature, the reaction was quenched with 1N aqueous hydrochloric acid and the resulting precipitate was filtered, washed with methanol, and dried in vacuo to afford the title compound (0.114 g, 87%) as a pale orange solid. ¹H NMR (400 MHz, DMSO-d₆) δ ppm 11.5 (t, J=5.1 Hz, 1H), 9.56 (s, 1H), 8.89 (d, J=4.8 Hz, 1H), 8.65 (s, 1H), 8.37-8.44 (m, 2H), 8.30 (d, J=7.8 Hz, 1H), 8.26 (t, J=7.6 Hz, 1H), 7.74 (t, J=5.8 Hz, 1H), 7.59-7.67 (m, 3H), 4.38 (d, J=5.1 Hz, 2H). MS (ES+) m/e 401 [M+H]⁺.

### Example 26

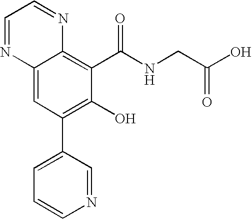

### N-{[6-hydroxy-7-(3-pyridinyl)-5-quinoxalinyl]carbonyl}glycine

A solution of ethyl N-[(7-bromo-6-hydroxy-5-quinoxalinyl)carbonyl]glycinate (prepared as in Example 22a) (0.044 g, 0.124 mmol), 3-pyridylboronic acid (0.015 g, 0.124 mmol), potassium carbonate (0.052 g, 0.373 mmol), and tetrakis(triphenylphosphine)palladium(0) (0.0043 g, 0.0037 mmol) in 1,4-dioxane (1.0 mL) and water (0.330 mL) was heated to 100° C. for 20 min. in a Biotage Initiator® microwave synthesizer. Upon cooling, the reaction mixture was filtered through Celite®, washed through with ethyl acetate, and concentrated in vacuo. The residue was dissolved in ethanol (3.0 mL) and treated with 1N aqueous sodium hydroxide (1.242 mL). Following stirring at ambient temperature for 1 h, the reaction mixture was concentrated in vacuo, and the residue was dissolved in water and acidified with 1N aqueous hydrochloric acid (2.0 mL). The resulting precipitate was filtered, washed with water, and dried in vacuo to afford the title compound (0.012 g, 30%) as a beige solid. ¹H NMR (400 MHz, DMSO-d6) δ ppm 16.3 (s, 1H), 13.0 (br. s., 1H), 11.6 (t, J=5.1 Hz, 1H), 8.97 (d, J=8.3 Hz, 2H), 8.90 (br. s., 1H), 8.66 (br. s., 1H), 8.31 (s, 1H), 8.16 (d, J=7.3 Hz, 1H), 7.56 (br. s., 1H), 4.28 (d, J=5.1 Hz, 2H). MS (ES+) m/e 325 [M+H]⁺.

### Example 27

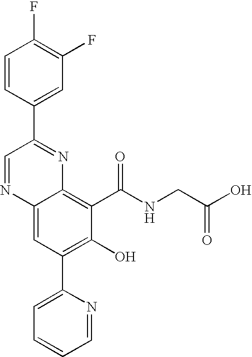

### N-{[3-(3,4-difluorophenyl)-6-hydroxy-7-(2-pyridinyl)-5-quinoxalinyl]carbonyl}glycine

### 27a) Methyl 7-bromo-3-(3,4-difluorophenyl)-6-(methyloxy)-5-quinoxalinecarboxylate

To a solution of methyl 2-amino-5-bromo-6-(methyloxy)-3-nitrobenzoate (prepared as in Example 13a) (0.675 g, 2.213 mmol) in ethyl acetate (10.0 mL) was added 10% palladium on charcoal (0.165 g, 0.155 mmol), followed by evacuation of the reaction vessel and purging with 1 atmosphere of hydrogen. Following stirring at ambient temperature for 2 h, the reaction mixture was filtered through Celite®, washed through with ethyl acetate, and concentrated in vacuo. The resulting residue was dissolved in methanol (2.0 mL), treated with 3,4-difluorophenylglyoxal hydrate (0.458 g, 2.434 mmol), and heated to 100° C. for 20 min. in a Biotage Initiator® microwave synthesizer. Upon cooling, the reaction mixture was filtered, washed with methanol, and dried in vacuo to afford the title compound (0.561 g, 62%) as a beige solid. ¹H NMR (400 MHz, DMSO-d₆) δ ppm 9.64 (s, 1H), 8.60 (s, 1H), 8.35 (ddd, J=11.9, 7.9, 2.1 Hz, 1H), 8.15-8.22 (m, 1H), 7.71 (dt, J=10.4, 8.6 Hz, 1H), 4.04 (s, 3H), 4.00 (s, 3H). MS (ES+) m/e 409/411 [M+H]⁺.

### 27b) 7-Bromo-3-(3,4-difluorophenyl)-6-hydroxy-5-quinoxalinecarboxylic acid

To a solution of the compound from Example 27a) (0.555 g, 1.356 mmol) in dichloromethane (5.0 mL) was added boron tribromide (1M solution in dichloromethane) (5.0 mL, 5.00 mmol). The reaction mixture was stirred overnight at ambient temperature, quenched by water, filtered, washed with diethyl ether, and dried in vacuo to afford the title compound (0.465 g, 90%) as a pale yellow solid. ¹H NMR (400 MHz, DMSO-d₆) δ ppm 13.6 (br. s., 1H), 9.60 (s, 1H), 8.73 (s, 1H), 8.37 (ddd, J=11.8, 7.8, 2.1 Hz, 1H), 8.09-8.15 (m, 1H), 7.78 (dt, J=10.4, 8.6 Hz, 1H). MS (ES+) m/e 381/383 [M+H]⁺.

27c) Ethyl N-{[7-bromo-3-(3,4-difluorophenyl)-6-hydroxy-5-quinoxalinyl]carbonyl}glycinate

To a solution of the compound from Example 27b) (0.460 g, 1.207 mmol) and glycine ethyl ester hydrochloride (0.505 g, 3.62 mmol) in dichloromethane (5.0 mL) were added triethylamine (0.680 mL, 4.88 mmol) and PyBOP (1.260 g, 2.421 mmol). The reaction mixture was stirred overnight at ambient temperature, quenched by water, filtered, washed with water and ethyl acetate, and dried in vacuo to afford the title compound (0.522 g, 93%) as a pale yellow solid. ¹H NMR (400 MHz, CHLOROFORM-d) δ ppm 16.4 (s, 1H), 11.6 (t, J=5.0 Hz, 1H), 9.14 (s, 1 H), 8.53 (s, 1H), 8.14 (ddd, J=11.1, 7.6, 2.3 Hz, 1H), 7.90-8.01 (m, 1H), 7.41 (dt, J=9.6, 8.4 Hz, 1H), 4.44 (d, J=5.0 Hz, 2H), 4.34 (q, J=7.1 Hz, 2H), 1.36 (t, J=7.1 Hz, 3H). MS (ES+) m/e 466/468 [M+H]⁺.

### 27d) Ethyl N-{[3-(3,4-difluorophenyl)-6-hydroxy-7-(2-pyridinyl)-5-quinoxalinyl]carbonyl}glycinate

To a solution of the compound from Example 27c) (0.150 g, 0.322 mmol) in 1,4-dioxane (2.0 mL) was added 2-(tributylstannyl)pyridine (0.120 mL, 0.365 mmol) and tetrakis(triphenylphosphine)palladium(0) (0.010 g, 8.65 mmol) followed by heating to 150° C. for 20 min. in a Biotage Initiator® microwave synthesizer. Upon cooling, the reaction mixture was filtered, washed with methanol and dichloromethane, and dried in vacuo to afford the title compound (0.126 g, 84%) as a light yellow solid. ¹H NMR (400 MHz, DMSO-d₆) δ ppm 16.2 (s, 1H), 11.0 (t, J=5.6 Hz, 1H), 9.57 (s, 1H), 8.79 (ddd, J=4.8, 1.8, 1.0 Hz, 1H), 8.69 (s, 1H), 8.50 (ddd, J=11.8, 7.8, 2.1 Hz, 1H), 8.23-8.32 (m, 2H), 8.01 (dt, J=7.8, 1.9 Hz, 1H), 7.71 (dt, J=10.4, 8.6 Hz, 1H), 7.53 (ddd, J=7.6, 4.8, 1.0 Hz, 1H), 4.42 (d, J=5.6 Hz, 2H), 4.24 (q, J=7.1 Hz, 2H), 1.26 (t, J=7.1 Hz, 3H). MS (ES+) m/e 465 [M+H]⁺.

### 27e) N-{[3-(3,4-difluorophenyl)-6-hydroxy-7-(2-pyridinyl)-5-quinoxalinyl]carbonyl}glycine

To a suspension of the compound from Example 27d) (0.121 g, 0.261 mmol) in methanol (2.0 mL) and tetrahydrofuran (2.0 mL) was added 1N aqueous sodium hydroxide (1.0 mL, 1.00 mmol). After stirring 30 min. at ambient temperature, the reaction was quenched with 1N aqueous hydrochloric acid and the resulting precipitate was filtered, washed with methanol, and dried in vacuo to afford the title compound (0.104 g, 91%) as a light orange solid. ¹H NMR (400 MHz, CHLOROFORM-d/METHANOL-d₄) δ ppm 9.16 (s, 1H), 8.86 (ddd, J=4.8, 1.8, 1.0 Hz, 1H), 8.53 (s, 1H), 8.30 (ddd, J=11.1, 7.6, 2.3 Hz, 1H), 8.21 (d, J=8.1 Hz, 1H), 8.12 (dt, J=7.8, 1.9 Hz, 1H), 7.91-7.97 (m, 1H), 7.79 (dt, J=9.6, 8.4 Hz, 1H), 7.34 (ddd, J=7.6, 4.8, 1.0 Hz, 1H), 4.33 (s, 2H). MS (ES+) m/e 437 [M+H]⁺.

### Example 28

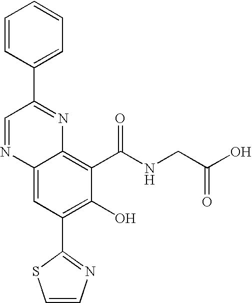

### N-{[6-hydroxy-3-phenyl-7-(1,3-thiazol-2-yl)-5-quinoxalinyl]carbonyl}glycine

### 28a) Ethyl N-{[6-hydroxy-3-phenyl-7-(1,3-thiazol-2-yl)-5-quinoxalinyl]carbonyl}glycinate

To a solution of the compound from Example 25c) (0.150 g, 0.349 mmol) in 1,4-dioxane (2.0 mL) was added 2-tributylstannanylthiazole (0.120 mL, 0.382 mmol) and tetrakis(triphenylphosphine)palladium(0) (0.020 g, 0.017 mmol) followed by heating to 150° C. for 20 min. in a Biotage Initiator® microwave synthesizer. Upon cooling, the reaction mixture was concentrated in vacuo and purified via flash column chromatography (0-10% methanol in dichloromethane) followed by trituration with dichloromethane to afford the title compound (0.061 g, 40%) as a pale orange solid. ¹H NMR (400 MHz, DMSO-d₆) δ ppm 11.7 (t, J=5.6 Hz, 1H), 11.5 (br. s., 1H), 9.57 (s, 1H), 9.08 (s, 1H), 8.32-8.38 (m, 2H), 8.15 (d, J=3.3 Hz, 1H), 8.02 (d, J=3.3 Hz, 1H), 7.62-7.68 (m, 3H), 4.50 (d, J=5.6 Hz, 2H), 4.22 (q, J=7.1 Hz, 2H), 1.24 (t, J=7.1 Hz, 3H). MS (ES+) m/e 435 [M+H]⁺.

### 28b) N-{[6-hydroxy-3-phenyl-7-(1,3-thiazol-2-yl)-5-quinoxalinyl]carbonyl}glycine

To a suspension of the compound from Example 28a) (0.061 g, 0.140 mmol) in methanol (2.0 mL) and tetrahydrofuran (2.0 mL) was added 1N aqueous sodium hydroxide (1.0 mL, 1.000 mmol). After stirring 30 min. at ambient temperature, the reaction was quenched with 1N aqueous hydrochloric acid and the resulting precipitate was filtered, washed with methanol, and dried in vacuo to afford the title compound (0.055 g, 96%) as an orange solid. ¹H NMR (400 MHz, DMSO-d₆) δ ppm 13.2 (br. s., 1H), 11.6 (t, J=5.1 Hz, 1H), 9.53 (s, 1H), 9.01 (s, 1H), 8.30-8.43 (m, 2H), 8.12 (d, J=3.3 Hz, 1H), 8.01 (d, J=3.3 Hz, 1H), 7.54-7.67 (m, 3H), 4.39 (d, J=5.1 Hz, 2H). MS (ES+) m/e 407 [M+H]⁺.

### Example 29

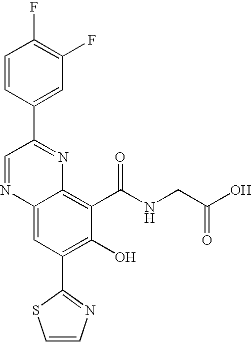

### N-{[3-(3,4-difluorophenyl)-6-hydroxy-7-(1,3-thiazol-2-yl)-5-quinoxalinyl]carbonyl}glycine

### 29a) Ethyl N-{[3-(3,4-difluorophenyl)-6-hydroxy-7-(1,3-thiazol-2-yl)-5-quinoxalinyl]carbonyl}glycinate

To a solution of the compound from Example 27c) (0.150 g, 0.322 mmol) in 1,4-dioxane (2.0 mL) was added 2-tributylstannanylthiazole (0.120 mL, 0.382 mmol) and tetrakis(triphenylphosphine)palladium(0) (0.010 g, 8.65 mmol) followed by heating to 150° C. for 20 min. in a Biotage Initiator® microwave synthesizer. Upon cooling, the reaction mixture was concentrated in vacuo, triturated with dichloromethane, filtered, and dried in vacuo to afford the title compound (0.116 g, 77%) as a light yellow solid. ¹H NMR (400 MHz, DMSO-d₆) δ ppm 15.8 (s, 1H), 11.5 (t, J=5.0 Hz, 1H), 9.60 (s, 1H), 9.08 (s, 1H), 8.48 (ddd, J=11.1, 7.6, 2.3 Hz, 1H), 8.23-8.29 (m, 1H), 8.16 (d, J=3.3 Hz, 1H), 8.05 (d, J=3.3 Hz, 1H), 7.71 (dt, J=9.6, 8.4 Hz, 1H), 4.50 (d, J=5.0 Hz, 2H), 4.24 (q, J=7.1 Hz, 2H), 1.26 (t, J=7.1 Hz, 3H). MS (ES+) m/e 471 [M+H]⁺.

### 29b) N-{[3-(3,4-difluorophenyl)-6-hydroxy-7-(1,3-thiazol-2-yl)-5-quinoxalinyl]carbonyl}glycine

To a suspension of the compound from Example 29a) (0.112 g, 0.238 mmol) in methanol (1.0 mL) and tetrahydrofuran (1.0 mL) was added 1N aqueous sodium hydroxide (0.500 mL, 0.500 mmol). After stirring 30 min. at ambient temperature, the reaction was quenched with 1N aqueous hydrochloric acid and the resulting precipitate was filtered, washed with methanol, and dried in vacuo to afford the title compound (0.102 g, 97%) as a light orange solid. ¹H NMR (400 MHz, DMSO-d₆) δ ppm 13.3 (br. s., 1H), 11.2 (t, J=4.8 Hz, 1H), 9.48 (s, 1H), 8.94 (s, 1H), 8.41 (ddd, J=11.7, 7.8, 2.0 Hz, 1H), 8.16-8.26 (m, 1H), 8.10 (d, J=3.3 Hz, 1H), 7.99 (d, J=3.3 Hz, 1H), 7.61 (dt, J=10.3, 8.6 Hz, 1H), 4.35 (d, J=4.8 Hz, 2H). MS (ES+) m/e 443 [M+H]⁺.

### Example 30

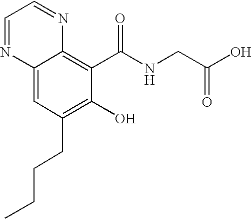

### N-[(7-butyl-6-hydroxy-5-quinoxalinyl)carbonyl]glycine

A solution of ethyl N-[(7-bromo-6-hydroxy-5-quinoxalinyl)carbonyl]glycinate (prepared as in Example 22a) (0.116 g, 0.328 mmol), 2-(tributylstannanyl)-1,3-oxazole (0.123 g, 0.342 mmol), and tetrakis(triphenylphosphine)palladium(0) (0.017 g, 0.015 mmol) in 1,4-dioxane (2.0 mL) was heated to 100° C. overnight in a sealed tube. Upon cooling, the reaction mixture was concentrated in vacuo and purified via flash column chromatography (80-100% ethyl acetate in hexanes). The resulting amber oil was dissolved in ethanol (2.0 mL) and treated with 1N aqueous sodium hydroxide (1.638 mL). Following stirring at ambient temperature for 20 min., the reaction mixture was acidified with 1N aqueous hydrochloric acid. The resulting precipitate was filtered, washed with water, and dried in vacuo to afford the title compound (0.010 g, 10%) as a pale yellow solid. ¹H NMR (400 MHz, DMSO-d₆) δ ppm 15.9 (br. s., 1H), 11.5 (t, J=5.6 Hz, 1H), 8.88 (d, J=2.2 Hz, 1H), 8.86 (d, J=2.2 Hz, 1H), 8.02 (s, 1H), 4.24 (d, J=5.6 Hz, 2H), 2.82 (t, J=7.6 Hz, 2H), 1.58-1.75 (m, 2H), 1.30-1.45 (m, 2H), 0.93 (t, J=7.3 Hz, 3H). MS (ES+) m/e 304 [M+H]⁺.

### Example 31

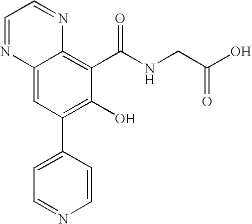

### N-{[6-hydroxy-7-(4-pyridinyl)-5-quinoxalinyl]carbonyl}glycine

A solution of ethyl N-[(7-bromo-6-hydroxy-5-quinoxalinyl)carbonyl]glycinate (prepared as in Example 22a) (0.088 g, 0.248 mmol), 4-pyridylboronic acid (0.032 g, 0.260 mmol), potassium carbonate (0.103 g, 0.745 mmol), and tetrakis(triphenylphosphine)palladium(0) (0.013 g, 0.011 mmol) in 1,4-dioxane (2.0 mL) and water (0.667 mL) was heated to 100° C. overnight in a sealed tube. Upon cooling, the reaction mixture was diluted with water and extracted with ethyl acetate. The aqueous layer was acidified with 1N aqueous hydrochloric acid and the resulting precipitate was filtered, washed with water, and dried in vacuo to afford the title compound (0.026 g, 32%) as a dark grey solid. ¹H NMR (400 MHz, DMSO-d₆) δ ppm 16.3 (s, 1H), 11.6 (t, J=5.6 Hz, 1H), 9.01 (d, J=2.0 Hz, 1H), 8.98 (d, J=2.0 Hz, 1H), 8.74 (d, J=5.6 Hz, 2H), 8.35 (s, 1H), 7.79 (d, J=5.6 Hz, 2H), 4.28 (d, J=5.6 Hz, 2H). MS (ES+) m/e 325 [M+H]⁺.

### Example 32

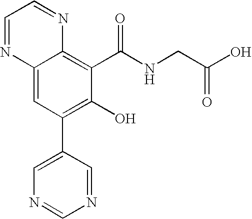

### N-{[6-hydroxy-7-(5-pyrimidinyl)-5-quinoxalinyl]carbonyl}glycine

A solution of ethyl N-[(7-bromo-6-hydroxy-5-quinoxalinyl)carbonyl]glycinate (prepared as in Example 22a) (0.090 g, 0.254 mmol), 5-pyrimidinylboronic acid (0.031 g, 0.254 mmol), potassium carbonate (0.105 g, 0.762 mmol), and tetrakis(triphenylphosphine)palladium(0) (0.0088 g, 0.0076 mmol) in 1,4-dioxane (2.0 mL) and water (0.667 mL) was heated to 150° C. for 20 min. in a Biotage Initiator® microwave synthesizer. Upon cooling, the reaction mixture was concentrated in vacuo, dissolved in water, and acidified with 1N aqueous hydrochloric acid. The resulting precipitate was filtered, washed with water, and dried in vacuo to afford the title compound (0.030 g, 36%) as a brown solid. ¹H NMR (400 MHz, DMSO-d₆) δ ppm 11.6 (t, J=5.3 Hz, 1H), 9.27 (s, 1H), 9.17 (br. s., 2H), 9.00 (d, J=2.0 Hz, 1H), 8.98 (d, J=2.0 Hz, 1H), 8.47 (s, 1H), 4.27 (d, J=5.3 Hz, 2H). MS (ES+) m/e 326 [M+H]⁺.

### Example 33

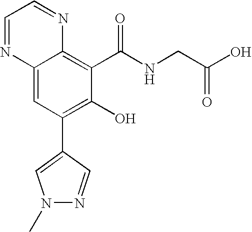

### N-{[6-hydroxy-7-(1-methyl-1H-pyrazol-4-yl)-5-quinoxalinyl]carbonyl}glycine

A solution of ethyl N-[(7-bromo-6-hydroxy-5-quinoxalinyl)carbonyl]glycinate (example 22(a), 0.03 g, 0.085 mmol), 1-methyl-4-(4,4,5,5-tetramethyl-1,3,2-dioxaborolan-2-yl)-1H-pyrazole (0.018 g, 0.085 mmol), potassium carbonate (0.035 g, 0.254 mmol), and tetrakis(triphenylphosphine)palladium(0) (3.00 mg, 2.60 mmol) in N,N-Dimethylformamide (1.0 ml) and water (1.000 ml) was heated to 100° C. for 20 min. in a Biotage Initiator® microwave synthesizer. Upon cooling, the reaction mixture was diluted with ethyl acetate, filtered through Celite®, washed through with ethyl acetate, and concentrated in vacuo. The residue was acidified with 1N aqueous hydrochloric acid and the resulting precipitate was filtered, washed with water, and dried in vacuo to obtain N-{[6-hydroxy-7-(1-methyl-1H-pyrazol-4-yl)-5-quinoxalinyl]carbonyl}glycine (0.018 g, 0.055 mmol, 64.9% yield) as a dark brown solid. ¹H NMR (400 MHz, DMSO-d₆) δ ppm 11.62 (t, J=5.6 Hz, 1H), 8.88 (d, J=2.0 Hz, 1H), 8.86 (d, J=2.0 Hz, 1H), 8.48 (s, 1H), 8.47 (s, 1H), 8.25 (s, 1H), 4.27 (d, J=5.6 Hz, 2H), 3.93 (s, 3H). MS (ES+) m/e 328 [M+H]⁺.

### Example 34

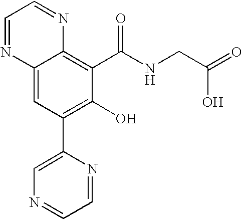

### N-{[6-hydroxy-7-(2-pyrazinyl)-5-quinoxalinyl]carbonyl}glycine

34(a) Ethyl N-{[6-hydroxy-7-(2-pyrazinyl)-5-quinoxalinyl]carbonyl}glycinate. To a solution of ethyl N-[(7-bromo-6-hydroxy-5-quinoxalinyl)carbonyl]glycinate (0.06 g, 0.169 mmol) in 1,4-dioxane (1.5 ml) was added 2-(tributylstannanyl)pyrazine (0.063 g, 0.169 mmol) and tetrakis(triphenylphosphine)palladium(0) (10 mg, 8.65 μmol) followed by heating to 150° C. for 20 min. in a Biotage Initiator® microwave synthesizer. Upon cooling, the reaction mixture was concentrated in vacuo and purified via flash column chromatography (0-10% methanol in ethyl acetate) to obtain ethyl N-{[6-hydroxy-7-(2-pyrazinyl)-5-quinoxalinyl]carbonyl}glycinate (0.022 g, 0.062 mmol, 36.8% yield) as a pale orange solid. ¹H NMR (400 MHz, DMSO-d6) □ ppm 16.40 (s, 1H), 11.62 (t, J=5.6 Hz, 1H), 9.34 (d, J=1.3 Hz, 1H), 9.02 (d, J=1.8 Hz, 1H), 8.99 (d, J=1.5 Hz, 1H), 8.87 (dd, J=2.4, 1.6 Hz, 1H), 8.74 (d, J=2.5 Hz, 1H), 8.62 (s, 1H), 4.37 (d, J=5.6 Hz, 2H), 4.19 (q, J=7.1 Hz, 2H), 1.25 (t, J=7.2 Hz, 3H). MS (ES+) m/e 354 [M+H]⁺.

34(b) N-{[6-hydroxy-7-(2-pyrazinyl)-5-quinoxalinyl]carbonyl}glycine. To a suspension of ethyl N-{[6-hydroxy-7-(2-pyrazinyl)-5-quinoxalinyl]carbonyl}glycinate (0.022 g, 0.062 mmol) in ethanol (1.0 mL) was added 1N aqueous sodium hydroxide (1.0 mL, 1.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(2-pyrazinyl)-5-quinoxalinyl]carbonyl}glycine (0.011 g, 0.034 mmol, 54.3% yield) as a peach solid. ¹H NMR (400 MHz, DMSO-d₆) δ ppm 11.56 (t, J=5.6 Hz, 1H), 9.32 (d, J=1.5 Hz, 1H), 8.99 (d, J=2.0 Hz, 1H), 8.96 (d, J=2.0 Hz, 1H), 8.86 (dd, J=2.4, 1.6 Hz, 1H), 8.72 (d, J=2.5 Hz, 1H), 8.59 (s, 1H), 4.28 (d, J=5.6 Hz, 2H). MS (ES+) m/e 326 [M+H]⁺.

### Example 35

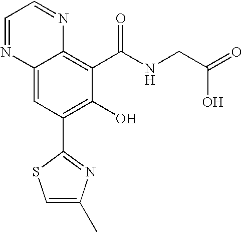

### N-{[6-hydroxy-7-(4-methyl-1,3-thiazol-2-yl)-5-quinoxalinyl]carbonyl}glycine

35(a) ethyl N-{[6-hydroxy-7-(4-methyl-1,3-thiazol-2-yl)-5-quinoxalinyl]carbonyl}glycinate. To a solution of ethyl N-[(7-bromo-6-hydroxy-5-quinoxalinyl)carbonyl]glycinate (0.06 g, 0.169 mmol) in 1,4-dioxane (1.5 ml) was added 4-methyl-2-(tributylstannanyl)-1,3-thiazole (0.066 g, 0.169 mmol) and tetrakis(triphenylphosphine)palladium(0) (9.79 mg, 8.47 μmol) followed by heating to 150° C. for 20 min. in a Biotage Initiator® microwave synthesizer. Upon cooling, the reaction mixture was concentrated in vacuo and purified via flash column chromatography (0-10% methanol in ethyl acetate) to obtain ethyl N-{[6-hydroxy-7-(4-methyl-1,3-thiazol-2-yl)-5-quinoxalinyl]carbonyl}glycinate (0.012 g, 0.032 mmol, 19.02% yield) as a dark yellow solid. ¹H NMR (400 MHz, DMSO-d₆) δ ppm 11.62 (t, J=5.7 Hz, 1H), 9.02 (s, 1H), 9.00 (d, J=1.0 Hz, 1H), 8.99 (d, J=0.8 Hz, 1H), 7.59 (s, 1H), 4.37 (d, J=5.1 Hz, 2H), 4.19 (q, J=7.1 Hz, 2H), 2.52 (s, 3H), 1.25 (t, J=7.2 Hz, 3H). MS (ES+) m/e 373 [M+H]⁺

35(b) N-{[6-hydroxy-7-(4-methyl-1,3-thiazol-2-yl)-5-quinoxalinyl]carbonyl}glycine. To a suspension of ethyl N-{[6-hydroxy-7-(4-methyl-1,3-thiazol-2-yl)-5-quinoxalinyl]carbonyl}glycinate (0.012 g, 0.032 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(4-methyl-1,3-thiazol-2-yl)-5-quinoxalinyl]carbonyl}glycine (0.006 g, 0.017 mmol, 54.1% yield) as a yellow solid. ¹H NMR (400 MHz, DMSO-d₆) δ ppm 11.56 (t, J=4.8 Hz, 1H), 8.99 (s, 1H), 8.97 (s, 2H), 7.57 (s, 1H), 4.29 (d, J=5.6 Hz, 2H), 2.52 (s, 3H). MS (ES+) m/e 345 [M+H]⁺.

### Example 36

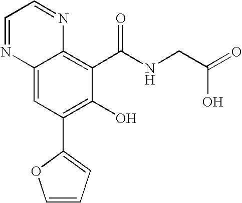

### N-{[7-(2-furanyl)-6-hydroxy-5-quinoxalinyl]carbonyl}glycine

36(a) ethyl N-{[7-(2-furanyl)-6-hydroxy-5-quinoxalinyl]carbonyl}glycinate. To a solution of ethyl N-[(7-bromo-6-hydroxy-5-quinoxalinyl)carbonyl]glycinate (0.051 g, 0.144 mmol) in 1,4-dioxane (1.5 ml) was added tributyl(2-furanyl)stannane (0.052 g, 0.145 mmol) and tetrakis(triphenylphosphine)palladium(0) (8.0 mg, 6.92 μmol) followed by heating to 150° C. for 20 min. in a Biotage Initiator® microwave synthesizer. Upon cooling, the reaction mixture was concentrated in vacuo and purified via flash column chromatography (0-10% methanol in dichloromethane) to obtain ethyl N-{[7-(2-furanyl)-6-hydroxy-5-quinoxalinyl]carbonyl}glycinate (0.03 g, 0.088 mmol, 61.0% yield) as an orange solid. ¹H NMR (400 MHz, DMSO-d₆) δ ppm 11.66 (t, J=5.8 Hz, 1H), 8.94 (s, 1H), 8.92 (s, 1H), 8.47 (s, 1H), 7.97 (s, 1H), 7.41 (d, J=3.0 Hz, 1H), 6.74 (s, 1H), 4.36 (d, J=5.1 Hz, 2H), 4.19 (q, J=7.2 Hz, 2H), 1.25 (t, J=7.1 Hz, 3H). MS (ES+) m/e 342 [M+H]⁺.

36(b) N-{[7-(2-furanyl)-6-hydroxy-5-quinoxalinyl]carbonyl}glycine. To a suspension of ethyl N-[(7-bromo-6-hydroxy-5-quinoxalinyl)carbonyl]glycinate (0.03 g, 0.088 mmol) in ethanol (2.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[7-(2-furanyl)-6-hydroxy-5-quinoxalinyl]carbonyl}glycine (0.024 g, 0.077 mmol, 87% yield) as a dark yellow solid. ¹H NMR (400 MHz, DMSO-d₆) δ ppm 11.61 (t, J=5.8 Hz, 1H), 8.93 (d, J=2.0 Hz, 1H), 8.91 (d, J=2.0 Hz, 1H), 8.45 (s, 1H), 7.96 (dd, J=1.8, 0.5 Hz, 1H), 7.40 (d, J=3.0 Hz, 1H), 6.74 (dd, J=3.3, 1.8 Hz, 1H), 4.27 (d, J=5.6 Hz, 1H). MS (ES+) m/e 314 [M+H]⁺.

### Example 37

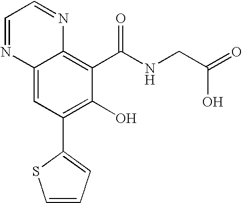

### N-{[6-hydroxy-7-(2-thienyl)-5-quinoxalinyl]carbonyl}glycine

37(a) ethyl N-{[6-hydroxy-7-(2-thienyl)-5-quinoxalinyl]carbonyl}glycinate. To a solution of ethyl N-[(7-bromo-6-hydroxy-5-quinoxalinyl)carbonyl]glycinate (0.05 g, 0.141 mmol) in 1,4-dioxane (1.5 ml) was added tributyl(2-thienyl)stannane (0.061 g, 0.162 mmol) and tetrakis(triphenylphosphine)palladium(0) (10 mg, 8.65 mmol) followed by heating to 150° C. for 20 min. in a Biotage Initiator® microwave synthesizer. Upon cooling, the reaction mixture was concentrated in vacuo and purified via flash column chromatography (0-10% methanol in dichloromethane) to obtain ethyl N-{[6-hydroxy-7-(2-thienyl)-5-quinoxalinyl]carbonyl}glycinate (0.038 g, 0.106 mmol, 75% yield) as a yellow solid. ¹H NMR (400 MHz, DMSO-d₆) δ ppm 11.65 (t, J=5.4 Hz, 1H), 8.93 (d, J=1.5 Hz, 1H), 8.91 (d, J=1.5 Hz, 1H), 8.59 (s, 1H), 8.04 (dd, J=3.8, 0.8 Hz, 1H), 7.79 (dd, J=5.2, 0.9 Hz, 1H), 7.25 (dd, J=5.1, 3.8 Hz, 1H), 4.36 (d, J=5.6 Hz, 2H), 4.19 (q, J=7.1 Hz, 2H), 1.24 (t, J=7.1 Hz, 3H). MS (ES+) m/e 358 [M+H]⁺.

37(b) N-{[6-hydroxy-7-(2-thienyl)-5-quinoxalinyl]carbonyl}glycine. To a suspension of ethyl N-{[6-hydroxy-7-(2-thienyl)-5-quinoxalinyl]carbonyl}glycinate (0.038 g, 0.106 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(2-thienyl)-5-quinoxalinyl]carbonyl}glycine (0.024 g, 0.073 mmol, 68.5% yield) as an orange solid. ¹H NMR (400 MHz, DMSO-d₆) δ ppm 12.96 (br. s., 1H), 11.61 (t, J=5.7 Hz, 1H), 8.93 (d, J=2.0 Hz, 1H), 8.92 (d, J=2.0 Hz, 1H), 8.60 (s, 1H), 8.05 (dd, J=3.7, 1.1 Hz, 1H), 7.79 (dd, J=5.2, 1.1 Hz, 1H), 7.25 (dd, J=5.2, 3.7 Hz, 1H), 4.28 (d, J=5.6 Hz, 2H). MS (ES+) m/e 330 [M+H]⁺.

### Example 38

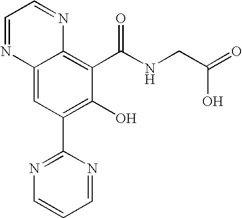

### N-{[6-hydroxy-7-(2-pyrimidinyl)-5-quinoxalinyl]carbonyl}glycine

38(a) ethyl N-{[6-hydroxy-7-(2-pyrimidinyl)-5-quinoxalinyl]carbonyl}glycinate. To a solution of ethyl N-[(7-bromo-6-hydroxy-5-quinoxalinyl)carbonyl]glycinate (0.120 g, 0.339 mmol) (prepared as in N2843-54-A1) in 1,4-dioxane (1.5 ml) was added 2-(tributylstannanyl)pyrimidine (0.138 g, 0.374 mmol) and tetrakis(triphenylphosphine)palladium(0) (0.020 g, 0.017 mmol) followed by heating to 150° C. for 60 min. in a Biotage Initiator® microwave synthesizer. Upon cooling, the reaction mixture was concentrated in vacuo and purified via flash column chromatography (0-100% ethyl acetate in hexane) to obtain ethyl N-{[6-hydroxy-7-(2-pyrimidinyl)-5-quinoxalinyl]carbonyl}glycinate (0.083 g, 0.235 mmol, 69.3% yield) as a pale orange solid. ¹H NMR (400 MHz, DMSO-d₆) δ ppm 14.90 (s, 1H), 10.12 (t, J=5.8 Hz, 1H), 9.09 (s, 1H), 9.07 (s, 1H), 8.97 (d, J=1.8 Hz, 1H), 8.91 (d, J=2.0 Hz, 1H), 8.81 (s, 1H), 7.68 (t, J=4.9 Hz, 1H), 4.23 (d, J=5.8 Hz, 2H), 4.18 (q, J=7.2 Hz, 2H), 1.26 (t, J=7.2 Hz, 3H). MS (ES+) m/e 354 [M+H]⁺.

38(b) N-{[6-hydroxy-7-(2-pyrimidinyl)-5-quinoxalinyl]carbonyl}glycine. To a suspension of ethyl N-{[6-hydroxy-7-(2-pyrimidinyl)-5-quinoxalinyl]carbonyl}glycinate (0.083 g, 0.235 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(2-pyrimidinyl)-5-quinoxalinyl]carbonyl}glycine (0.051 g, 0.157 mmol, 66.7% yield) as a yellow solid. ¹H NMR (400 MHz, DMSO-d₆) δ ppm 10.28 (t, J=5.6 Hz, 1H), 9.05 (d, J=5.1 Hz, 2H), 8.96 (d, J=1.8 Hz, 1H), 8.91 (d, J=1.8 Hz, 1H), 8.72 (s, 1H), 7.65 (t, J=4.9 Hz, 1H), 4.18 (d, J=5.6 Hz, 2H). MS (ES+) m/e 326 [M+H]⁺.

### Example 39

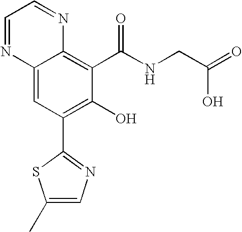

### N-{[6-hydroxy-7-(5-methyl-1,3-thiazol-2-yl)-5-quinoxalinyl]carbonyl}glycine

39(a) ethyl N-{[6-hydroxy-7-(5-methyl-1,3-thiazol-2-O-5-quinoxalinyl]carbonyl}glycinate. To a solution of ethyl N-[(7-bromo-6-hydroxy-5-quinoxalinyl)carbonyl]glycinate (0.075 g, 0.212 mmol) in 1,4-dioxane (2.0 ml) was added 5-methyl-2-(tributylstannanyl)-1,3-thiazole (0.082 g, 0.212 mmol) and tetrakis(triphenylphosphine)palladium(0) (0.012 g, 10.59 μmol) followed by heating to 150° C. for 45 min. in a Biotage Initiator® microwave synthesizer. Upon cooling, the reaction mixture was concentrated in vacuo and purified via flash column chromatography (0-100% ethyl acetate in hexanes) to obtain ethyl N-{[6-hydroxy-7-(5-methyl-1,3-thiazol-2-yl)-5-quinoxalinyl]carbonyl}glycinate (0.03 g, 0.081 mmol, 38.0% yield) as a pale orange solid. ¹H NMR (400 MHz, METHANOL-d₄) δ ppm 9.05 (s, 1H), 8.91 (d, J=2.0 Hz, 1H), 8.88 (d, J=1.8 Hz, 1H), 7.74 (d, J=1.0 Hz, 1H), 4.39 (s, 2H), 4.30 (q, J=7.2 Hz, 2H), 2.61 (d, J=1.0 Hz, 3H), 1.35 (t, J=7.2 Hz, 3H). MS (ES+) m/e 373 [M+H]⁺.

39(b) N-{[6-hydroxy-7-(5-methyl-1,3-thiazol-2-O-5-quinoxalinyl]carbonyl}glycine. To a suspension of ethyl N-{[6-hydroxy-7-(5-methyl-1,3-thiazol-2-yl)-5-quinoxalinyl]carbonyl}glycinate (0.03 g, 0.081 mmol) in ethanol (1.0 mL) was added sodium hydroxide (1N aqueous solution) (2.0 ml, 2.000 mmol). After stirring overnight at ambient temperature, the reaction was quenched with 1N aqueous hydrochloric acid and the resulting precipitate was filtered, washed with water, and dried in vacuo to obtain N-{[6-hydroxy-7-(5-methyl-1,3-thiazol-2-yl)-5-quinoxalinyl]carbonyl}glycine (0.021 g, 0.061 mmol, 76% yield) as a yellow solid. ¹H NMR (400 MHz, DMSO-d₆) δ ppm 13.00 (br. s., 1H), 11.58 (t, J=5.2 Hz, 1H), 8.98 (s, 1H), 8.98 (d, J=2.0 Hz, 2H), 7.83 (d, J=1.0 Hz, 1H), 4.29 (d, J=5.6 Hz, 2H), 2.56 (d, J=0.8 Hz, 3H). MS (ES+) m/e 345 [M+H]⁺.

### Example 40

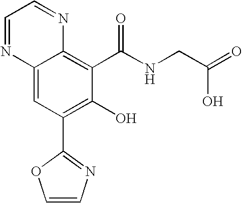

### N-{[6-hydroxy-7-(1,3-oxazol-2-yl)-5-quinoxalinyl]carbonyl}glycine

40(a) methyl 6-(methyloxy)-7-(1,3-oxazol-2-yl)-5-quinoxalinecarboxylate. To a solution of methyl 7-bromo-6-(methyloxy)-5-quinoxalinecarboxylate (example 10(b), 0.480 g, 1.616 mmol) in 1,4-dioxane (1.0 ml) was added 2-(tributylstannanyl)-1,3-oxazole (0.338 ml, 1.616 mmol) and tetrakis(triphenylphosphine)palladium(0) (0.093 g, 0.081 mmol) followed by heating to 150° C. for 1.0 h in a Biotage Initiator® microwave synthesizer. Upon cooling, the reaction mixture was diluted with ethyl acetate and concentrated in vacuo. The residue was purified via flash column chromatography (0-100% ethyl acetate in hexane) to obtain methyl 6-(methyloxy)-7-(1,3-oxazol-2-yl)-5-quinoxalinecarboxylate (0.124 g, 0.435 mmol, 26.9% yield) as a pale yellow solid. ¹H NMR (400 MHz, DMSO-d₆) δ ppm 9.03 (d, J=1.8 Hz, 1H), 9.02 (d, J=1.8 Hz, 1H), 8.69 (s, 1H), 8.43 (d, J=0.8 Hz, 1H), 7.58 (d, J=0.8 Hz, 1H), 3.99 (s, 3H), 3.91 (s, 3H). MS (ES+) m/e 286 [M+H]⁺.

40(b) 6-hydroxy-7-(1,3-oxazol-2-yl)-5-quinoxalinecarboxylic acid. A solution of methyl 6-(methyloxy)-7-(1,3-oxazol-2-yl)-5-quinoxalinecarboxylate (0.124 g, 0.435 mmol) in dichloromethane (10 mL) was treated with boron tribromide (1M solution in dichloromethane) (2.173 mL, 2.173 mmol) at room temperature overnight. The reaction mixture was poured into water and extracted twice with ethyl acetate. The combined organic portions were dried over magnesium sulfate, filtered and concentrated to give 6-hydroxy-7-(1,3-oxazol-2-yl)-5-quinoxalinecarboxylic acid (0.03 g, 0.117 mmol, 26.8% yield) as a yellow solid. ¹H NMR (400 MHz, DMSO-d₆) δ ppm 14.41 (br. s., 1H), 13.06 (br. s., 1H), 8.95-9.02 (m, 2H), 8.66 (s, 1H), 8.50 (d, J=0.8 Hz, 1H), 7.66 (d, J=1.0 Hz, 1H). MS (ES+) m/e 258 [M+H]⁺.

40(c) ethyl N-{[6-hydroxy-7-(1,3-oxazol-2-yl)-5-quinoxalinyl]carbonyl}glycinate. To a solution of 6-hydroxy-7-(1,3-oxazol-2-yl)-5-quinoxalinecarboxylic acid (0.03 g, 0.117 mmol) and glycine ethyl ester hydrochloride (0.065 g, 0.467 mmol) in dichloromethane (2.0 mL) were added triethylamine (0.098 mL, 0.700 mmol) and PyBOP (0.134 g, 0.257 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (10-40% ethyl acetate in hexanes) to afford ethyl N-{[6-hydroxy-7-(1,3-oxazol-2-yl)-5-quinoxalinyl]carbonyl}glycinate (0.027 g, 0.079 mmol, 67.6% yield) as a brown solid. ¹H NMR (400 MHz, DMSO-d₆) δ ppm 11.41 (t, J=5.4 Hz, 1H), 9.01 (d, J=2.0 Hz, 1H), 8.99 (d, J=2.0 Hz, 1H), 8.69 (s, 1H), 8.41 (d, J=0.8 Hz, 1H), 7.55 (d, J=0.8 Hz, 1H), 4.34 (d, J=5.6 Hz, 1H), 4.20 (q, J=6.6 Hz, 2H), 1.24 (t, J=3.5 Hz, 3H). MS (ES+) m/e 343 [M+H]⁺.

40(d) N-{[6-hydroxy-7-(1,3-oxazol-2-yl)-5-quinoxalinyl]carbonyl}glycine. To a suspension of ethyl N-{[6-hydroxy-7-(1,3-oxazol-2-yl)-5-quinoxalinyl]carbonyl}glycinate (0.027 g, 0.079 mmol) in ethanol (1.0 mL) was added 1N aqueous sodium hydroxide (1.0 ml, 1.000 mmol). Following stirring at ambient temperature for 1 h, the reaction mixture was concentrated in vacuo, and the residue was dissolved in water and acidified with 1N aqueous hydrochloric acid. The reaction mixture was concentrated in vacuo and purified via C-18 reverse phase flash column chromatography (0-100% acetonitrile in water) to afford N-{[6-hydroxy-7-(1,3-oxazol-2-yl)-5-quinoxalinyl]carbonyl}glycine (0.004 g, 0.013 mmol, 16.14% yield) s a yellow solid. ¹HNMR (400 MHz, DMSO-d₆) δ ppm 16.19 (br. s., 1H), 11.41 (t, J=5.6 Hz, 1H), 9.00 (d, J=2.0 Hz, 1H), 8.97 (d, J=2.0 Hz, 1H), 8.67 (s, 1H), 8.40 (d, J=0.5 Hz, 1H), 7.54 (d, J=0.8 Hz, 1H), 4.26 (d, J=5.6 Hz, 2H). MS (ES+) m/e 315 [M+H]⁺.

### Example 41

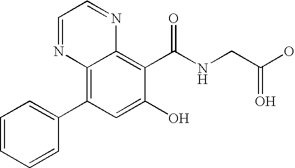

### N-[(6-hydroxy-8-phenyl-5-quinoxalinyl)carbonyl]glycine

41(a) methyl 4-bromo-2,6-difluorobenzoate. To a solution of 4-bromo-2,6-difluorobenzoic acid (5.0 g, 21.10 mmol) in dichloromethane (32.0 mL) and methanol (8.0 mL) was added (trimethylsilyl)diazomethane (2.0 M solution in diethyl ether) (15.0 mL, 30.0 mmol) slowly, dropwise via addition funnel. After stirring 15 min. at ambient temperature, the reaction mixture was concentrated in vacuo to afford methyl 4-bromo-2,6-difluorobenzoate (5.30 g, 21.11 mmol, 100% yield) as a clear, light orange oil. ¹H NMR (400 MHz, CHLOROFORM-d) δ ppm 7.17 (dd, J=8.8, 1.5 Hz, 2H), 3.95 (s, 3H). MS (ES+) m/e 251/253 [M+H]⁺.

41(b) methyl 4-bromo-2,6-difluoro-3-nitrobenzoate. To fuming nitric acid (3.0 ml, 67.1 mmol) at 0° C. was added concentrated sulfuric acid (5.6 ml, 105 mmol) dropwise via addition funnel. After stirring 5 min. at 0° C., methyl 4-bromo-2,6-difluorobenzoate (5.25 g, 20.91 mmol) was added portionwise. Following removal of the ice bath, the reaction mixture was stirred 1 h at ambient temperature and then poured into ice-water. The resulting precipitate was collected by filtration, washed with water, and dried in vacuo to afford methyl 4-bromo-2,6-difluoro-3-nitrobenzoate (5.81 g, 19.63 mmol, 94% yield) as a white solid. ¹H NMR (400 MHz, CHLOROFORM-d) δ ppm 7.37 (dd, J=8.5, 2.1 Hz, 1H), 4.00 (s, 3H). MS (ES+) m/e 296/298 [M+H]⁺.

41(c) methyl 2-amino-4-bromo-6-fluoro-3-nitrobenzoate. To a solution of methyl 4-bromo-2,6-difluoro-3-nitrobenzoate (1.00 g, 3.38 mmol) in methanol (7.0 mL) was added ammonium hydroxide (29% aqueous solution) (0.460 mL, 3.43 mmol). After stirring overnight at ambient temperature, the reaction mixture was concentrated in vacuo, triturated with methanol, filtered, and dried in vacuo to afford methyl 2-amino-4-bromo-6-fluoro-3-nitrobenzoate (0.705 g, 2.406 mmol, 71.2% yield) as a light yellow solid. ¹H NMR (400 MHz, DMSO-d₆) δ ppm 7.12 (br. s., 2H), 7.03 (d, J=10.4 Hz, 1H), 3.85 (s, 3H). MS (ES+) m/e 293/295 [M+H]⁺.

41(d) methyl 2-amino-4-bromo-6-(methyloxy)-3-nitrobenzoate. To a solution of sodium methoxide (25% in MeOH) (0.380 mL, 1.662 mmol) in methanol (5.0 mL) at 0° C. was added methyl 2-amino-4-bromo-6-fluoro-3-nitrobenzoate (0.400 g, 1.365 mmol). Following removal of the ice bath, the reaction mixture was stirred at ambient temperature for 3 h and then quenched with 1N aqueous hydrochloric acid. The resulting precipitate was filtered, washed with water, and dried in vacuo to afford methyl 2-amino-4-bromo-6-(methyloxy)-3-nitrobenzoate (0.340 g, 1.114 mmol, 82% yield) as a yellow solid. ¹H NMR (400 MHz, DMSO-d₆) δ ppm 6.74 (s, 1H), 6.31 (br. s., 2H), 3.81 (s, 3H), 3.79 (s, 3H). MS (ES+) m/e 305/307 [M+H]⁺.

41(e) methyl 3-amino-5-(methyloxy)-2-nitro-4-biphenylcarboxylate. A solution of methyl 2-amino-4-bromo-6-(methyloxy)-3-nitrobenzoate (0.305 g, 1.000 mmol), phenylboronic acid (0.146 g, 1.200 mmol), potassium carbonate (0.276 g, 1.999 mmol), and tetrakis(triphenylphosphine)palladium(0) (0.023 g, 0.020 mmol) in 1,4-dioxane (1.5 mL) and water (0.5 mL) was heated to 120° C. for 30 min in a Biotage Initiator® microwave synthesizer. Upon cooling, the reaction mixture was treated with water, diluted with brine, and extracted twice with ethyl acetate. The combined organic layers were dried over MgSO₄, filtered, concentrated in vacuo, and purified via flash column chromatography (10-30% ethyl acetate in hexanes) to afford methyl 3-amino-5-(methyloxy)-2-nitro-4-biphenylcarboxylate (0.279 g, 0.923 mmol, 92% yield) as a yellow solid. ¹H NMR (400 MHz, DMSO-d₆) δ ppm 7.39-7.47 (m, 3H), 7.33 (dd, J=7.8, 1.8 Hz, 2H), 6.34 (s, 3H), 3.85 (s, 3H), 3.83 (s, 3H). MS (ES+) m/e 303 [M+H]⁺.

41(f) 6-hydroxy-8-phenyl-5-quinoxalinecarboxylic acid. To a solution of methyl 3-amino-5-(methyloxy)-2-nitro-4-biphenylcarboxylate (0.240 g, 0.794 mmol) in ethyl acetate (3.0 mL) was added 10% palladium on charcoal (0.084 g, 0.079 mmol) followed by evacuation of the reaction vessel and purging with 1 atmosphere of hydrogen. Following stirring at ambient temperature for 24 h, the reaction mixture was filtered through Celite®, washed through with ethyl acetate, and concentrated in vacuo. The resulting residue was dissolved in acetonitrile (2.0 mL), treated with glyoxal (40% aqueous solution) (0.100 mL, 0.872 mmol), and heated to 120° C. for 20 min. in a Biotage Initiator® microwave synthesizer. Upon cooling, the reaction mixture was concentrated in vacuo, diluted with dichloromethane (5.0 mL), and treated with boron tribromide (1M solution in dichloromethane) (3.00 mL, 3.00 mmol). The reaction mixture was stirred overnight at ambient temperature, quenched by water, diluted with brine, and extracted thrice with dichloromethane. The combined organic layers were dried over MgSO₄, filtered, concentrated in vacuo, and purified via flash column chromatography (0-4% methanol in dichloromethane) to afford 6-hydroxy-8-phenyl-5-quinoxalinecarboxylic acid (0.086 g, 0.323 mmol, 40.7% yield) as a beige solid. ¹H NMR (400 MHz, DMSO-d₆) δ ppm 15.9 (br. s., 1H), 13.2 (br. s., 1H), 9.01 (s, 2H), 7.70 (dd, J=7.8, 1.8 Hz, 2H), 7.66 (s, 1H), 7.48-7.56 (m, 3H). MS (ES+) m/e 267 [M+H]⁺.

41(g) ethyl N-[(6-hydroxy-8-phenyl-5-quinoxalinyl)carbonyl]glycinate. To a solution of 6-hydroxy-8-phenyl-5-quinoxalinecarboxylic acid (0.081 g, 0.304 mmol) and glycine ethyl ester hydrochloride (0.085 g, 0.608 mmol) in N,N-dimethylformamide (3.0 mL) were added triethylamine (0.130 mL, 0.933 mmol) and PyBOP (0.174 g, 0.335 mmol). The reaction mixture was stirred overnight at ambient temperature, quenched by water, diluted with brine, and extracted twice with EtOAc. The combined organic layers were dried over MgSO₄, filtered, concentrated in vacuo, and purified via flash column chromatography (20-50% ethyl acetate in hexanes) to afford ethyl N-[(6-hydroxy-8-phenyl-5-quinoxalinyl)carbonyl]glycinate (0.095 g, 0.270 mmol, 89% yield) as a white solid. ¹H NMR (400 MHz, DMSO-d₆) δ ppm 15.3 (s, 1H), 11.5 (t, J=5.6 Hz, 1H), 8.96 (d, J=2.0 Hz, 1H), 8.90 (d, J=2.0 Hz, 1H), 7.68 (dd, J=8.0, 1.6 Hz, 2H), 7.54 (s, 1H), 7.45-7.53 (m, 3H), 4.33 (d, J=5.6 Hz, 2H), 4.19 (q, J=7.1 Hz, 2H), 1.24 (t, J=7.1 Hz, 3H). MS (ES+) m/e 352 [M+H]⁺.

41(h) N-[(6-hydroxy-8-phenyl-5-quinoxalinyl)carbonyl]glycine. To a suspension of ethyl N-[(6-hydroxy-8-phenyl-5-quinoxalinyl)carbonyl]glycinate (0.090 g, 0.256 mmol) in methanol (2.0 mL) and tetrahydrofuran (2.0 mL) was added 1N aqueous sodium hydroxide (1.0 mL, 1.000 mmol). After stirring 30 min. at ambient temperature, the reaction was quenched with 1N aqueous hydrochloric acid and the resulting precipitate was filtered, washed with water, and dried in vacuo to afford N-[(6-hydroxy-8-phenyl-5-quinoxalinyl)carbonyl]glycine (0.077 g, 0.238 mmol, 93% yield) as an off-white solid. ¹H NMR (400 MHz, DMSO-d₆) δ ppm 15.4 (br. s., 1H), 12.9 (br. s., 1H), 11.5 (t, J=5.6 Hz, 1H), 8.97 (d, J=2.0 Hz, 1H), 8.89 (d, J=2.0 Hz, 1H), 7.68 (dd, J=7.8, 1.5 Hz, 2H), 7.54 (s, 1H), 7.46-7.53 (m, 3H), 4.26 (d, J=5.6 Hz, 2H). MS (ES+) m/e 324 [M+H]⁺.

### Example 42

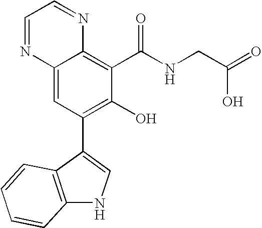

### N-{[6-hydroxy-7-(1H-indol-3-yl)-5-quinoxalinyl]carbonyl}glycine

42(a) ethyl N-({6-hydroxy-7-[1-(phenylsulfonyl)-1H-indol-3-yl]-5-quinoxalinyl}carbonyl)glycinate. A solution of ethyl N-[(7-bromo-6-hydroxy-5-quinoxalinyl)carbonyl]glycinate (0.063 g, 0.178 mmol), 1-(phenylsulfonyl)-3-(4,4,5,5-tetramethyl-1,3,2-dioxaborolan-2-yl)-1H-indole (0.068 g, 0.178 mmol), potassium carbonate (0.074 g, 0.534 mmol), and tetrakis(triphenylphosphine)palladium(0) (6.0 mg, 5.19 μmol) in 1,4-dioxane (2.0 ml) and water (0.667 ml) was heated to 100° C. for 20 min. in a Biotage Initiator® microwave synthesizer. Upon cooling, the reaction mixture was filtered through Celite®, washed through with ethyl acetate, and concentrated in vacuo. The residue was purified via flash column chromatography (10% methanol in dichloromethane). ¹H NMR (400 MHz, DMSO-d₆) δ ppm 11.63 (br. s., 1H), 8.21-8.54 (m, 2H), 8.11 (d, J=7.8 Hz, 2H), 8.05 (d, J=8.3 Hz, 1H), 7.69-7.77 (m, 2H), 7.64 (t, J=7.6 Hz, 2H), 7.39-7.50 (m, 1H), 7.28-7.39 (m, 1H), 4.35 (dd, J=10.6, 4.3 Hz, 2H), 4.19 (q, J=7.2 Hz, 2H), 1.25 (t, J=7.1 Hz, 3H). MS (ES+) m/e 531 [M+H]⁺.

42(b) N-{[6-hydroxy-7-(1H-indol-3-yl)-5-quinoxalinyl]carbonyl}glycine. To a suspension of ethyl N-({6-hydroxy-7-[1-(phenylsulfonyl)-1H-indol-3-yl]-5-quinoxalinyl}carbonyl)glycinate (0.033 g, 0.062 mmol) in ethanol (1.0 mL) was added 1N aqueous sodium hydroxide (1.0 ml, 1.000 mmol). After stirring at ambient temperature overnight, the reaction was refluxed for 1 h, then cooled down to ambient temperature and quenched with 1N aqueous hydrochloric acid. The resulting precipitate was filtered, washed with water, and dried in vacuo to obtain N-{[6-hydroxy-7-(1H-indol-3-yl)-5-quinoxalinyl]carbonyl}glycine (11 mg, 0.030 mmol, 48.8% yield) as a red solid. ¹H NMR (400 MHz, DMSO-d₆) δ ppm 11.68 (t, J=5.3 Hz, 1H), 11.67 (br. s., 1H), 8.90 (d, J=2.0 Hz, 1H), 8.88 (d, J=2.0 Hz, 1H), 8.39 (s, 1H), 8.07 (d, J=2.8 Hz, 1H), 7.86-7.91 (m, 1H), 7.50-7.55 (m, 1H), 7.21 (td, J=7.5, 1.4 Hz, 1H), 7.16 (td, J=7.4, 1.1 Hz, 1H), 4.28 (d, J=5.6 Hz, 2H). MS (ES+) m/e 363 [M+H]⁺.

### Example 43

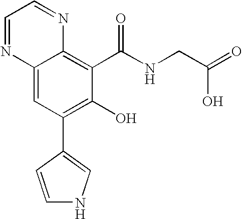

### N-{[6-hydroxy-7-(1H-pyrrol-3-yl)-5-quinoxalinyl]carbonyl}glycine

43(a) N-[(6-hydroxy-7-{1-[tris(1-methylethyl)silyl]-1H-pyrrol-3-yl}-5-quinoxalinyl)carbonyl]glycine. A solution of ethyl N-[(7-bromo-6-hydroxy-5-quinoxalinyl)carbonyl]glycinate (0.085 g, 0.240 mmol), 1-(triisopropylsilyl)pyrrole-3-boronic acid (0.064 g, 0.240 mmol), potassium carbonate (0.100 g, 0.720 mmol), and tetrakis(triphenylphosphine)palladium(0) (12 mg, 10.38 μmol) in 1,4-dioxane (2.0 ml) and water (0.667 ml) was heated to 100° C. for 1 h. in a Biotage Initiator® microwave synthesizer. The reaction mixture was diluted with brine and extracted thrice with ethyl acetate. The organic portions were dried over magnesium sulfate, filtered and concentrated. The residue was purified via flash column chromatography (10% methanol in dichloromethane) to obtain N-[(6-hydroxy-7-{1-[tris(1-methylethyl)silyl]-1H-pyrrol-3-yl}-5-quinoxalinyl)carbonyl]glycine (0.05 g, 0.107 mmol, 44.5% yield). ¹H NMR (400 MHz, DMSO-d₆) δ ppm 11.66 (t, J=5.4 Hz, 1H), 8.86 (d, J=2.3 Hz, 1H), 8.83 (d, J=2.0 Hz, 1H), 8.38 (s, 1H), 7.79-7.84 (m, 1H), 7.01 (dd, J=2.9, 1.4 Hz, 1H), 6.93-6.99 (m, 1H), 4.26 (d, J=5.6 Hz, 2H), 1.44-1.64 (m, 3H), 1.10 (s, 9H), 1.08 (s, 9H). MS (ES+) m/e 469 [M+H]⁺.

43(b) N-{[6-hydroxy-7-(1H-pyrrol-3-yl)-5-quinoxalinyl]carbonyl}glycine. To a suspension of N-[(6-hydroxy-7-{1-[tris(1-methylethyl)silyl]-1H-pyrrol-3-yl}-5-quinoxalinyl)carbonyl]glycine (0.05 g, 0.107 mmol) in ethanol (1.0 mL) was added 1N aqueous sodium hydroxide (1.0 ml, 1.000 mmol). After stirring at ambient temperature, the reaction was quenched with 1N aqueous hydrochloric acid and the resulting precipitate was filtered, washed with water, and dried in vacuo to obtain N-{[6-hydroxy-7-(1H-pyrrol-3-yl)-5-quinoxalinyl]carbonyl}glycine (27 mg, 0.086 mmol, 81% yield) as a dark yellow solid. ¹H NMR (400 MHz, DMSO-d₆) δ ppm 16.49 (s, 1H), 12.93 (br. s., 1H), 11.68 (t, J=5.4 Hz, 1H), 11.20 (br. s., 1H), 8.84 (d, J=1.8 Hz, 1H), 8.81 (d, J=2.0 Hz, 1H), 8.33 (s, 1H), 7.63-7.75 (m, 1H), 6.90 (q, J=2.5 Hz, 1H), 6.78-6.85 (m, 1H), 4.27 (d, J=5.6 Hz, 2H). MS (ES+) m/e 313 [M+H]⁺.

### Example 44

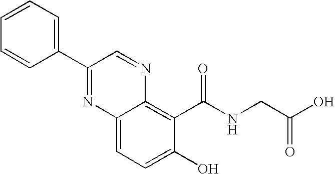

### N-[(6-hydroxy-2-phenyl-5-quinoxalinyl)carbonyl]glycine

44(a) methyl 6-(methyloxy)-2-phenyl-5-quinoxalinecarboxylate. A solution of methyl 2-chloro-6-(methyloxy)-5-quinoxalinecarboxylate (example 4(c), 0.250 g, 0.989 mmol), phenylboronic acid (0.145 g, 1.187 mmol), potassium carbonate (0.274 g, 1.979 mmol), and tetrakis(triphenylphosphine)palladium(0) (0.023 g, 0.020 mmol) in 1,4-dioxane (1.5 mL) and water (0.5 mL) was heated to 120° C. for 30 min in a Biotage Initiator® microwave synthesizer. Upon cooling, the reaction mixture was treated with water, diluted with brine, and extracted twice with ethyl acetate. The combined organic layers were dried over MgSO₄, filtered, concentrated in vacuo, and purified via flash column chromatography (10-40% ethyl acetate in hexanes) to afford methyl 6-(methyloxy)-2-phenyl-5-quinoxalinecarboxylate (0.267 g, 0.907 mmol, 92% yield) as a white solid. ¹H NMR (400 MHz, CHLOROFORM-d) δ ppm 9.32 (s, 1H), 8.22 (d, J=9.3 Hz, 1H), 8.15 (dd, J=8.2, 1.4 Hz, 2H), 7.59 (d, J=9.3 Hz, 1H), 7.50-7.60 (m, 3H), 4.10 (s, 3H), 4.06 (s, 3H). MS (ES+) m/e 295 [M+H]⁺.

44(b) 6-hydroxy-2-phenyl-5-quinoxalinecarboxylic acid. To a solution of methyl 6-(methyloxy)-2-phenyl-5-quinoxalinecarboxylate (0.260 g, 0.883 mmol) in dichloromethane (5.0 mL) was added boron tribromide (1M solution in dichloromethane) (3.00 mL, 3.00 mmol). The reaction mixture was stirred overnight at ambient temperature, quenched by water, filtered, washed with water, and dried in vacuo to afford 6-hydroxy-2-phenyl-5-quinoxalinecarboxylic acid (0.230 g, 0.864 mmol, 98% yield) as a yellow solid. ¹H NMR (400 MHz, DMSO-d₆) δ ppm 15.6 (br. s., 1H), 12.7 (br. s., 1H), 9.62 (s, 1H), 8.31 (d, J=9.3 Hz, 1H), 8.30 (dd, J=8.2, 1.5 Hz, 2H), 7.68 (d, J=9.3 Hz, 1H), 7.55-7.65 (m, 3H). MS (ES+) m/e 267 [M+H]⁺.

44(c) ethyl N-[(6-hydroxy-2-phenyl-5-quinoxalinyl)carbonyl]glycinate. To a solution of 6-hydroxy-2-phenyl-5-quinoxalinecarboxylic acid (0.200 g, 0.751 mmol) and glycine ethyl ester hydrochloride (0.315 g, 2.254 mmol) in N,N-dimethylformamide (5.0 mL) were added triethylamine (0.420 mL, 3.01 mmol) and PyBOP (0.586 g, 1.127 mmol). The reaction mixture was stirred overnight at ambient temperature, quenched by water, diluted with brine, and extracted twice with EtOAc. The combined organic layers were dried over MgSO₄, filtered, concentrated in vacuo, and purified via flash column chromatography (20-50% ethyl acetate in hexanes) to afford ethyl N-[(6-hydroxy-2-phenyl-5-quinoxalinyl)carbonyl]glycinate (0.223 g, 0.635 mmol, 84% yield) as a white solid. ¹H NMR (400 MHz, DMSO-d₆) δ ppm 15.1 (s, 1H), 11.4 (t, J=5.6 Hz, 1H), 9.54 (s, 1H), 8.31 (dd, J=8.1, 1.5 Hz, 2H), 8.24 (d, J=9.3 Hz, 1H), 7.59 (d, J=9.3 Hz, 1H), 7.54-7.64 (m, 3H), 4.35 (d, J=5.6 Hz, 2H), 4.19 (q, J=7.1 Hz, 2H), 1.25 (t, J=7.1 Hz, 3H). MS (ES+) m/e 352 [M+H]⁺.

44(d) N-[(6-hydroxy-2-phenyl-5-quinoxalinyl)carbonyl]glycine. To a solution of ethyl N-[(6-hydroxy-2-phenyl-5-quinoxalinyl)carbonyl]glycinate (0.076 g, 0.216 mmol) in methanol (1.0 mL) and tetrahydrofuran (1.0 mL) was added 1N aqueous sodium hydroxide (1.00 mL, 1.000 mmol). After stirring 15 min. at ambient temperature, the reaction was quenched with 1N aqueous hydrochloric acid and the resulting precipitate was filtered, washed with methanol, and dried in vacuo to afford N-[(6-hydroxy-2-phenyl-5-quinoxalinyl)carbonyl]glycine (0.066 g, 0.204 mmol, 94% yield) as a light, pale yellow solid. ¹H NMR (400 MHz, DMSO-d₆) δ ppm 15.2 (s, 1H), 13.0 (br. s., 1H), 11.4 (t, J=5.6 Hz, 1H), 9.52 (s, 1H), 8.30 (d, J=7.1 Hz, 2H), 8.22 (d, J=9.3 Hz, 1H), 7.57 (d, J=9.3 Hz, 1H), 7.52-7.64 (m, 3H), 4.27 (d, J=5.6 Hz, 2H). MS (ES+) m/e 324 [M+H]⁺.

### Example 45

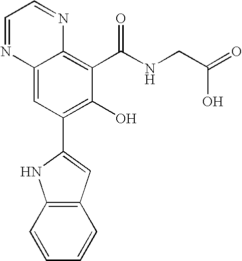

### N-{[6-hydroxy-7-(1H-indol-2-yl)-5-quinoxalinyl]carbonyl}glycine

45(a) 1,1-dimethylethyl 2-[8-({[2-(ethyloxy)-2-oxoethyl]amino}carbonyl)-7-hydroxy-6-quinoxalinyl]-1H-indole-1-carboxylate. A solution of ethyl N-[(7-bromo-6-hydroxy-5-quinoxalinyl)carbonyl]glycinate (0.086 g, 0.243 mmol), 1-(tert-butoxycarbonyl)indole)-2-boronic acid (0.063 g, 0.243 mmol), potassium carbonate (0.101 g, 0.728 mmol), and tetrakis(triphenylphosphine)palladium(0) (8.42 mg, 7.28 mmol) in 1,4-dioxane (2.0 ml) and water (0.667 ml) was heated to 100° C. for 20 min. in a Biotage Initiator® microwave synthesizer. Upon cooling, the reaction mixture was filtered through Celite®, washed through with ethyl acetate, and concentrated in vacuo. The residue was purified via flash column chromatography (0-10% methanol in dichloromethane) to obtain 1,1-dimethylethyl 2-[8-({[2-(ethyloxy)-2-oxoethyl]amino}carbonyl)-7-hydroxy-6-quinoxalinyl]-1H-indole-1-carboxylate (0.08 g, 0.139 mmol, 57.1% yield). ¹H NMR (400 MHz, CHLOROFORM-d) δ ppm 15.76 (s, 1H), 11.74 (t, J=4.9 Hz, 1H), 8.80 (d, J=2.0 Hz, 1H), 8.78 (d, J=2.0 Hz, 1H), 8.22 (s, 1H), 7.65-7.75 (m, 1H), 7.63 (d, J=7.8 Hz, 1H), 7.33-7.44 (m, 1H), 7.25-7.31 (m, 1H), 6.74 (s, 1H), 4.38 (d, J=5.3 Hz, 2H), 4.30 (q, J=7.1 Hz, 2H), 1.38 (s, 9H), 1.35 (t, J=7.3 Hz, 3H). MS (ES+) m/e 491 [M+H]⁺.

45(b) N-{[6-hydroxy-7-(1H-indol-2-yl)-5-quinoxalinyl]carbonyl}glycine. To a suspension of 1,1-dimethylethyl 2-[8-({[2-(ethyloxy)-2-oxoethyl]amino}carbonyl)-7-hydroxy-6-quinoxalinyl]-1H-indole-1-carboxylate (0.08 g, 0.163 mmol) in ethanol (1.0 mL) was added 1N aqueous sodium hydroxide (1.0 ml, 1.000 mmol). After stirring 30 min. at ambient temperature, the reaction was refluxed to 78° C. for 30 min., then cooled down to ambient temperature and quenched with 1N aqueous hydrochloric acid. The resulting precipitate was filtered, washed with water, and dried in vacuo to obtain N-{[6-hydroxy-7-(1H-indol-2-yl)-5-quinoxalinyl]carbonyl}glycine (0.012 g, 0.033 mmol, 20.31% yield) as a red brick solid. ¹H NMR (400 MHz, DMSO-d₆) δ ppm 11.75 (br. s., 1H), 11.67 (t, J=5.8 Hz, 1H), 8.94 (d, J=2.0 Hz, 1H), 8.91 (d, J=2.0 Hz, 1H), 8.71 (s, 1H), 7.64 (d, J=7.6 Hz, 1H), 7.50 (dd, J=8.1, 0.8 Hz, 1H), 7.48 (d, J=1.5 Hz, 1H), 7.14-7.23 (m, 1H), 6.99-7.08 (m, 1H), 4.28 (d, J=5.6 Hz, 2H). MS (ES+) m/e 363 [M+H]⁺.

### Example 46

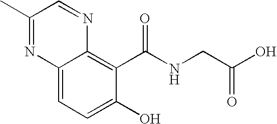

### N-[(6-hydroxy-2-methyl-5-quinoxalinyl)carbonyl]glycine

46(a) methyl 2-methyl-6-(methyloxy)-5-quinoxalinecarboxylate. A solution of methyl 2-chloro-6-(methyloxy)-5-quinoxalinecarboxylate (example 4(c), 0.140 g, 0.554 mmol), trimethylboroxine (0.080 mL, 0.575 mmol), potassium carbonate (0.153 g, 1.108 mmol), and tetrakis(triphenylphosphine)palladium(0) (0.013 g, 0.011 mmol) in 1,4-dioxane (1.5 mL) and water (0.5 mL) was heated to 120° C. for 30 min in a Biotage Initiator® microwave synthesizer. Upon cooling, the reaction mixture was treated with water, diluted with brine, and extracted twice with ethyl acetate. The combined organic layers were dried over MgSO₄, filtered, concentrated in vacuo, and purified via flash column chromatography (20-60% ethyl acetate in hexanes) to afford methyl 2-methyl-6-(methyloxy)-5-quinoxalinecarboxylate (0.121 g, 0.521 mmol, 94% yield) as a white solid. ¹H NMR (400 MHz, CHLOROFORM-d) δ ppm 8.72 (s, 1H), 8.07 (d, J=9.3 Hz, 1H), 7.53 (d, J=9.3 Hz, 1H), 4.06 (s, 3H), 4.02 (s, 3H), 2.74 (s, 3H). MS (ES+) m/e 233 [M+H]⁺.

46(b) 6-hydroxy-2-methyl-5-quinoxalinecarboxylic acid. To a solution of methyl 2-methyl-6-(methyloxy)-5-quinoxalinecarboxylate (0.121 g, 0.521 mmol) in dichloromethane (5.0 mL) was added boron tribromide (1M solution in dichloromethane) (1.50 mL, 1.500 mmol). The reaction mixture was overnight at ambient temperature, quenched by water, diluted with brine, and extracted twice with dichloromethane. The combined organic layers were dried over MgSO₄, filtered, concentrated in vacuo, and purified via flash column chromatography (20-100% ethyl acetate in hexanes) to afford 6-hydroxy-2-methyl-5-quinoxalinecarboxylic acid (0.092 g, 0.451 mmol, 86% yield) as a light yellow solid. ¹H NMR (400 MHz, DMSO-d₆) δ ppm 15.7 (br. s., 1H), 12.8 (br. s., 1H), 8.90 (s, 1H), 8.18 (d, J=9.3 Hz, 1H), 7.61 (d, J=9.3 Hz, 1H), 2.73 (s, 3H). MS (ES+) m/e 205 [M+H]⁺.

46(c) ethyl N-[(6-hydroxy-2-methyl-5-quinoxalinyl)carbonyl]glycinate. To a solution of 6-hydroxy-2-methyl-5-quinoxalinecarboxylic acid (0.088 g, 0.431 mmol) and glycine ethyl ester hydrochloride (0.180 g, 1.293 mmol) in N,N-dimethylformamide (5.0 mL) were added triethylamine (0.240 mL, 1.724 mmol) and PyBOP (0.336 g, 0.646 mmol). The reaction mixture was stirred overnight at ambient temperature, quenched by water, diluted with brine, and extracted twice with EtOAc. The combined organic layers were dried over MgSO₄, filtered, concentrated in vacuo, and purified via flash column chromatography (20-50% ethyl acetate in hexanes) to afford ethyl N-[(6-hydroxy-2-methyl-5-quinoxalinyl)carbonyl]glycinate (0.110 g, 0.380 mmol, 88% yield) as a white solid. ¹H NMR (400 MHz, CHLOROFORM-d) δ ppm 15.0 (s, 1H), 11.5 (t, J=5.3 Hz, 1H), 8.77 (s, 1H), 8.19 (d, J=9.3 Hz, 1H), 7.51 (d, J=9.3 Hz, 1H), 4.36 (d, J=5.3 Hz, 2H), 4.30 (q, J=7.2 Hz, 2H), 2.83 (s, 3H), 1.35 (t, J=7.2 Hz, 3H). MS (ES+) m/e 290 [M+H]⁺.

46(d) N-[(6-hydroxy-2-methyl-5-quinoxalinyl)carbonyl]glycine. To a solution of ethyl N-[(6-hydroxy-2-methyl-5-quinoxalinyl)carbonyl]glycinate (0.110 g, 0.380 mmol) in methanol (1.0 mL) and tetrahydrofuran (1.0 mL) was added 1N aqueous sodium hydroxide (1.00 mL, 1.000 mmol). After stirring 15 min. at ambient temperature, the reaction was quenched with 1N aqueous hydrochloric acid and the resulting precipitate was filtered, washed with water, and dried in vacuo to afford N-[(6-hydroxy-2-methyl-5-quinoxalinyl)carbonyl]glycine (0.088 g, 0.337 mmol, 89% yield) as an off-white solid. ¹H NMR (400 MHz, DMSO-d₆) δ ppm 15.1 (s, 1H), 12.9 (br. s., 1H), 11.4 (t, J=5.3 Hz, 1H), 8.85 (s, 1H), 8.08 (d, J=9.3 Hz, 1H), 7.50 (d, J=9.3 Hz, 1H), 4.24 (d, J=5.3 Hz, 2H), 2.70 (s, 3H). MS (ES+) m/e 262 [M+H]⁺.

### Example 47

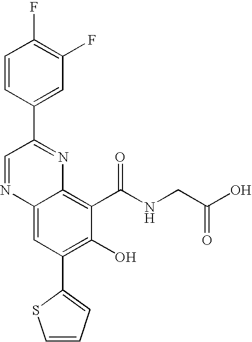

### N-{[3-(3,4-difluorophenyl)-6-hydroxy-7-(2-thienyl)-5-quinoxalinyl]carbonyl}glycine

47(a) ethyl N-{[3-(3,4-difluorophenyl)-6-hydroxy-7-(2-thienyl)-5-quinoxalinyl]carbonyl}glycinate. To a solution of ethyl N-{[7-bromo-3-(3,4-difluorophenyl)-6-hydroxy-5-quinoxalinyl]carbonyl}glycinate (example 27(c), 0.057 g, 0.122 mmol) in dioxane (2.0 mL) was added 2-tributylstannanylthiophene (0.050 mL, 0.157 mmol) and tetrakis(triphenylphosphine)palladium(0) (0.004 g, 3.46 mmol) followed by heating to 150° C. for 20 min. in a Biotage Initiator® microwave synthesizer. Upon cooling, the reaction mixture was concentrated in vacuo, triturated with dichloromethane, filtered, and dried in vacuo to afford ethyl N-{[3-(3,4-difluorophenyl)-6-hydroxy-7-(2-thienyl)-5-quinoxalinyl]carbonyl}glycinate (0.045 g, 0.096 mmol, 78% yield) as a mustard solid. ¹H NMR (400 MHz, DMSO-d₆) δ ppm 11.5 (t, J=5.2 Hz, 1H), 9.53 (s, 1H), 8.62 (s, 1H), 8.44 (ddd, J=11.8, 7.9, 2.0 Hz, 1H), 8.18-8.25 (m, 1H), 8.06 (d, J=3.0 Hz, 1H), 7.80 (d, J=5.1 Hz, 1H), 7.68 (dt, J=9.6, 8.8 Hz, 1H), 7.25 (dd, J=4.7, 4.2 Hz, 1H), 4.47 (d, J=5.2 Hz, 2H), 4.23 (q, J=7.1 Hz, 2H), 1.25 (t, J=7.1 Hz, 3H). MS (ES+) m/e 470 [M+H]⁺.

47(b) N-{[3-(3,4-difluorophenyl)-6-hydroxy-7-(2-thienyl)-5-quinoxalinyl]carbonyl}glycine. To a suspension of ethyl N-{[3-(3,4-difluorophenyl)-6-hydroxy-7-(2-thienyl)-5-quinoxalinyl]carbonyl}glycinate (0.043 g, 0.092 mmol) in methanol (1.0 mL) and tetrahydrofuran (1.0 mL) was added 1N aqueous sodium hydroxide (0.500 mL, 0.500 mmol). After stirring 30 min. at ambient temperature, the reaction was quenched with 1N aqueous hydrochloric acid and the resulting precipitate was filtered, washed with water, and dried in vacuo to afford N-{[3-(3,4-difluorophenyl)-6-hydroxy-7-(2-thienyl)-5-quinoxalinyl]carbonyl}glycine (0.036 g, 0.082 mmol, 89% yield) as a pale orange solid. ¹H NMR (400 MHz, DMSO-d₆) δ ppm 13.3 (br. s., 1H), 11.4 (t, J=5.0 Hz, 1H), 9.50 (s, 1H), 8.60 (s, 1H), 8.46 (ddd, J=11.7, 7.8, 2.0 Hz, 1H), 8.18-8.27 (m, 1H), 8.05 (dd, J=3.8, 1.0 Hz, 1H), 7.79 (dd, J=5.1, 1.0 Hz, 1H), 7.64 (dt, J=10.2, 8.5 Hz, 1H), 7.25 (dd, J=5.1, 3.8 Hz, 1H), 4.38 (d, J=5.0 Hz, 2H). MS (ES+) m/e 442 [M+H]⁺.

### Example 48

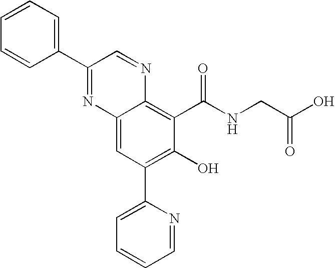

### N-{[6-hydroxy-2-phenyl-7-(2-pyridinyl)-5-quinoxalinyl]carbonyl}glycine

48(a) methyl 7-bromo-6-(methyloxy)-2-oxo-1,2-dihydro-5-quinoxalinecarboxylate. To a solution of methyl 2-amino-5-bromo-6-(methyloxy)-3-nitrobenzoate (example 13(a), 1.08 g, 3.54 mmol) in ethyl acetate (10.0 mL) was added 10% palladium on charcoal (0.264 g, 0.248 mmol) followed by evacuation of the reaction vessel and purging with 1 atmosphere of hydrogen. Following stirring at ambient temperature for 2 h, the reaction mixture was filtered through Celite®, washed through with ethyl acetate, and concentrated in vacuo. The resulting residue was dissolved in acetonitrile (10.00 mL), treated with ethyl glyoxalate (˜50% solution in toluene) (0.795 g, 3.89 mmol), and stirred overnight at room temperature. The resulting precipitate was filtered, washed with acetonitrile, and dried in vacuo to afford methyl 7-bromo-6-(methyloxy)-2-oxo-1,2-dihydro-5-quinoxalinecarboxylate (0.729 g, 2.328 mmol, 65.8% yield) as a pale pink solid. ¹H NMR (400 MHz, DMSO-d₆) δ ppm 12.56 (br. s., 1H), 8.23 (s, 1H), 7.59 (s, 1H), 3.91 (s, 3H), 3.81 (s, 3H). MS (ES+) m/e 313/315 [M+H]⁺.

48(b) methyl 6-(methyloxy)-2-oxo-7-(2-pyridinyl)-1,2-dihydro-5-quinoxalinecarboxylate. To a solution of methyl 7-bromo-6-(methyloxy)-2-oxo-1,2-dihydro-5-quinoxalinecarboxylate (0.729 g, 2.328 mmol) in 1,4-dioxane (5.0 ml) was added 2-(tributylstannanyl)pyridine (0.981 g, 2.67 mmol) and tetrakis(triphenylphosphine)palladium(0) (0.126 g, 0.109 mmol) followed by heating to 150° C. for 120 min. in a Biotage Initiator® microwave synthesizer. Upon cooling, the reaction mixture was concentrated in vacuo and purified via flash column chromatography (0-100% ethyl acetate in hexanes) to obtain methyl 6-(methyloxy)-2-oxo-7-(2-pyridinyl)-1,2-dihydro-5-quinoxalinecarboxylate (0.483 g, 1.552 mmol, 66.6% yield) as a light green solid. ¹H NMR (400 MHz, DMSO-d₆) δ ppm 12.61 (br. s., 1H), 8.76 (dt, J=3.0, 1.5 Hz, 1H), 8.24 (s, 1H), 7.93-7.98 (m, 2H), 7.80 (s, 1H), 7.42-7.51 (m, 1H), 3.93 (s, 3H), 3.50 (s, 3H). MS (ES+) m/e 312 [M+H]⁺.

48(c) methyl 2-chloro-6-(methyloxy)-7-(2-pyridinyl)-5-quinoxalinecarboxylate. To a solution of methyl 6-(methyloxy)-2-oxo-7-(2-pyridinyl)-1,2-dihydro-5-quinoxalinecarboxylate (0.483 g, 1.552 mmol) was added phosphorus oxychloride (1.446 ml, 15.52 mmol). After heating to reflux for 4 h, the reaction mixture was carefully treated with ice water. The aqueous phase was made basic using saturated aqueous sodium bicarbonate and then it was extracted 5 times with ethyl acetate. The organic phase was dried over magnesium sulfate, filtered, concentrated and purified via flash column chromatography (0-100% ethyl acetate in hexanes) to obtain methyl 2-chloro-6-(methyloxy)-7-(2-pyridinyl)-5-quinoxalinecarboxylate (0.08 g, 0.243 mmol, 15.64% yield). ¹H NMR (400 MHz, CHLOROFORM-d) δ ppm 8.79 (dq, J=4.9, 0.9 Hz, 1H), 8.77 (s, 1H), 8.45 (s, 1H), 7.91 (dt, J=8.0, 1.0 Hz, 1H), 7.81 (td, J=7.8, 1.9 Hz, 1H), 7.34-7.39 (m, 1H), 4.09 (s, 3H), 3.69 (s, 3H). MS (ES+) m/e 330 [M+H]⁺.

48(d) methyl 6-(methyloxy)-2-phenyl-7-(2-pyridinyl)-5-quinoxalinecarboxylate. A solution of methyl 2-chloro-6-(methyloxy)-7-(2-pyridinyl)-5-quinoxalinecarboxylate (0.08 g, 0.243 mmol), phenylboronic acid (0.030 g, 0.243 mmol), potassium carbonate (0.101 g, 0.728 mmol), and tetrakis(triphenylphosphine)palladium(0) (8.41 mg, 7.28 mmol) in 1,4-dioxane (2.0 ml) and water (0.667 ml) was heated to 100° C. for 20 min. in a Biotage Initiator® microwave synthesizer. Upon cooling, the reaction mixture was filtered through Celite®, washed through with ethyl acetate, and concentrated in vacuo. The residue was purified via flash column chromatography (0-100% ethyl acetate in hexanes) to obtain methyl 6-(methyloxy)-2-phenyl-7-(2-pyridinyl)-5-quinoxalinecarboxylate (81 mg, 0.218 mmol, 90% yield) as a yellow solid. ¹H NMR (400 MHz, CHLOROFORM-d) δ ppm 9.35 (s, 1H), 8.82 (dq, J=4.9, 0.8 Hz, 1H), 8.61 (s, 1H), 8.18-8.21 (m, 1H), 8.17 (t, J=1.4 Hz, 1H), 7.95 (dt, J=8.1, 1.0 Hz, 1H), 7.83 (td, J=7.7, 1.8 Hz, 1H), 7.50-7.62 (m, 3H), 7.34-7.41 (m, 1H), 4.13 (s, 3H), 3.72 (s, 3H). MS (ES+) m/e 324 [M+H]⁺.

48(e) 6-hydroxy-2-phenyl-7-(2-pyridinyl)-5-quinoxalinecarboxylic acid. A solution of methyl 6-(methyloxy)-2-phenyl-7-(2-pyridinyl)-5-quinoxalinecarboxylate (0.081 g, 0.218 mmol) in dichloromethane (10 mL) was treated with boron tribromide (1M solution in dichloromethane) (0.654 mL, 0.654 mmol) at room temperature overnight. The reaction mixture was poured into water and filtered. The aqueous mother liquor was extracted twice with ethyl acetate. The combined organic portions were dried over magnesium sulfate, filtered, concentrated and combined with the initially filtered solid to give 6-hydroxy-2-phenyl-7-(2-pyridinyl)-5-quinoxalinecarboxylic acid (0.063 g, 0.183 mmol, 84% yield) as an orange solid. ¹H NMR (400 MHz, CHLOROFORM-d) δ ppm 9.24 (s, 1H), 8.96 (s, 1H), 8.84-8.90 (m, 1H), 8.14-8.25 (m, 3H), 7.96 (td, J=7.8, 1.6 Hz, 1H), 7.54-7.65 (m, 3H), 7.45-7.52 (m, 1H). MS (ES+) m/e 344 [M+H]⁺.

48(f) ethyl N-{[6-hydroxy-2-phenyl-7-(2-pyridinyl)-5-quinoxalinyl]carbonyl}glycinate. A solution of 6-hydroxy-2-phenyl-7-(2-pyridinyl)-5-quinoxalinecarboxylic acid (0.063 g, 0.183 mmol) in N,N-Dimethylformamide (DMF) (5 mL) was treated with ethyl glycine hydrochloride (0.051 g, 0.367 mmol), triethylamine (0.077 mL, 0.550 mmol), and PyBOP (0.105 g, 0.202 mmol). The reaction mixture was stirred overnight at ambient temperature, quenched by water, and filtered. The mother liquor obtained was further extracted with ethyl acetate, dried over magnesium sulfate, filtered, concentrated and combined with the previously filtered solid to obtain ethyl N-{[6-hydroxy-2-phenyl-7-(2-pyridinyl)-5-quinoxalinyl]carbonyl}glycinate (0.045 g, 0.105 mmol, 57.2% yield) as a yellow solid. ¹H NMR (400 MHz, DMSO-d₆) δ ppm 16.18 (s, 1H), 11.36 (t, J=5.6 Hz, 1H), 9.57 (s, 1H), 8.79 (ddd, J=4.8, 1.8, 1.0 Hz, 1H), 8.67 (s, 1H), 8.30-8.37 (m, 2H), 8.20 (td, J=8.1, 1.0 Hz, 1H), 7.98 (dt, J=7.7, 1.8 Hz, 1H), 7.56-7.67 (m, 3H), 7.51 (ddd, J=7.6, 5.1, 1.3 Hz, 1H), 4.37 (d, J=5.6 Hz, 2H), 4.20 (q, J=7.1 Hz, 2H), 1.26 (t, J=7.2 Hz, 3H). MS (ES+) m/e 429 [M+H]⁺.

48(g) N-{[6-hydroxy-2-phenyl-7-(2-pyridinyl)-5-quinoxalinyl]carbonyl}glycine. To a suspension of ethyl N-{[6-hydroxy-2-phenyl-7-(2-pyridinyl)-5-quinoxalinyl]carbonyl}glycinate (0.045 g, 0.105 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-2-phenyl-7-(2-pyridinyl)-5-quinoxalinyl]carbonyl}glycine (0.032 g, 0.080 mmol, 76% yield) as a yellow solid. ¹H NMR (400 MHz, DMSO-d₆) δ ppm 11.40 (t, J=5.7 Hz, 1H), 9.58 (s, 1H), 8.82 (ddd, J=4.9, 1.7, 1.0 Hz, 1H), 8.68 (s, 1H), 8.36 (d, J=1.5 Hz, 1H), 8.34 (d, J=1.3 Hz, 1H), 8.21 (td, J=7.8, 1.0 Hz, 1H), 8.04 (dt, J=7.8, 1.8 Hz, 1H), 7.52-7.68 (m, 4H), 4.30 (d, J=5.6 Hz, 2H). MS (ES+) m/e 401 [M+H]⁺.

### Example 49

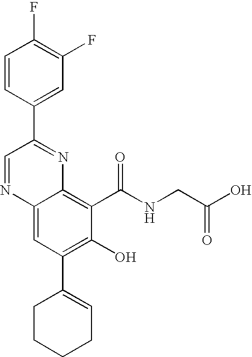

### N-{[7-(1-cyclohexen-1-yl)-3-(3,4-difluorophenyl)-6-hydroxy-5-quinoxalinyl]carbonyl}glycine

To a suspension of ethyl N-{[7-bromo-3-(3,4-difluorophenyl)-6-hydroxy-5-quinoxalinyl]carbonyl}glycinate (0.075 g, 0.161 mmol) in dioxane (3.0 mL) and water (1.0 mL) was added 2-(1-cyclohexen-1-yl)-4,4,5,5-tetramethyl-1,3,2-dioxaborolane (0.040 mL, 0.177 mmol), potassium carbonate (0.044 g, 0.322 mmol), and tetrakis(triphenylphosphine)palladium(0) (0.004 g, 3.46 μmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid, diluted with brine, and extracted thrice with EtOAc. The combined organic layers were dried over MgSO₄, filtered, concentrated in vacuo, and purified via flash column chromatography (1-10% methanol in dichloromethane) to afford N-{[7-(1-cyclohexen-1-yl)-3-(3,4-difluorophenyl)-6-hydroxy-5-quinoxalinyl]carbonyl}glycine (0.051 g, 0.116 mmol, 72.2% yield) as a light yellow solid. ¹H NMR (400 MHz, DMSO-d₆) δ ppm 16.1 (s, 1H), 13.2 (br. s., 1H), 11.4 (t, J=5.1 Hz, 1H), 9.46 (s, 1H), 8.45 (ddd, J=11.9, 7.9, 2.1 Hz, 1H), 8.17-8.27 (m, 1H), 7.94 (s, 1H), 7.65 (dt, J=10.4, 8.6 Hz, 1H), 6.06 (ddd, J=3.5, 2.0, 1.8 Hz, 1H), 4.34 (d, J=5.1 Hz, 2H), 2.41-2.47 (m, 2H), 2.16-2.27 (m, 2H), 1.71-1.78 (m, 2H), 1.63-1.71 (m, 2H). MS (ES+) m/e 440 [M+H]⁺.

### Example 50

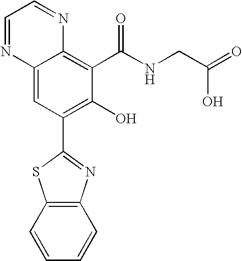

### N-{[7-(1,3-benzothiazol-2-yl)-6-hydroxy-5-quinoxalinyl]carbonyl}glycine

50(a) ethyl N-{[7-(1,3-benzothiazol-2-yl)-6-hydroxy-5-quinoxalinyl]carbonyl}glycinate. To a solution of ethyl N-[(7-bromo-6-hydroxy-5-quinoxalinyl)carbonyl]glycinate (0.085 g, 0.240 mmol) in 1,4-dioxane (1.5 ml) was added 2-(tributylstannanyl)-1,3-benzothiazole (0.102 g, 0.240 mmol) and tetrakis(triphenylphosphine)palladium(0) (0.028 g, 0.024 mmol) followed by heating to 150° C. for 20 min., then 200° C. for 30 min. in a Biotage Initiator® microwave synthesizer. Upon cooling, the reaction mixture was concentrated in vacuo and purified via flash column chromatography (0-100% ethyl acetate in hexanes) to obtain ethyl N-{[7-(1,3-benzothiazol-2-yl)-6-hydroxy-5-quinoxalinyl]carbonyl}glycinate (0.015 g, 0.037 mmol, 15.30% yield) as an orange solid. ¹H NMR (400 MHz, DMSO-d₆) δ ppm 15.80 (s, 1H), 11.58 (t, J=5.8 Hz, 1H), 9.02-9.07 (m, 1H), 9.02 (d, J=2.0 Hz, 1H), 8.98 (d, J=2.0 Hz, 1H), 8.34 (s, 1H), 8.22 (dd, J=15.3, 8.2 Hz, 1H), 7.62 (t, J=8.0 Hz, 1H), 7.46-7.58 (m, 1H), 4.35 (d, J=5.8 Hz, 2H), 4.18 (q, J=7.1 Hz, 2H), 1.24 (t, J=7.1 Hz, 3H). MS (ES+) m/e 409 [M+H]⁺.

50(b) N-{[7-(1,3-benzothiazol-2-yl)-6-hydroxy-5-quinoxalinyl]carbonyl}glycine. To a suspension of ethyl N-{[7-(1,3-benzothiazol-2-yl)-6-hydroxy-5-quinoxalinyl]carbonyl}glycinate (0.015 g, 0.037 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[7-(1,3-benzothiazol-2-yl)-6-hydroxy-5-quinoxalinyl]carbonyl}glycine (0.008 g, 0.021 mmol, 57.3% yield) as an orange solid. ¹H NMR (400 MHz, DMSO-d₆) δ ppm 11.58 (br. s., 1H), 9.21 (s, 1H), 9.03 (br. s., 1H), 9.01 (d, J=2.0 Hz, 1H), 8.23 (d, J=7.6 Hz, 1H), 8.19 (d, J=8.1 Hz, 1H), 7.62 (dt, J=7.6, 1.1 Hz, 1H), 7.53 (dt, J=7.1, 1.0 Hz, 1H), 4.31 (d, J=5.6 Hz, 2H). MS (ES+) m/e 381 [M+H]⁺.

### Example 51

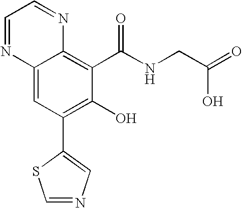

### N-{[6-hydroxy-7-(1,3-thiazol-5-yl)-5-quinoxalinyl]carbonyl}glycine

51(a) ethyl N-{[6-hydroxy-7-(1,3-thiazol-5-yl)-5-quinoxalinyl]carbonyl}glycinate. To a solution of ethyl N-[(7-bromo-6-hydroxy-5-quinoxalinyl)carbonyl]glycinate (0.11 g, 0.311 mmol) in 1,4-dioxane (1.5 ml) was added 5-(tributylstannanyl)-1,3-thiazole (0.116 g, 0.311 mmol) and tetrakis(triphenylphosphine)palladium(0) (0.036 g, 0.031 mmol) followed by heating to 150° C. for 20 min. in a Biotage Initiator® microwave synthesizer. Upon cooling, the reaction mixture was concentrated in vacuo and purified via flash column chromatography (0-10% methanol in ethyl acetate) to obtain ethyl N-{[6-hydroxy-7-(1,3-thiazol-5-yl)-5-quinoxalinyl]carbonyl}glycinate (0.033 g, 0.092 mmol, 29.6% yield) as an orange solid. ¹H NMR (400 MHz, DMSO-d₆) δ ppm 11.63 (br. s., 1H), 9.27 (s, 1H), 8.97 (s, 1H), 8.95 (s, 1H), 8.82 (s, 1H), 8.76 (s, 1H), 4.36 (d, J=5.3 Hz, 2H), 4.19 (q, J=7.1 Hz, 2H), 1.24 (t, J=7.1 Hz, 3H). MS (ES+) m/e 359 [M+H]⁺.

51(b) N-{[6-hydroxy-7-(1,3-thiazol-5-yl)-5-quinoxalinyl]carbonyl}glycine. To a suspension of ethyl N-{[6-hydroxy-7-(1,3-thiazol-5-yl)-5-quinoxalinyl]carbonyl}glycinate (0.033 g, 0.092 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(1,3-thiazol-5-yl)-5-quinoxalinyl]carbonyl}glycine (0.026 g, 0.079 mmol, 85% yield) as a yellow solid. ¹H NMR (400 MHz, DMSO-d₆) δ ppm 13.00 (br. s., 1H), 11.58 (t, J=5.4 Hz, 1H), 9.27 (s, 1H), 8.96 (d, J=1.8 Hz, 1H), 8.94 (d, J=2.0 Hz, 1H), 8.81 (s, 1H), 8.74 (s, 1H), 4.28 (d, J=5.6 Hz, 2H). MS (ES+) m/e 331 [M+H]⁺.

### Example 52

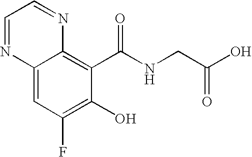

### N-[(7-fluoro-6-hydroxy-5-quinoxalinyl)carbonyl]glycine

52(a) methyl 2,3,6-trifluoro-5-nitrobenzoate. In a 250 mL round-bottomed flask, fuming nitric acid (5.29 ml, 118 mmol) was cooled at 0° C. Concentrated sulfuric acid (12.62 ml, 237 mmol) was slowly added. After the mixture was stirred for 10 minutes, methyl 2,3,6-trifluorobenzoate (4.5 g, 23.67 mmol) was added and the mixture was raised up to the ambient temperature. The reaction was kept stirring overnight and quenched with ice water. The mixture was extracted with ethyl acetate. The extract was dried over MgSO4, concentrated under vacuo and purified via flash chromatography (0-100% ethyl acetate in hexane) to afford methyl 2,3,6-trifluoro-5-nitrobenzoate (4.0 g, 17.01 mmol, 71.9% yield) as a yellow oil. 1H NMR (400 MHz, CHLOROFORM-d) δ ppm 8.10 (td, J=8.1, 7.2 Hz, 1H), 4.02 (s, 3H). MS (ES+) m/e 236[M+H]⁺.

52(b) methyl 2-amino-5-fluoro-6-(methyloxy)-3-nitrobenzoate. In a 50 mL round-bottomed flask, methyl 2,3,6-trifluoro-5-nitrobenzoate (1 g, 4.25 mmol) was dissolved in methanol (20 ml) to give a yellow solution. Methanolic sodium methoxide (4.37 M, 0.973 ml, 4.25 mmol) was added. The reaction was kept stirring at ambient temperature for one hour. Ammonia in methanol (7.0N, 0.608 ml, 4.25 mmol) was added. The mixture was kept stirred at ambient temperature overnight, concentrated and purified through flash chromatography (0-100% ethyl acetate in hexane) to afford methyl 2-amino-5-fluoro-6-(methyloxy)-3-nitrobenzoate (310 mg, 1.270 mmol, 29.8% yield) as a yellow solid. 1H NMR (400 MHz, DMSO-d6) δ ppm 8.10 (d, J=12.6 Hz, 1H), 7.35 (s, 2H), 3.96 (d, J=3.0 Hz, 3H), 3.88 (s, 3H). MS (ES+) m/e 245[M+H]⁺.

52(c) methyl 7-fluoro-6-(methyloxy)-5-quinoxalinecarboxylate. A 500 mL hydrogenation flask was charged with methyl 2-amino-5-fluoro-6-(methyloxy)-3-nitrobenzoate (310 mg, 1.270 mmol) and 5% palladium-on-charcoal (135 mg, 0.065 mmol) in methanol (25 ml) to give a black suspension. The mixture was hydrogenated under hydrogen balloon for 3 hours, then filtered. Glyoxal (40% aqueous solution) (161 mg, 1.270 mmol) was added. The reaction was refluxed for 2 hours, concentrated and purified via preparative HPLC (YMC 75×30 mm column, 0.1% TFA in water and 0.1% TFA in acetonitrile) to afford methyl 7-fluoro-6-(methyloxy)-5-quinoxalinecarboxylate (160 mg, 0.677 mmol, 53.4% yield) as a brown oil. ¹H NMR (400 MHz, CHLOROFORM-d) δ ppm 13.05 (br. s., 1H), 8.93 (d, J=1.8 Hz, 1H), 8.83 (d, J=1.8 Hz, 1H), 7.89 (d, J=11.4 Hz, 1H), 4.13 (d, J=2.3 Hz, 3H), 4.07 (s, 3H). MS (ES+) m/e 237[M+H]⁺.

52(d) 7-fluoro-6-hydroxy-5-quinoxalinecarboxylic acid. In a 100 mL round-bottomed flask was placed methyl 7-fluoro-6-(methyloxy)-5-quinoxalinecarboxylate (150 mg, 0.635 mmol) in dichloromethane (25 ml) to give a yellow solution. Boron tribromide (50% in toluene) (0.480 ml, 2.54 mmol) was added. The mixture was kept stirring overnight under ambient temperature. The reaction was quenched by water and extracted by dichloromethane. The extract was dried over MgSO₄, filtered, concentrated under vacuo and purified through flash chromatography (0-10% methanol in dichloromethane) to afford 7-fluoro-6-hydroxy-5-quinoxalinecarboxylic acid (100 mg, 0.480 mmol, 76% yield) as yellow solid. ¹H NMR (400 MHz, DMSO-d₆) δ ppm 8.98 (d, J=2.5 Hz, 1H), 8.86 (d, J=2.5 Hz, 1H), 8.16 (d, J=10.9 Hz, 1H). MS (ES+) m/e 209[M+H]⁺.

52(e) N-[(7-fluoro-6-hydroxy-5-quinoxalinyl)carbonyl]glycine. In a 100 mL round-bottomed flask was placed 7-fluoro-6-hydroxy-5-quinoxalinecarboxylic acid (100 mg, 0.480 mmol) and glycine ethyl ester hydrochloride (74 mg, 0.528 mmol) in N,N-dimethylformamide (10 ml) to give a yellow solution. Triethylamine (0.201 ml, 1.441 mmol) and PyBOP (275 mg, 0.528 mmol) were added. The mixture was kept stirring overnight, then concentrated under vacuo. The residue was dissolved in methanol (10.00 ml) and sodium hydroxide (6M, 0.080 ml, 0.480 mmol) was added. The mixture was kept stirring for half hour. The precipitate was collected, acidified with 1N HCl, washed with water and dried to afford N-[(7-fluoro-6-hydroxy-5-quinoxalinyl)carbonyl]glycine (30 mg, 0.113 mmol, 23.55% yield) as a yellow solid. ¹H NMR (400 MHz, DMSO-d6) δ ppm 8.65 (br. s., 1H), 8.39 (s, 1H), 8.14 (s, 1H), 7.26 (d, J=11.6 Hz, 1H), 3.53 (d, J=4.5 Hz, 2H). MS (ES+) m/e 266[M+H]⁺.

### Example 53

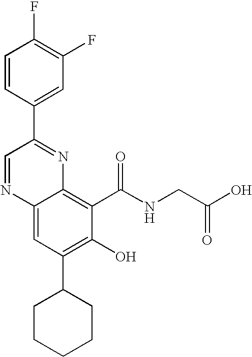

### N-{[7-cyclohexyl-3-(3,4-difluorophenyl)-6-hydroxy-5-quinoxalinyl]carbonyl}glycine

To a solution of N-{[7-(1-cyclohexen-1-yl)-3-(3,4-difluorophenyl)-6-hydroxy-5-quinoxalinyl]carbonyl}glycine (0.034 g, 0.077 mmol) in tetrahydrofuran (3.0 mL) and methanol (3.0 mL) was added 10% palladium on charcoal (4.0 mg, 3.76 μmol) followed by evacuation of the reaction vessel and purging with 1 atmosphere of hydrogen. Following stirring at ambient temperature for 24 h, additional 10% palladium on charcoal (4.0 mg, 3.76 μmol) was added. Following stirring at ambient temperature for an additional 24 h under 1 atmosphere of hydrogen, the reaction mixture was filtered through Celite®, washed through with methanol, concentrated in vacuo, and purified via flash column chromatography (1-10% methanol in dichloromethane) to afford N-{[7-cyclohexyl-3-(3,4-difluorophenyl)-6-hydroxy-5-quinoxalinyl]carbonyl}glycine (0.028 g, 0.063 mmol, 82% yield) as a light, pale yellow solid. ¹H NMR (400 MHz, DMSO-d₆) δ ppm 16.2 (s, 1H), 13.2 (br. s., 1H), 11.4 (t, J=5.1 Hz, 1H), 9.47 (s, 1H), 8.45 (ddd, J=11.7, 7.8, 2.3 Hz, 1H), 8.15-8.28 (m, 1H), 8.01 (s, 1H), 7.65 (dt, J=10.2, 8.6 Hz, 1H), 4.35 (d, J=5.1 Hz, 2H), 3.05-3.14 (m, 1H), 1.91-1.97 (m, 2H), 1.82-1.88 (m, 2H), 1.72-1.79 (m, 1H), 1.40-1.56 (m, 4H), 1.27-1.38 (m, 1H). MS (ES+) m/e 442 [M+H]⁺.

### Example 54

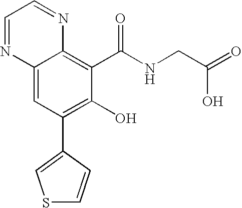

### N-{[6-hydroxy-7-(3-thienyl)-5-quinoxalinyl]carbonyl}glycine

54(a) ethyl N-{[6-hydroxy-7-(3-thienyl)-5-quinoxalinyl]carbonyl}glycinate. A solution of ethyl N-[(7-bromo-6-hydroxy-5-quinoxalinyl)carbonyl]glycinate (0.110 g, 0.311 mmol), 3-thienylboronic acid (0.040 g, 0.311 mmol), potassium carbonate (0.129 g, 0.932 mmol), and tetrakis(triphenylphosphine)palladium(0) (11 mg, 9.52 μmol) in 1,4-dioxane (2.0 ml) and water (0.667 ml) was heated to 100° C. for 20 min. in a Biotage Initiator® microwave synthesizer. Upon cooling, the reaction mixture was filtered through Celite®, washed through with ethyl acetate, and concentrated in vacuo. The crude consisted of a mixture of desired product (M+358) plus hydrolized ester (M+330). The resulting solid was washed with diethyl ether, filtered and dried in vacuo to obtain ethyl N-{[6-hydroxy-7-(3-thienyl)-5-quinoxalinyl]carbonyl}glycinate (0.048 g, 0.134 mmol, 43.2% yield) as a pale yellow solid. ¹H NMR (400 MHz, DMSO-d₆) δ ppm 16.38 (s, 1H), 11.66 (t, J=5.6 Hz, 1H), 8.92 (d, J=2.0 Hz, 1H), 8.91 (d, J=2.0 Hz, 1H), 8.42 (s, 1H), 8.23 (dd, J=2.8, 1.3 Hz, 1H), 7.76 (dd, J=5.2, 1.4 Hz, 1H), 7.69 (dd, J=5.1, 3.0 Hz, 1H), 4.35 (d, J=5.8 Hz, 2H), 4.19 (q, J=7.2 Hz, 2H), 1.24 (t, J=7.2 Hz, 3H). MS (ES+) m/e 358 [M+H]⁺.

54(b) N-{[6-hydroxy-7-(3-thienyl)-5-quinoxalinyl]carbonyl}glycine. To a suspension of ethyl N-{[6-hydroxy-7-(3-thienyl)-5-quinoxalinyl]carbonyl}glycinate (0.048 g, 0.134 mmol) in ethanol (2.0 mL) was added 1N aqueous sodium hydroxide (3.0 ml, 3.00 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(3-thienyl)-5-quinoxalinyl]carbonyl}glycine (0.026 g, 0.079 mmol, 58.8% yield) as an orange solid. ¹H NMR (400 MHz, DMSO-d₆) δ ppm 12.95 (br. s., 1H), 11.63 (t, J=5.4 Hz, 1H), 8.93 (s, 2H), 8.44 (s, 1H), 8.25 (dd, J=3.0, 1.3 Hz, 1H), 7.77 (dd, J=5.1, 1.3 Hz, 1H), 7.69 (dd, J=5.2, 2.9 Hz, 1H), 4.28 (d, J=5.6 Hz, 2H). MS (ES+) m/e 330 [M+H]⁺.

### Example 55

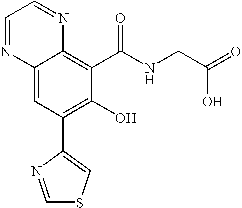

### N-{[6-hydroxy-7-(1,3-thiazol-4-yl)-5-quinoxalinyl]carbonyl}glycine

55(a) ethyl N-{[6-hydroxy-7-(1,3-thiazol-4-yl)-5-quinoxalinyl]carbonyl}glycinate. To a solution of ethyl N-[(7-bromo-6-hydroxy-5-quinoxalinyl)carbonyl]glycinate (0.110 g, 0.311 mmol) in 1,4-dioxane (2.0 ml) was added 4-(tributylstannanyl)-1,3-thiazole (0.116 g, 0.311 mmol) and tetrakis(triphenylphosphine)palladium(0) (0.108 g, 0.093 mmol) followed by heating to 150° C. for 20 min. in a Biotage Initiator® microwave synthesizer. Upon cooling, the reaction mixture was filtered through Celite®, washed through with ethyl acetate, and concentrated in vacuo. The solid obtained was washed with ethyl ether, filtered and dried under vacuum to obtain ethyl N-{[6-hydroxy-7-(1,3-thiazol-4-yl)-5-quinoxalinyl]carbonyl}glycinate (0.059 g, 0.165 mmol, 53.0% yield) as a pale yellow solid. ¹H NMR (400 MHz, DMSO-d₆) δ ppm 11.69 (t, J=5.7 Hz, 1H), 9.32 (d, J=2.0 Hz, 1H), 9.00 (s, 1H), 8.97 (d, J=2.0 Hz, 1H), 8.96 (d, J=2.0 Hz, 1H), 8.63 (d, J=2.0 Hz, 1H), 4.37 (d, J=5.8 Hz, 2H), 4.20 (q, J=7.2 Hz, 2H), 1.25 (t, J=7.1 Hz, 3H). MS (ES+) m/e 359 [M+H]⁺.

55(b) N-{[6-hydroxy-7-(1,3-thiazol-4-yl)-5-quinoxalinyl]carbonyl}glycine. To a suspension of ethyl N-{[6-hydroxy-7-(1,3-thiazol-4-yl)-5-quinoxalinyl]carbonyl}glycinate (0.059 g, 0.165 mmol) in ethanol (3.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-7-(1,3-thiazol-4-yl)-5-quinoxalinyl]carbonyl}glycine (0.045 g, 0.136 mmol, 83% yield) as an orange solid. ¹H NMR (400 MHz, DMSO-d₆) δ ppm 12.97 (br. s., 1H), 11.65 (t, J=5.6 Hz, 1H), 9.32 (d, J=2.0 Hz, 1H), 8.99 (s, 1H), 8.96 (s, 2H), 8.63 (d, J=2.0 Hz, 1H), 4.29 (d, J=5.6 Hz, 2H). MS (ES+) m/e 331 [M+H]⁺.

### Example 56

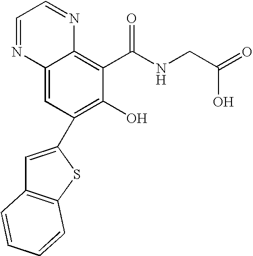

### N-{[7-(1-benzothien-2-yl)-6-hydroxy-5-quinoxalinyl]carbonyl}glycine

56(a) ethyl N-{[7-(1-benzothien-2-yl)-6-hydroxy-5-quinoxalinyl]carbonyl}glycinate. A solution of ethyl N-[(7-bromo-6-hydroxy-5-quinoxalinyl)carbonyl]glycinate (0.110 g, 0.311 mmol), 1-benzothien-2-ylboronic acid (0.055 g, 0.311 mmol), potassium carbonate (0.129 g, 0.932 mmol), and tetrakis(triphenylphosphine)palladium(0) (10.77 mg, 9.32 mmol) in 1,4-dioxane (2.0 ml) and water (0.667 ml) was heated to 100° C. for 20 min. in a Biotage Initiator® microwave synthesizer. Upon cooling, the reaction mixture was dissolved in water, extracted with ethyl acetate, dried over magnesium sulfate and concentrated in vacuo. The residue was purified via flash column chromatography (10% methanol in dichloromethane) to obtain ethyl N-{[7-(1-benzothien-2-yl)-6-hydroxy-5-quinoxalinyl]carbonyl}glycinate (0.074 g, 0.182 mmol, 58.5% yield) as a bright yellow solid. ¹H NMR (400 MHz, DMSO-d₆) δ ppm 11.67 (t, J=5.2 Hz, 1H), 8.97 (d, J=1.6 Hz, 1H), 8.96 (d, J=1.6 Hz, 1H), 8.64 (s, 1H), 8.38 (s, 1H), 8.05 (dd, J=4.9, 4.2 Hz, 1H), 7.91-8.00 (m, 1H), 7.38-7.48 (m, 2H), 4.38 (d, J=5.6 Hz, 2H), 4.20 (q, J=7.1 Hz, 2H), 1.25 (t, J=7.1 Hz, 3H). MS (ES+) m/e 408 [M+H]⁺.

56(b) N-{[7-(1-benzothien-2-yl)-6-hydroxy-5-quinoxalinyl]carbonyl}glycine. To a suspension of ethyl N-{[7-(1-benzothien-2-yl)-6-hydroxy-5-quinoxalinyl]carbonyl}glycinate (0.074 g, 0.182 mmol) in ethanol (1.0 mL) was added 1N aqueous sodium hydroxide (4.0 ml, 4.00 mmol). After stirring 30 min. at ambient temperature, the reaction was quenched with 1N aqueous hydrochloric acid and the resulting precipitate was filtered, washed with water, and dried in vacuo to obtain N-{[7-(1-benzothien-2-yl)-6-hydroxy-5-quinoxalinyl]carbonyl}glycine (0.045 g, 0.119 mmol, 65.3% yield) as an orange solid. ¹H NMR (400 MHz, DMSO-d₆) δ ppm 13.00 (br. s., 1H), 11.63 (t, J=5.3 Hz, 1H), 8.97 (d, J=2.0 Hz, 1H), 8.96 (d, J=2.0 Hz, 1H), 8.63 (s, 1H), 8.38 (s, 1H), 8.01-8.09 (m, 1H), 7.91-8.01 (m, 1H), 7.36-7.51 (m, 2H), 4.30 (d, J=5.6 Hz, 2H). MS (ES+) m/e 380 [M+H]⁺.

### Example 57

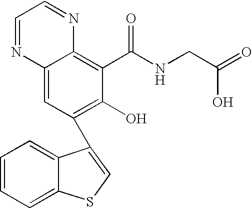

### N-{[7-(1-benzothien-3-yl)-6-hydroxy-5-quinoxalinyl]carbonyl}glycine

57(a) ethyl N-{[7-(1-benzothien-3-yl)-6-hydroxy-5-quinoxalinyl]carbonyl}glycinate. A solution of ethyl N-[(7-bromo-6-hydroxy-5-quinoxalinyl)carbonyl]glycinate (0.120 g, 0.339 mmol), 1-benzothien-3-ylboronic acid (0.066 g, 0.373 mmol), potassium carbonate (0.140 g, 1.017 mmol), and tetrakis(triphenylphosphine)palladium(0) (0.078 g, 0.068 mmol) in 1,4-dioxane (2.0 ml) and water (0.667 ml) was heated to 100° C. for 20 min. in a Biotage Initiator® microwave synthesizer. Upon cooling, the reaction mixture was dissolved in water and extracted thrice with ethyl acetate. The combined organic portions were dried over magnesium sulfate, filtered and concentrated in vacuo. The residue was purified via flash column chromatography (10% methanol in dichloromethane) to obtain ethyl N-{[7-(1-benzothien-3-yl)-6-hydroxy-5-quinoxalinyl]carbonyl}glycinate (0.056 g, 0.137 mmol, 40.6% yield) as an orange solid. ¹H NMR (400 MHz, DMSO-d₆) δ ppm 15.95 (s, 1H), 11.65 (t, J=5.4 Hz, 1H), 9.00 (d, J=2.0 Hz, 1H), 8.97 (d, J=2.0 Hz, 1H), 8.28 (s, 1H), 8.10 (dd, J=7.1, 1.5 Hz, 1H), 8.04 (s, 1H), 7.64-7.69 (m, 1H), 7.35-7.48 (m, 2H), 4.36 (d, J=5.8 Hz, 2H), 4.19 (q, J=7.1 Hz, 2H), 1.25 (t, J=7.1 Hz, 3H). MS (ES+) m/e 408 [M+H]⁺.

57(b) N-{[7-(1-benzothien-3-yl)-6-hydroxy-5-quinoxalinyl]carbonyl}glycine. To a suspension of ethyl N-{[7-(1-benzothien-3-yl)-6-hydroxy-5-quinoxalinyl]carbonyl}glycinate (0.056 g, 0.137 mmol) in ethanol (2.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[7-(1-benzothien-3-yl)-6-hydroxy-5-quinoxalinyl]carbonyl}glycine (0.026 g, 0.069 mmol, 49.9% yield) as a bright yellow solid. ¹H NMR (400 MHz, DMSO-d₆) δ ppm 11.23 (br. s., 1H), 8.92 (s, 1H), 8.88 (s, 1H), 8.17 (s, 1H), 8.08 (dd, J=6.9, 1.6 Hz, 1H), 8.01 (s, 1H), 7.67 (dd, J=7.1, 1.8 Hz, 1H), 7.35-7.47 (m, 2H), 3.74 (d, J=4.3 Hz, 2H). MS (ES+) m/e 380 [M+H]⁺.

### Example 58

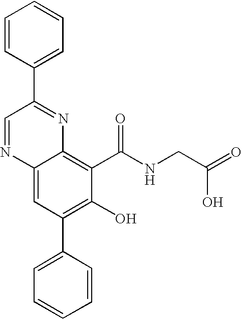

### N-[(6-hydroxy-3,7-diphenyl-5-quinoxalinyl)carbonyl]glycine

58(a) methyl 6-(methyloxy)-3,7-diphenyl-5-quinoxalinecarboxylate. A solution of methyl 7-bromo-6-(methyloxy)-3-phenyl-5-quinoxalinecarboxylate (example 15(a), 0.170 g, 0.456 mmol), phenylboronic acid (0.056 g, 0.456 mmol), potassium carbonate (0.189 g, 1.367 mmol), and tetrakis(triphenylphosphine)palladium(0) (0.105 g, 0.091 mmol) in 1,4-dioxane (2.0 ml) and water (0.667 ml) was heated to 100° C. for 20 min. in a Biotage Initiator® microwave synthesizer. Upon cooling, the reaction mixture was dissolved in water, extracted with ethyl acetate, dried over magnesium sulfate and concentrated in vacuo. The residue was purified via flash column chromatography (10% methanol in dichloromethane) to obtain methyl 6-(methyloxy)-3,7-diphenyl-5-quinoxalinecarboxylate (0.100 g, 0.270 mmol, 59.3% yield) as a yellow solid. ¹H NMR (400 MHz, DMSO-d₆) δ ppm 9.62 (s, 1H), 8.33-8.35 (m, 1H), 8.31 (t, J=2.3 Hz, 1H), 8.15 (s, 1H), 7.73 (t, J=1.8 Hz, 1H), 7.70-7.72 (m, 1H), 7.59-7.67 (m, 3H), 7.53-7.59 (m, 2H), 7.46-7.52 (m, 1H), 4.04 (s, 3H), 3.56 (s, 3H). MS (ES+) m/e 371 [M+H]⁺.

58(b) 6-hydroxy-3,7-diphenyl-5-quinoxalinecarboxylic acid. A solution of methyl 6-(methyloxy)-3,7-diphenyl-5-quinoxalinecarboxylate (0.100 g, 0.270 mmol) in dichloromethane (3.00 mL) was treated with boron tribromide (1M solution in dichloromethane) (0.810 ml, 0.810 mmol) at room temperature overnight. The reaction mixture was treated with water and extracted twice with dichloromethane. The combined organic portions were dried over magnesium sulfate, filtered and concentrated to obtain 6-hydroxy-3,7-diphenyl-5-quinoxalinecarboxylic acid (0.09 g, 0.263 mmol, 97% yield) as an orange solid. ¹H NMR (400 MHz, DMSO-d₆) δ ppm 13.94 (br. s., 1H), 9.68 (s, 1H), 8.36 (s, 1H), 8.25-8.28 (m, 1H), 8.23-8.26 (m, 1H), 7.68-7.78 (m, 5H), 7.50-7.59 (m, 3H). MS (ES+) m/e 343 [M+H]⁺.

58(c) ethyl N-[(6-hydroxy-3,7-diphenyl-5-quinoxalinyl)carbonyl]glycinate. A solution of 6-hydroxy-3,7-diphenyl-5-quinoxalinecarboxylic acid (0.09 g, 0.263 mmol) and ethyl glycine hydrochloride (0.073 g, 0.526 mmol) in N,N-Dimethylformamide (DMF) (3.0 mL) was treated with triethylamine (0.110 mL, 0.789 mmol) and PyBOP (0.150 g, 0.289 mmol). The reaction mixture was stirred overnight at ambient temperature, quenched by water, filtered and dried in vacuo to obtain ethyl N-[(6-hydroxy-3,7-diphenyl-5-quinoxalinyl)carbonyl]glycinate (0.093 g, 0.218 mmol, 83% yield) as a yellow solid. ¹H NMR (400 MHz, DMSO-d₆) δ ppm 16.05 (s, 1H), 11.74 (t, J=5.6 Hz, 1H), 9.53 (s, 1H), 8.35 (d, J=3.5 Hz, 1H), 8.34 (d, J=2.3 Hz, 1H), 8.21 (s, 1H), 7.74 (d, J=1.5 Hz, 1H), 7.72 (d, J=1.3 Hz, 1H), 7.62-7.67 (m, 3H), 7.50-7.57 (m, 2H), 7.43-7.50 (m, 1H), 4.48 (d, J=5.3 Hz, 2H), 4.21 (q, J=7.1 Hz, 2H), 1.23 (t, J=7.2 Hz, 3H). MS (ES+) m/e 428 [M+H]⁺.

58(d) N-[(6-hydroxy-3,7-diphenyl-5-quinoxalinyl)carbonyl]glycine. To a suspension of ethyl N-[(6-hydroxy-3,7-diphenyl-5-quinoxalinyl)carbonyl]glycinate (0.093 g, 0.218 mmol) in ethanol (2.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-[(6-hydroxy-3,7-diphenyl-5-quinoxalinyl)carbonyl]glycine (0.078 g, 0.196 mmol, 90% yield) as a pale yellow solid. ¹H NMR (400 MHz, DMSO-d₆) δ ppm 16.22 (s, 1H), 13.17 (br. s., 1H), 11.65 (t, J=5.2 Hz, 1H), 9.52 (s, 1H), 8.38-8.41 (m, 1H), 8.35-8.38 (m, 1H), 8.19 (s, 1H), 7.73 (t, J=1.8 Hz, 1H), 7.70-7.72 (m, 1H), 7.60-7.66 (m, 3H), 7.53 (tt, J=7.1, 1.5 Hz, 2H), 7.47 (tt, J=7.3, 1.3 Hz, 1H), 4.39 (d, J=5.1 Hz, 2H). MS (ES+) m/e 400 [M+H]⁺.

### Example 59

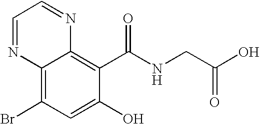

### N-[(8-bromo-6-hydroxy-5-quinoxalinyl)carbonyl]glycine

59(a) methyl 8-bromo-6-(methyloxy)-5-quinoxalinecarboxylate. In a 100 mL round-bottomed flask was placed methyl 2-amino-4-bromo-6-(methyloxy)-3-nitrobenzoate (example 41(d), 850 mg, 2.79 mmol) in methanol (25 ml) to give a yellow suspension. Palladium-on-carbon (29.7 mg, 0.279 mmol) was added and the mixture hydrogenated under hydrogen ballon for two hours, then filtered. Glyoxal (40% in water) (354 mg, 2.79 mmol) was added. The reaction was refluxed for two hours, concentrated under vacuo and purified via flash chromatography (0-100% ethyl acetate in hexane) to afford methyl 8-bromo-6-(methyloxy)-5-quinoxalinecarboxylate (60 mg, 0.202 mmol, 7.25% yield) as a brown solid. ¹H NMR (400 MHz, DMSO-d6) δ ppm 8.96 (d, J=3.0 Hz, 2H), 8.28 (s, 1H), 4.05 (s, 3H), 3.90 (s, 3H). MS (ES+) m/e 299[M+H]⁺.

59(b) N-[(8-bromo-6-hydroxy-5-quinoxalinyl)carbonyl]glycine. In a 25 mL round-bottomed flask was placed borontribromide (50% in toluene) (0.051 ml, 0.269 mmol) in dichloromethane (5 ml) to give a yellow solution. Methyl 8-bromo-6-(methyloxy)-5-quinoxalinecarboxylate (0.020 g, 0.067 mmol) was added. The mixture was kept stirring at ambient temperature overnight, quenched with ice water and extracted with dichloromethane. The extract was dried over MgSO₄, filtered and concentrated under vacuo. The resulting yellow solid was dissolve in N,N-dimethylformamide (5.00 ml), ethyl glycine hydrochloride (10.33 mg, 0.074 mmol), triethylamine (0.028 ml, 0.202 mmol) and PyBOP (38.5 mg, 0.074 mmol) were added. The mixture was kept stirring overnight at ambient temperature, then concentrated under vacuo. The resulting yellow oil was dissolved in methanol (5.00 ml) and sodium hydroxide (6.0N in water) (0.011 ml, 0.067 mmol) was added. The reaction was kept stirring for half hour and purified via preparative HPLC (YMC 75×30 mm column, 0.1% TFA in water and 0.1% TFA in acetonitrile) to afford N-[(8-bromo-6-hydroxy-5-quinoxalinyl)carbonyl]glycine (7.0 mg, 0.021 mmol, 31.9% yield) as a brown solid. ¹H NMR (400 MHz, DMSO-d6) δ ppm 15.48 (br. s., 1H), 11.29 (t, J=5.6 Hz, 1H), 8.99 (q, J=2.0 Hz, 2H), 8.01 (s, 1H), 4.23 (d, J=5.6 Hz, 2H). MS (ES+) m/e 327[M+H]⁺.

### Example 60

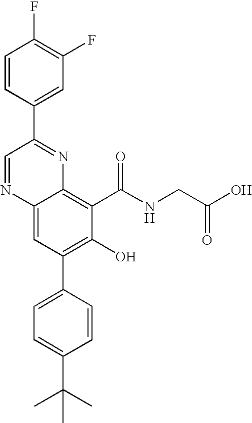

### N-({3-(3,4-difluorophenyl)-7-[4-(1,1-dimethylethyl)phenyl]-6-hydroxy-5-quinoxalinyl}carbonyl)glycine

To a suspension of ethyl N-{[7-bromo-3-(3,4-difluorophenyl)-6-hydroxy-5-quinoxalinyl]carbonyl}glycinate (0.100 g, 0.214 mmol) in dioxane (3.0 mL) and water (1.0 mL) was added 4-tert-butylphenylboronic acid (0.042 g, 0.236 mmol), potassium carbonate (0.059 g, 0.429 mmol), and tetrakis(triphenylphosphine)palladium(0) (0.006 g, 5.19 mmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid and the resulting precipitate was filtered, washed with water, and dried in vacuo to afford N-({3-(3,4-difluorophenyl)-7-[4-(1,1-dimethylethyl)phenyl]-6-hydroxy-5-quinoxalinyl}carbonyl)glycine (0.093 g, 0.189 mmol, 88% yield) as a yellow solid. ¹H NMR (400 MHz, DMSO-d₆) δ ppm 16.2 (s, 1H), 13.2 (br. s., 1H), 11.4 (t, J=5.1 Hz, 1H), 9.49 (s, 1H), 8.46 (ddd, J=11.7, 7.9, 2.1 Hz, 1H), 8.20-8.27 (m, 1H), 8.16 (s, 1H), 7.65 (d, J=8.6 Hz, 2H), 7.65 (dt, J=10.4, 8.6 Hz, 1H), 7.53 (d, J=8.3 Hz, 2H), 4.37 (d, J=5.1 Hz, 2H), 1.35 (s, 9H). MS (ES+) m/e 492 [M+H]⁺.

### Example 61

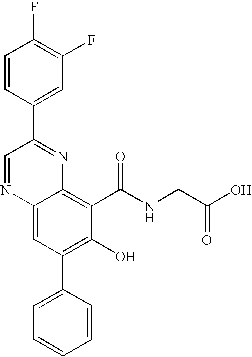

### N-{[3-(3,4-difluorophenyl)-6-hydroxy-7-phenyl-5-quinoxalinyl]carbonyl}glycine

To a suspension of ethyl N-{[7-bromo-3-(3,4-difluorophenyl)-6-hydroxy-5-quinoxalinyl]carbonyl}glycinate (0.100 g, 0.214 mmol) in dioxane (3.0 mL) and water (1.0 mL) was added phenylboronic acid (0.029 g, 0.236 mmol), potassium carbonate (0.059 g, 0.429 mmol), and tetrakis(triphenylphosphine)palladium(0) (0.006 g, 5.19 μmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid and the resulting precipitate was filtered, washed with water, and dried in vacuo to afford N-{[3-(3,4-difluorophenyl)-6-hydroxy-7-phenyl-5-quinoxalinyl]carbonyl}glycine (0.085 g, 0.195 mmol, 91% yield) as a yellow solid. ¹H NMR (400 MHz, DMSO-d₆) δ ppm 16.2 (s, 1H), 13.2 (br. s., 1H), 11.4 (t, J=5.1 Hz, 1H), 9.50 (s, 1H), 8.46 (ddd, J=11.7, 7.9, 1.9 Hz, 1H), 8.21-8.27 (m, 1H), 8.18 (s, 1H), 7.71 (d, J=6.8 Hz, 2H), 7.65 (dt, J=10.4, 8.6 Hz, 1H), 7.52 (t, J=7.2 Hz, 2H), 7.47 (t, J=7.1 Hz, 1H), 4.37 (d, J=5.1 Hz, 2H). MS (ES+) m/e 436 [M+H]⁺.

### Example 62

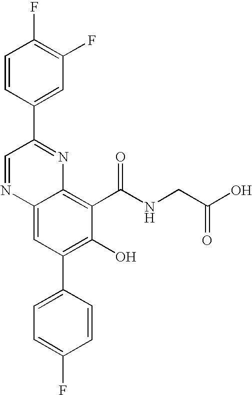

### N-{[3-(3,4-difluorophenyl)-7-(4-fluorophenyl)-6-hydroxy-5-quinoxalinyl]carbonyl}glycine

To a suspension of ethyl N-{[7-bromo-3-(3,4-difluorophenyl)-6-hydroxy-5-quinoxalinyl]carbonyl}glycinate (0.100 g, 0.214 mmol) in dioxane (3.0 mL) and water (1.0 mL) was added 4-fluorophenylboronic acid (0.033 g, 0.236 mmol), potassium carbonate (0.059 g, 0.429 mmol), and tetrakis(triphenylphosphine)palladium(0) (0.006 g, 5.19 μmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid and the resulting precipitate was filtered, washed with water, and dried in vacuo to afford N-{[3-(3,4-difluorophenyl)-7-(4-fluorophenyl)-6-hydroxy-5-quinoxalinyl]carbonyl}glycine (0.086 g, 0.190 mmol, 88% yield) as a pale yellow solid. ¹H NMR (400 MHz, DMSO-d₆) δ ppm 16.3 (s, 1H), 13.2 (br. s., 1H), 11.4 (t, J=5.1 Hz, 1H), 9.50 (s, 1H), 8.46 (ddd, J=11.8, 7.9, 2.0 Hz, 1H), 8.21-8.26 (m, 1H), 8.19 (s, 1H), 7.77 (dt, J=6.1, 2.8 Hz, 2H), 7.65 (dt, J=10.3, 8.5 Hz, 1H), 7.35 (t, J=9.0 Hz, 2H), 4.37 (d, J=5.1 Hz, 2H). MS (ES+) m/e 454 [M+H]⁺.

### Example 63

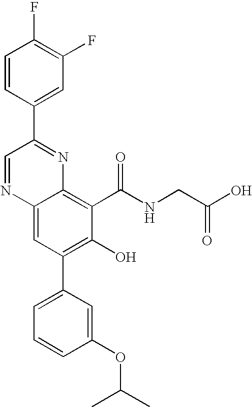

### N-[(3-(3,4-difluorophenyl)-6-hydroxy-7-{3-[(1-methylethyl)oxy]phenyl}-5-quinoxalinyl)carbonyl]glycine

To a suspension of ethyl N-{[7-bromo-3-(3,4-difluorophenyl)-6-hydroxy-5-quinoxalinyl]carbonyl}glycinate (0.100 g, 0.214 mmol) in dioxane (3.0 mL) and water (1.0 mL) was added 3-isopropoxyphenylboronic acid (0.042 g, 0.236 mmol), potassium carbonate (0.059 g, 0.429 mmol), and tetrakis(triphenylphosphine)palladium(0) (0.006 g, 5.19 μmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid and the resulting precipitate was filtered, washed with water, and dried in vacuo to afford N-[(3-(3,4-difluorophenyl)-6-hydroxy-7-{3-[(1-methylethyl)oxy]phenyl}-5-quinoxalinyl)carbonyl]glycine (0.080 g, 0.162 mmol, 76% yield) as a yellow solid. ¹H NMR (400 MHz, DMSO-d₆) δ ppm 16.2 (s, 1H), 13.2 (br. s., 1H), 11.4 (t, J=4.8 Hz, 1H), 9.50 (s, 1H), 8.46 (ddd, J=11.6, 7.8, 2.3 Hz, 1H), 8.21-8.27 (m, 1H), 8.18 (s, 1H), 7.66 (dt, J=10.3, 8.5 Hz, 1H), 7.40 (t, J=8.1 Hz, 1H), 7.21-7.25 (m, 2H), 7.01 (ddd, J=8.3, 2.3, 1.0 Hz, 1H), 4.70 (qq, J=6.1 Hz, 1H), 4.37 (d, J=4.8 Hz, 2H), 1.31 (d, J=6.1 Hz, 6H). MS (ES+) m/e 494 [M+H]⁺.

### Example 64

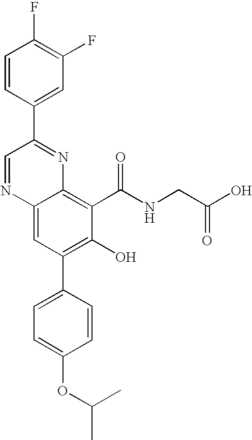

### N-[(3-(3,4-difluorophenyl)-6-hydroxy-7-{4-[(1-methylethyl)oxy]phenyl}-5-quinoxalinyl)carbonyl]glycine

To a suspension of ethyl N-{[7-bromo-3-(3,4-difluorophenyl)-6-hydroxy-5-quinoxalinyl]carbonyl}glycinate (0.100 g, 0.214 mmol) in dioxane (3.0 mL) and water (1.0 mL) was added 4-isopropoxyphenylboronic acid (0.042 g, 0.236 mmol), potassium carbonate (0.059 g, 0.429 mmol), and tetrakis(triphenylphosphine)palladium(0) (0.006 g, 5.19 mmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid and the resulting precipitate was filtered, washed with water, and dried in vacuo to afford N-[(3-(3,4-difluorophenyl)-6-hydroxy-7-{4-[(1-methylethyl)oxy]phenyl}-5-quinoxalinyl)carbonyl]glycine (0.091 g, 0.184 mmol, 86% yield) as a yellow solid. ¹H NMR (400 MHz, DMSO-d₆) δ ppm 16.3 (s, 1H), 13.2 (br. s., 1H), 11.4 (t, J=4.8 Hz, 1H), 9.47 (s, 1H), 8.44 (ddd, J=11.8, 7.8, 2.1 Hz, 1H), 8.18-8.27 (m, 1H), 8.12 (s, 1H), 7.65 (d, J=8.8 Hz, 2H), 7.64 (dt, J=10.3, 8.4 Hz, 1H), 7.03 (d, J=8.8 Hz, 2H), 4.71 (qq, J=6.1 Hz, 1H), 4.36 (d, J=4.8 Hz, 2H), 1.32 (d, J=6.1 Hz, 6H). MS (ES+) m/e 494 [M+H]⁺.

### Example 65

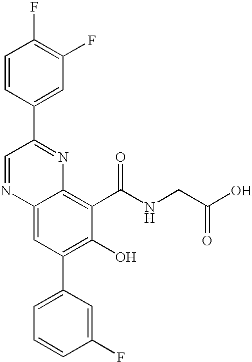

### N-{[3-(3,4-difluorophenyl)-7-(3-fluorophenyl)-6-hydroxy-5-quinoxalinyl]carbonyl}glycine

To a suspension of ethyl N-{[7-bromo-3-(3,4-difluorophenyl)-6-hydroxy-5-quinoxalinyl]carbonyl}glycinate (0.100 g, 0.214 mmol) in dioxane (3.0 mL) and water (1.0 mL) was added 3-fluorophenylboronic acid (0.033 g, 0.236 mmol), potassium carbonate (0.059 g, 0.429 mmol), and tetrakis(triphenylphosphine)palladium(0) (0.006 g, 5.19 μmol) followed by heating to 120° C. for 20 min. in a Biotage Initiator® microwave synthesizer. Upon cooling, the reaction mixture was diluted with methanol (1.0 mL) and treated with 1N aqueous sodium hydroxide (0.500 mL, 0.500 mmol). After stirring 15 min. at ambient temperature, the reaction was quenched with 1N aqueous hydrochloric acid and the resulting precipitate was filtered, washed with water, and dried in vacuo to afford N-{[3-(3,4-difluorophenyl)-7-(3-fluorophenyl)-6-hydroxy-5-quinoxalinyl]carbonyl}glycine (0.082 g, 0.181 mmol, 84% yield) as a light orange solid. ¹H NMR (400 MHz, DMSO-d₆) δ ppm 11.2 (t, J=4.5 Hz, 1H), 9.44 (s, 1H), 8.53 (ddd, J=11.7, 7.9, 1.9 Hz, 1H), 8.34-8.40 (m, 1H), 8.16 (s, 1H), 7.59 (dt, J=10.4, 8.6 Hz, 1H), 7.51-7.56 (m, 3H), 7.29 (ddd, J=11.9, 5.4, 3.4 Hz, 1H), 4.15 (d, J=4.5 Hz, 2H). MS (ES+) m/e 454 [M+H]⁺.

### Example 66

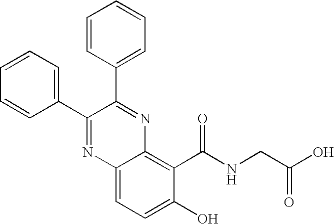

### N-[(6-hydroxy-2,3-diphenyl-5-quinoxalinyl)carbonyl]glycine

66(a) methyl 6-(methyloxy)-2,3-diphenyl-5-quinoxalinecarboxylate. To a solution of methyl 2-amino-6-(methyloxy)-3-nitrobenzoate (example 1(b), 0.500 g, 2.211 mmol) in ethyl acetate (10.0 mL) was added 10% palladium on charcoal (0.235 g, 0.221 mmol) followed by evacuation of the reaction vessel and purging with 1 atmosphere of hydrogen. After stirring overnight at ambient temperature, the reaction mixture was filtered through Celite®, washed through with ethyl acetate, and concentrated in vacuo. The resulting residue was dissolved in methanol (2.0 mL), treated with benzil (0.500 g, 2.378 mmol), and heated to 100° C. for 20 min. in a Biotage Initiator® microwave synthesizer. Upon cooling, the reaction mixture was filtered, washed with methanol, and dried in vacuo to afford methyl 6-(methyloxy)-2,3-diphenyl-5-quinoxalinecarboxylate (0.628 g, 1.695 mmol, 77% yield) as a pale yellow solid. ¹H NMR (400 MHz, CHLOROFORM-d) δ ppm 8.22 (d, J=9.3 Hz, 1H), 7.56 (d, J=9.3 Hz, 1H), 7.48-7.55 (m, 4H), 7.28-7.38 (m, 6H), 4.07 (s, 3H), 4.07 (s, 3H). MS (ES+) m/e 371 [M+H]⁺.

66(b) 6-hydroxy-2,3-diphenyl-5-quinoxalinecarboxylic acid. To a solution of methyl 6-(methyloxy)-2,3-diphenyl-5-quinoxalinecarboxylate (0.628 g, 1.695 mmol) in dichloromethane (6.0 mL) was added boron tribromide (1M solution in dichloromethane) (6.00 mL, 6.00 mmol). The reaction mixture was stirred overnight at ambient temperature, quenched by water, diluted with brine, and extracted thrice with dichloromethane. The combined organic layers were dried over MgSO₄, filtered, concentrated in vacuo, triturated with ethyl acetate, filtered, and dried in vacuo to afford 6-hydroxy-2,3-diphenyl-5-quinoxalinecarboxylic acid (0.512 g, 1.496 mmol, 88% yield) as a beige solid. ¹H NMR (400 MHz, DMSO-d₆) δ ppm 15.8 (br. s., 1H), 12.7 (br. s., 1H), 8.36 (d, J=9.3 Hz, 1H), 7.70 (d, J=9.3 Hz, 1H), 7.36-7.53 (m, 10H). MS (ES+) m/e 343 [M+H]⁺.

66(c) ethyl N-[(6-hydroxy-2,3-diphenyl-5-quinoxalinyl)carbonyl]glycinate. To a solution of 6-hydroxy-2,3-diphenyl-5-quinoxalinecarboxylic acid (0.506 g, 1.478 mmol) and glycine ethyl ester hydrochloride (0.620 g, 4.44 mmol) in dichloromethane (5.0 mL) were added triethylamine (0.820 mL, 5.88 mmol) and PyBOP (1.15 g, 2.210 mmol). The reaction mixture was stirred overnight at ambient temperature, quenched by water, diluted with brine, and extracted thrice with dichloromethane. The combined organic layers were dried over MgSO₄, filtered, concentrated in vacuo, and purified via flash column chromatography (10-100% ethyl acetate in hexanes) to afford ethyl N-[(6-hydroxy-2,3-diphenyl-5-quinoxalinyl)carbonyl]glycinate (0.528 g, 1.235 mmol, 84% yield) as a pale yellow solid. ¹H NMR analysis showed ˜1:1 ratio of rotomers in DMSO-d₆. ¹H NMR (400 MHz, DMSO-d₆) δ ppm 15.1 (s, 1H), 11.4 (t, J=5.6 Hz, 1H), 9.03 (t, J=6.1 Hz, 1H), 8.26 (d, J=9.3 Hz, 1H), 7.98 (d, J=10.1 Hz, 1H), 7.59 (d, J=9.4 Hz, 1H), 7.55-7.60 (m, 2H), 7.32-7.50 (m, 18H), 6.72 (d, J=10.1 Hz, 1H), 4.37 (d, J=5.6 Hz, 2H), 4.16 (q, J=7.1 Hz, 2H), 4.05 (dq, J=7.1, 1.5 Hz, 2H), 3.91 (d, J=6.1 Hz, 2H), 1.19 (t, J=7.1 Hz, 3H), 1.12 (t, J=7.1 Hz, 3H) MS (ES+) m/e 428 [M+H]⁺.

66(d) N-[(6-hydroxy-2,3-diphenyl-5-quinoxalinyl)carbonyl]glycine. To a suspension of ethyl N-[(6-hydroxy-2,3-diphenyl-5-quinoxalinyl)carbonyl]glycinate (0.150 g, 0.351 mmol) in methanol (1.0 mL) and tetrahydrofuran (1.0 mL) was added 1N aqueous sodium hydroxide (0.500 mL, 0.500 mmol). After stirring 30 min. at ambient temperature, the reaction was quenched with 1N aqueous hydrochloric acid and the resulting precipitate was filtered, washed with water, and dried in vacuo to afford N-[(6-hydroxy-2,3-diphenyl-5-quinoxalinyl)carbonyl]glycine (0.130 g, 0.325 mmol, 93% yield) as a pale yellow solid. ¹H NMR (400 MHz, DMSO-d₆) δ ppm 15.3 (s, 1H), 13.1 (br. s., 1H), 11.4 (t, J=5.3 Hz, 1H), 8.25 (d, J=9.3 Hz, 1H), 7.57-7.61 (m, 2H), 7.57 (d, J=9.3 Hz, 1H), 7.48 (dd, J=7.8, 1.5 Hz, 2H), 7.34-7.45 (m, 6H), 4.28 (d, J=5.3 Hz, 1H). MS (ES+) m/e 400 [M+H]⁺.

### Example 67

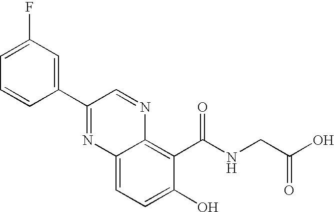

### N-{[2-(3-fluorophenyl)-6-hydroxy-5-quinoxalinyl]carbonyl}glycine

67(a) Ethyl N-{[2-(3-fluorophenyl)-6-hydroxy-5-quinoxalinyl]carbonyl}glycinate. To a mixture of ethyl N-[(2-bromo-6-hydroxy-5-quinoxalinyl)carbonyl]glycinate (example 5(a), 0.500 g, 1.41 mmol), 3-fluorophenylboronic acid (0.237 g, 1.69 mmol) and potassium carbonate (0.390 g, 2.82 mmol) in 1,4-dioxane (3.0 mL) and water (1.0 mL) was added tetra-kis(triphenylphosphine)palladium (0.016 g, 0.014 mmol) followed by evacuation of the reaction vessel and purging with nitrogen. The reaction mixture was then heated in a Biotage Initiator® microwave synthesizer at 120° C. for 30 min. Upon cooling, the mixture was diluted with EtOAc (10 mL) and water (20 mL), and the aqueous phase was extracted with EtOAc (10 mL*3). The combined organic phases were dried (Na₂SO₄) and concentrated in vacuo to afford the title compound (0.600 g, 114.94% yield, crude) as a yellow solid, MS (ES+) m/e 370 [M+H]⁺, used in the next step without further purification.

67(b) N-{[2-(3-fluorophenyl)-6-hydroxy-5-quinoxalinyl]carbonyl}glycine. To ethyl N-{[2-(3-fluorophenyl)-6-hydroxy-5-quinoxalinyl]carbonyl}glycinate (0.600 g, 1.62 mmol) was added aqueous sodium hydroxide (1N, 8.0 mL), methanol (8.0 mL) and tetrahydrofuran (8.0 mL). The mixture was stirred at ambient temperature for 20 min and quenched with 1N hydrochloric acid. The precipitate was collected by filtration, washed through with methanol (10.0 mL) and EtOAc (15.0 mL), dried in vacuo to afford the title compound (0.062 g, 11.2% yield) as a yellow solid. ¹H NMR (300 MHz, DMSO-d6) δ ppm 15.26 (s, 1H, br), 12.92 (s, 1H, br), 11.31 (t, 1H, br, J=5.1 Hz), 9.50 (s, 1H), 8.18 (d, 1H, J=9.3 Hz), 8.13 (d, 1H, J=7.8 Hz), 8.08 (m, 1H), 7.61 (m, 1H), 7.54 (d, 1H, J=9.6 Hz), 7.37 (m, 1H), 4.25 (d, 2H, J=5.7 Hz). MS (ES+) m/e 342 [M+H]⁺.

### Example 68

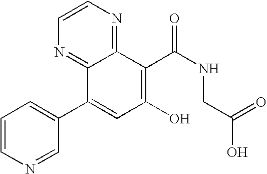

### N-{[6-hydroxy-8-(3-pyridinyl)-5-quinoxalinyl]carbonyl}glycine

68(a) 8-bromo-6-hydroxy-5-quinoxalinecarboxylic acid. In a 100 mL round-bottomed flask was placed methyl 8-bromo-6-(methyloxy)-5-quinoxalinecarboxylate (example 59(a), 600 mg, 2.019 mmol) in dichloromethane (20 ml) to give a yellow solution. Boron tribromide (1.0 M in dichloromethane) (6.06 ml, 6.06 mmol) was added. The reaction was kept stirring at ambient temperature overnight and quenched with water. Precipitate was collected, washed with water and dried under vacuo to afford 8-bromo-6-hydroxy-5-quinoxalinecarboxylic acid (480 mg, 1.784 mmol, 88% yield) as a yellow solid. 1H NMR (400 MHz, DMSO-d6) δ ppm 12.66 (br. s., 1H), 9.03 (d, J=2.3 Hz, 1H), 9.01 (d, J=2.3 Hz, 1H), 8.06 (s, 1H). MS (ES+) m/e 269[M+H]⁺.

68(b) ethyl N-[(8-bromo-6-hydroxy-5-quinoxalinyl)carbonyl]glycinate. In a 100 mL round-bottomed flask was placed 8-bromo-6-hydroxy-5-quinoxalinecarboxylic acid (480 mg, 1.784 mmol), triethylamine (0.746 ml, 5.35 mmol), ethyl glycine hydrochloride (498 mg, 3.57 mmol) in N,N-dimethylformamide (10 ml) to give a yellow solution. PyBOP (1021 mg, 1.962 mmol) was added. The reaction was kept stirring at ambient temperature for 2 hours and concentrated under vacuo. The precipitate was collected, washed with water and ethyl acetate and dried under high vacuum to give ethyl N-[(8-bromo-6-hydroxy-5-quinoxalinyl)carbonyl]glycinate (350 mg, 0.988 mmol, 55.4% yield) as a brown solid. 1H NMR (400 MHz, DMSO-d6) δ ppm 15.34 (s, 1H), 11.32 (t, J=5.6 Hz, 1H), 8.99 (d, J=5.1 Hz, 2H), 8.03 (s, 1H), 4.30 (d, J=5.6 Hz, 2H), 4.17 (q, J=7.2 Hz, 2H), 1.23 (t, J=7.1 Hz, 3H). MS (ES+) m/e 356[M+H]⁺.

68(c) N-{[6-hydroxy-8-(3-pyridinyl)-5-quinoxalinyl]carbonyl}glycine. In a 10 mL microwave vial was placed ethyl N-[(8-bromo-6-hydroxy-5-quinoxalinyl)carbonyl]glycinate (40 mg, 0.113 mmol), 3-pyridyl boronic acid (15.27 mg, 0.124 mmol), tetrakis(triphenylphosphine)palladium(0) (3.26 mg, 2.82 μmol), and potassium carbonate (31.2 mg, 0.226 mmol) in 1,4-dioxane (3.0 ml) and water (1.000 ml) to give a yellow suspension. The mixture was heated to 120° C. for 30 min. in a Biotage Initiator® microwave synthesizer and was diluted with methanol. Sodium hydroxide (1.0 N in water) (0.452 ml, 0.452 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[6-hydroxy-8-(3-pyridinyl)-5-quinoxalinyl]carbonyl}glycine (20 mg, 0.046 mmol, 40.4% yield) as a yellow solid. 1H NMR (400 MHz, DMSO-d6) δ ppm 15.44 (s, 1H), 12.93 (br. s., 1H), 11.45 (t, J=5.6 Hz, 1H), 9.01 (d, J=2.0 Hz, 1H), 8.98 (s, 1H), 8.93 (d, J=2.0 Hz, 1H), 8.76 (d, J=4.0 Hz, 1H), 8.32 (d, J=7.8 Hz, 1H), 7.75 (s, 1H), 7.72 (d, J=2.8 Hz, 1H), 4.27 (d, J=5.6 Hz, 2H). MS (ES+) m/e 325[M+H]⁺.

### Example 69

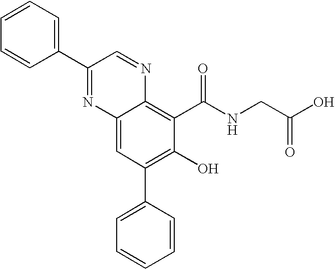

### N-[(6-hydroxy-2,7-diphenyl-5-quinoxalinyl)carbonyl]glycine

69(a) methyl 7-bromo-2-chloro-6-(methyloxy)-5-quinoxalinecarboxylate. To a solution of methyl 7-bromo-6-(methyloxy)-2-oxo-1,2-dihydro-5-quinoxalinecarboxylate (1.22 g, 3.90 mmol) was added phosphorus oxychloride (3.0 ml, 32.2 mmol). After heating to reflux for 2 h, the reaction mixture was carefully treated with ice water. The resulting precipitate was filtered, washed with water, and concentrated in vacuo to afford methyl 7-bromo-2-chloro-6-(methyloxy)-5-quinoxalinecarboxylate (0.897 g, 2.435 mmol, 62.5% yield) as a light gray solid. ¹H NMR (400 MHz, DMSO-d₆) δ ppm 9.04 (s, 1H), 8.57 (s, 1H), 3.97 (s, 3H), 3.97 (s, 3H). MS (ES+) m/e 331/333 [M+H]⁺.

69(b) methyl 6-(methyloxy)-2,7-diphenyl-5-quinoxalinecarboxylate. A solution of methyl 7-bromo-2-chloro-6-(methyloxy)-5-quinoxalinecarboxylate (0.255 g, 0.769 mmol), phenylboronic acid (0.206 g, 1.692 mmol), potassium carbonate (0.319 g, 2.307 mmol), and tetrakis(triphenylphosphine)palladium(0) (0.027 g, 0.023 mmol) in 1,4-dioxane (2.0 ml) and water (0.667 ml) was heated to 100° C. for 20 min. in a Biotage Initiator® microwave synthesizer and then to 106° C. under conventional heating for two days. Upon cooling, the reaction mixture was filtered through Celite®, washed through with ethyl acetate, and concentrated in vacuo. The residue was purified via flash column chromatography (0-60% ethyl acetate in hexanes) to obtain methyl 6-(methyloxy)-2,7-diphenyl-5-quinoxalinecarboxylate (0.266 g, 0.718 mmol, 93% yield) as a yellow solid. ¹H NMR (400 MHz, DMSO-d₆) δ ppm 9.60 (s, 1H), 8.34 (d, J=2.0 Hz, 1H), 8.32 (d, J=1.5 Hz, 1H), 8.17 (s, 1H), 7.74 (t, J=1.8 Hz, 1H), 7.70-7.73 (m, 1H), 7.53-7.66 (m, 5H), 7.47-7.53 (m, 1H), 3.99 (s, 3H), 3.54 (s, 3H). MS (ES+) m/e 371 [M+H]⁺.

69(c) 6-hydroxy-2,7-diphenyl-5-quinoxalinecarboxylic acid. A solution of methyl 6-(methyloxy)-2,7-diphenyl-5-quinoxalinecarboxylate (0.266 g, 0.718 mmol) in dichloromethane (3.0 mL) was treated with boron tribromide (1M solution in dichloromethane) (2.154 mL, 2.154 mmol) at room temperature overnight. The reaction mixture was quenched by water and the layers were separated. The aqueous phase was extracted with ethyl acetate. The combined organic portions were dried over magnesium sulfate, filtered and concentrated in vacuo. The resulting solid was washed with ether and dried in vacuo to give 6-hydroxy-2,7-diphenyl-5-quinoxalinecarboxylic acid (0.201 g, 0.587 mmol, 82% yield) as an orange solid. ¹H NMR (400 MHz, DMSO-d₆) δ ppm 14.25 (br. s., 1H), 9.62 (s, 1H), 8.33 (s, 1H), 8.31 (d, J=1.8 Hz, 1H), 8.29 (d, J=1.3 Hz, 1H), 7.76 (t, J=1.8 Hz, 1H), 7.73-7.75 (m, 1H), 7.57-7.66 (m, 3H), 7.46-7.58 (m, 3H). MS (ES+) m/e 343 [M+H]⁺.

69(d) ethyl N-[(6-hydroxy-2,7-diphenyl-5-quinoxalinyl)carbonyl]glycinate. A solution of 6-hydroxy-2,7-diphenyl-5-quinoxalinecarboxylic acid (0.201 g, 0.587 mmol) and ethyl glycine hydrochloride (0.164 g, 1.174 mmol) in N,N-Dimethylformamide (DMF) (3.0 mL) was treated with triethylamine (0.250 mL, 1.794 mmol) and PyBOP (0.336 g, 0.646 mmol). The reaction mixture was stirred overnight at ambient temperature, quenched by water, filtered and dried in vacuo to obtain ethyl N-[(6-hydroxy-2,7-diphenyl-5-quinoxalinyl)carbonyl]glycinate (0.206 g, 0.482 mmol, 82% yield) as an orange solid. ¹H NMR (400 MHz, DMSO-d₆) δ ppm 16.00 (s, 1H), 11.64 (t, J=5.7 Hz, 1H), 9.55 (s, 1H), 8.34 (d, J=1.5 Hz, 1H), 8.32 (d, J=1.3 Hz, 1H), 8.23 (s, 1H), 7.74 (t, J=1.8 Hz, 1H), 7.73 (t, J=1.4 Hz, 1H), 7.56-7.65 (m, 3H), 7.47-7.56 (m, 3H), 4.39 (d, J=5.6 Hz, 2H), 4.20 (q, J=7.1 Hz, 2H), 1.26 (t, J=7.1 Hz, 3H). MS (ES+) m/e 428 [M+H]⁺.

69(e) N-[(6-hydroxy-2,7-diphenyl-5-quinoxalinyl)carbonyl]glycine. To a suspension of ethyl N-[(6-hydroxy-2,7-diphenyl-5-quinoxalinyl)carbonyl]glycinate (0.206 g, 0.482 mmol) in ethanol (3.0 mL) was added 1N aqueous sodium hydroxide (3.00 ml, 3.00 mmol). After stirring overnight at ambient temperature, the reaction was quenched with 1N aqueous hydrochloric acid and the resulting precipitate was filtered, washed with water, and dried in vacuo to obtain N-[(6-hydroxy-2,7-diphenyl-5-quinoxalinyl)carbonyl]glycine (0.134 g, 0.336 mmol, 69.6% yield) as a yellow solid. ¹H NMR (400 MHz, DMSO-d₆) δ ppm 16.09 (s, 1H), 12.98 (br. s., 1H), 11.57 (t, J=5.6 Hz, 1H), 9.51 (s, 1H), 8.31 (d, J=1.5 Hz, 1H), 8.29 (br. s., 1H), 8.17 (s, 1H), 7.73 (d, J=1.5 Hz, 1H), 7.71 (d, J=1.0 Hz, 1H), 7.56-7.64 (m, 3H), 7.45-7.55 (m, 3H), 4.29 (d, J=5.6 Hz, 2H). MS (ES+) m/e 400M+H]⁺.

### Example 70

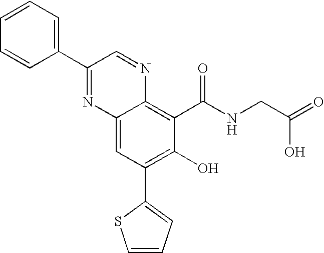

### N-{[6-hydroxy-2-phenyl-7-(2-thienyl)-5-quinoxalinyl]carbonyl}glycine

70(a) methyl 6-(methyloxy)-2-oxo-7-(2-thienyl)-1,2-dihydro-5-quinoxalinecarboxylate. To a solution of methyl 7-bromo-6-(methyloxy)-2-oxo-1,2-dihydro-5-quinoxalinecarboxylate (1.23 g, 3.93 mmol) in 1,4-dioxane (5.0 ml) was added tributyl(2-thienyl)stannane (1.466 g, 3.93 mmol) and tetrakis(triphenylphosphine)palladium(0) (0.272 g, 0.236 mmol) followed by heating to 150° C. for 40 min. in a Biotage Initiator® microwave synthesizer. Upon cooling, the reaction mixture was filtered through celite, washed with ethyl acetate and concentrated in vacuo. The obtained residue was purified via flash column chromatography (50-70% ethyl acetate in hexanes) to obtain methyl 6-(methyloxy)-2-oxo-7-(2-thienyl)-1,2-dihydro-5-quinoxalinecarboxylate (1.17 g, 3.70 mmol, 94% yield) as a yellow solid. ¹H NMR (400 MHz, DMSO-d₆) δ ppm 12.50 (br. s., 1H), 8.18 (s, 1H), 7.78 (dd, J=5.2, 1.1 Hz, 1H), 7.63 (d, J=1.3 Hz, 1H), 7.62 (s, 1H), 7.24 (dd, J=5.2, 3.7 Hz, 1H), 3.93 (s, 3H), 3.65 (s, 3H). MS (ES+) m/e 317 [M+H]⁺.

70(b) methyl 2-chloro-6-(methyloxy)-7-(2-thienyl)-5-quinoxalinecarboxylate. To a solution of methyl 6-(methyloxy)-2-oxo-7-(2-thienyl)-1,2-dihydro-5-quinoxalinecarboxylate (1.17 g, 3.70 mmol) was added phosphorus oxychloride (3.0 ml, 32.2 mmol). After heating to reflux for 3 h, the reaction mixture was carefully treated with ice water. The resulting dark precipitate was filtered and washed with water. The mother liquor was extracted several times with ethyl acetate, dried over magnesium sulfate, filtered and concentrated. The resulting dark syrup was purified via flash column chromatography (0-100% ethyl acetate in hexanes) to obtain methyl 2-chloro-6-(methyloxy)-7-(2-thienyl)-5-quinoxalinecarboxylate (0.379 g, 1.132 mmol, 30.6% yield) as a bright yellow solid. ¹H NMR (400 MHz, DMSO-d₆) δ ppm 8.97 (s, 1H), 8.51 (s, 1H), 7.97 (dd, J=3.8, 1.3 Hz, 1H), 7.84 (dd, J=5.2, 1.1 Hz, 1H), 7.27 (dd, J=5.1, 3.8 Hz, 1H), 3.99 (s, 3H), 3.85 (s, 3H). MS (ES+) m/e 335 [M+H]⁺.

70(c) methyl 6-(methyloxy)-2-phenyl-7-(2-thienyl)-5-quinoxalinecarboxylate. To a suspension of methyl 2-chloro-6-(methyloxy)-7-(2-thienyl)-5-quinoxalinecarboxylate (0.220 g, 0.657 mmol) in dioxane (3.0 mL) and water (1.0 mL) was added phenylboronic acid (0.080 g, 0.657 mmol), potassium carbonate (0.272 g, 1.971 mmol), and tetrakis(triphenylphosphine)palladium(0) (0.023 g, 0.020 mmol) followed by heating to 120° C. for 20 min. in a Biotage Initiator® microwave synthesizer. Upon cooling, the reaction mixture was poured into water, extracted thrice with ethyl acetate, dried over magnesium sulfate, filtered and concentrated in vacuo. The residue was purified via flash column chromatography (0-100% ethyl acetate in hexanes) to obtain methyl 6-(methyloxy)-2-phenyl-7-(2-thienyl)-5-quinoxalinecarboxylate (0.245 g, 0.651 mmol, 99% yield) as an orange solid. ¹H NMR (400 MHz, DMSO-d₆) δ ppm 9.56 (s, 1H), 8.55 (s, 1H), 8.35 (d, J=1.8 Hz, 1H), 8.33 (d, J=1.5 Hz, 1H), 7.97 (dd, J=3.7, 1.1 Hz, 1H), 7.83 (dd, J=5.2, 1.1 Hz, 1H), 7.55-7.67 (m, 3H), 7.27 (dd, J=5.1, 3.8 Hz, 1H), 4.01 (s, 3H), 3.85 (s, 3H). MS (ES+) m/e 377 [M+H]⁺.

70(d) 6-hydroxy-2-phenyl-7-(2-thienyl)-5-quinoxalinecarboxylic acid. A solution of methyl 6-(methyloxy)-2-phenyl-7-(2-thienyl)-5-quinoxalinecarboxylate (0.245 g, 0.651 mmol) in dichloromethane (10 mL) was treated with boron tribromide (1M solution in dichloromethane) (2.60 mL, 2.60 mmol) at room temperature overnight. The reaction mixture was quenched by water and extracted twice with ethyl acetate. The combined organic portions were dried over magnesium sulfate, filtered, concentrated and purified via flash column chromatography (0-100% ethyl acetate in hexanes) to give 6-hydroxy-2-phenyl-7-(2-thienyl)-5-quinoxalinecarboxylic acid (0.176 g, 0.505 mmol, 78% yield) as a deep orange solid. ¹H NMR (400 MHz, DMSO-d₆) δ ppm 15.78 (s, 1H), 9.42 (s, 1H), 8.73 (s, 1H), 8.27 (d, J=1.5 Hz, 1H), 8.25 (br. s., 1H), 8.17 (dd, J=3.8, 1.0 Hz, 1H), 7.85 (dd, J=5.1, 1.0 Hz, 1H), 7.55-7.67 (m, 3H), 7.28 (dd, J=5.1, 3.8 Hz, 1H). MS (ES+) m/e 349 [M+H]⁺.

70(e) ethyl N-{[6-hydroxy-2-phenyl-7-(2-thienyl)-5-quinoxalinyl]carbonyl}glycinate. A solution of 6-hydroxy-2-phenyl-7-(2-thienyl)-5-quinoxalinecarboxylic acid (0.176 g, 0.505 mmol) and ethyl glycine hydrochloride (0.141 g, 1.010 mmol) in N,N-Dimethylformamide (DMF) (3.0 mL) was treated with triethylamine (0.211 mL, 1.516 mmol) and PyBOP (0.289 g, 0.556 mmol). The reaction mixture was stirred overnight at ambient temperature, quenched by water, extracted thrice with ethyl acetate, dried over magnesium sulfate, filtered and concentrated in vacuo. The deep purple residue was decanted in water as a precipitate appeared. The precipitate was filtered, washed with water and dried in vacuo to obtain ethyl N-{[6-hydroxy-2-phenyl-7-(2-thienyl)-5-quinoxalinyl]carbonyl}glycinate (0.08 g, 0.185 mmol, 36.5% yield) as a deep red solid. ¹H NMR (400 MHz, DMSO-d₆) δ ppm 11.66 (t, J=5.7 Hz, 1H), 9.51 (s, 1H), 8.66 (s, 1H), 8.35 (br. s., 1H), 8.33 (d, J=1.0 Hz, 1H), 8.09 (dd, J=3.9, 1.1 Hz, 1H), 7.81 (dd, J=5.1, 1.3 Hz, 1H), 7.57-7.67 (m, 3H), 7.26 (dd, J=5.3, 3.8 Hz, 1H), 4.40 (d, J=5.3 Hz, 2H), 4.21 (q, J=7.1 Hz, 2H), 1.26 (t, J=7.1 Hz, 3H). MS (ES+) m/e 434 [M+H]⁺.

70(f) N-{[6-hydroxy-2-phenyl-7-(2-thienyl)-5-quinoxalinyl]carbonyl}glycine. To a suspension of ethyl N-{[6-hydroxy-2-phenyl-7-(2-thienyl)-5-quinoxalinyl]carbonyl}glycinate (0.08 g, 0.185 mmol) in ethanol (1.0 mL) was added 1N aqueous sodium hydroxide (0.185 ml, 0.185 mmol). After stirring 30 min. at ambient temperature, the reaction was quenched with 1N aqueous hydrochloric acid and the resulting precipitate was filtered, washed with water, and dried in vacuo to obtain N-{[6-hydroxy-2-phenyl-7-(2-thienyl)-5-quinoxalinyl]carbonyl}glycine (0.024 g, 0.059 mmol, 32.1% yield) as a brown solid. ¹H NMR (400 MHz, DMSO-d₆) δ ppm 13.01 (br. s., 1H), 9.51 (s, 1H), 8.65 (s, 1H), 8.35 (t, J=1.8 Hz, 1H), 8.33 (d, J=1.3 Hz, 1H), 8.09 (dd, J=3.8, 1.3 Hz, 1H), 7.80 (dd, J=5.1, 1.0 Hz, 1H), 7.61-7.66 (m, 1H), 7.56-7.62 (m, 2H), 7.26 (dd, J=5.1, 3.8 Hz, 1H), 4.32 (d, J=5.6 Hz, 2H). MS (ES+) m/e 406 [M+H]⁺.

### Example 71

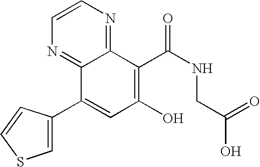

### N-{[6-hydroxy-8-(3-thienyl)-5-quinoxalinyl]carbonyl}glycine

A solution of ethyl N-[(8-bromo-6-hydroxy-5-quinoxalinyl)carbonyl]glycinate (20 mg, 0.056 mmol), 3-thienylboronic acid (7.22 mg, 0.056 mmol), potassium carbonate (14.19 mg, 0.103 mmol), and tetrakis(triphenylphosphine)palladium(0) (2.97 mg, 2.57 μmol) in 1,4-dioxane (3 ml) and water (1.000 ml) was heated to 120° C. for 30 min. in a Biotage Initiator® microwave synthesizer. The reaction was diluted with methanol. Sodium hydroxide (1.0 N in water) (0.205 ml, 0.205 mmol) was added. The mixture was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[6-hydroxy-8-(3-thienyl)-5-quinoxalinyl]carbonyl}glycine (9.0 mg, 0.020 mmol, 39.5% yield) as a yellow solid. 1H NMR (400 MHz, DMSO-d6) δ ppm 15.36 (s, 1H), 12.92 (s, 1H), 11.44 (t, J=5.6 Hz, 1H), 8.97 (d, J=2.0 Hz, 1H), 8.95 (d, J=2.0 Hz, 1H), 8.35 (d, J=1.8 Hz, 1H), 7.78 (dd, J=5.1, 1.0 Hz, 1H), 7.75 (s, 1H), 7.70 (dd, J=5.1, 3.0 Hz, 1H), 4.25 (d, J=5.6 Hz, 2H). MS (ES+) m/e 330[M+H]⁺

### Example 72

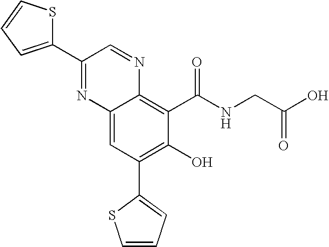

### N-[(6-hydroxy-2,7-di-2-thienyl-5-quinoxalinyl)carbonyl]glycine

72(a) methyl 6-(methyloxy)-2,7-di-2-thienyl-5-quinoxalinecarboxylate. To a solution of methyl 7-bromo-2-chloro-6-(methyloxy)-5-quinoxalinecarboxylate (0.264 g, 0.796 mmol) in 1,4-dioxane (1.5 ml) was added tributyl(2-thienyl)stannane (0.654 g, 1.752 mmol) and tetrakis(triphenylphosphine)palladium(0) (0.028 g, 0.024 mmol) followed by heating to 150° C. for 20 min. in a Biotage Initiator® microwave synthesizer. Upon cooling, the reaction mixture was filtered through celite, washed through with ethyl acetate and concentrated in vacuo. The obtained residue was purified via flash column chromatography (0-40% ethyl acetate in hexanes) to obtain methyl 6-(methyloxy)-2,7-di-2-thienyl-5-quinoxalinecarboxylate (0.280 g, 0.732 mmol, 92% yield) as a pale tan solid. ¹H NMR (400 MHz, DMSO-d₆) δ ppm 9.53 (s, 1H), 8.43 (s, 1H), 8.22 (dd, J=3.8, 1.0 Hz, 1H), 7.99 (dd, J=3.8, 1.0 Hz, 1H), 7.89 (dd, J=4.9, 1.1 Hz, 1H), 7.82 (dd, J=5.2, 1.1 Hz, 1H), 7.32 (dd, J=5.1, 3.8 Hz, 1H), 7.25 (dd, J=5.2, 3.7 Hz, 1H), 4.00 (s, 3H), 3.83 (s, 3H). MS (ES+) m/e 383 [M+H]⁺.

72(b) 6-hydroxy-2,7-di-2-thienyl-5-quinoxalinecarboxylic acid. A solution of methyl 6-(methyloxy)-2,7-di-2-thienyl-5-quinoxalinecarboxylate (0.280 g, 0.732 mmol) in dichloromethane (10 mL) was treated with boron tribromide (1M solution in dichloromethane) (2.2 mL, 2.200 mmol) at room temperature overnight. The reaction mixture was quenched by water and separated layers. The aqueous phase was extracted with ethyl acetate. The combined organic portions were dried over magnesium sulfate, filtered and concentrated. The resulting solid was washed with ethyl ether and dried in vacuo to obtain 6-hydroxy-2,7-di-2-thienyl-5-quinoxalinecarboxylic acid (0.180 g, 0.508 mmol, 69.4% yield) as a red solid. ¹H NMR (400 MHz, DMSO-d₆) δ ppm 15.05 (br. s., 1H), 9.49 (s, 1H), 8.63 (s, 1H), 8.17 (dd, J=3.8, 1.3 Hz, 1H), 8.13 (dd, J=3.8, 1.0 Hz, 1H), 7.88 (dd, J=4.9, 1.1 Hz, 1H), 7.84 (dd, J=5.1, 1.3 Hz, 1H), 7.32 (dd, J=5.1, 3.8 Hz, 1H), 7.27 (dd, J=5.3, 3.8 Hz, 1H). MS (ES+) m/e 355 [M+H]⁺.

72(c) ethyl N-[(6-hydroxy-2,7-di-2-thienyl-5-quinoxalinyl)carbonyl]glycinate. A solution of 6-hydroxy-2,7-di-2-thienyl-5-quinoxalinecarboxylic acid (0.180 g, 0.508 mmol) and ethyl glycine hydrochloride (0.142 g, 1.016 mmol) in N,N-Dimethylformamide (DMF) (3.0 mL) was treated with triethylamine (0.212 mL, 1.524 mmol) and PyBOP (0.291 g, 0.559 mmol). The reaction mixture was stirred overnight at ambient temperature, quenched by water, filtered and dried in vacuo to obtain ethyl N-[(6-hydroxy-2,7-di-2-thienyl-5-quinoxalinyl)carbonyl]glycinate (0.170 g, 0.387 mmol, 76% yield) as a deep orange solid. ¹H NMR (400 MHz, DMSO-d₆) δ ppm 11.60 (t, J=5.6 Hz, 1H), 9.50 (s, 1H), 8.54 (s, 1H), 8.21 (dd, J=3.8, 1.0 Hz, 1H), 8.11 (dd, J=3.8, 1.0 Hz, 1H), 7.86 (dd, J=4.9, 1.1 Hz, 1H), 7.80 (dd, J=5.2, 1.1 Hz, 1H), 7.31 (dd, J=5.1, 3.8 Hz, 1H), 7.26 (dd, J=5.1, 3.8 Hz, 1H), 4.39 (d, J=5.6 Hz, 2H), 4.21 (q, J=7.1 Hz, 2H), 1.26 (t, J=7.1 Hz, 3H). MS (ES+) m/e 440 [M+H]⁺.

72(d) N-[(6-hydroxy-2,7-di-2-thienyl-5-quinoxalinyl)carbonyl]glycine. To a suspension of ethyl N-[(6-hydroxy-2,7-di-2-thienyl-5-quinoxalinyl)carbonyl]glycinate (0.170 g, 0.387 mmol) in ethanol (5.0 mL) was added sodium hydroxide (1N aqueous solution) (3.00 ml, 3.00 mmol). After stirring overnight at ambient temperature, the reaction was quenched with 1N aqueous hydrochloric acid and the resulting precipitate was filtered, washed with water, and dried in vacuo to obtain N-[(6-hydroxy-2,7-di-2-thienyl-5-quinoxalinyl)carbonyl]glycine (0.118 g, 0.287 mmol, 74.1% yield) as a brown solid. ¹H NMR (400 MHz, DMSO-d₆) δ ppm 13.02 (br. s., 1H), 11.52 (t, J=5.4 Hz, 1H), 9.46 (s, 1H), 8.49 (s, 1H), 8.19 (dd, J=3.8, 1.0 Hz, 1H), 8.08 (dd, J=3.7, 1.1 Hz, 1H), 7.84 (dd, J=5.1, 1.0 Hz, 1H), 7.78 (dd, J=5.1, 1.0 Hz, 1H), 7.29 (dd, J=5.1, 3.8 Hz, 1H), 7.24 (dd, J=5.3, 3.8 Hz, 1H), 4.29 (d, J=5.6 Hz, 2H). MS (ES+) m/e 412 [M+H]⁺.

### Example 73

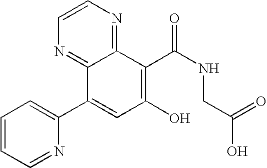

### N-{[6-hydroxy-8-(2-pyridinyl)-5-quinoxalinyl]carbonyl}glycine

In a 10 mL microwave vial was placed 2-(tributylstannanyl)pyridine (22.87 mg, 0.062 mmol), tetrakis(triphenylphosphine)palladium(0) (3.26 mg, 2.82 μmol) and ethyl N-[(8-bromo-6-hydroxy-5-quinoxalinyl)carbonyl]glycinate (20 mg, 0.056 mmol) in 1,4-dioxane (4.0 ml) to give a yellow suspension. The mixture was heated to 150° C. for 60 min. in a Biotage Initiator® microwave synthesizer and was diluted with methanol. Sodium hydroxide (1.0 N in water) (0.452 ml, 0.452 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[6-hydroxy-8-(2-pyridinyl)-5-quinoxalinyl]carbonyl}glycine (10 mg, 0.023 mmol, 40.4% yield) as a yellow solid. 1H NMR (400 MHz, DMSO-d6) δ ppm 15.36 (br. s., 1H), 11.46 (t, J=5.4 Hz, 1H), 9.01 (d, J=1.8 Hz, 1H), 8.94 (d, J=1.5 Hz, 1H), 8.81 (d, J=4.5 Hz, 1H), 8.09-8.16 (m, 1H), 8.04 (t, J=7.7 Hz, 1H), 7.86 (s, 1H), 7.58 (d, J=5.3 Hz, 1H), 4.27 (d, J=5.6 Hz, 2H). MS (ES+) m/e 325[M+H]+

### Example 74

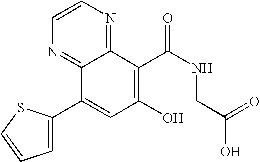

### N-{[6-hydroxy-8-(2-thienyl)-5-quinoxalinyl]carbonyl}glycine

In a 10 mL microwave vial was placed ethyl N-[(8-bromo-6-hydroxy-5-quinoxalinyl)carbonyl]glycinate (example 68(b), 20 mg, 0.056 mmol), 2-thienylboronic acid (7.95 mg, 0.062 mmol), and potassium carbonate (15.61 mg, 0.113 mmol) in 1,4-dioxane (3.0 ml) and water (1.0 ml) to give a yellow suspension. Tetrakis(triphenylphosphine)palladium(0) (6.53 mg, 5.65 μmol) was added. The mixture was heated to 120° C. for 60 min. in a Biotage Initiator® microwave synthesizer, then cooled and diluted with methanol. Sodium hydroxide (1.0 N in water) (0.226 ml, 0.226 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[6-hydroxy-8-(2-thienyl)-5-quinoxalinyl]carbonyl}glycine (5 mg, 0.011 mmol, 19.97% yield) as a yellow solid. 1H NMR (400 MHz, DMSO-d6) δ ppm 15.40 (br. s., 1H), 11.35 (t, J=5.3 Hz, 1H), 8.88-9.16 (m, 2H), 8.16 (d, J=3.8 Hz, 1H), 8.04 (s, 1H), 7.87 (dd, J=5.1, 1.0 Hz, 1H), 7.25 (dd, J=5.1, 3.8 Hz, 1H), 4.21 (d, J=5.6 Hz, 2H). MS (ES+) m/e 330[M+H]⁺

### Example 75

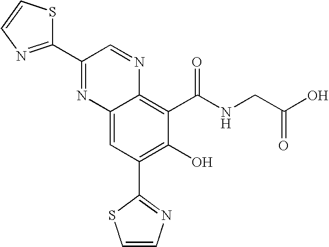

### N-[(6-hydroxy-2,7-di-1,3-thiazol-2-yl-5-quinoxalinyl)carbonyl]glycine

75(a) methyl 6-(methyloxy)-2,7-di-1,3-thiazol-2-yl-5-quinoxalinecarboxylate. To a solution of methyl 7-bromo-2-chloro-6-(methyloxy)-5-quinoxalinecarboxylate (0.330 g, 0.995 mmol) in 1,4-dioxane (1.5 ml) was added 2-(tributylstannanyl)-1,3-thiazole (0.819 g, 2.190 mmol) and tetrakis(triphenylphosphine)palladium(0) (0.035 g, 0.030 mmol) followed by heating to 150° C. for 20 min. in a Biotage Initiator® microwave synthesizer. Upon cooling, the reaction mixture was filtered through celite, washed with ethyl acetate and concentrated in vacuo. The resulting solid was purified via flash column chromatography (20-60% ethyl acetate in hexanes) to obtain methyl 6-(methyloxy)-2,7-di-1,3-thiazol-2-yl-5-quinoxalinecarboxylate (0.227 g, 0.590 mmol, 59.3% yield) as a pale yellow solid. ¹H NMR (400 MHz, DMSO-d₆) δ ppm 9.64 (s, 1H), 8.98 (s, 1H), 8.19 (d, J=3.0 Hz, 1H), 8.17 (d, J=3.0 Hz, 1H), 8.11 (d, J=3.3 Hz, 1H), 8.08 (d, J=3.3 Hz, 1H), 4.07 (s, 3H), 4.04 (s, 3H). MS (ES+) m/e 385 [M+H]⁺.

75(b) 6-hydroxy-2,7-di-1,3-thiazol-2-yl-5-quinoxalinecarboxylic acid. A solution of methyl 6-(methyloxy)-2,7-di-1,3-thiazol-2-yl-5-quinoxalinecarboxylate (0.227 g, 0.590 mmol) in dichloromethane (10 mL) was treated with boron tribromide (1M solution in dichloromethane) (2.0 mL, 2.000 mmol) at room temperature overnight. The reaction mixture was quenched by water and filtered. The solid was washed with water and dried in vacuo to obtain 6-hydroxy-2,7-di-1,3-thiazol-2-yl-5-quinoxalinecarboxylic acid (0.170 g, 0.477 mmol, 81% yield) as an orange solid. ¹H NMR (400 MHz, DMSO-d₆) δ ppm 9.52 (s, 1H), 8.97 (s, 1H), 8.19 (d, J=3.3 Hz, 1H), 8.16 (d, J=3.0 Hz, 1H), 8.08 (d, J=0.8 Hz, 1H), 8.08 (d, J=1.0 Hz, 1H). MS (ES+) m/e 357 M+H]⁺.

75(c) ethyl N-[(6-hydroxy-2,7-di-1,3-thiazol-2-yl-5-quinoxalinyl)carbonyl]glycinate. A solution of 6-hydroxy-2,7-di-1,3-thiazol-2-yl-5-quinoxalinecarboxylic acid (0.170 g, 0.477 mmol) and ethyl glycine hydrochloride (0.133 g, 0.954 mmol) in N,N-Dimethylformamide (DMF) (3.0 mL) was treated with triethylamine (0.199 mL, 1.431 mmol) and PyBOP (0.273 g, 0.525 mmol). The reaction mixture was stirred overnight at ambient temperature, quenched by water, filtered and dried in vacuo to obtain ethyl N-[(6-hydroxy-2,7-di-1,3-thiazol-2-yl-5-quinoxalinyl)carbonyl]glycinate (0.116 g, 0.263 mmol, 55.1% yield) as a yellow solid. ¹H NMR (400 MHz, DMSO-d₆) δ ppm 11.32-11.66 (m, 1H), 9.62 (br. s., 1H), 9.09 (br. s., 1H), 8.18 (s, 2H), 8.09 (s, 1H), 8.07 (s, 1H), 4.39 (d, J=6.6 Hz, 2H), 4.20 (q, J=7.2 Hz, 2H), 1.26 (t, J=7.1 Hz, 3H). MS (ES+) m/e 442 [M+H]⁺.

75(d) N-[(6-hydroxy-2,7-di-1,3-thiazol-2-yl-5-quinoxalinyl)carbonyl]glycine. To a suspension of ethyl N-[(6-hydroxy-2,7-di-1,3-thiazol-2-yl-5-quinoxalinyl)carbonyl]glycinate (0.116 g, 0.263 mmol) in ethanol (3.00 ml) was added 1N aqueous sodium hydroxide (3.00 ml, 3.00 mmol). After stirring 30 min. at ambient temperature, the reaction was quenched with 1N aqueous hydrochloric acid and the resulting precipitate was filtered, washed with water, and dried in vacuo to obtain N-[(6-hydroxy-2,7-di-1,3-thiazol-2-yl-5-quinoxalinyl)carbonyl]glycine (0.109 g, 0.264 mmol, 100% yield) as a bright orange solid. ¹H NMR (400 MHz, DMSO-d₆) δ ppm 9.60 (br. s., 1H), 9.06 (br. s., 1H), 8.18 (s, 1H), 8.17 (s, 1H), 8.08 (d, J=3.0 Hz, 1H), 8.06 (d, J=3.3 Hz, 1H), 4.29 (d, J=5.3 Hz, 2H). MS (ES+) m/e 414 [M+H]⁺.

### Example 76

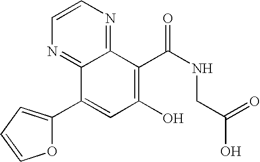

### N-{[8-(2-furanyl)-6-hydroxy-5-quinoxalinyl]carbonyl}glycine

In a 10 mL microwave vial was placed 2-furanylboronic acid (7.58 mg, 0.068 mmol), tetrakis(triphenylphosphine)palladium(0) (3.26 mg, 2.82 μmol), potassium carbonate (15.61 mg, 0.113 mmol) and ethyl N-[(8-bromo-6-hydroxy-5-quinoxalinyl)carbonyl]glycinate (20 mg, 0.056 mmol) in 1,4-dioxane (3.0 ml) and water (1.000 ml) to give a yellow suspension. The mixture was heated to 120° C. for 30 min. in a Biotage Initiator® microwave synthesizer and was diluted with methanol. Sodium hydroxide (1.0 N in water) (0.056 ml, 0.056 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[8-(2-furanyl)-6-hydroxy-5-quinoxalinyl]carbonyl}glycine (5.5 mg, 0.018 mmol, 31.1% yield) as a yellow solid. 1H NMR (400 MHz, DMSO-d6) δ ppm 15.36 (s, 1H), 12.91 (s, 1H), 11.37 (t, J=5.7 Hz, 1H), 8.81-9.11 (m, 2H), 7.99 (d, J=1.3 Hz, 1H), 7.89 (d, J=3.3 Hz, 1H), 7.77 (s, 1H), 6.78 (dd, J=3.4, 1.9 Hz, 1H), 4.24 (d, J=5.6 Hz, 2H). MS (ES+) m/e 314[M+H]⁺.

### Example 77

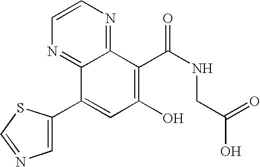

### N-{[6-hydroxy-8-(1,3-thiazol-5-yl)-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (13.05 mg, 0.011 mmol), and 5-(tributylstannanyl)-1,3-thiazole (42.3 mg, 0.113 mmol) in 1,4-dioxane (3.0 ml) to give a yellow suspension. The mixture was heated to 150° C. for 60 min. in a Biotage Initiator® microwave synthesizer, then cooled and diluted with methanol. Sodium hydroxide (1.0 N in water) (0.113 ml, 0.113 mmol) was added. The reaction was kept stirring at ambient temperature for half hour and quenched with 5 ml hydrochloric acid (1N in water). The resulting precipitate was collected, washed with water and dried under high vacuum to afford N-{[6-hydroxy-8-(1,3-thiazol-5-yl)-5-quinoxalinyl]carbonyl}glycine (26 mg, 0.071 mmol, 62.8% yield) as a yellow solid. 1H NMR (400 MHz, DMSO-d6) δ ppm 15.38 (s, 1H), 12.93 (br. s., 1H), 11.36 (t, J=5.6 Hz, 1H), 9.31 (s, 1H), 9.03 (d, J=2.0 Hz, 1H), 9.00 (d, J=2.0 Hz, 1H), 8.92 (s, 1H), 8.20 (s, 1H), 4.25 (d, J=5.6 Hz, 2H). MS (ES+) m/e 331[M+H]⁺

### Example 78

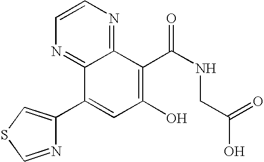

### N-{[6-hydroxy-8-(1,3-thiazol-4-yl)-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), 4-(tributylstannanyl)-1,3-thiazole (46.5 mg, 0.124 mmol) and tetrakis(triphenylphosphine)palladium(0) (13.05 mg, 0.011 mmol) in 1,4-dioxane (3.0 ml) to give a yellow suspension. The mixture was heated to 150° C. for 60 min. in a Biotage Initiator® microwave synthesizer, then cooled and diluted with methanol. Sodium hydroxide (1.0N in water) (0.113 ml, 0.113 mmol) was added. The reaction was kept stirring at ambient temperature for half hour and quenched with 5 ml hydrochloric acid (1N in water). The resulting precipitate was collected, washed with water and dried under high vacuum to afford N-{[6-hydroxy-8-(1,3-thiazol-4-yl)-5-quinoxalinyl]carbonyl}glycine (26 mg, 0.071 mmol, 62.8% yield) as a yellow solid. 1H NMR (400 MHz, DMSO-d6) δ ppm 15.33 (s, 1H), 12.93 (br. s., 1H), 11.42 (t, J=5.6 Hz, 1H), 9.32 (d, J=2.0 Hz, 1H), 9.09 (d, J=2.0 Hz, 1H), 9.02 (s, 2H), 8.29 (s, 1H), 4.25 (d, J=5.6 Hz, 2H). MS (ES+) m/e 331[M+H]+

### Example 79

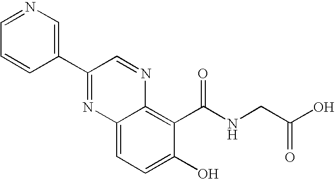

### N-{[6-hydroxy-2-(3-pyridinyl)-5-quinoxalinyl]carbonyl}glycine

79(a) ethyl N-{[6-hydroxy-2-(3-pyridinyl)-5-quinoxalinyl]carbonyl}glycinate. To a mixture of ethyl N-[(2-bromo-6-hydroxy-5-quinoxalinyl)carbonyl]glycinate (example 5(a), 0.500 g, 1.41 mmol), pyridin-3-ylboronic acid (0.208 g, 1.69 mmol) and potassium carbonate (0.390 g, 2.82 mmol) in 1,4-dioxane (3.0 mL) and water (1.0 mL) was added tetra-kis(triphenylphosphine)palladium (0.016 g, 0.014 mmol) followed by evacuation of the reaction vessel and purging with nitrogen. The reaction mixture was heated in a Biotage Initiator® microwave synthesizer at 120° C. for 30 min. Upon cooling, the mixture was diluted with EtOAc (10.0 mL) and water (20.0 mL), and water phase was extracted with EtOAc. The combined organic phases were dried (Na₂SO₄) and concentrated in vacuo to afford the title compound (0.650 g, 130.5% yield, crude) as a yellow solid, MS (ES+) m/e 353 [M+H]⁺, used in the next step without further purification.

79(b) N-{[6-hydroxy-2-(3-pyridinyl)-5-quinoxalinyl]carbonyl}glycine. To the above crude ester (0.650 g, 1.85 mmol) was added aqueous sodium hydroxide (1N, 8.0 mL), methanol (10.0 mL) and tetrahydrofuran (8.0 mL). The mixture was stirred at ambient temperature for 20 min and quenched with 1N hydrochloric acid. The precipitate was collected by filtration, recrystallized with DMSO (15.0 mL) and DMF (5.0 mL) to afford the pure 2-(6-hydroxy-2-(pyridin-3-yl)quinoxaline-5-carboxamido)acetic acid (0.093 g, 15.6% yield) as a yellow solid. ¹H NMR (300 MHz, DMSO-d6) δ ppm 15.32 (s, 1H, br), 12.94 (s, 1H, br), 11.35 (t, 1H, br, J=5.4 Hz), 9.60 (d, 1H, J=3.0 Hz), 9.47 (s, 1H), 8.75 (m, 1H), 8.65 (m, 1H), 8.26 (m, 1H), 7.61 (m, 2H), 4.28 (d, 2H, J=5.4 Hz). MS (ES+) m/e 325 [M+H]⁺.

### Example 80

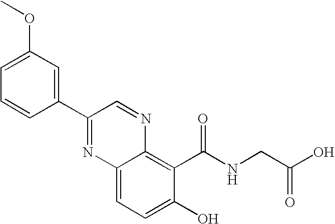

### N-({6-hydroxy-2-[3-(methyloxy)phenyl]-5-quinoxalinyl}carbonyl)glycine

80(a) Ethyl N-({6-hydroxy-2-[3-(methyloxy)phenyl]-5-quinoxalinyl}carbonyl)glycinate. To a mixture of the compound of example 5(a) (0.500 g, 1.41 mmol), 3-methoxyphenylboronic acid (0.257 g, 1.69 mmol) and potassium carbonate (0.390 g, 2.82 mmol) in 1,4-dioxane (2.5 mL) and water (1.5 mL) was added tetrakis(triphenylphosphine)palladium (0.032 g, 0.028 mmol) followed by evacuation of the reaction vessel and purging with nitrogen. The reaction mixture was heated in a Biotage Initiator® microwave synthesizer at 120° C. for 30 min. Upon cooling, the mixture was diluted with EtOAc (10.0 mL) and water (20.0 mL), and water phase was extracted with EtOAc (10.0 mL*3). The combined organic phases were dried (Na₂SO₄) and concentrated in vacuo to afford the title compound (0.421 g, 78.3% yield) as a brown solid, MS (ES+) m/e 382 [M+H]⁺, used in the next step without further purification.

80(b) N-({6-hydroxy-2-[3-(methyloxy)phenyl]-5-quinoxalinyl}carbonyl)glycine. To the above crude ester (0.421 g, 1.02 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.295 g, 75.6% yield) as a pale yellow solid. ¹H NMR (300 MHz, DMSO-d6) δ ppm 15.24 (s, 1H, br), 12.94 (s, 1H, br), 11.39 (t, 1H, br, J=7.2 Hz), 9.54 (s, 1H), 8.23 (d, 1H, J=12.4 Hz), 7.87 (d, 1H, J=8.4 Hz), 7.84 (d, 1H, J=2.4 Hz), 7.57 (d, 1H, J=12.8 Hz), 7.51 (t, 1H, J=10.4 Hz), 7.13 (m, 1H), 4.27 (d, 2H, J=6.8 Hz), 3.89 (s, 1H). MS (ES+) m/e 354 [M+H]⁺.

### Example 81

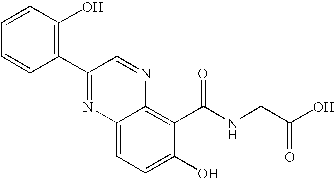

### N-{[6-hydroxy-2-(2-hydroxyphenyl)-5-quinoxalinyl]carbonyl}glycine

81(a) Ethyl N-{[6-hydroxy-2-(2-hydroxyphenyl)-5-quinoxalinyl]carbonyl}glycinate. To a mixture of ethyl N-[(2-bromo-6-hydroxy-5-quinoxalinyl)carbonyl]glycinate (0.500 g, 1.41 mmol), 2-hydroxyphenylboronic acid (0.233 g, 1.69 mmol) and potassium carbonate (0.390 g, 2.82 mmol) in 1,4-dioxane (2.5 mL) and water (1.5 mL) was added tetra-kis(triphenylphosphine)palladium (0.032 g, 0.028 mmol) followed by evacuation of the reaction vessel and purging with nitrogen. The reaction mixture was heated in a Biotage Initiator® microwave synthesizer at 120° C. for 30 min. Upon cooling, the mixture was diluted with EtOAc (10.0 mL) and water (20.0 mL), and water phase was extracted with EtOAc (10.0 mL*3). The combined organic phases were dried (Na₂SO₄) and concentrated in vacuo to afford the title compound (0.376 g, 72.6% yield) as a brown solid, MS (ES+) m/e 368 [M+H]⁺, used in the next step without further purification.

81(b) N-{[6-hydroxy-2-(2-hydroxyphenyl)-5-quinoxalinyl]carbonyl}glycine. To the above crude ester (0.376 g, 1.02 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC to afford the title compound (0.261 g, 75.2% yield) as an orange solid. ¹H NMR (300 MHz, DMSO-d6) δ ppm 15.20 (s, 1H, br), 12.91 (s, 1H, br), 11.38 (t, 2H, br, J=8 Hz), 9.62 (s, 1H), 8.26 (d, 1H, J=12.4 Hz), 8.10 (m, 1H), 7.56 (d, 1H, J=12.4 Hz), 7.40 (m, 1H), 7.05 (m, 2H), 4.26 (d, 2H, J=7.2 Hz). MS (ES+) m/e 340 [M+H]⁺.

### Example 82

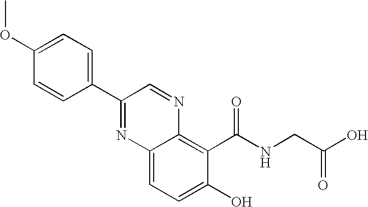

### N-({6-hydroxy-2-[4-(methyloxy)phenyl]-5-quinoxalinyl}carbonyl)glycine

To a mixture of the compound from example 5(a) (0.300 g, 0.85 mmol), 4-methoxyphenylboronic acid (0.154 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min and upon cooling, tetrahydrofuran (6.0 mL) and 1N aqueous sodium hydroxide (10.0 mL) were added. After stirring for 15 min at ambient temperature, the mixture was quenched with 1N aqueous hydrochloric acid and the resulting precipitate was filtered, washed through with methanol (15.0 mL) to afford the title compound (0.281 g, 94.1% yield) as a yellow solid. ¹H NMR (300 MHz, DMSO-d6) □ ppm 15.15 (s, 1H, br), 12.93 (s, 1H, br), 11.39 (t, 1H, J=5.4 Hz), 9.49 (s, 1H), 8.28 (m, 2H), 8.19 (d, 1H, J=9.3 Hz), 7.55 (d, 1H, J=9.6 Hz), 7.15 (m, 2H), 4.27 (d, 2H, J=5.7 Hz), 3.86 (s, 3H). MS (ES+) m/e 354 [M+H]⁺.

### Example 83

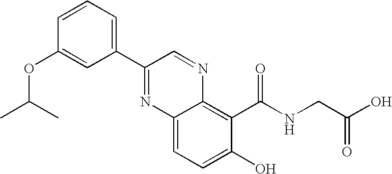

### N-[(6-hydr oxy-2-{3-[(1-methylethyl)oxy]phenyl}-5-quinoxalinyl)carbonyl]glycine

83(a) Ethyl N-[(6-hydroxy-2-{3-[(1-methylethyl)oxy]phenyl}-5-quinoxalinyl)carbonyl]glycinate. To a mixture of the compound from example 5(a) (0.300 g, 0.85 mmol), 3-isopropoxyphenylboronic acid (0.183 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. Upon cooling, the mixture was diluted with EtOAc (10.0 mL) and water (20.0 mL), and water phase was extracted with EtOAc (10.0 mL×3). The combined organic phases were dried (Na₂SO₄) and concentrated in vacuo to afford the title compound (0.183 g, 52.7% yield) as a brown solid, MS (ES+) m/e 410 [M+H]⁺, used in the next step without further purification.

83(b) N-[(6-hydroxy-2-{3-[(1-methylethyl)oxy]phenyl}-5-quinoxalinyl)carbonyl]glycine. To the above crude ester (0.183 g, 0.45 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by Pre-HPLC to afford the title compound (0.067 g, 39.4% yield) as a white solid. ¹H NMR (300 MHz, DMSO-d6) δ ppm 15.24 (s, 1H, br), 12.96 (s, 1H, br), 11.38 (t, 1H, br, J=8 Hz), 9.52 (s, 1H), 8.21 (d, 1H, J=12.4 Hz), 7.83 (m, 2H), 7.56 (d, 1H, J=12.8 Hz), 7.48 (t, 1H, J=10 Hz), 7.11 (m, 1H), 4.77 (m, 1H), 4.26 (d, 1H, J=7.2 Hz), 1.33 (d, 6H). MS (ES+) m/e 382 [M+H]⁺.

### Example 84

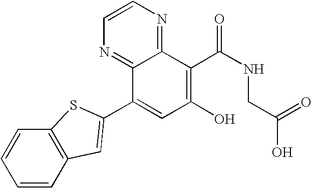

### N-{[8-(1-benzothien-2-yl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), 1-benzothien-2-yl(tributyl)stannane (47.8 mg, 0.113 mmol), and tetrakis(triphenylphosphine)palladium(0) (13.05 mg, 0.011 mmol) in 1,4-dioxane (3.0 ml) to give a yellow suspension. The mixture was heated to 150° C. for 60 min. in a Biotage Initiator® microwave synthesizer, then cooled and diluted with methanol. Sodium hydroxide (0.226 ml, 0.226 mmol) was added. The reaction was kept stirring at ambient temperature for half hour and quenched with 5 ml hydrochloric acid (1N in water). The resulting precipitate was collected, washed with water and dried under high vacuum to afford N-{[8-(1-benzothien-2-yl)-6-hydroxy-5-quinoxalinyl]carbonyl}glycine (28 mg, 0.074 mmol, 65.3% yield) as a yellow solid. 1H NMR (400 MHz, DMSO-d6) δ ppm 15.28 (br. s., 1H), 12.97 (br. s., 1H), 11.33 (t, J=5.4 Hz, 1H), 9.08 (s, 2H), 8.54 (s, 1H), 8.22 (d, J=7.8 Hz, 1H), 8.16 (d, J=8.1 Hz, 1H), 7.60 (dd, J=15.2, 1.3 Hz, 1H), 7.52 (t, J=7.1 Hz, 1H), 4.25 (d, J=5.6 Hz, 2H). MS (ES+) m/e 381[M+H]⁺

### Example 85

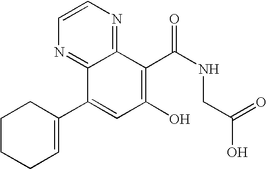

### N-{[8-(1-cyclohexen-1-yl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (6.53 mg, 5.65 μmol), 2-(1-cyclohexen-1-yl)-4,4,5,5-tetramethyl-1,3,2-dioxaborolane (23.50 mg, 0.113 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (0.226 ml, 0.226 mmol) was added. The reaction was kept stirring at ambient temperature for half hour and quenched with 5 ml hydrochloric acid (1N in water). The resulting precipitate was collected, washed with water, methylene chloride and dried to afford N-{[8-(1-cyclohexen-1-yl)-6-hydroxy-5-quinoxalinyl]carbonyl}glycine (28 mg, 0.086 mmol, 76% yield) as a yellow solid. 1H NMR (400 MHz, DMSO-d6) δ ppm 15.30 (s, 1H), 12.90 (br. s., 1H), 11.39 (t, J=5.6 Hz, 1H), 8.92 (d, J=2.0 Hz, 1H), 8.89 (d, J=2.0 Hz, 1H), 7.28 (s, 1H), 6.01 (ddd, J=3.5, 2.0, 1.8 Hz, 1H), 4.23 (d, J=5.8 Hz, 2H), 2.53-2.57 (m, 2H), 2.16-2.27 (m, 1H), 1.64-1.81 (m, 4H). MS (ES+) m/e 328[M+H]⁺

### Example 86

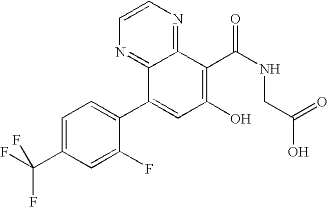

### N-({8-[2-fluoro-4-(trifluoromethyl)phenyl]-6-hydroxy-5-quinoxalinyl}carbonyl)glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (13.05 mg, 0.011 mmol), potassium carbonate (46.8 mg, 0.339 mmol), and [2-fluoro-4-(trifluoromethyl)phenyl]boronic acid (25.8 mg, 0.124 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (1.0 N in water) (0.226 ml, 0.226 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[6-hydroxy-8-(2-thienyl)-5-quinoxalinyl]carbonyl}glycine (5 mg, 0.011 mmol, 19.97% yield) as a yellow solid. 1H NMR (400 MHz, CHLOROFORM-d) δ ppm 15.11 (br. s., 1H), 11.67 (t, J=4.8 Hz, 1H), 8.81 (d, J=1.8 Hz, 1H), 8.76 (d, J=1.5 Hz, 1H), 7.57-7.65 (m, 2H), 7.47-7.55 (m, 2H), 4.44 (d, J=5.3 Hz, 2H). MS (ES+) m/e 410[M+H]⁺

### Example 87

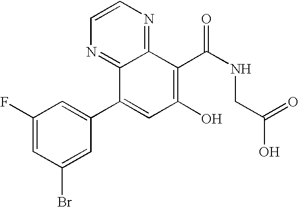

### N-{[8-(3-bromo-5-fluorophenyl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), (3-bromo-5-fluorophenyl)boronic acid (24.71 mg, 0.113 mmol), tetrakis(triphenylphosphine)palladium(0) (13.05 mg, 0.011 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (1N in water) (0.226 ml, 0.226 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[8-(3-bromo-5-fluorophenyl)-6-hydroxy-5-quinoxalinyl]carbonyl}glycine (34 mg, 0.060 mmol, 53.5% yield) as a yellow solid. ¹H NMR (400 MHz, DMSO-d6) δ ppm 15.40 (s, 1H), 12.96 (br. s., 1H), 11.45 (t, J=5.7 Hz, 1H), 8.99 (d, J=2.0 Hz, 1H), 8.93 (d, J=1.8 Hz, 1H), 7.74-7.77 (m, 1H), 7.70 (dt, J=8.5, 2.1 Hz, 1H), 7.66 (s, 1H), 7.61 (dd, J=9.6, 1.3 Hz, 1H), 4.26 (d, J=5.8 Hz, 2H). MS (ES+) m/e 420[M+H]⁺

### Example 88

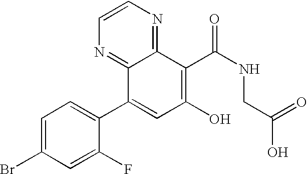

### N-{[8-(4-bromo-2-fluorophenyl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (13.05 mg, 0.011 mmol), (4-bromo-2-fluorophenyl)boronic acid (24.71 mg, 0.113 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (0.226 ml, 0.226 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[8-(4-bromo-2-fluorophenyl)-6-hydroxy-5-quinoxalinyl]carbonyl}glycine (8.5 mg, 0.016 mmol, 14.09% yield) as a yellow solid. ¹H NMR (400 MHz, CHLOROFORM-d) δ ppm 11.64 (br. s., 1H), 8.78 (d, J=2.0 Hz, 1H), 8.72 (d, J=1.8 Hz, 1H), 7.48 (s, 1H), 7.41 (ddd, J=14.9, 8.6, 1.8 Hz, 2H), 7.32 (t, J=7.7 Hz, 1H), 4.35 (d, J=5.1 Hz, 2H). MS (ES+) m/e 420[M+H]⁺

### Example 89

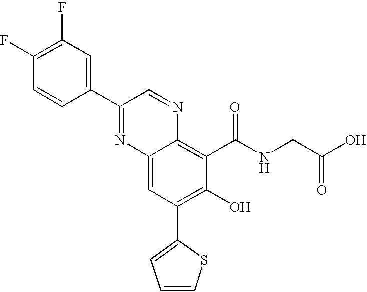

### N-{[2-(3,4-difluorophenyl)-6-hydroxy-7-(2-thienyl)-5-quinoxalinyl]carbonyl}glycine

89(a) methyl 2-(3,4-difluorophenyl)-6-(methyloxy)-7-(2-thienyl)-5-quinoxalinecarboxylate. To a solution of methyl 2-chloro-6-(methyloxy)-7-(2-thienyl)-5-quinoxalinecarboxylate (example 70(b), 0.180 g, 0.538 mmol) in 1,4-dioxane (3.00 ml) and water (1.000 ml) was added 3,4-difluorophenyl)boronic acid (0.093 g, 0.591 mmol), potassium carbonate (0.223 g, 1.613 mmol) and tetrakis(triphenylphosphine)palladium(0) (0.019 g, 0.016 mmol) followed by heating to 105° C. overnight in an oil bath. Upon cooling, the reaction mixture was filtered through celite, washed with ethyl acetate and concentrated in vacuo. The resulting residue was washed with water and dried in vacuo to obtain methyl 2-(3,4-difluorophenyl)-6-(methyloxy)-7-(2-thienyl)-5-quinoxalinecarboxylate (0.187 g, 0.453 mmol, 84% yield) as a yellow solid. ¹H NMR (400 MHz, DMSO-d₆) δ ppm 9.57 (s, 1H), 8.56 (s, 1H), 8.34-8.45 (m, 1H), 8.19-8.28 (m, 1H), 7.96 (dd, J=3.7, 1.1 Hz, 1H), 7.83 (dd, J=5.2, 1.1 Hz, 1H), 7.67-7.76 (m, 1H), 7.27 (dd, J=5.1, 3.8 Hz, 1H), 4.01 (s, 3H), 3.86 (s, 3H). MS (ES+) m/e 413 [M+H]⁺.

89(b) 2-(3,4-difluorophenyl)-6-hydroxy-7-(2-thienyl)-5-quinoxalinecarboxylic acid. A solution of methyl 2-(3,4-difluorophenyl)-6-(methyloxy)-7-(2-thienyl)-5-quinoxalinecarboxylate (0.187 g, 0.453 mmol) in dichloromethane (10 mL) was treated with boron tribromide (1M solution in dichloromethane) (2.267 mL, 2.267 mmol) at room temperature overnight. The reaction mixture was quenched with water, filtered and washed with more water. The solid was dried in vacuo to give 2-(3,4-difluorophenyl)-6-hydroxy-7-(2-thienyl)-5-quinoxalinecarboxylic acid (0.119 g, 0.310 mmol, 68.3% yield) as an orange solid. ¹H NMR (400 MHz, DMSO-d₆) δ ppm 15.48 (br. s., 1H), 9.44 (s, 1H), 8.71 (s, 1H), 8.26-8.38 (m, 1H), 8.15 (d, J=1.0 Hz, 1H), 8.14 (d, J=1.0 Hz, 1H), 7.85 (dd, J=5.1, 1.0 Hz, 1H), 7.66-7.78 (m, 1H), 7.27 (dd, J=5.1, 3.8 Hz, 1H). MS (ES+) m/e 385 [M+H]⁺.

89(c) ethyl N-{[2-(3,4-difluorophenyl)-6-hydroxy-7-(2-thienyl)-5-quinoxalinyl]carbonyl}glycinate. A solution of 2-(3,4-difluorophenyl)-6-hydroxy-7-(2-thienyl)-5-quinoxalinecarboxylic acid (0.119 g, 0.310 mmol) and ethyl glycine hydrochloride (0.086 g, 0.619 mmol) in N,N-Dimethylformamide (DMF) (3.0 mL) was treated with triethylamine (0.129 mL, 0.929 mmol) and PyBOP (0.177 g, 0.341 mmol). The reaction mixture was stirred overnight at ambient temperature, quenched by water and extracted twice with ethyl acetate. The combined organic portions were dried over magnesium sulfate, filtered and concentrated in vacuo. The oily residue was decanted in water, filtered and dried in vacuo to obtain ethyl N-{[2-(3,4-difluorophenyl)-6-hydroxy-7-(2-thienyl)-5-quinoxalinyl]carbonyl}glycinate (0.121 g, 0.258 mmol, 83% yield) as an oranges solid. ¹H NMR (400 MHz, DMSO-d₆) δ ppm 11.61 (t, J=5.6 Hz, 1H), 9.54 (s, 1H), 8.66 (s, 1H), 8.34-8.48 (m, 1H), 8.16-8.30 (m, 1H), 8.08 (dd, J=3.7, 0.9 Hz, 1H), 7.81 (dd, J=5.2, 1.1 Hz, 1H), 7.63-7.77 (m, 1H), 7.27 (dd, J=5.1, 3.8 Hz, 1H), 4.40 (d, J=5.8 Hz, 2H), 4.21 (q, J=7.1 Hz, 2H), 1.26 (t, J=7.2 Hz, 3H). MS (ES+) m/e 470 [M+H]⁺.

89(d) N-{[2-(3,4-difluorophenyl)-6-hydroxy-7-(2-thienyl)-5-quinoxalinyl]carbonyl}glycine. To a suspension of ethyl N-{[2-(3,4-difluorophenyl)-6-hydroxy-7-(2-thienyl)-5-quinoxalinyl]carbonyl}glycinate (0.121 g, 0.258 mmol) in ethanol (1.0 mL) was added 1N aqueous sodium hydroxide (2.00 ml, 2.000 mmol). After stirring 30 min. at ambient temperature, the reaction was quenched with 1N aqueous hydrochloric acid and the resulting precipitate was filtered, washed with water, and dried in vacuo to obtain N-{[2-(3,4-difluorophenyl)-6-hydroxy-7-(2-thienyl)-5-quinoxalinyl]carbonyl}glycine (0.03 g, 0.068 mmol, 26.4% yield) as an orange solid. ¹H NMR (400 MHz, DMSO-d₆) δ ppm 11.53 (t, J=5.1 Hz, 1H), 9.50 (s, 1H), 8.60 (s, 1H), 8.31-8.45 (m, 1H), 8.17-8.25 (m, 1H), 8.06 (dd, J=3.8, 1.0 Hz, 1H), 7.80 (dd, J=5.1, 1.0 Hz, 1H), 7.62-7.74 (m, 1H), 7.25 (dd, J=5.2, 3.7 Hz, 1H), 4.30 (d, J=5.6 Hz, 2H). MS (ES+) m/e 442 [M+H]⁺.

### Example 90

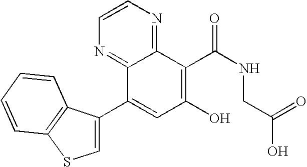

### N-{[8-(1-benzothien-3-yl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (6.53 mg, 5.65 μmol), 1-benzothien-3-ylboronic acid (20.11 mg, 0.113 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (0.226 ml, 0.226 mmol) was added. The reaction was kept stirring at ambient temperature for half hour and quenched with 5 ml hydrochloric acid (1N in water). The precipitate was collected, washed with water and methylene chloride and dried to afford N-{[8-(1-benzothien-3-yl)-6-hydroxy-5-quinoxalinyl]carbonyl}glycine (32 mg, 0.084 mmol, 74.7% yield) as a yellow solid. 1H NMR (400 MHz, DMSO-d6) δ ppm 15.43 (s, 1H), 12.94 (br. s., 1H), 11.49 (t, J=5.6 Hz, 1H), 8.98 (d, J=2.0 Hz, 1H), 8.81 (d, J=2.0 Hz, 1H), 8.10 (d, J=7.8 Hz, 1H), 8.03 (s, 1H), 7.63 (s, 1H), 7.38-7.50 (m, 2H), 7.27-7.36 (m, 1H), 4.28 (d, J=5.6 Hz, 2H). MS (ES+) m/e 380[M+H]⁺

### Example 91

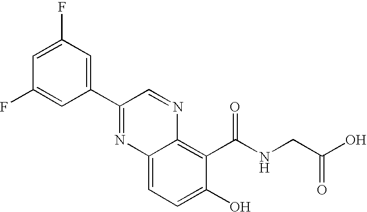

### N-{[2-(3,5-difluorophenyl)-6-hydroxy-5-quinoxalinyl]carbonyl}glycine

To a mixture of the compound from example 5(a) (0.300 g, 0.85 mmol), 3,5-difluorophenylboronic acid (0.160 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min and upon cooling, tetrahydrofuran (6.0 mL) and 1N aqueous sodium hydroxide (10.0 mL) was added. After stirring for 15 min at ambient temperature, the mixture was quenched with 1N aqueous hydrochloric acid and the resulting precipitate was filtered, purified via rp-HPLC to afford the title compound (0.141 g, 46.5% yield) as a yellow solid. ¹H NMR (300 MHz, DMSO-d6) δ ppm 15.36 (s, 1H, br), 12.97 (s, 1H, br), 11.33 (t, 1H, J=5.1 Hz), 9.61 (s, 1H), 8.27 (d, 1H, J=9.6 Hz), 8.07 (d, 2H, J=6.6 Hz), 7.60 (d, 1H, J=9.3 Hz), 7.47 (t, 1H, J=9.0 Hz), 4.29 (d, 2H, J=5.4 Hz). MS (ES+) m/e 360 [M+H]⁺.

### Example 92

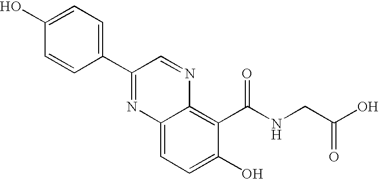

### N-{[6-hydroxy-2-(4-hydroxyphenyl)-5-quinoxalinyl]carbonyl}glycine

To a mixture of the compound from example 5(a) (0.300 g, 0.85 mmol), 4-hydroxyphenylboronic acid (0.151 g, 1.11 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min and upon cooling, tetrahydrofuran (8.0 mL) and 1N aqueous sodium hydroxide (10.0 mL) were added. After stirring for 10 min at ambient temperature, the mixture was quenched with 1N aqueous hydrochloric acid and the resulting precipitate was filtered, washed with methanol to afford the title compound (0.200 g, 63.0% yield) as a green solid. ¹H NMR (300 MHz, DMSO-d6) δ ppm 15.11 (s, 1H, br), 12.92 (s, 1H, br), 11.38 (t, 1H, J=5.4 Hz), 10.01 (s, 1H), 9.42 (s, 1H), 8.15 (m, 2H), 7.51 (d, 1H, J=9.0 Hz), 6.95 (d, 2H, J=8.4 Hz), 4.25 (d, 2H, J=5.4 Hz). MS (ES+) m/e 370 [M+H]⁺.

### Example 93

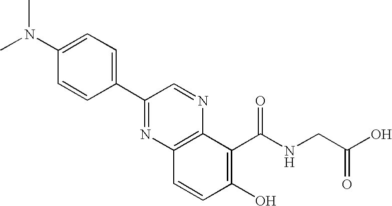

### N-({2-[4-(dimethylamino)phenyl]-6-hydroxy-5-quinoxalinyl}carbonyl)glycine

To a mixture of the compound from example 5(a) (0.300 g, 0.85 mmol), 4-dimethylaminophenylboronic acid (0.167 g, 1.02 mmol) and potassium carbonate (0.234 g, 1.69 mmol) in 1,4-dioxane (3.0 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min and upon cooling, the mixture was diluted with brine and EtOAc, and extracted twice with EtOAc. The extracts were evaporated in vacuo to afford the crude ester. Tetrahydrofuran (15.0 mL) and 1N aqueous sodium hydroxide (10.0 mL) were added to the compound. After stirring for 10 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.226 g, 72.9% yield) as a pale red solid. ¹H NMR (300 MHz, DMSO-d6) δ ppm 11.30 (s, 1H), 9.39 (s, 1H), 8.08 (m, 3H), 7.45 (d, 1H, J=9.6 Hz), 6.83 (d, 2H, J=9.0 Hz), 3.98 (d, 2H, J=5.1 Hz), 3.00 (s, 6H). MS (ES+) m/e 367 [M+H]⁺.

### Example 94

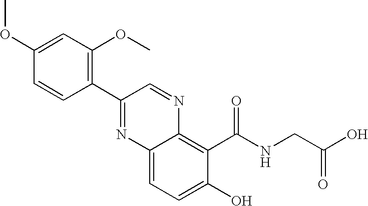

### N-({2-[2,4-bis(methyloxy)phenyl]-6-hydroxy-5-quinoxalinyl}carbonyl)glycine

To a mixture of the compound from example 5(a) (0.300 g, 0.85 mmol), 2,4-dimethoxyphenylboronic acid (0.185 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min and upon cooling, tetrahydrofuran (6.0 mL) and 1N aqueous sodium hydroxide (10.0 mL) were added. After stirring for 15 min at ambient temperature, the mixture was quenched with 1N aqueous hydrochloric acid and the resulting precipitate was filtered, washed through with methanol (10.0 mL) to afford the title compound (0.062 g, 19.1% yield) as a yellow solid. ¹H NMR (300 MHz, DMSO-d6) δ ppm 15.17 (s, 1H, br), 12.87 (s, 1H, br), 11.42 (t, 1H, J=5.7 Hz), 9.34 (s, 1H), 8.18 (d, 1H, J=9.0 Hz), 7.89 (d, 1H, J=8.1 Hz), 7.53 (d, 1H, J=9.6 Hz), 6.77 (m, 2H), 4.25 (d, 2H, J=5.1 Hz), 3.94 (s, 3H), 3.87 (s, 3H). MS (ES+) m/e 384 [M+H]⁺.

### Example 95

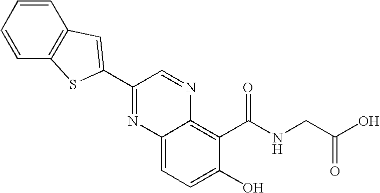

### N-{[2-(1-benzothien-2-yl)-6-hydroxy-5-quinoxalinyl]carbonyl}glycine

To a mixture of the compound from example 5(a) (0.300 g, 0.85 mmol), 1-benzothien-2-ylboronic acid (0.181 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.278 g, 86.7% yield) as an orange solid. ¹H NMR (300 MHz, DMSO-d6) δ ppm 10.98 (s, 1H), 9.61 (s, 1H), 8.51 (s, 1H), 8.03 (m, 2H), 7.91 (t, 1H, J=2.7 Hz), 7.43 (m, 3H), 3.73 (d, 2H, J=3.9 Hz). MS (ES+) m/e 380 [M+H]⁺.

### Example 96

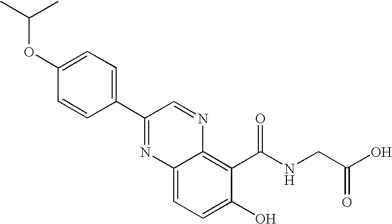

### N-[(6-hydr oxy-2-{4-[(1-methylethyl)oxy]phenyl}-5-quinoxalinyl)carbonyl]glycine

96(a) Ethyl N-[(6-hydroxy-2-{4-[(1-methylethyl)oxy]phenyl}-5-quinoxalinyl)carbonyl]glycinate. To a mixture of the compound from example 5(a) (0.300 g, 0.85 mmol), 4-isopropoxyphenylboronic acid (0.183 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. Upon cooling, the mixture was diluted with EtOAc (10.0 mL) and water (20.0 mL), and extracted with EtOAc (10.0 mL×3). The combined organic phases were dried (Na₂SO₄) and concentrated in vacuo to afford the title compound (0.256 g, 73.8% yield) as a yellow solid, MS (ES+) m/e 410 [M+H]⁺, used in the next step without further purification.

96(b) N-[(6-hydr oxy-2-{4-[(1-methylethyl)oxy]phenyl}-5-quinoxalinyl)carbonyl]glycine. To the above ester (0.256 g, 0.63 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.161 g, 67.6% yield) as a bright yellow solid. ¹H NMR (300 MHz, DMSO-d6) □ ppm 15.15 (s, 1H, br), 12.92 (s, 1H, br), 11.39 (t, 1H, br, J=6.3 Hz), 9.48 (s, 1H), 8.21 (m, 3H), 7.54 (d, 1H, J=9.9 Hz), 7.11 (m, 2H), 4.75 (m, 1H), 4.26 (d, 1H, J=5.4 Hz), 1.32 (d, 6H, J=6.3 Hz). MS (ES+) m/e 382 [M+H]⁺.

### Example 97

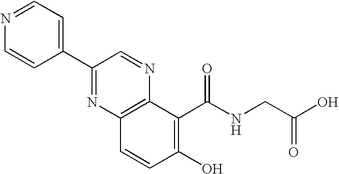

### N-{[6-hydroxy-2-(4-pyridinyl)-5-quinoxalinyl]carbonyl}glycine

To a mixture of the compound from example 5(a) (0.500 g, 1.41 mmol), pyridin-4-ylboronic acid (0.268 g, 2.19 mmol) and potassium carbonate (0.392 g, 2.84 mmol) in 1,4-dioxane (2.0 mL) was added tetrakis(triphenylphosphine)palladium (0.033 g, 0.028 mmol) followed by evacuation of the reaction vessel and purging with nitrogen. The reaction mixture was heated in a Biotage Initiator® microwave synthesizer at 120° C. for 30 min and upon cooling, the mixture was diluted with brine and EtOAc, and the organic phase was extracted twice by EtOAc, dried in vacuo to afford intermediate ethyl 2-(6-hydroxy-2-(pyridin-4-yl)quinoxaline-5-carboxamido)acetate and tetrahydrofuran (15.0 mL) and 1N aqueous sodium hydroxide (10.0 mL) was added to the compound. After stirring for 10 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.070 g, 14.0% yield) as a pale yellow solid. ¹H NMR (300 MHz, DMSO-d6) δ ppm 15.41 (s, 1H, br), 12.89 (s, 1H, br), 11.36 (t, 1H, J=5.2 Hz), 9.62 (s, 1H), 8.80 (d, 2H, J=5.7 Hz), 8.28 (m, 3H), 7.62 (d, 1H, J=9.0 Hz), 4.27 (d, 2H, J=5.7 Hz). MS (ES+) m/e 353 [M+H]⁺.

### Example 98

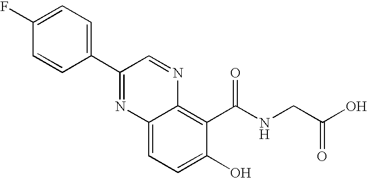

### N-{[2-(4-fluorophenyl)-6-hydroxy-5-quinoxalinyl]carbonyl}glycine

98(a) Ethyl N-{[2-(4-fluorophenyl)-6-hydroxy-5-quinoxalinyl]carbonyl}glycinate. To a mixture of the compound from example 5(a) (0.300 g, 0.85 mmol), 4-fluorophenylboronic acid (0.142 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. Upon cooling, the mixture was diluted with EtOAc (10.0 mL) and concentrated in vacuo. The resulting solid was dissolved in tetrahydrofuran and concentrated to afford the title compound (0.313 g, 100.0% yield, crude) as a yellow solid, MS (ES+) m/e 370 [M+H]⁺, used in the next step without further purification.

98(b) N-{[2-(4-(4-fluorophenyl)-6-hydroxy-5-quinoxalinyl]carbonyl}glycine. To the above crude ester (0.313 g, 0.85 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get the crude product, which was purified by rp-HPLC (acetonitrile/water+0.1% trifluoroacetic acid) to afford the title compound (0.161 g, 55.7% yield) as a gray solid. ¹H NMR (300 MHz, DMSO-d6) δ ppm 15.24 (s, 1H, br), 12.94 (s, 1H, br), 11.37 (t, 1H, J=5.1 Hz), 9.53 (s, 1H), 8.37 (m, 2H), 8.22 (d, 1H, J=9.6 Hz), 7.58 (d, 1H, J=9.3 Hz), 7.43 (t, 2H, J=9.0 Hz), 4.26 (d, 2H, J=5.4 Hz). MS (ES+) m/e 342 [M+H]⁺.

### Example 99

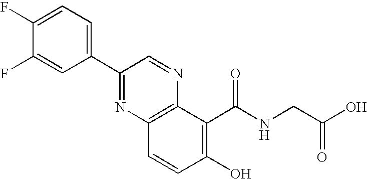

### N-{[2-(3,4-difluorophenyl)-6-hydroxy-5-quinoxalinyl]carbonyl}glycine

To a mixture of the compound from example 5(a) (0.300 g, 0.85 mmol), 3,4-difluorophenylboronic acid (0.160 g, 1.02 mmol) and potassium carbonate (0.234 g, 1.69 mmol) in 1,4-dioxane (3.0 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min and upon cooling, the mixture was diluted with brine and EtOAc, and the organic phase was extracted twice by EtOAc, dried in vacuo and purified by rp-HPLC (acetonitrile/water+0.1% trifluoroacetic acid) to afford the intermediate ester. Tetrahydrofuran (15.0 mL) and 1N aqueous sodium hydroxide (10.0 mL) were added to the compound. After stirring for 10 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.138 g, 75.0% yield) as a pale yellow solid. ¹H NMR (300 MHz, DMSO-d6) δ ppm 15.84 (s, 1H), 12.92 (s, 1H), 11.32 (s, 1H), 9.54 (s, 1H), 8.27 (m, 3H), 7.56 (m, 2H), 4.27 (s, 2H). MS (ES+) m/e 330 [M+H]⁺.

### Example 100

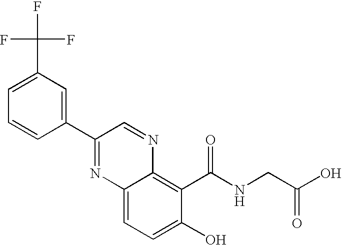

### N-({6-hydroxy-2-[3-(trifluoromethyl)phenyl]-5-quinoxalinyl}carbonyl)glycine

100(a) Ethyl N-({6-hydroxy-2-[3-(trifluoromethyl)phenyl]-5-quinoxalinyl}carbonyl)glycinate. To a mixture of the compound from example 5(a) (0.300 g, 0.85 mmol), 3-(trifluoromethyl)phenylboronic acid (0.193 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuran. The organic phase was concentrated in vacuo to afford the title compound (0.257 g, 72.4% yield) as a yellow solid, MS (ES+) m/e 420 [M+H]⁺, used in the next step without further purification.

100(b) N-({6-hydroxy-2-[3-(trifluoromethyl)phenyl]-5-quinoxalinyl}carbonyl)glycine. To the above crude ester (0.257 g, 0.61 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added to adjust pH to 3. The precipitate was collected by filtration to get crude product, which was purified by rp-HPLC (acetonitrile/water+0.1% trifluoroacetic acid) to afford the title compound (0.106 g, 44.2% yield) as a pale yellow solid. ¹H NMR (300 MHz, DMSO-d6) δ ppm 15.32 (s, 1H, br), 12.96 (s, 1H, br), 11.37 (t, 1H, br, J=5.7 Hz), 9.65 (s, 1H), 8.62 (d, 2H, J=6.0 Hz), 8.28 (d, 1H, J=9.0 Hz), 7.92 (d, 1H, J=7.8 Hz), 7.84 (m, 1H), 7.60 (d, 1H, J=9.6 Hz), 4.28 (d, 2H, J=5.7 Hz). MS (ES+) m/e 392 [M+H]⁺.

### Example 101

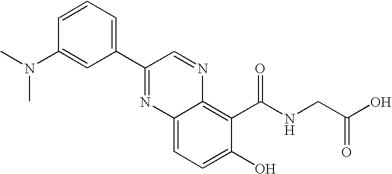

### N-({2-[3-(dimethylamino)phenyl]-6-hydroxy-5-quinoxalinyl}carbonyl)glycine

To a mixture of the compound from example 5(a) (0.300 g, 0.85 mmol), 3-(dimethylamino)phenylboronic acid (0.168 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, washed through with methanol (20.0 mL) to afford the title compound (0.158 g, 51.0% yield) as an orange solid. ¹H NMR (300 MHz, DMSO-d6) δ ppm 15.20 (s, 1H, br), 12.92 (s, 1H, br), 11.41 (t, 1H, J=4.8 Hz), 9.50 (s, 1H), 7.55 (d, 3H, J=9.3 Hz), 7.39 (t, 1H, J=8.1 Hz), 6.92 (m, 1H), 4.26 (d, 2H, J=5.1 Hz), 3.02 (s, 6H). MS (ES+) m/e 367 [M+H]⁺.

### Example 102

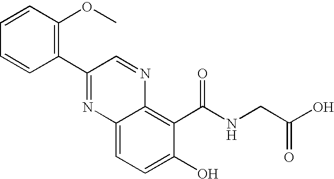

### N-({6-hydroxy-2-[2-(methyloxy)phenyl]-5-quinoxalinyl}1 carbonyl)glycine

To a mixture of the compound from example 5(a) (0.300 g, 0.85 mmol), 2-methoxyphenylboronic acid (0.154 g, 1.02 mmol) and potassium carbonate (0.234 g, 1.69 mmol) in 1,4-dioxane (4.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get intermediate ester and upon cooling, tetrahydrofuran (5.0 mL), methanol (5.0 mL) and 1N aqueous sodium hydroxide (8.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.050 g, 16.8% yield) as a yellow solid. ¹H NMR (300 MHz, DMSO-d6) δ ppm 15.24 (s, 1H, br), 11.41 (t, 1H, J=5.4 Hz), 9.35 (s, 1H), 8.22 (d, 1H, J=9.6 Hz), 7.88 (d, 1H, J=6.3 Hz), 7.54 (t, 2H, J=9.0 Hz), 7.15 (m, 2H), 4.26 (d, 2H, J=5.4 Hz), 3.92 (s, 3H). MS (ES+) m/e 354 [M+H]⁺.

### Example 103

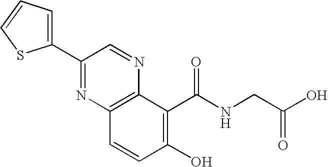

### N-{[6-hydroxy-2-(2-thienyl)-5-quinoxalinyl]carbonyl}glycine

To a mixture of the compound from example 5(a) (0.300 g, 0.85 mmol), thiophen-2-ylboronic acid (0.130 g, 1.02 mmol) and potassium carbonate (0.235 g, 1.70 mmol) in 1,4-dioxane (2.0 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min and upon cooling, the mixture was diluted with brine and EtOAc, and the organic phase was extracted twice by EtOAc, dried in vacuo to afford the intermediate ester. Tetrahydrofuran (15.0 mL) and 1N aqueous sodium hydroxide (10.0 mL) were added to the compound. After stirring for 10 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.060 g, 21.8% yield) as a yellow solid. ¹H NMR (300 MHz, DMSO-d6) δ ppm 15.60 (s, 1H), 11.12 (s, 1H), 9.37 (s, 1H), 7.99 (m, 2H), 7.73 (s, 1H), 7.28 (m, 2H), 3.88 (s, 2H). MS (ES+) m/e 330 [M+H]⁺.

### Example 104

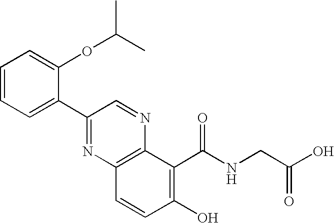

### N-[(6-hydr oxy-2-{2-[(1-methylethyl)oxy]phenyl}-5-quinoxalinyl)carbonyl]glycine

To a mixture of the compound from example 5(a) (0.300 g, 0.85 mmol), 2-isopropoxyphenylboronic acid (0.183 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (5.0 mL) and 1N aqueous sodium hydroxide (8.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to the title compound (0.163 g, 50.5% yield) as a yellow solid. ¹H NMR (300 MHz, DMSO-d6) δ ppm 15.23 (s, 1H, br), 12.90 (s, 1H, br), 11.41 (t, 1H, J=6.0 Hz), 9.39 (s, 1H), 7.88 (m, 1H), 7.52 (m, 2H), 7.27 (d, 1H, J=8.7 Hz), 7.15 (t, 1H, J=7.5 Hz), 4.80 (m, 1H), 4.26 (d, 2H, J=6.0 Hz), 1.33 (s, 3H), 1.31 (s, 3H). MS (ES+) m/e 382 [M+H]⁺.

### Example 105

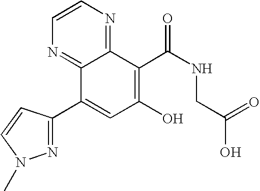

### N-{[6-hydroxy-8-(1-methyl-1H-pyrazol-3-yl)-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (6.53 mg, 5.65 μmol), 1-methyl-3-(4,4,5,5-tetramethyl-1,3,2-dioxaborolan-2-yl)-1H-pyrazole (25.8 mg, 0.124 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (1.0 N in water) (0.226 ml, 0.226 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[6-hydroxy-8-(1-methyl-1H-pyrazol-3-yl)-5-quinoxalinyl]carbonyl}glycine (13 mg, 0.029 mmol, 26.1% yield) as a yellow solid. ¹H NMR (400 MHz, DMSO-d6) δ ppm 15.32 (br. s., 1H), 11.38 (t, J=5.3 Hz, 1H), 8.95 (d, J=4.5 Hz, 2H), 8.74 (s, 1H), 8.35 (s, 1H), 7.79 (s, 1H), 4.23 (d, J=5.6 Hz, 2H), 3.95 (s, 3H). MS (ES+) m/e 328[M+H]⁺

### Example 106

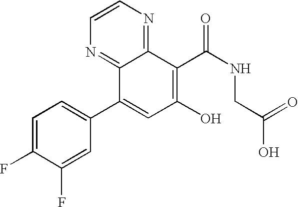

### N-{[8-(3,4-difluorophenyl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (6.53 mg, 5.65 μmol), (3,4-difluorophenyl)boronic acid (19.62 mg, 0.124 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (1.0 N in water) (0.113 ml, 0.113 mmol) was added. The reaction was kept stirring at ambient temperature for half hour and quenched with 5 ml hydrochloric acid (1N in water). The resulting solution was purified via preparative HPLC (YMC 75×30 mm column, 0.1% TFA in water and 0.1% TFA in acetonitrile) to afford N-{[8-(3,4-difluorophenyl)-6-hydroxy-5-quinoxalinyl]carbonyl}glycine (10 mg, 0.021 mmol, 18.71% yield) as a yellow solid. ¹H NMR (400 MHz, DMSO-d6) δ ppm 15.40 (s, 1H), 12.93 (br. s., 1H), 11.45 (t, J=5.7 Hz, 1H), 8.99 (d, J=1.8 Hz, 1H), 8.93 (d, J=1.8 Hz, 1H), 7.83 (dd, J=10.5, 8.2 Hz, 1H), 7.63 (s, 1H), 7.54-7.62 (m, 2H), 4.26 (d, J=5.8 Hz, 2H). MS (ES+) m/e 360[M+H]⁺

### Example 107

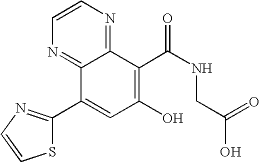

### N-{[6-hydroxy-8-(1,3-thiazol-2-yl)-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), 2-(tributylstannanyl)-1,3-thiazole (85 mg, 0.226 mmol) and tetrakis(triphenylphosphine)palladium(0) (19.58 mg, 0.017 mmol) in 1,4-dioxane (3.0 ml) to give a yellow suspension. The mixture was heated to 150° C. for 60 min. in a Biotage Initiator® microwave synthesizer, then cooled and diluted with methanol. Sodium hydroxide (1.0N in water) (0.113 ml, 0.113 mmol) was added. The reaction was kept stirring at ambient temperature for half hour and quenched with 5 ml hydrochloric acid (1.0 N in water). The resulting solution was purified via preparative HPLC (YMC 75×30 mm column, 0.1% TFA in water and 0.1% TFA in acetonitrile) to afford N-{[6-hydroxy-8-(1,3-thiazol-2-yl)-5-quinoxalinyl]carbonyl}glycine (8.0 mg, 0.018 mmol, 15.94% yield) as a yellow solid. ¹H NMR (400 MHz, DMSO-d6) δ ppm 15.33 (br. s., 1H), 12.81 (br. s., 1H), 11.35 (t, J=5.6 Hz, 1H), 9.08 (d, J=1.8 Hz, 2H), 8.40 (s, 1H), 8.16 (d, J=3.3 Hz, 1H), 8.09 (d, J=3.3 Hz, 1H), 7.13 (br. s., 2H), 4.25 (d, J=5.6 Hz, 2H). MS (ES+) m/e 331[M+H]⁺.

### Example 108

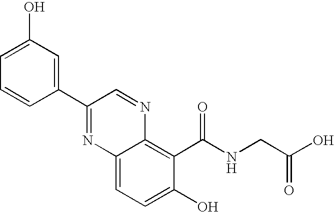

### N-{[6-hydroxy-2-(3-hydroxyphenyl)-5-quinoxalinyl]carbonyl}glycine

108(a) Ethyl N-{[6-hydroxy-2-(3-hydroxyphenyl)-5-quinoxalinyl]carbonyl}glycinate. To a mixture of the compound from example 5(a) (0.300 g, 0.85 mmol), 3-hydroxyphenylboronic acid (0.140 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuran. The organic phase was concentrated in vacuo to afford the title compound (0.251 g, 80.7% yield) as a brown solid, MS (ES+) m/e 368 [M+H]⁺, used in the next step without further purification.

108(b) N-{[6-hydroxy-2-(3-hydroxyphenyl)-5-quinoxalinyl]carbonyl}glycine. To the above crude ester (0.251 g, 0.68 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.227 g, 97.8% yield) as a pale yellow solid. ¹H NMR (300 MHz, DMSO-d6) δ ppm 15.25 (s, 1H, br), 11.40 (t, 1H, br, J=4.8 Hz), 9.46 (s, 1H), 8.22 (d, 1H, J=8.7 Hz), 7.73 (m, 2H), 7.58 (d, 1H, J=9.6 Hz), 7.41 (t, 1H, J=8.1 Hz), 6.96 (m, 1H), 4.28 (d, 2H, J=5.7 Hz). MS (ES+) m/e 340 [M+H]⁺.

### Example 109

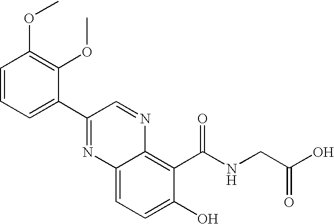

### N-({2-[2,3-bis(methyloxy)phenyl]-6-hydroxy-5-quinoxalinyl}carbonyl)glycine

To a mixture of the compound from example 5(a) (0.300 g, 0.85 mmol), 2,3-dimethoxyphenylboronic acid (0.201 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, washed through with methanol (20.0 mL) to afford the title compound (0.221 g, 68.1% yield) as a green solid. ¹H NMR (300 MHz, DMSO-d6) δ ppm 15.27 (s, 1H), 12.89 (s, 1H), 11.38 (t, 1H, J=6.0 Hz), 9.26 (s, 1H), 8.21 (d, 1H, J=9.0 Hz), 7.56 (d, 1H, J=9.6 Hz), 7.42 (m, 1H), 7.25 (m, 2H), 4.25 (d, 2H, J=6.0 Hz), 3.90 (s, 3H), 3.74 (s, 3H). MS (ES+) m/e 384 [M+H]⁺.

### Example 110

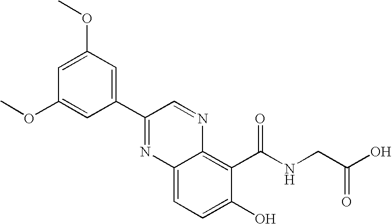

### N-({2-[3,5-bis(methyloxy)phenyl]-6-hydroxy-5-quinoxalinyl}carbonyl)glycine

110(a) Ethyl N-({2-[3,5-bis(methyloxy)phenyl]-6-hydroxy-5-quinoxalinyl}carbonyl)glycinate. To a mixture of the compound from example 5(a) (0.300 g, 0.85 mmol), 3,5-dimethoxyphenylboronic acid (0.185 g, 1.02 mmol) and potassium carbonate (0.234 g, 1.70 mmol) in 1,4-dioxane (2.5 mL) and water (1.5 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuan. The mixture was concentrated in vacuo to afford the title compound (0.323 g, 92.8% yield) as a brown solid, MS (ES+) m/e 412 [M+H]⁺, used in the next step without further purification.

110(b) N-(2-[3,5-bis(methyloxy)phenyl]-6-hydroxy-5-quinoxalinyl 1 carbonyl)glycine. To the above crude ester (0.323 g, 0.79 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford title compound (0.165 g, 54.8% yield) as a pale solid. ¹H NMR (300 MHz, DMSO-d6) δ ppm 15.25 (s, 1H, br), 12.97 (s, 1H, br), 11.39 (t, 1H, br, J=5.4 Hz), 9.54 (s, 1H), 8.23 (d, 1H, J=9.3 Hz), 7.56 (d, 1H, J=9.3 Hz), 7.44 (m, 2H), 6.68 (m, 1H), 4.27 (d, 2H, J=5.4 Hz). MS (ES+) m/e 384 [M+H]⁺.

### Example 111

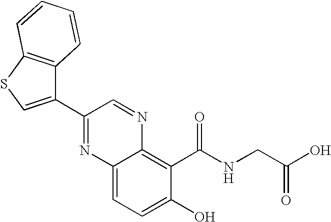

### N-{[2-(1-benzothien-3-yl)-6-hydroxy-5-quinoxalinyl]carbonyl}glycine

To a mixture of the compound from example 5(a) (0.300 g, 0.85 mmol), benzo[b]thiophen-3-ylboronic acid (0.181 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, washed with methanol (25.0 mL) to afford the title compound (0.162 g, 50.5% yield) as a yellow solid. ¹H NMR (300 MHz, DMSO-d6) δ ppm 15.20 (s, 1H), 12.98 (s, 1H), 11.40 (t, 1H, J=5.4 Hz), 9.54 (s, 1H), 9.04 (d, 1H, J=8.7 Hz), 8.87 (s, 1H), 8.32 (d, 1H, J=9.0 Hz), 8.14 (t, 1H, J=7.2 Hz), 7.54 (m, 3H), 4.28 (d, 2H, J=5.4 Hz). MS (ES+) m/e 380 [M+H]⁺.

### Example 112

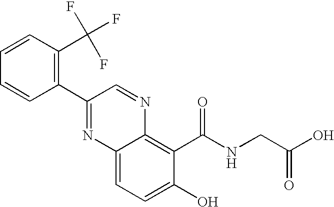

### N-({6-hydroxy-2-[2-(trifluoromethyl)phenyl]-5-quinoxalinyl}carbonyl)glycine

112(a) Ethyl N-({6-hydroxy-2-[2-(trifluoromethyl)phenyl]-5-quinoxalinyl}carbonyl)glycinate. To a mixture of the compound from example 5(a) (0.300 g, 0.85 mmol), 2-(trifluoromethyl)phenylboronic acid (0.193 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuran. The organic phase was concentrated in vacuo to afford the title compound (0.265 g, 74.6% yield) as a green solid, MS (ES+) m/e 420 [M+H]⁺, used in the next step without further purification.

112(b) N-({6-hydroxy-2-[2-(trifluoromethyl)phenyl]-5-quinoxalinyl}carbonyl)glycine. To the above crude ester (0.265 g, 0.63 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added to adjust pH to 3. The precipitate was collected by filtration to get crude product, which was purified by rp-HPLC (acetonitrile/water+0.1% trifluoroacetic acid) to afford the title compound (0.137 g, 55.5% yield) as a white solid. ¹H NMR (300 MHz, DMSO-d6) δ ppm 15.39 (s, 1H, br), 12.94 (s, 1H, br), 11.32 (t, 1H, br, J=5.7 Hz), 9.08 (s, 1H), 8.21 (d, 1H, J=9.0 Hz), 7.99 (m, 1H), 7.86 (m, 1H), 7.71 (m, 2H), 7.63 (d, 1H, J=9.9 Hz), 4.26 (d, 2H, J=5.4 Hz). MS (ES+) m/e 392 [M+H]⁺.

### Example 113

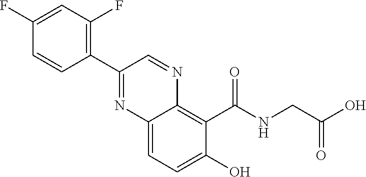

### N-{[2-(2,4-difluorophenyl)-6-hydroxy-5-quinoxalinyl]carbonyl}glycine

To a mixture of the compound from example 5(a) (0.300 g, 0.85 mmol), 2,4-difluorophenylboronic acid (0.160 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.160 g, 52.6% yield) as a pale yellow solid. ¹H NMR (300 MHz, DMSO-d6) δ ppm 15.36 (s, 1H), 12.94 (s, 1H), 11.34 (t, 1H, J=5.7 Hz), 9.28 (d, 1H, J=3.3 Hz), 8.24 (d, 1H, J=9.6 Hz), 8.16 (m, 1H), 7.55 (m, 2H), 7.35 (m, 1H), 4.25 (d, 2H, J=5.7 Hz). MS (ES+) m/e 360 [M+H]⁺.

### Example 114

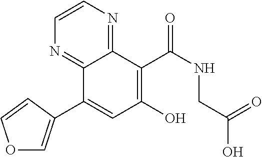

### N-{[8-(3-furanyl)-6-hydroxy-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (13.05 mg, 0.011 mmol), 3-furanylboronic acid (13.90 mg, 0.124 mmol) and potassium carbonate (31.2 mg, 0.226 mmol) in 1,4-dioxane (3.0 ml) and water (1.0 ml) to give a yellow suspension. The mixture was heated to 120° C. for 60 min in a Biotage Initiator® microwave synthesizer, then cooled and diluted with methanol. Sodium hydroxide (1.0N in water) (0.226 ml, 0.226 mmol) was added. The reaction was kept stirring at ambient temperature for half hour and quenched with 5 ml hydrochloric acid (1N in water). The precipitate was collected, washed with water and methylene chloride and dried to afford N-{[8-(3-furanyl)-6-hydroxy-5-quinoxalinyl]carbonyl}glycine (25 mg, 0.080 mmol, 70.7% yield) as a yellow solid. ¹H NMR (400 MHz, DMSO-d6) δ ppm 15.36 (s, 1H), 12.89 (br. s., 1H), 11.40 (t, J=5.4 Hz, 1H), 8.97 (d, J=9.1 Hz, 2H), 8.84 (s, 1H), 7.86 (t, J=1.6 Hz, 2H), 7.36 (s, 1H), 4.24 (d, J=5.6 Hz, 3H). MS (ES+) m/e 314[M+H]⁺.

### Example 115

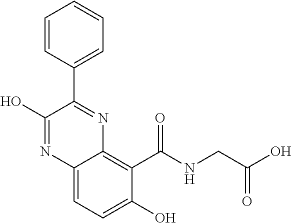

### N-[(6-hydroxy-2-oxo-3-phenyl-1,2-dihydro-5-quinoxalinyl)carbonyl]glycine

115(a) methyl 6-(methyloxy)-2-oxo-3-phenyl-1,2-dihydro-5-quinoxalinecarboxylate. To a solution of methyl 2-amino-6-(methyloxy)-3-nitrobenzoate (example 1(b), 4.0 g, 17.68 mmol) in ethyl acetate (25 mL) was added 10% palladium on charcoal (0.941 g, 0.884 mmol) followed by evacuation of the reaction vessel and purging with 50 psi of hydrogen. Following hydrogenation in the Parr Shaker overnight, the reaction mixture was filtered through Celite®, washed through with ethyl acetate, and concentrated in vacuo. The resulting residue was dissolved in acetonitrile (25.00 mL), treated with ethyl oxo(phenyl)acetate (3.47 g, 19.45 mmol), and stirred at room temperature overnight. The resulting solid was filtered and dried in vacuo to obtain methyl 6-(methyloxy)-2-oxo-3-phenyl-1,2-dihydro-5-quinoxalinecarboxylate (2.21 g, 7.12 mmol, 40.3% yield) as a bright yellow solid. ¹H NMR (400 MHz, DMSO-d6) δ ppm 12.58 (br. s., 1H), 8.30 (d, J=1.5 Hz, 1H), 8.27-8.29 (m, 1H), 7.47-7.54 (m, 3H), 7.45 (d, J=9.1 Hz, 1H), 7.38 (d, J=9.1 Hz, 1H), 3.89 (s, 3H), 3.85 (s, 3H). MS (ES+) m/e 311 [M+H]⁺.

115(b) 6-hydroxy-2-oxo-3-phenyl-1,2-dihydro-5-quinoxalinecarboxylic acid. A solution of methyl 6-(methyloxy)-2-oxo-3-phenyl-1,2-dihydro-5-quinoxalinecarboxylate (0.120 g, 0.387 mmol) in dichloromethane (10 mL) was treated with boron tribromide (1M solution in dichloromethane) (1.934 mL, 1.934 mmol) at room temperature overnight. The reaction mixture was poured into water and extracted twice with ethyl acetate. The combined organic portions were dried over magnesium sulfate, filtered and concentrated to give 6-hydroxy-2-oxo-3-phenyl-1,2-dihydro-5-quinoxalinecarboxylic acid (0.08 g, 0.283 mmol, 73.3% yield) as an orange solid. ¹H NMR (400 MHz, DMSO-d6) δ ppm 14.79 (s, 1H), 11.24 (s, 1H), 8.25 (d, J=1.5 Hz, 1H), 7.51-7.60 (m, 3H), 7.44 (d, J=9.1 Hz, 1H), 7.30 (d, J=9.1 Hz, 1H). MS (ES+) m/e 283 [M+H]⁺.

115(c) ethyl N-[(6-hydroxy-2-oxo-3-phenyl-1,2-dihydro-5-quinoxalinyl)carbonyl]glycinate. A solution of 6-hydroxy-2-oxo-3-phenyl-1,2-dihydro-5-quinoxalinecarboxylic acid (0.08 g, 0.283 mmol) and ethyl glycine hydrochloride (0.079 g, 0.567 mmol) in N,N-Dimethylformamide (DMF) (3.0 mL) was treated with triethylamine (0.119 mL, 0.850 mmol) and PyBOP (0.162 g, 0.312 mmol). The reaction mixture was stirred overnight at ambient temperature, quenched by water, filtered and dried in vacuo. The resulting solid was purified via flash column chromatography (0-100% ethyl acetate in hexanes) to obtain ethyl N-[(6-hydroxy-2-oxo-3-phenyl-1,2-dihydro-5-quinoxalinyl)carbonyl]glycinate (0.076 g, 0.207 mmol, 73.0% yield) as a yellow solid. ¹H NMR (400 MHz, DMSO-d₆) δ ppm 13.72 (s, 1H), 12.86 (br. s., 1H), 11.06 (t, J=5.3 Hz, 1H), 8.29 (s, 1H), 8.27 (d, J=1.5 Hz, 1H), 7.39-7.69 (m, 4H), 7.27 (d, J=9.1 Hz, 1H), 4.33 (d, J=5.8 Hz, 2H), 4.16 (q, J=7.1 Hz, 2H), 1.19 (t, J=7.2 Hz, 3H). MS (ES+) m/e 368 [M+H]⁺.

115(d) N-[(6-hydroxy-2-oxo-3-phenyl-1,2-dihydro-5-quinoxalinyl)carbonyl]glycine. To a suspension of ethyl N-[(6-hydroxy-2-oxo-3-phenyl-1,2-dihydro-5-quinoxalinyl)carbonyl]glycinate (0.076 g, 0.207 mmol) in ethanol (2.0 mL) was added 1N aqueous sodium hydroxide (3.00 ml, 3.00 mmol). After stirring 30 min. at ambient temperature, the reaction was quenched with 1N aqueous hydrochloric acid and the resulting precipitate was filtered, washed with water, and dried in vacuo. The residue was purified using C-18 reverse phase column (0-100% acetonitrile water) to obtain N-[(6-hydroxy-2-oxo-3-phenyl-1,2-dihydro-5-quinoxalinyl)carbonyl]glycine (0.038 g, 0.112 mmol, 54.1% yield) as dark yellow solid ¹H NMR (400 MHz, DMSO-d₆) δ ppm 13.90 (s, 1H), 12.94 (s, 1H), 10.99 (t, J=5.3 Hz, 1H), 8.29 (s, 1H), 8.27 (d, J=1.5 Hz, 1H), 7.44-7.61 (m, 4H), 7.24 (d, J=9.1 Hz, 1H), 4.24 (d, J=5.3 Hz, 2H). MS (ES+) m/e 340 [M+H]⁺.

### Example 116

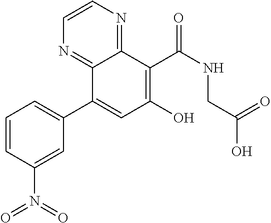

### N-{[6-hydroxy-8-(3-nitrophenyl)-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (13.05 mg, 0.011 mmol), (3-nitrophenyl)boronic acid (20.74 mg, 0.124 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (1.0 N in water) (0.226 ml, 0.226 mmol) was added. The reaction was kept stirring at ambient temperature for half hour and quenched with 5 ml hydrochloric acid (1N in water). The precipitate was collected, washed with water and methylene chloride and dried to afford N-{[6-hydroxy-8-(3-nitrophenyl)-5-quinoxalinyl]carbonyl}glycine (26 mg, 0.071 mmol, 62.5% yield) as a yellow solid. ¹H NMR (400 MHz, DMSO-d6) δ ppm 15.44 (br. s., 1H), 12.97 (br. s., 1H), 11.45 (t, J=5.6 Hz, 1H), 9.00 (d, J=1.5 Hz, 1H), 8.92 (d, J=1.3 Hz, 1H), 8.54 (s, 1H), 8.36 (dd, J=8.1, 2.3 Hz, 1H), 8.16 (d, J=7.8 Hz, 1H), 7.83 (t, J=8.0 Hz, 1H), 7.72 (s, 1H), 4.27 (d, J=5.6 Hz, 2H). MS (ES+) m/e 369[M+H]⁺.

### Example 117

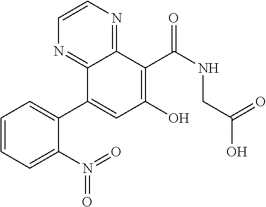

### N-{[6-hydroxy-8-(2-nitrophenyl)-5-quinoxalinyl]carbonyl}glycine

In a 10 mL microwave vial was placed ethyl N-[(8-bromo-6-hydroxy-5-quinoxalinyl)carbonyl]glycinate (40 mg, 0.113 mmol), tetrakis(triphenylphosphine)palladium(0) (13.05 mg, 0.011 mmol), (2-nitrophenyl)boronic acid (20.74 mg, 0.124 mmol) and potassium carbonate (31.2 mg, 0.226 mmol) in 1,4-dioxane (3.0 ml) and water (1.0 ml) to give a yellow suspension. The mixture was heated to 120° C. for 60 min. in a Biotage Initiator® microwave synthesizer, then cooled and diluted with methanol. Sodium hydroxide (1.0 N in water) (0.226 ml, 0.226 mmol) was added. The reaction was kept stirring at ambient temperature for half hour and quenched with 5 ml hydrochloric acid (1N in water). The precipitate was collected, washed with water and methylene chloride and dried to afford N-{[6-hydroxy-8-(2-nitrophenyl)-5-quinoxalinyl]carbonyl}glycine (18 mg, 0.049 mmol, 43.3% yield) as a yellow solid. ¹H NMR (400 MHz, DMSO-d6) δ ppm 15.49 (s, 1H), 12.93 (br. s., 1H), 11.37 (t, J=5.6 Hz, 1H), 8.95 (d, J=2.0 Hz, 1H), 8.73 (d, J=2.0 Hz, 1H), 8.21 (d, J=1.0 Hz, 1H), 7.91 (t, J=6.9 Hz, 1H), 7.79 (dd, J=15.5, 1.4 Hz, 1H), 7.72 (dd, J=7.6, 1.3 Hz, 1H), 7.69 (s, 1H), 4.27 (d, J=5.6 Hz, 2H. MS (ES+) m/e 369[M+H]⁺.

### Example 118

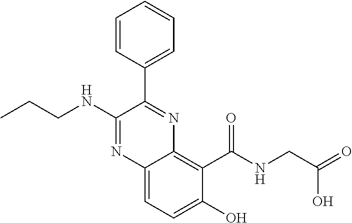

### N-{[6-hydroxy-3-phenyl-2-(propylamino)-5-quinoxalinyl]carbonyl}glycine

118(a) methyl 2-chloro-6-(methyloxy)-3-phenyl-5-quinoxalinecarboxylate. To a solution of methyl 6-(methyloxy)-2-oxo-3-phenyl-1,2-dihydro-5-quinoxalinecarboxylate (0.320 g, 1.031 mmol) was added phosphorus oxychloride (3.00 ml, 32.2 mmol). After heating to reflux for 4 h, the reaction mixture was carefully treated with ice water. The resulting precipitate was filtered, washed with water, and concentrated in vacuo to afford methyl 2-chloro-6-(methyloxy)-3-phenyl-5-quinoxalinecarboxylate (0.225 g, 0.684 mmol, 66.4% yield) as a yellow solid. ¹H NMR (400 MHz, DMSO-d₆) δ ppm 8.22 (d, J=9.0 Hz, 1H), 7.92 (d, J=9.0 Hz, 1H), 7.76-7.85 (m, 2H), 7.51-7.60 (m, 3H), 4.03 (s, 3H), 3.86 (s, 3H). MS (ES+) m/e 329/331 [M+H]⁺.

118(b) methyl 6-(methyloxy)-3-phenyl-2-(propylamino)-5-quinoxalinecarboxylate. A solution of methyl 2-chloro-6-(methyloxy)-3-phenyl-5-quinoxalinecarboxylate (0.225 g, 0.684 mmol), n-propylamine (1.0 ml, 12.01 mmol) and triethylamine (0.286 ml, 2.053 mmol) in tetrahydrofuran (3.0 ml) was heated to 100° C. overnight in an oil bath After cooling, the reaction mixture was concentrated in vacuo and purified via flash column chromatography (0-10% ethyl acetate in hexanes) to afford methyl 6-(methyloxy)-3-phenyl-2-(propylamino)-5-quinoxalinecarboxylate (0.160 g, 0.455 mmol, 66.5% yield) as an orange oil. ¹H NMR (400 MHz, DMSO-d₆) δ ppm 7.66-7.74 (m, 2H), 7.70 (d, J=9.1 Hz, 1H), 7.53-7.59 (m, 3H), 6.57 (t, J=5.6 Hz, 1H), 3.89 (s, 3H), 3.82 (s, 3H), 3.34-3.41 (m, 2H), 1.52-1.82 (m, 2H), 0.91 (t, J=7.5 Hz, 3H). MS (ES+) m/e 352 [M+H]⁺.

118(c) 6-hydroxy-3-phenyl-2-(propylamino)-5-quinoxalinecarboxylic acid. A solution of methyl 6-(methyloxy)-3-phenyl-2-(propylamino)-5-quinoxalinecarboxylate (0.160 g, 0.455 mmol) in dichloromethane (10 mL) was treated with boron tribromide (1M solution in dichloromethane) (2.3 mL, 2.300 mmol) at room temperature overnight. The reaction mixture was poured into water and extracted twice with ethyl acetate. The combined organic portions were dried over magnesium sulfate, filtered and concentrated to give 6-hydroxy-3-phenyl-2-(propylamino)-5-quinoxalinecarboxylic acid (0.108 g, 0.334 mmol, 73.4% yield) as a red solid. ¹H NMR (400 MHz, DMSO-d₆) δ ppm 12.22 (br. s., 1H), 7.88 (d, J=9.1 Hz, 1H), 7.78-7.84 (m, 2H), 7.60-7.68 (m, 3H), 7.40 (d, J=9.1 Hz, 1H), 6.95 (t, J=5.6 Hz, 1H), 3.26-3.47 (m, 2H), 1.50-1.72 (m, 2H), 0.92 (t, J=7.5 Hz, 3H). MS (ES+) m/e 324 [M+H]⁺.

118(d) ethyl N-{[6-hydroxy-3-phenyl-2-(propylamino)-5-quinoxalinyl]carbonyl}glycinate. A solution of 6-hydroxy-3-phenyl-2-(propylamino)-5-quinoxalinecarboxylic acid (0.108 g, 0.334 mmol) and ethyl glycine hydrochloride (0.093 g, 0.668 mmol) in N,N-Dimethylformamide (DMF) (3.0 mL) was treated with triethylamine (0.140 mL, 1.002 mmol) and PyBOP (0.195 g, 0.375 mmol). The reaction mixture was stirred overnight at ambient temperature, quenched by water, filtered and dried in vacuo to obtain ethyl N-{[6-hydroxy-3-phenyl-2-(propylamino)-5-quinoxalinyl]carbonyl}glycinate (0.085 g, 0.208 mmol, 62.3% yield) as an orange solid. ¹H NMR (400 MHz, DMSO-d₆) δ ppm 14.20 (s, 1H), 11.35 (t, J=5.4 Hz, 1H), 7.87 (dd, J=3.3, 1.0 Hz, 1H), 7.86 (d, J=2.0 Hz, 1H), 7.77 (d, J=9.1 Hz, 1H), 7.58 (d, J=2.0 Hz, 1H), 7.57-7.62 (m, 2H), 7.28 (d, J=9.1 Hz, 1H), 6.70 (t, J=5.6 Hz, 1H), 4.27 (d, J=5.4 Hz, 2H), 4.12 (q, J=7.2 Hz, 2H), 3.34-3.41 (m, 2H), 1.55-1.71 (m, 2H), 1.17 (t, J=7.1 Hz, 3H), 0.92 (t, J=7.3 Hz, 3H). MS (ES+) m/e 409 [M+H]⁺.

118(e) N-{[6-hydroxy-3-phenyl-2-(propylamino)-5-quinoxalinyl]carbonyl}glycine. To a suspension of ethyl N-{[6-hydroxy-3-phenyl-2-(propylamino)-5-quinoxalinyl]carbonyl}glycinate (0.085 g, 0.208 mmol) in ethanol (2.0 ml) was added 1N aqueous sodium hydroxide (2.0 ml, 2.000 mmol). After stirring 30 min. at ambient temperature, the reaction was quenched with 1N aqueous hydrochloric acid and extracted twice using ethyl acetate. The combined organic portions were dried over magensium sulfate, filtered and concentrated in vacuo to obtain N-{[6-hydroxy-3-phenyl-2-(propylamino)-5-quinoxalinyl]carbonyl}glycine (0.079 g, 0.208 mmol, 100% yield) as a bright yellow solid. ¹H NMR (400 MHz, DMSO-d₆) δ ppm 14.34 (br. s., 1H), 11.26 (t, J=5.3 Hz, 1H), 7.87 (dd, J=6.6, 3.0 Hz, 2H), 7.77 (d, J=9.1 Hz, 1H), 7.54-7.62 (m, 3H), 7.28 (d, J=9.1 Hz, 1H), 6.72 (br. s., 1H), 4.19 (d, J=5.3 Hz, 2H), 3.32-3.43 (m, 2H), 1.57-1.72 (m, 2H), 0.92 (t, J=7.5 Hz, 3H). MS (ES+) m/e 381 [M+H]⁺.

### Example 119

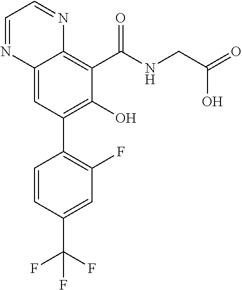

### N-({7-[2-fluoro-4-(trifluoromethyl)phenyl]-6-hydroxy-5-quinoxalinyl}carbonyl)glycine

A solution of ethyl N-[(7-bromo-6-hydroxy-5-quinoxalinyl)carbonyl]glycinate (0.140 g, 0.395 mmol), [2-fluoro-4-(trifluoromethyl)phenyl]boronic acid (0.082 g, 0.395 mmol), potassium carbonate (0.164 g, 1.186 mmol), and tetrakis(triphenylphosphine)palladium(0) (10 mg, 8.65 μmol) in 1,4-dioxane (2.0 ml) and water (0.667 ml) was heated to 100° C. for 20 min. in a Biotage Initiator® microwave synthesizer. Upon cooling, the reaction mixture was treated with water and extracted twice with ethyl acetate. The combined organic portions were dried over magnesium sulfate, filtered and concentrated in vacuo. The residue was purified using C-18 Reverse phase flash column chromatography to obtain N-({7-[2-fluoro-4-(trifluoromethyl)phenyl]-6-hydroxy-5-quinoxalinyl}carbonyl)glycine (0.015 g, 0.037 mmol, 9.27% yield). ¹H NMR (400 MHz, DMSO-d₆) δ ppm 12.95 (br. s., 1H), 11.53 (t, J=5.4 Hz, 1H), 9.02 (d, J=2.0 Hz, 1H), 8.98 (d, J=2.0 Hz, 1H), 8.32 (s, 1H), 7.80-7.91 (m, 2H), 7.76 (dd, J=7.8, 1.5 Hz, 1H), 4.27 (d, J=5.6 Hz, 1H). MS (ES+) m/e 410 [M+H]⁺.

### Example 120

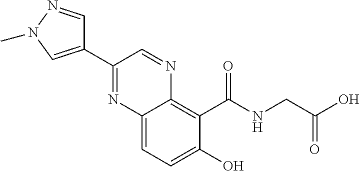

### N-{[6-hydroxy-2-(1-methyl-1H-pyrazol-4-yl)-5-quinoxalinyl]carbonyl}glycine

To a mixture of the compound from example 5(a) (0.300 g, 0.85 mmol), 1-methyl-1H-pyrazol-4-ylboronic acid (0.128 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, washed with methanol (15.0 mL) to afford the title compound (0.239 g, 86.5% yield) as a pale yellow solid. ¹H NMR (300 MHz, DMSO-d6) δ ppm 15.09 (s, 1H), 12.97 (s, 1H), 11.35 (t, 1H, J=4.8 Hz), 9.24 (s, 1H), 8.57 (s, 1H), 8.24 (s, 1H), 8.06 (d, 1H, J=8.7 Hz), 7.49 (d, 1H, J=9.6 Hz), 4.25 (d, 2H, J=5.4 Hz), 3.95 (s, 3H). MS (ES+) m/e 328 [M+H]⁺.

### Example 121

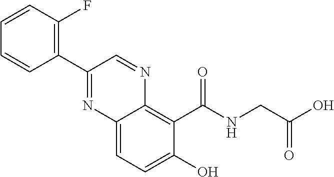

### N-{[2-(2-fluorophenyl)-6-hydroxy-5-quinoxalinyl]carbonyl}glycine

To a mixture of the compound from example 5(a) (0.300 g, 0.85 mmol), 2-fluorophenylboronic acid (0.142 g, 1.02 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.098 g, 33.8% yield) as a yellow solid. ¹H NMR (300 MHz, DMSO-d6) δ ppm 15.36 (s, 1H), 12.94 (s, 1H), 11.36 (t, 1H, J=5.4 Hz), 9.31 (d, 1H, J=2.4 Hz), 8.25 (d, 1H, J=9.0 Hz), 8.11 (m, 1H), 7.62 (m, 2H), 7.45 (m, 2H), 4.25 (d, 2H, J=5.7 Hz). MS (ES+) m/e 342 [M+H]⁺.

### Example 122

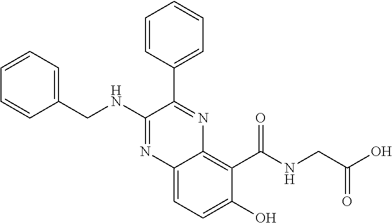

### N-({6-hydroxy-3-phenyl-2-[(phenylmethyl)amino]-5-quinoxalinyl}carbonyl)glycine

122(a) methyl 6-(methyloxy)-3-phenyl-2-[(phenylmethyl)amino]-5-quinoxalinecarboxylate. A solution of methyl 2-chloro-6-(methyloxy)-3-phenyl-5-quinoxalinecarboxylate (example 118(a), 0.208 g, 0.633 mmol), benzylamine (2.0 ml, 18.29 mmol) and triethylamine (1.0 ml, 7.17 mmol) in tetrahydrofuran (3.0 ml) and methanol (3.00 ml) was heated to 100° C. overnight in an oil bath. After cooling, the reaction mixture was concentrated and purified via flash column chromatography (0-100% ethyl acetate in hexanes) to give methyl 6-(methyloxy)-3-phenyl-2-[(phenylmethyl)amino]-5-quinoxalinecarboxylate (0.138 g, 0.345 mmol, 54.6% yield) as an orange solid. ¹H NMR (400 MHz, DMSO-d₆) δ ppm 7.70-7.77 (m, 2H), 7.65 (d, J=9.1 Hz, 1H), 7.54-7.61 (m, 3H), 7.49 (d, J=9.1 Hz, 1H), 7.39-7.41 (m, 1H), 7.38 (s, 1H), 7.29 (t, J=7.6 Hz, 2H), 7.16-7.23 (m, 2H), 4.63 (d, J=6.1 Hz, 2H), 3.87 (s, 3H), 3.81 (s, 3H). MS (ES+) m/e 400 [M+H]⁺.

122(b) 6-hydroxy-3-phenyl-2-[(phenylmethyl)amino]-5-quinoxalinecarboxylic acid. A solution of methyl 6-(methyloxy)-3-phenyl-2-[(phenylmethyl)amino]-5-quinoxalinecarboxylate (0.138 g, 0.345 mmol) in dichloromethane (10 ml) was treated with boron tribromide (1M solution in dichloromethane) (1.727 ml, 1.727 mmol) dropwise at room temperature. The solution was stirred at room temperature overnight. Then it was quenched by water and extracted with dichloromethane, dried over magnesium sulfate, filtered and concentrated to obtain 6-hydroxy-3-phenyl-2-[(phenylmethyl)amino]-5-quinoxalinecarboxylic acid (0.104 g, 0.280 mmol, 81% yield) as an orange solid. ¹H NMR (400 MHz, DMSO-d₆) δ ppm 12.22 (br. s., 1H), 7.83-7.88 (m, 2H), 7.84 (d, J=9.1 Hz, 1H), 7.61-7.69 (m, 3H), 7.58 (t, J=6.1 Hz, 1H), 7.38-7.45 (m, 2H), 7.39 (d, J=9.1 Hz, 1H), 7.30 (t, J=7.8 Hz, 2H), 7.20 (tt, J=7.3, 1.8 Hz, 1H), 4.65 (d, J=6.1 Hz, 2H). MS (ES+) m/e 372 [M+H]⁺.

122(c) ethyl N-({6-hydroxy-3-phenyl-2-[(phenylmethyl)amino]-5-quinoxalinyl}carbonyl)glycinate. A solution of 6-hydroxy-3-phenyl-2-[(phenylmethyl)amino]-5-quinoxalinecarboxylic acid (0.104 g, 0.280 mmol) and ethyl glycine hydrochloride (0.078 g, 0.560 mmol) in N,N-Dimethylformamide (DMF) (3.0 mL) was treated with triethylamine (0.117 mL, 0.840 mmol) and PyBOP (0.160 g, 0.308 mmol). The reaction mixture was stirred overnight at ambient temperature, quenched by water, filtered and dried in vacuo to obtain ethyl N-({6-hydroxy-3-phenyl-2-[(phenylmethyl)amino]-5-quinoxalinyl}carbonyl)glycinate (0.120 g, 0.263 mmol, 94% yield) as an orange solid. ¹H NMR (400 MHz, DMSO-d₆) δ ppm 14.23 (s, 1H), 11.34 (t, J=5.6 Hz, 1H), 7.90-7.92 (m, 1H), 7.89 (d, J=2.0 Hz, 1H), 7.73 (d, J=9.1 Hz, 1H), 7.61 (d, J=2.0 Hz, 1H), 7.57-7.64 (m, 2H), 7.43 (d, J=1.3 Hz, 1H), 7.41 (d, J=0.5 Hz, 1H), 7.29-7.37 (m, 3H), 7.27 (d, J=9.1 Hz, 1H), 7.20 (tt, J=7.3, 1.3 Hz, 1H), 4.63 (d, J=5.8 Hz, 2H), 4.26 (d, J=5.3 Hz, 2H), 4.12 (q, J=7.2 Hz, 2H), 1.17 (t, J=7.1 Hz, 3H). MS (ES+) m/e 457 [M+H]⁺.

122(d) N-({6-hydroxy-3-phenyl-2-[(phenylmethyl)amino]-5-quinoxalinyl}carbonyl)glycine. To a suspension of ethyl N-({6-hydroxy-3-phenyl-2-[(phenylmethyl)amino]-5-quinoxalinyl}carbonyl)glycinate (0.120 g, 0.263 mmol) in ethanol (1.0 mL) was added 1N aqueous sodium hydroxide (2.00 ml, 2.000 mmol). After stirring overnight at ambient temperature, the reaction was quenched with 1N aqueous hydrochloric acid and the resulting precipitate was filtered, washed with water, and dried in vacuo to obtain N-({6-hydroxy-3-phenyl-2-[(phenylmethyl)amino]-5-quinoxalinyl}carbonyl)glycine (0.0997 g, 0.209 mmol, 80% yield) as a yellow solid. ¹H NMR (400 MHz, DMSO-d₆) δ ppm 14.37 (s, 1H), 11.27 (t, J=5.3 Hz, 1H), 7.91-7.94 (m, 1H), 7.90 (d, J=1.8 Hz, 1H), 7.72 (d, J=9.1 Hz, 1H), 7.59 (d, J=1.8 Hz, 1H), 7.56-7.61 (m, 2H), 7.42 (s, 1H), 7.41 (d, J=0.5 Hz, 1H), 7.28-7.34 (m, 3H), 7.27 (d, J=9.1 Hz, 1H), 7.20 (tt, J=7.3, 1.3 Hz, 1H), 4.63 (d, J=6.1 Hz, 2H), 4.19 (d, J=5.3 Hz, 2H). MS (ES+) m/e 429 [M+H]⁺.

### Example 123

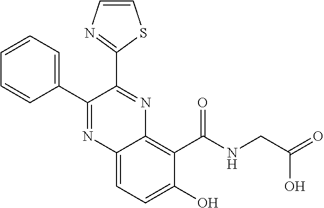

### N-{[6-hydroxy-2-phenyl-3-(1,3-thiazol-2-yl)-5-quinoxalinyl]carbonyl}glycine

123(a) methyl 6-(methyloxy)-3-oxo-2-phenyl-3,4-dihydro-5-quinoxalinecarboxylate. To a solution of methyl 2-amino-6-(methyloxy)-3-nitrobenzoate (1.0 g, 4.42 mmol) in ethyl acetate (10.0 mL) was added 10% palladium on charcoal (0.235 g, 0.221 mmol) followed by evacuation of the reaction vessel and purging with 50 psi of hydrogen. Following hydrogenation in the Parr Shaker overnight, the reaction mixture was filtered through Celite®, washed through with ethyl acetate, and concentrated in vacuo. The resulting residue was dissolved in acetonitrile (10.00 mL), treated with ethyl oxo(phenyl)acetate (0.867 g, 4.86 mmol), and stirred under reflux overnight. After cooling, the reaction mixture was filtered and washed with acetonitrile to obtain methyl 6-(methyloxy)-3-oxo-2-phenyl-3,4-dihydro-5-quinoxalinecarboxylate (0.703 g, 2.266 mmol, 51.2% yield) as a pale yellow solid. ¹H NMR (400 MHz, DMSO-d₆) δ ppm 12.04 (br. s., 1H), 8.28 (d, J=2.0 Hz, 1H), 8.23-8.27 (m, 1H), 7.96 (d, J=9.1 Hz, 1H), 7.45-7.54 (m, 3H), 7.19 (d, J=9.1 Hz, 1H), 3.92 (s, 3H), 3.88 (s, 3H). MS (ES+) m/e 311 [M+H]⁺.

123(b) methyl 3-chloro-6-(methyloxy)-2-phenyl-5-quinoxalinecarboxylate. To a solution of methyl 6-(methyloxy)-3-oxo-2-phenyl-3,4-dihydro-5-quinoxalinecarboxylate (0.355 g, 1.144 mmol) was added phosphorus oxychloride (1.066 ml, 11.44 mmol). After heating to reflux for 2 h, the reaction mixture was carefully treated with ice water. The resulting precipitate was filtered, washed with water, and concentrated in vacuo to afford methyl 3-chloro-6-(methyloxy)-2-phenyl-5-quinoxalinecarboxylate (0.353 g, 1.074 mmol, 94% yield) as a pale yellow solid. ¹H NMR (400 MHz, DMSO-d₆) δ ppm 8.28 (d, J=9.3 Hz, 1H), 7.91 (d, J=9.3 Hz, 1H), 7.78-7.84 (m, 2H), 7.51-7.60 (m, 3H), 4.04 (s, 3H), 3.93 (s, 3H). MS (ES+) m/e 329/331 [M+H]⁺.

123(c) methyl 6-(methyloxy)-2-phenyl-3-(1,3-thiazol-2-yl)-5-quinoxalinecarboxylate. To a solution of methyl 3-chloro-6-(methyloxy)-2-phenyl-5-quinoxalinecarboxylate (0.123 g, 0.374 mmol) in 1,4-dioxane (1.5 ml) was added 2-(tributylstannanyl)-1,3-thiazole (0.168 g, 0.449 mmol) and tetrakis(triphenylphosphine)palladium(0) (0.013 g, 0.011 mmol) followed by heating to 150° C. for 100 min in a Biotage Initiator® microwave synthesizer. Upon cooling, the reaction mixture was filtered through a celite pad, washed with ethyl acetate and concentrated in vacuo. The residue was purified via flash column chromatography (0-100% ethyl acetate in hexanes) to obtain methyl 6-(methyloxy)-2-phenyl-3-(1,3-thiazol-2-yl)-5-quinoxalinecarboxylate (0.110 g, 0.291 mmol, 78% yield) as a yellow solid. ¹H NMR (400 MHz, DMSO-d₆) δ ppm 8.25 (d, J=9.3 Hz, 1H), 7.91 (d, J=3.3 Hz, 1H), 7.91 (d, J=9.4 Hz, 1H), 7.78 (d, J=3.3 Hz, 1H), 7.55 (d, J=1.3 Hz, 1H), 7.53 (d, J=1.8 Hz, 1H), 7.37-7.45 (m, 3H), 4.03 (s, 3H), 3.96 (s, 3H). MS (ES+) m/e 378 [M+H]⁺.

123(d) 6-hydroxy-2-phenyl-3-(1,3-thiazol-2-yl)-5-quinoxalinecarboxylic acid. A solution of methyl 6-(methyloxy)-2-phenyl-3-(1,3-thiazol-2-yl)-5-quinoxalinecarboxylate (0.110 g, 0.291 mmol) in dichloromethane (10 mL) was treated with boron tribromide (1M solution in dichloromethane) (1.166 mL, 1.166 mmol) at room temperature overnight. The reaction mixture was poured into water and extracted twice with ethyl acetate. The combined organic portions were dried over magnesium sulfate, filtered and concentrated to give 6-hydroxy-2-phenyl-3-(1,3-thiazol-2-yl)-5-quinoxalinecarboxylic acid (0.157 g, 0.449 mmol, 154% yield) as a yellow solid. ¹H NMR (400 MHz, DMSO-d₆) δ ppm 8.25 (d, J=9.3 Hz, 1H), 8.00 (d, J=3.3 Hz, 1H), 7.93 (d, J=3.0 Hz, 1H), 7.68 (d, J=9.3 Hz, 1H), 7.58 (d, J=1.5 Hz, 1H), 7.56 (d, J=2.0 Hz, 1H), 7.44-7.50 (m, 3H). MS (ES+) m/e 350 [M+H]⁺.

123(e) ethyl N-{[6-hydroxy-2-phenyl-3-(1,3-thiazol-2-yl)-5-quinoxalinyl]carbonyl}glycinate. A solution of 6-hydroxy-2-phenyl-3-(1,3-thiazol-2-yl)-5-quinoxalinecarboxylic acid (0.157 g, 0.449 mmol) and ethyl glycine hydrochloride (0.125 g, 0.899 mmol) in N,N-Dimethylformamide (DMF) (3.0 mL) was treated with triethylamine (0.188 mL, 1.348 mmol) and PyBOP (0.257 g, 0.494 mmol). The reaction mixture was stirred overnight at ambient temperature, quenched by water, filtered and dried in vacuo to obtain ethyl N-{[6-hydroxy-2-phenyl-3-(1,3-thiazol-2-yl)-5-quinoxalinyl]carbonyl}glycinate (0.06 g, 0.138 mmol, 30.7% yield) as a yellow solid. ¹H NMR (400 MHz, DMSO-d₆) δ ppm 15.04 (br. s., 1H), 11.14 (t, J=6.1 Hz, 1H), 8.25 (d, J=9.6 Hz, 1H), 8.03 (d, J=3.3 Hz, 1H), 7.89 (d, J=3.0 Hz, 1H), 7.63 (d, J=9.6 Hz, 1H), 7.58 (d, J=1.5 Hz, 1H), 7.56 (d, J=2.0 Hz, 1H), 7.36-7.49 (m, 3H), 4.41 (d, J=6.1 Hz, 2H), 4.18 (q, J=7.1 Hz, 2H), 1.22 (t, J=7.1 Hz, 3H). MS (ES+) m/e 435 [M+H]⁺.

123(f) N-{[6-hydroxy-2-phenyl-3-(1,3-thiazol-2-yl)-5-quinoxalinyl]carbonyl}glycine. To a suspension of ethyl N-{[6-hydroxy-2-phenyl-3-(1,3-thiazol-2-yl)-5-quinoxalinyl]carbonyl}glycinate (0.06 g, 0.138 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-2-phenyl-3-(1,3-thiazol-2-yl)-5-quinoxalinyl]carbonyl}glycine (0.039 g, 0.096 mmol, 69.5% yield) as a yellow solid. ¹H NMR (400 MHz, DMSO-d₆) δ ppm 15.21 (br. s., 1H), 11.08 (t, J=5.8 Hz, 1H), 8.24 (d, J=9.3 Hz, 1H), 8.02 (d, J=3.3 Hz, 1H), 7.88 (d, J=3.3 Hz, 1H), 7.62 (d, J=9.3 Hz, 1H), 7.57 (d, J=1.5 Hz, 1H), 7.55 (d, J=2.0 Hz, 1H), 7.38-7.49 (m, 3H), 4.32 (d, J=5.8 Hz, 2H). MS (ES+) m/e 407 [M+H]⁺.

### Example 124

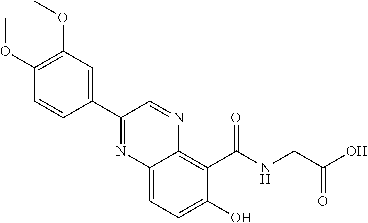

### N-({2-[3,4-bis(methyloxy)phenyl]-6-hydroxy-5-quinoxalinyl}carbonyl)glycine

To a mixture of the compound from example 5(a) (0.300 g, 0.85 mmol), 3,4-dimethoxyphenylboronic acid (0.201 g, 1.11 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.125 g, 38.4% yield) as a yellow solid. ¹H NMR (400 MHz, DMSO-d6) δ ppm 15.15 (s, 1H), 12.95 (s, 1H), 11.41 (t, 1H, J=5.2 Hz), 9.53 (s, 1H), 8.19 (d, 1H, J=9.2 Hz), 7.89 (m, 2H), 7.54 (d, 1H, J=9.2 Hz), 7.16 (d, 1H, J=8.0 Hz), 4.27 (d, 2H, J=5.6 Hz), 3.91 (s, 3H), 3.86 (s, 3H). MS (ES+) m/e 384 [M+H]⁺.

### Example 125

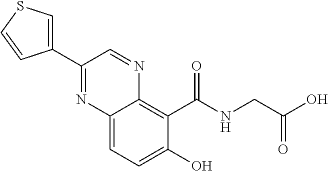

### N-{[6-hydroxy-2-(3-thienyl)-5-quinoxalinyl]carbonyl}glycine

125(a) Ethyl N-{[6-hydroxy-2-(3-thienyl)-5-quinoxalinyl]carbonyl}glycinate. To a mixture of the compound from example 5(a) (0.300 g, 0.85 mmol), thiophen-3-ylboronic acid (0.130 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuan. The mixture was concentrated in vacuo to afford the title compound (0.367 g, 121.1% yield, crude) as an orange solid, MS (ES+) m/e 358 [M+H]⁺, used in the next step without further purification.

125(b) N-{[6-hydroxy-2-(3-thienyl)-5-quinoxalinyl]carbonyl}glycine. To the above crude ester (0.367 g, 1.03 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was filtered, washed through with hexane, dried to afford the title compound (0.217 g, 64.2% yield) as a brown solid. ¹H NMR (400 MHz, DMSO-d6) δ ppm 15.71 (s, 1H, br), 12.95 (s, 1H, br), 11.36 (t, 1H, br, J=5.2 Hz), 9.47 (s, 1H), 8.56 (t, 1H, J=1.2 Hz) 8.15 (d, 1H, J=9.2 Hz), 7.95 (d, 1H, J=5.2 Hz), 7.78 (m, 1H), 7.53 (d, 1H, J=9.2 Hz), 4.27 (d, 2H, J=5.6 Hz). MS (ES+) m/e 330 [M+H]⁺.

### Example 126

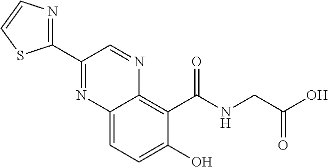

### N-{[6-hydroxy-2-(1,3-thiazol-2-yl)-5-quinoxalinyl]carbonyl}glycine

126(a) Ethyl N-{[6-hydroxy-2-(1,3-thiazol-2-yl)-5-quinoxalinyl]carbonyl}glycinate. To a mixture of the compound from example 5(a) (0.300 g, 0.85 mmol) and 2-(tributylstannyl)thiazole (0.479 g, 1.28 mmol) in 1,4-dioxane (4 mL) was added tetrakis(triphenylphosphine)palladium (0.046 g, 0.040 mmol) followed by evacuation of the reaction vessel and purging with nitrogen. The reaction mixture was heated in a Biotage Initiator® microwave synthesizer at 150° C. for 20 min. Upon cooling, the mixture was concentrated in vacuo to afford the title compound (0.304 g, 100.0% yield), MS (ES+) m/e 359 [M+H]⁺, used in the next step without further purification.

126(b) N-{[6-hydroxy-2-(1,3-thiazol-2-yl)-5-quinoxalinyl]carbonyl}glycine. To the above ester (0.304 g, 0.85 mmol) was added aqueous sodium hydroxide (1N, 10.0 mL) and tetrahydrofuran (10.0 mL). After stirring at ambient temperature for 15 min, the mixture was quenched with 1N hydrochloric acid and the resulting precipitate was filtered and purified by via rp-HPLC (acetonitrile/water+0.1% trifluoroacetic acid) to afford the title compound (0.091 g, 32.5% yield) as a yellow solid. ¹H NMR (400 MHz, DMSO-d6) δ ppm 11.142 (s, 1H), 9.502 (s, 1H), 8.095-8.000 (m, 4H), 7.522 (s, 1H), 3.941 (s, 3H). MS (ES+) m/e 331 [M+H]⁺.

### Example 127

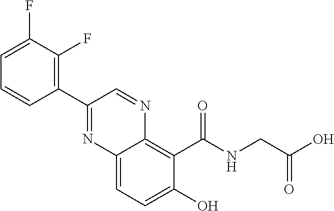

### N-{[2(2,3-difluorophenyl)-6-hydroxy-5-quinoxalinyl]carbonyl}glycine

127(a) Ethyl N-{[2-(2,3-difluorophenyl)-6-hydroxy-5-quinoxalinyl]carbonyl}glycinate. To a mixture of the compound from example 5(a) (0.300 g, 0.85 mmol), 2,3-difluorophenyl boronic acid (0.161 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuan. The mixture was concentrated in vacuo to afford the title compound (0.353 g, 107.6% yield) as a yellow solid, MS (ES+) m/e 358 [M+H]⁺, used in the next step without further purification.

127(b) N-{[2-(2,3-difluorophenyl)-6-hydroxy-5-quinoxalinyl]carbonyl}glycine. To the above crude ester (0.353 g, 0.91 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.231 g, 70.6% yield) as a pale yellow solid. ¹H NMR (400 MHz, DMSO-d6) δ ppm 15.38 (s, 1H, br), 11.32 (t, 1H, br, J=5.4 Hz), 9.30 (m, 1H, J=2.4 Hz), 8.23 (d, 1H, J=9.6 Hz) 7.89 (t, 1H, J=7.6 Hz), 7.65 (m, 2H), 7.46 (m, 1H), 4.26 (d, 2H, J=5.2 Hz). MS (ES+) m/e 330 [M+H]⁺.

### Example 128

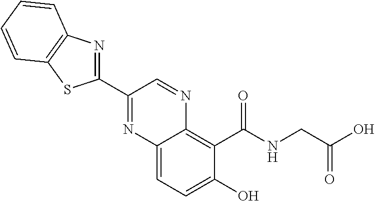

### N-{[2-(1,3-benzothiazol-2-yl)-6-hydroxy-5-quinoxalinyl]carbonyl}glycine

128(a) Ethyl N-{[2-(1,3-benzothiazol-2-yl)-6-hydroxy-5-quinoxalinyl]carbonyl}glycinate. To a mixture of the compound from example 5(a) (0.200 g, 0.56 mmol) and 2-(tributylstannyl)benzo[d]thiazole (0.356 g, 0.84 mmol) in 1,4-dioxane (4 mL) was added tetrakis(triphenylphosphine)palladium (0.035 g, 0.030 mmol) followed by evacuation of the reaction vessel and purging with nitrogen. The reaction mixture was heated in a Biotage Initiator® microwave synthesizer at 150° C. for 20 min. Upon cooling, the mixture was concentrated in vacuo to afford the title compound (0.228 g, 100.0% yield), MS (ES+) m/e 409 [M+H]⁺, used in the next step without further purification.

128(b) N-{[2-(1,3-benzothiazol-2-yl)-6-hydroxy-5-quinoxalinyl]carbonyl}glycine. To the above ester (0.228 g, 0.56 mmol) was added aqueous sodium hydroxide (1N, 10.0 mL) and tetrahydrofuran (10.0 mL). After stirring at ambient temperature for 15 min, the mixture was quenched with 1N hydrochloric acid and the resulting precipitate was filtered and purified by rp-HPLC (acetonitrile/water+0.1% trifluoroacetic acid) to afford the title compound (0.060 g, 28.0% yield) as a yellow solid. ¹H NMR (400 MHz, DMSO-d6) δ ppm 11.585-11.115 (d, 1H, J=188 Hz), 9.624-9.422 (d, 1H, J=80.8 Hz), 8.170-8.119 (d, 3H, J=20.4 Hz), 7.788-7.135 (m, 4H), 4.142-4.003 (m, 3H). MS (ES+) m/e 381 [M+H]⁺.

### Example 129

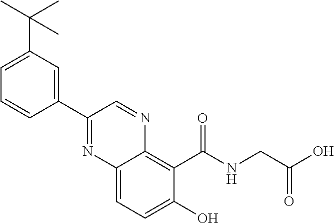

### N-({2-[3-(1,1-dimethylethyl)phenyl]-6-hydroxy-5-quinoxalinyl}carbonyl)glycine

To a mixture of the compound from example 5(a) (0.300 g, 0.85 mmol), 3-tert-butylphenylboronic acid pinacol ester (0.288 g, 1.11 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.115 g, 35.7% yield) as a yellow solid. ¹H NMR (400 MHz, DMSO-d6) δ ppm 15.23 (s, 1H), 12.94 (s, 1H), 11.43 (t, 1H, J=5.2 Hz), 9.56 (s, 1H), 8.30 (s, 1H), 8.25 (d, 1H, J=9.2 Hz), 8.11 (d, 1H, J=7.6 Hz), 7.56 (m, 3H), 4.28 (d, 2H, J=5.2 Hz), 1.39 (s, 9H). MS (ES+) m/e 380 [M+H]⁺.

### Example 130

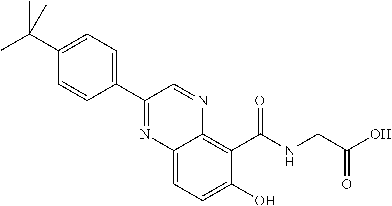

### N-({2-[4-(1,1-dimethylethyl)phenyl]-6-hydroxy-5-quinoxalinyl}carbonyl)glycine

130(a) Ethyl N-({2-[4-(1,1-dimethylethyl)phenyl]-6-hydroxy-5-quinoxalinyl}carbonyl)glycinate. To a mixture of the compound from example 5(a) (0.300 g, 0.85 mmol), 4-tert-butylphenylboronic acid (0.154 g, 1.02 mmol) and potassium carbonate (0.234 g, 1.70 mmol) in 1,4-dioxane (2.5 mL) and water (1.0 mL) was added tetrakis(triphenylphosphine)palladium (0.020 g, 0.017 mmol) followed by evacuation of the reaction vessel and purging with nitrogen. The reaction mixture was heated in a Biotage Initiator® microwave synthesizer at 120° C. for 30 min. The reaction mixture was filtered and washed through with tetrahydrofuan. The mixture was concentrated in vacuo to afford the title compound (0.270 g, 78.3% yield) as a brown solid, MS (ES+) m/e 408 [M+H]⁺, used in the next step without further purification.

130(b) N-({2-[4-(1,1-dimethylethyl)phenyl]-6-hydroxy-5-quinoxalinyl}carbonyl)glycine. To the above ester (0.270 g, 0.66 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.107 g, 53.2% yield) as a yellow solid. ¹H NMR (400 MHz, DMSO-d6) δ ppm 15.21 (s, 1H, br), 12.93 (s, 1H, br), 11.40 (t, 1H, br, J=5.4 Hz), 9.51 (s, 1H), 8.23 (m, 3H), 7.62 (d, 2H, J=8.4 Hz), 7.56 (d, 1H, 9.2 Hz), 4.27 (d, 2H, J=5.6 Hz), 1.35 (s, 9H). MS (ES+) m/e 380 [M+H]⁺.

### Example 131

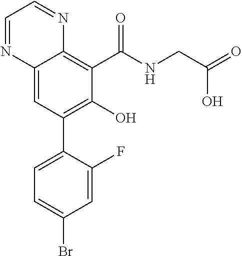

### N-{[7-(4-bromo-2-fluorophenyl)-6-hydroxy-5-quinoxalinyl]carbonyl}glycine

131(a) ethyl N-{[7-(4-bromo-2-fluorophenyl)-6-hydroxy-5-quinoxalinyl]carbonyl}glycinate. A solution of ethyl N-[(7-bromo-6-hydroxy-5-quinoxalinyl)carbonyl]glycinate (0.120 g, 0.339 mmol), (4-bromo-2-fluorophenyl)boronic acid (0.082 g, 0.373 mmol), potassium carbonate (0.140 g, 1.017 mmol), and tetrakis(triphenylphosphine)palladium(0) (0.018 g, 0.015 mmol) in 1,4-dioxane (2.0 ml) and water (0.667 ml) was heated to 100° C. for 60 min. in a Biotage Initiator® microwave synthesizer. Upon cooling, the reaction mixture was diluted with water. The resulting solid was filtered, washed with water and dried in vacuo to obtain ethyl N-{[7-(4-bromo-2-fluorophenyl)-6-hydroxy-5-quinoxalinyl]carbonyl}glycinate (0.05 g, 0.081 mmol, 24.03% yield) as a beige solid. ¹H NMR (400 MHz, DMSO-d₆) δ ppm 15.91 (s, 1H), 11.57 (t, J=5.4 Hz, 1H), 9.00 (d, J=1.5 Hz, 1H), 8.97 (s, 1H), 8.25 (s, 1H), 7.73 (dd, J=9.7, 1.4 Hz, 1H), 7.50-7.62 (m, 2H), 4.34 (d, J=5.4 Hz, 2H), 4.18 (q, J=7.1 Hz, 2H), 1.24 (t, J=7.1 Hz, 3H). MS (ES+) m/e 448/450 [M+H]⁺.

131(b) N-{[7-(4-bromo-2-fluorophenyl)-6-hydroxy-5-quinoxalinyl]carbonyl}glycine. To a suspension of ethyl N-{[7-(4-bromo-2-fluorophenyl)-6-hydroxy-5-quinoxalinyl]carbonyl}glycinate (0.05 g, 0.112 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, dried in vacuo and purified via Gilson (10-95% acetonitrile/water in TFA) to obtain N-{[7-(4-bromo-2-fluorophenyl)-6-hydroxy-5-quinoxalinyl]carbonyl}glycine (0.032 g, 0.076 mmol, 68.3% yield) as pale yellow solid. ¹H NMR (400 MHz, DMSO-d₆) δ ppm 16.03 (s, 1H), 12.96 (br. s., 1H), 11.53 (t, J=5.7 Hz, 1H), 9.00 (d, J=2.0 Hz, 1H), 8.96 (d, J=2.0 Hz, 1H), 8.24 (s, 1H), 7.73 (dd, J=9.5, 1.6 Hz, 1H), 7.58 (dd, J=7.3, 1.8 Hz, 1H), 7.57 (d, J=1.8 Hz, 1H), 7.55 (d, J=7.3 Hz, 1H), 4.26 (d, J=5.7 Hz, 2H). MS (ES+) m/e 420/422 [M+H]⁺.

### Example 132

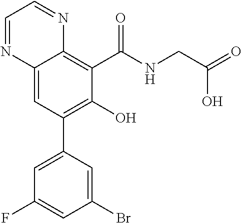

### N-{[7-(3-bromo-5-fluorophenyl)-6-hydroxy-5-quinoxalinyl]carbonyl}glycine

132(a) ethyl N-{[7-(3-bromo-5-fluorophenyl)-6-hydroxy-5-quinoxalinyl]carbonyl}glycinate. A solution of ethyl N-[(7-bromo-6-hydroxy-5-quinoxalinyl)carbonyl]glycinate (0.135 g, 0.381 mmol), (3-bromo-5-fluorophenyl)boronic acid (0.092 g, 0.419 mmol), potassium carbonate (0.158 g, 1.144 mmol), and tetrakis(triphenylphosphine)palladium(0) (0.020 g, 0.017 mmol) in 1,4-dioxane (2.0 ml) and water (0.6 ml) was heated to 100° C. for 60 min. in a Biotage Initiator® microwave synthesizer. Upon cooling, the reaction mixture was diluted with water an the resulting precipitate was filtered, washed with water and dried in vacuo. The solid was purified via flash chromatography (0-100% ethyl acetate in hexanes) to obtain a mixture of ethyl N-{[7-(3′-bromo-5,5′-difluoro-3-biphenylyl)-6-hydroxy-5-quinoxalinyl]carbonyl}glycinate MS (ES+) m/e 542/544 [M+H⁺ and ethyl N-{[7-(3-bromo-5-fluorophenyl)-6-hydroxy-5-quinoxalinyl]carbonyl}glycinate (0.167 g, 0.298 mmol, 78% yield) as a yellow solid. ¹H NMR (400 MHz, DMSO-d₆) δ ppm 16.16 (br. s., 1H), 11.62 (t, J=6.6 Hz, 1H), 8.99 (d, J=1.0 Hz, 1H), 8.97 (d, J=1.8 Hz, 1H), 8.31 (s, 1H), 7.78 (t, J=1.5 Hz, 1H), 7.67 (dd, J=8.8, 2.0 Hz, 1H), 7.63 (dd, J=9.2, 1.9 Hz, 1H), 4.35 (d, J=5.6 Hz, 2H), 4.18 (q, J=7.1 Hz, 2H), 1.24 (t, J=7.1 Hz, 3H). MS (ES+) m/e 448/450 [M+H]⁺.

132(b) N-{[7-(3-bromo-5-fluorophenyl)-6-hydroxy-5-quinoxalinyl]carbonyl}glycine. To a suspension of ethyl N-{[7-(3-bromo-5-fluorophenyl)-6-hydroxy-5-quinoxalinyl]carbonyl}glycinate (0.167 g, 0.373 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo. The resulting solid was purified via reverse-phase HPLC (10-95% acetonitrile/water in TFA) to obtain N-{[7-(3-bromo-5-fluorophenyl)-6-hydroxy-5-quinoxalinyl]carbonyl}glycine (0.005 g, 0.012 mmol, 3.19% yield) as a yellow solid. ¹H NMR (400 MHz, DMSO-d₆) δ ppm 11.55 (br. s., 1H), 8.97 (br. s., 1H), 8.95 (br. s., 1H), 8.28 (s, 1H), 7.78 (s, 1H), 7.67 (dt, J=8.4, 2.0 Hz, 1H), 7.63 (dd, J=9.9, 1.3 Hz, 1H), 4.24 (br. s., 2H). MS (ES+) m/e 420/422 [M+H]⁺.

### Example 133

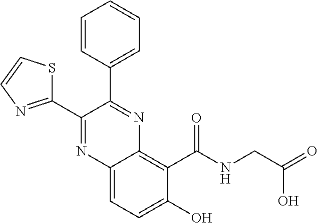

### N-{[6-hydroxy-3-phenyl-2-(1,3-thiazol-2-yl)-5-quinoxalinyl]carbonyl}glycine

133(a) methyl 6-(methyloxy)-3-phenyl-2-(1,3-thiazol-2-yl)-5-quinoxalinecarboxylate. To a solution of methyl 2-chloro-6-(methyloxy)-3-phenyl-5-quinoxalinecarboxylate (example 118(a), 0.104 g, 0.316 mmol) in 1,4-dioxane (1.5 ml) was added 2-(tributylstannanyl)-1,3-thiazole (0.130 g, 0.348 mmol) and tetrakis(triphenylphosphine)palladium(0) (0.016 g, 0.014 mmol) followed by heating to 150° C. for 20 min. in a Biotage Initiator® microwave synthesizer. Upon cooling, the reaction mixture was concentrated in vacuo, washed through with ethyl ether and filtered to obtain methyl 6-(methyloxy)-3-phenyl-2-(1,3-thiazol-2-yl)-5-quinoxalinecarboxylate (0.066 g, 0.175 mmol, 55.3% yield) as a pale yellow solid. ¹H NMR (400 MHz, DMSO-d₆) δ ppm 8.30 (d, J=9.3 Hz, 1H), 7.94 (d, J=3.3 Hz, 1H), 7.92 (d, J=9.4 Hz, 1H), 7.79 (d, J=3.3 Hz, 1H), 7.52 (t, J=1.6 Hz, 1H), 7.50 (t, J=2.0 Hz, 1H), 7.36-7.47 (m, 3H), 4.05 (s, 3H), 3.89 (s, 3H). MS (ES+) m/e 378 [M+H]⁺.

133(b) 6-hydroxy-3-phenyl-2-(1,3-thiazol-2-yl)-5-quinoxalinecarboxylic acid. A solution of methyl 6-(methyloxy)-3-phenyl-2-(1,3-thiazol-2-yl)-5-quinoxalinecarboxylate (0.066 g, 0.175 mmol) in dichloromethane (10 mL) was treated with boron tribromide (1M solution in dichloromethane) (0.874 mL, 0.874 mmol) at room temperature overnight. The reaction mixture was poured into water and extracted twice with ethyl acetate. The combined organic portions were dried over magnesium sulfate, filtered and concentrated to give 6-hydroxy-3-phenyl-2-(1,3-thiazol-2-yl)-5-quinoxalinecarboxylic acid (0.06 g, 0.163 mmol, 93% yield) as an orange solid. ¹H NMR (400 MHz, DMSO-d₆) δ ppm 15.25 (br. s., 1H), 12.64 (br. s., 1H), 8.34 (d, J=9.3 Hz, 1H), 7.97 (d, J=3.3 Hz, 1H), 7.82 (d, J=3.3 Hz, 1H), 7.71 (d, J=9.3 Hz, 1H), 7.61 (t, J=1.6 Hz, 1H), 7.60 (d, J=1.8 Hz, 1H), 7.45-7.54 (m, 3H). MS (ES+) m/e 350+H]⁺.

133(c) ethyl N-{[6-hydroxy-3-phenyl-2-(1,3-thiazol-2-O-5-quinoxalinyl]carbonyl}glycinate. A solution of 6-hydroxy-3-phenyl-2-(1,3-thiazol-2-yl)-5-quinoxalinecarboxylic acid (0.066 g, 0.189 mmol) and ethyl glycine hydrochloride (0.053 g, 0.378 mmol) in N,N-Dimethylformamide (DMF) (3.0 mL) was treated with triethylamine (0.079 mL, 0.567 mmol) and PyBOP (0.108 g, 0.208 mmol). The reaction mixture was stirred overnight at ambient temperature, quenched by water, filtered, dried in vacuo and purified via flash column chromatography (0-100% ethyl acetate in hexanes) to obtain to obtain ethyl N-{[6-hydroxy-3-phenyl-2-(1,3-thiazol-2-yl)-5-quinoxalinyl]carbonyl}glycinate (0.021 g, 0.044 mmol, 23.03% yield) as a yellow solid. ¹H NMR (400 MHz, DMSO-d₆) δ ppm 15.26 (s, 1H), 11.26 (t, J=5.6 Hz, 1H), 8.28 (d, J=9.3 Hz, 1H), 7.96 (d, J=3.0 Hz, 1H), 7.81 (d, J=3.0 Hz, 1H), 7.68 (t, J=1.5 Hz, 1H), 7.67 (t, J=1.5 Hz, 1H), 7.62 (d, J=9.3 Hz, 1H), 7.48 (dt, J=7.1, 1.8 Hz, 1H), 7.45 (dt, J=7.3, 2.3 Hz, 1H), 7.39-7.43 (m, 1H), 4.34 (d, J=5.6 Hz, 1H), 4.15 (q, J=7.1 Hz, 2H), 1.19 (t, J=7.1 Hz, 3H). MS (ES+) m/e 435 [M+H]⁺.

133(d) N-{[6-hydroxy-3-phenyl-2-(1,3-thiazol-2-yl)-5-quinoxalinyl]carbonyl}glycine. To a suspension of ethyl N-{[6-hydroxy-3-phenyl-2-(1,3-thiazol-2-yl)-5-quinoxalinyl]carbonyl}glycinate (0.021 g, 0.048 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[6-hydroxy-3-phenyl-2-(1,3-thiazol-2-yl)-5-quinoxalinyl]carbonyl}glycine (0.012 g, 0.030 mmol, 61.1% yield) as a yellow solid. ¹H NMR (400 MHz, DMSO-d₆) δ ppm 15.44 (br. s., 1H), 13.08 (br. s., 1H), 11.20 (t, J=5.2 Hz, 1H), 8.27 (d, J=9.3 Hz, 1H), 7.95 (d, J=3.0 Hz, 1H), 7.80 (d, J=3.0 Hz, 1H), 7.69 (t, J=1.4 Hz, 1H), 7.67 (t, J=1.8 Hz, 1H), 7.61 (d, J=9.3 Hz, 1H), 7.47 (td, J=7.1, 1.5 Hz, 1H), 7.39-7.45 (m, 2H), 4.26 (d, J=5.2 Hz, 2H). MS (ES+) m/e 407 [M+H]⁺.

### Example 134

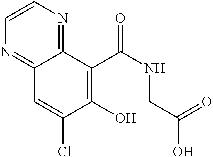

### N-[(7-chloro-6-hydroxy-5-quinoxalinyl)carbonyl]glycine

134(a) methyl 2-amino-5-chloro-6-fluoro-3-nitrobenzoate. In a 100 mL round-bottomed flask, fuming nitric acid (8.44 ml, 189 mmol) was cooled to 0° C. and concentrated sulfuric acid (15.77 ml, 296 mmol) was added slowly. After 5 minutes, methyl 3-chloro-2,6-difluorobenzoate (6.5 g, 31.5 mmol) was added to the reaction. The reaction was kept stirring overnight and quenched with water. The yellow precipitate was collected, washed with water, dried under reduced pressure and dissolved in methanol (20 ml) to give a yellow solution. Aqueous ammonia (30%, 1.362 ml, 24.0 mmol) was added to the yellow solution. The reaction was kept stirring overnight and quenched with 1N HCl (10 mL). The resulting solution was purified via preparative HPLC (YMC 75×30 mm column, 0.1% TFA in water and 0.1% TFA in acetonitrile) to afford methyl 2-amino-5-chloro-6-fluoro-3-nitrobenzoate (2.3 g, 9.25 mmol, 29.4% yield) as a yellow solid. 1H NMR (400 MHz, CHLOROFORM-d) δ ppm 8.47 (d, J=7.3 Hz, 1H), 8.38 (br. s., 2H), 3.99 (s, 3H). MS (ES+) m/e 249 [M+H]⁺.

134(b) methyl 7-chloro-6-(methyloxy)-5-quinoxalinecarboxylate. In a 100 mL round-bottomed flask was placed methyl 2-amino-5-chloro-6-fluoro-3-nitrobenzoate (2.3 g, 9.25 mmol) in methanol (50 mL) to give a yellow solution. Sodium methoxide in methanol (25%, 2.055 mL, 9.25 mmol) was added. The mixture was kept stirring overnight, quenched with water and extracted with ethyl acetate. The organic layer was collected, dried over MgSO₄ and concentrated under vacuum. The resulting yellow oil was dissolved in ethanol (50.0 mL), Raney nickel (0.054 g, 0.925 mmol) was added. The mixture was hydrogenated under a hydrogen ballon overnight. After filtration, glyoxal (40% in water) (1.175 g, 9.25 mmol) was added to the filtrate. The mixture was kept stirring for 3 hours, concentrated under vacuum and purified via flash chromatography (0-100% ethyl acetate in hexane) to afford methyl 7-chloro-6-(methyloxy)-5-quinoxalinecarboxylate (300 mg, 1.187 mmol, 12.83% yield) as a yellow solid. ¹H NMR (400 MHz, CHLOROFORM-d) δ ppm 8.86 (d, J=1.8 Hz, 1H), 8.83 (d, J=2.0 Hz, 1H), 8.25 (s, 1H), 4.11 (s, 3H), 4.10 (s, 3H). MS (ES+) m/e 253[M+H]⁺.

134(c) 7-chloro-6-hydroxy-5-quinoxalinecarboxylic acid. In a 50 mL round-bottomed flask was placed methyl 7-chloro-6-(methyloxy)-5-quinoxalinecarboxylate (0.3 g, 1.187 mmol) in dichloromethane (10 mL) to give a yellow solution. Boron tribromide (4.75 mL, 4.75 mmol) was added. The mixture was kept stirring overnight and quenched with ice water. The precipitate was collected and dried under vacuum to afford 7-chloro-6-hydroxy-5-quinoxalinecarboxylic acid (190 mg, 0.846 mmol, 71.2% yield) as a yellow solid. 1H NMR (400 MHz, DMSO-d6) δ ppm 15.38 (br. s., 1H), 9.01 (d, J=2.5 Hz, 1H), 8.90 (d, J=2.8 Hz, 1H), 8.53 (s, 1H). MS (ES+) m/e 225[M+H]⁺

134(d) N-[(7-chloro-6-hydroxy-5-quinoxalinyl)carbonyl]glycine. In a 100 mL round-bottomed flask was placed 7-chloro-6-hydroxy-5-quinoxalinecarboxylic acid (190 mg, 0.846 mmol), triethylamine (0.354 mL, 2.54 mmol) and PyBOP (484 mg, 0.931 mmol) in N,N-dimethylformamide (20 mL) to give a yellow solution. Ethyl glycine hydrochloride (236 mg, 1.692 mmol) was added. The reaction was kept stilling at ambient temperature for 3 hours and concentrated under vacuum. The resulting oil was dissolved in methanol (20.00 mL), sodium hydroxide (1.0 N in water) (3.38 mL, 3.38 mmol) was added. The mixture was kept stirring at ambient temperature for half hour and quenched with 1N HCl (20 ml). The precipitate was collected, washed with ether and dried to afford N-[(7-chloro-6-hydroxy-5-quinoxalinyl)carbonyl]glycine (150 mg, 0.533 mmol, 63.0% yield) as brown solid. 1H NMR (400 MHz, DMSO-d6) δ ppm 16.36 (s, 1H), 12.99 (br. s., 1H), 11.48 (br. s., 1H), 8.95 (d, J=8.6 Hz, 2H), 8.45 (s, 1H), 4.26 (d, J=5.6 Hz, 2H). MS (ES+) m/e 282[M+H]⁺

### Example 135

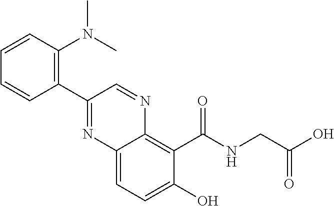

### N-({2-[2-(dimethylamino)phenyl]-6-hydroxy-5-quinoxalinyl}carbonyl)glycine

To a mixture of the compound from example 5(a) (0.137 g, 0.39 mmol), 2-(dimethylamino)phenylboronic acid (0.064 g, 0.39 mmol) and potassium carbonate (0.107 g, 0.78 mmol) in 1,4-dioxane (3.0 mL) and water (1.0 mL) was added tetrakis(triphenylphosphine)palladium (0.005 g, 3.88 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (5.0 mL) and 1N aqueous sodium hydroxide (10.0 mL) were added. After stirring for 10 min at ambient temperature, the mixture was quenched with 1N aqueous hydrochloric acid, evaporated the organic solvent and the resulting precipitate was filtered to afford the title compound (0.082 g, 57.6% yield) as a yellow solid. ¹H NMR (300 MHz, DMSO-d6) δ ppm 15.21 (s, 1H), 11.40 (t, 1H, J=5.4 Hz), 9.33 (s, 1H), 8.22 (d, 1H, J=9.3 Hz), 7.63 (dd, 1H, J₁=7.5 Hz, J₂=1.8 Hz), 7.54 (d, 1H, J=9.6 Hz), 7.45 (m, 1H), 7.27 (d, 1H, J=8.1 Hz), 7.18 (m, 1H), 4.24 (d, 2H, J=5.7 Hz), 2.560 (s, 6H). MS (ES+) m/e 367 [M+H]⁺.

### Example 136

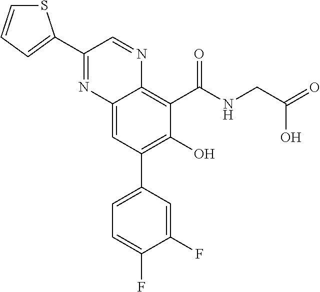

### N-{[7-(3,4-difluorophenyl)-6-hydroxy-2-(2-thienyl)-5-quinoxalinyl]carbonyl}glycine

136(a) methyl 7-(3,4-difluorophenyl)-6-(methyloxy)-2-oxo-1,2-dihydro-5-quinoxalinecarboxylate. A solution of methyl 7-bromo-6-(methyloxy)-2-oxo-1,2-dihydro-5-quinoxalinecarboxylate (example 48(a), 0.546 g, 1.744 mmol), (3,4-difluorophenyl)boronic acid (0.275 g, 1.744 mmol), potassium carbonate (0.723 g, 5.23 mmol), and tetrakis(triphenylphosphine)palladium(0) (0.060 g, 0.052 mmol) in 1,4-dioxane (2.0 ml) and water (0.667 ml) was heated to 105° C. overnight in an oil bath. Upon cooling, the reaction mixture was diluted with water and extracted with ethyl acetate. The combined organic portions were dried over magnesium sulfate, filtered and concentrated in vacuo to obtain a residue. The residue was triturated using ethyl ether and the solid was filtered and dried in vacuo to obtain methyl 7-(3,4-difluorophenyl)-6-(methyloxy)-2-oxo-1,2-dihydro-5-quinoxalinecarboxylate (0.130 g, 0.375 mmol, 21.53% yield) as a pale yellow solid. ¹H NMR (400 MHz, DMSO-d₆) δ ppm 12.60 (s, 1H), 8.24 (s, 1H), 7.70 (ddd, J=11.6, 7.8, 2.0 Hz, 1H), 7.60 (ddd, J=10.7, 8.6, 2.3 Hz, 1H), 7.39-7.48 (m, 1H), 7.31 (s, 1H), 3.91 (s, 3H), 3.40 (s, 3H). MS (ES+) m/e 347 [M+H]⁺.

136(b) methyl 2-chloro-7-(3,4-difluorophenyl)-6-(methyloxy)-5-quinoxalinecarboxylate. To a solution of methyl 7-(3,4-difluorophenyl)-6-(methyloxy)-2-oxo-1,2-dihydro-5-quinoxalinecarboxylate (130 mg, 0.375 mmol) was added phosphorus oxychloride (35.0 μl, 0.375 mmol). After heating to reflux for 2 h, the reaction mixture was carefully treated with ice water. The resulting precipitate was filtered, washed with water, and dried in vacuo to afford methyl 2-chloro-7-(3,4-difluorophenyl)-6-(methyloxy)-5-quinoxalinecarboxylate (128 mg, 0.351 mmol, 93% yield) as a dark brown solid. ¹H NMR (400 MHz, DMSO-d₆) δ ppm 9.03 (s, 1H), 8.18 (s, 1H), 7.81 (ddd, J=11.9, 7.8, 2.3 Hz, 1H), 7.62 (ddd, J=10.5, 8.4, 2.3 Hz, 1H), 7.52-7.58 (m, 1H), 3.97 (s, 3H), 3.59 (s, 3H). MS (ES+) m/e 365 [M+H]⁺.

136(c) methyl 7-(3,4-difluorophenyl)-6-(methyloxy)-2-(2-thienyl)-5-quinoxalinecarboxylate. To a solution of methyl 2-chloro-7-(3,4-difluorophenyl)-6-(methyloxy)-5-quinoxalinecarboxylate (128 mg, 0.351 mmol) in 1,4-dioxane (1.5 ml) was added tributyl(2-thienyl)stannane (131 mg, 0.351 mmol) and tetrakis(triphenylphosphine)palladium(0) (18.25 mg, 0.016 mmol) followed by heating to 120° C. for 30 min. in a Biotage Initiator® microwave synthesizer. Upon cooling, the reaction mixture was concentrated in vacuo and purified via flash column chromatography (0-10% methanol in dichloromethane) to obtain methyl 7-(3,4-difluorophenyl)-6-(methyloxy)-2-(2-thienyl)-5-quinoxalinecarboxylate (110 mg, 0.267 mmol, 76% yield) as a pale yellow solid. ¹H NMR (400 MHz, DMSO-d₆) δ ppm 9.59 (s, 1H), 8.22 (dd, J=3.8, 1.0 Hz, 1H), 8.13 (s, 1H), 7.88 (dd, J=4.9, 1.1 Hz, 1H), 7.81-7.87 (m, 1H), 7.60 (dd, J=4.9, 1.5 Hz, 1H), 7.57-7.63 (m, 1H), 7.31 (dd, J=4.9, 3.8 Hz, 1H), 3.98 (s, 3H), 3.57 (s, 3H). MS (ES+) m/e 413 [M+H]⁺.

136(d) 7-(3,4-difluorophenyl)-6-hydroxy-2-(2-thienyl)-5-quinoxalinecarboxylic acid. A solution of methyl 7-(3,4-difluorophenyl)-6-(methyloxy)-2-(2-thienyl)-5-quinoxalinecarboxylate (110 mg, 0.267 mmol) in dichloromethane (10 mL) was treated with boron tribromide (1M solution in dichloromethane) (1.334 mL, 1.334 mmol) at room temperature overnight. The reaction mixture was poured into water and extracted twice with ethyl acetate. The combined organic portions were dried over magnesium sulfate, filtered and concentrated to give 7-(3,4-difluorophenyl)-6-hydroxy-2-(2-thienyl)-5-quinoxalinecarboxylic acid (119 mg, 0.310 mmol, 116% yield) as a bright orange solid. ¹H NMR (400 MHz, DMSO-d₆) δ ppm 9.63 (s, 1H), 8.32 (s, 1H), 8.18 (dd, J=3.8, 1.0 Hz, 1H), 7.88 (dd, J=4.9, 1.1 Hz, 1H), 7.85-7.93 (m, 1H), 7.62-7.68 (m, 1H), 7.55-7.62 (m, 1H), 7.32 (dd, J=4.9, 3.7 Hz, 1H). MS (ES+) m/e 385 [M+H]⁺.

136(e) ethyl N-{[7-(3,4-difluorophenyl)-6-hydroxy-2-(2-thienyl)-5-quinoxalinyl]carbonyl}glycinate. A solution of 7-(3,4-difluorophenyl)-6-hydroxy-2-(2-thienyl)-5-quinoxalinecarboxylic acid (0.119 g, 0.310 mmol) and ethyl glycine hydrochloride (0.086 g, 0.619 mmol) in N,N-Dimethylformamide (DMF) (3.0 mL) was treated with triethylamine (0.129 mL, 0.929 mmol) and PyBOP (0.177 g, 0.341 mmol). The reaction mixture was stirred overnight at ambient temperature, quenched by water, filtered and dried in vacuo. The resulting orange solid was purified via flash column chromatography (0-10% methanol in dichloromethane) to obtain as a bright yellow solid. ¹H NMR (400 MHz, DMSO-d₆) δ ppm 16.00 (br. s., 1H), 11.55 (t, J=5.6 Hz, 1H), 9.54 (s, 1H), 8.21 (dd, J=3.7, 0.9 Hz, 1H), 8.19 (s, 1H), 7.85 (dd, J=5.1, 0.9 Hz, 1H), 7.86 (td, J=8.6, 2.0 Hz, 1H), 7.52-7.60 (m, 2H), 7.31 (dd, J=5.1, 3.8 Hz, 1H), 4.37 (d, J=5.6 Hz, 2H), 4.21 (q, J=7.1 Hz, 2H), 1.26 (t, J=7.1 Hz, 3H). MS (ES+) m/e 470 [M+H]⁺.

136(f) N-{[7-(3,4-difluorophenyl)-6-hydroxy-2-(2-thienyl)-5-quinoxalinyl]carbonyl}glycine. To a suspension of ethyl N-{[7-(3,4-difluorophenyl)-6-hydroxy-2-(2-thienyl)-5-quinoxalinyl]carbonyl}glycinate (80 mg, 0.170 mmol) in ethanol (1.0 mL) was added 1N aqueous sodium hydroxide (0.170 ml, 0.170 mmol). After stirring 30 min. at ambient temperature, the reaction was quenched with 1N aqueous hydrochloric acid and the resulting precipitate was filtered, washed with water, and dried in vacuo to obtain N-{[7-(3,4-difluorophenyl)-6-hydroxy-2-(2-thienyl)-5-quinoxalinyl]carbonyl}glycine (15 mg, 0.034 mmol, 19.94% yield) as a yellow solid. ¹H NMR (400 MHz, DMSO-d₆) δ ppm 16.13 (s, 1H), 13.00 (br. s., 1H), 11.50 (t, J=5.7 Hz, 1H), 9.55 (s, 1H), 8.21 (dd, J=3.7, 0.9 Hz, 1H), 8.19 (s, 1H), 7.85 (dd, J=5.1, 0.9 Hz, 1H), 7.82-7.90 (m, 1H), 7.45-7.67 (m, 2H), 7.30 (dd, J=5.1, 3.8 Hz, 1H), 4.29 (d, J=5.7 Hz, 2H). MS (ES+) m/e 442 [M+H]⁺.

### Example 137

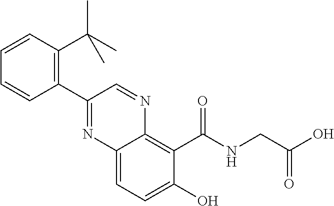

### N-({2-[2-(1,1-dimethylethyl)phenyl]-6-hydroxy-5-quinoxalinyl}carbonyl)glycine

To a mixture of the compound from example 5(a) (0.300 g, 0.85 mmol), 2-tert-butylphenylboronic acid (0.197 g, 1.11 mmol) and potassium carbonate (0.234 g, 1.69 mmol) in 1,4-dioxane (3.0 mL) and water (1.0 mL) was added tetrakis(triphenylphosphine)palladium (0.010 g, 8.47 mol) followed by evacuation of the reaction vessel and purging with nitrogen. The reaction mixture was heated in a Biotage Initiator® microwave synthesizer at 120° C. for 1 h to get the intermediate ester and upon cooling, tetrahydrofuran (8.0 mL) and 1N aqueous sodium hydroxide (10.0 mL) were added. After stirring for 10 min at ambient temperature, the mixture was quenched with 1N aqueous hydrochloric acid, then extracted with a mixture of EtOAc and tetrahydrofuran (3:1, v/v), dried, concentrated in vacuo and purified via rp-HPLC (acetonitrile/water+0.1% trifluoroacetic acid) to afford the title compound (0.032 g, 10.1% yield) as a pale yellow solid. ¹H NMR (300 MHz, DMSO-d6) δ ppm 15.27 (s, 1H), 12.89 (s, 1H), 11.35 (t, 1H, J=5.2 Hz), 8.95 (s, 1H), 7.66 (d, 1H, J=8.0 Hz), 7.60 (d, 1H, J=9.2 Hz), 7.48 (m, 1H), 7.34 (t, 1H, J=7.2 Hz), 7.25 (m, 1H), 4.25 (d, 2H, J=5.6 Hz), 1.15 (s, 9H). MS (ES+) m/e 380 [M+H]⁺.

### Example 138

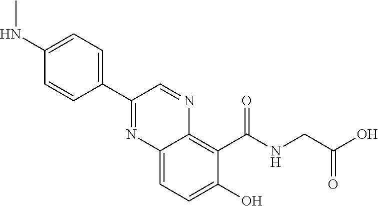

### N-({6-hydroxy-2-[4-(methylamino)phenyl]-5-quinoxalinyl}carbonyl)glycine

To a mixture of the compound from example 5(a) (0.300 g, 0.85 mmol), 4-(methylamino)phenylboronic acid pinacol ester (0.258 g, 1.11 mmol) and potassium carbonate (0.234 g, 1.69 mmol) in 1,4-dioxane (3.0 mL) and water (1.0 mL) was added tetrakis(triphenylphosphine)palladium (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 10 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.175 g, 58.7% yield) as an orange solid. ¹H NMR (400 MHz, DMSO-d6) δ ppm 11.27 (s, 1H), 9.36 (s, 1H), 8.06 (m, 3H), 7.45 (d, 1H, J=9.2 Hz), 6.69 (d, 2H, J=8.8 Hz), 6.28 (d, 1H, J=4.4 Hz), 3.91 (d, 2H, J=4.0 Hz), 2.76 (d, 3H, J=4.8 Hz). MS (ES+) m/e 353 [M+H]⁺.

### Example 139

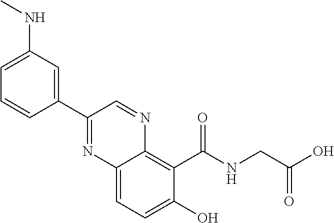

### N-({6-hydroxy-2-[3-(methylamino)phenyl]-5-quinoxalinyl}carbonyl)glycine

To a mixture of the compound from example 5(a) (0.300 g, 0.85 mmol), 3-(methylamino)phenylboronic acid pinacol ester (0.258 g, 1.11 mmol) and potassium carbonate (0.234 g, 1.69 mmol) in 1,4-dioxane (3.0 mL) and water (1.0 mL) was added tetrakis(triphenylphosphine)palladium (0) (0.010 g, 8.47 μmol) followed by evacuation of the reaction vessel and purging with nitrogen. The reaction mixture was heated in a Biotage Initiator® microwave synthesizer at 120° C. for 30 min to get the intermediate ester and upon cooling, tetrahydrofuran (8.0 mL) and 1N aqueous sodium hydroxide (10.0 mL) were added. After stirring for 15 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.233 g, 78.3% yield) as a yellow solid. ¹H NMR (400 MHz, DMSO-d6) δ ppm 15.21 (s, 1H), 11.41 (t, 1H, J=5.6 Hz), 9.45 (s, 1H), 8.21 (d, 1H, J=9.2 Hz), 7.56 (d, 1H, J=9.6 Hz), 7.51 (d, 2H, J=8.0 Hz), 7.34 (t, 1H, J=7.2 Hz), 6.79 (d, 1H, J=8.0 Hz), 4.27 (d, 2H, J=5.6 Hz), 2.81 (s, 3H). MS (ES+) m/e 353 [M+H]⁺.

### Example 140

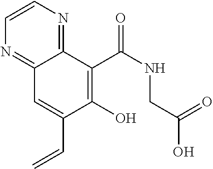

### N-[(7-ethenyl-6-hydroxy-5-quinoxalinyl)carbonyl]glycine

In a 10 mL microwavable vial was placed tributyl(ethenyl)stannane (98 mg, 0.311 mmol), ethyl N-[(7-bromo-6-hydroxy-5-quinoxalinyl)carbonyl]glycinate (example 22(a), 100 mg, 0.282 mmol), and tetrakis(triphenylphosphine)palladium (0) (32.6 mg, 0.028 mmol) in 1,4-dioxane (3 mL) to give a yellow suspension. The reaction was heated to 100° C. for an hour, and quenched with water. The mixture was extracted with ethyl acetate. The extract was dried over MgSO₄, filtered, concentrated under vacuum and purified via flash chromatography (0-100% ethyl acetate in hexane) to afford the intermediate ester as a yellow solid. The intermediate was dissolved in methanol (5 mL) and tetrahydrofuran (THF) (5.00 mL). Sodium hydroxide (6.0 N in water) (0.094 mL, 0.565 mmol) was added. The mixture was kept stirring for half hour and quenched with 1N HCl (10 mL). The precipitate was collected, washed with water and dried to afford N-[(7-ethenyl-6-hydroxy-5-quinoxalinyl)carbonyl]glycine (8 mg, 0.029 mmol, 10.37% yield) as yellow solid. 1H NMR (400 MHz, DMSO-d6) δ ppm 16.15 (s, 1H), 12.93 (br. s., 1H), 11.52 (t, J=5.4 Hz, 1H), 8.90 (s, 2H), 8.39 (s, 1H), 7.15 (dd, J=17.7, 11.1 Hz, 1H), 6.26 (d, J=17.9 Hz, 1H), 5.61 (d, J=11.4 Hz, 1H), 4.25 (d, J=5.6 Hz, 2H). MS (ES+) m/e 274[M+H]⁺.

### Example 141

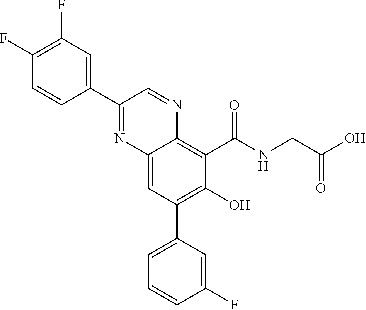

### N-{[2-(3,4-difluorophenyl)-7-(3-fluorophenyl)-6-hydroxy-5-quinoxalinyl]carbonyl}glycine

141(a) methyl 7-(3-fluorophenyl)-6-(methyloxy)-2-oxo-1,2-dihydro-5-quinoxalinecarboxylate. To a solution of methyl 7-bromo-6-(methyloxy)-2-oxo-1,2-dihydro-5-quinoxalinecarboxylate (example 48(a), 0.290 g, 0.926 mmol) and (3-fluorophenyl)boronic acid (0.130 g, 0.926 mmol) in 1,4-Dioxane (2.0 mL) and water (0.667 mL) was added palladium tetrakis (0.048 g, 0.042 mmol) and potassium carbonate (0.384 g, 2.78 mmol), followed by heating to 120° C. for 30 min. in a Biotage Initiator® microwave synthesizer, then to 105° C. in an oil bath. After cooling down to room temperature, the reaction mixture was purified via flash column chromatography (0-10% methanol in dichloromethane) to obtain methyl 7-(3-fluorophenyl)-6-(methyloxy)-2-oxo-1,2-dihydro-5-quinoxalinecarboxylate (0.160 g, 0.487 mmol, 52.6% yield) as a pale peach solid. ¹H NMR (400 MHz, DMSO-d₆) δ ppm 12.60 (br. s., 1H), 8.24 (s, 1H), 7.58 (td, J=8.1, 6.3 Hz, 1H), 7.40-7.47 (m, 2H), 7.29-7.36 (m, 2H), 3.92 (s, 3H), 3.39 (s, 3H). MS (ES+) m/e 329 [M+H]⁺.

141(b) methyl 7-(3-fluorophenyl)-6-(methyloxy)-2-{[(trifluoromethyl)sulfonyl]oxy}-5-quinoxalinecarboxylate. To a solution of methyl 7-(3-fluorophenyl)-6-(methyloxy)-2-oxo-1,2-dihydro-5-quinoxalinecarboxylate (160 mg, 0.487 mmol) and triethylamine (0.190 mL, 1.365 mmol) in dichloromethane (25 mL) at 0° C. was added triflic anhydride (0.115 mL, 0.682 mmol) dropwise. After stirring at 0° C. for 2 h, the reaction mixture was carefully treated with ice water, the layers were separated and the aqueous layer was further extracted twice with ethyl acetate. The combined organic portions were dried over magnesium sulfate, filtered and concentrated to obtain methyl 7-(3-fluorophenyl)-6-(methyloxy)-2-{[(trifluoromethyl)sulfonyl]oxy}-5-quinoxalinecarboxylate (0.220 g, 0.478 mmol, 98% yield) as a dark brown oil. ¹H NMR (400 MHz, DMSO-d₆) δ ppm 9.25 (s, 1H), 8.21 (s, 1H), 7.48-7.65 (m, 3H), 7.29-7.41 (m, 1H), 3.99 (s, 3H), 3.60 (s, 3H). MS (ES+) m/e 461 [M+H]⁺.

141(c) methyl 2-(3,4-difluorophenyl)-7-(3-fluorophenyl)-6-(methyloxy)-5-quinoxalinecarboxylate. A solution of methyl 7-(3-fluorophenyl)-6-(methyloxy)-2-{[(trifluoromethyl)sulfonyl]oxy}-5-quinoxalinecarboxylate (0.137 g, 0.298 mmol), (3,4-difluorophenyl)boronic acid (0.047 g, 0.298 mmol), potassium carbonate (0.123 g, 0.893 mmol), and tetrakis(triphenylphosphine)palladium(0) (10.32 mg, 8.93 mmol) in 1,4-dioxane (2.0 ml) and water (0.667 ml) was heated to 105° C. for 2 h in a Biotage Initiator® microwave synthesizer. Upon cooling, the reaction mixture was diluted with water and then extracted twice with ethyl acetate. The organic portions were dried over magnesium sulfate, filtered and concentrated to obtain a residue which was purified via flash column chromatography (0-100% ethyl acetate in hexanes) to obtain methyl 2-(3,4-difluorophenyl)-7-(3-fluorophenyl)-6-(methyloxy)-5-quinoxalinecarboxylate (109 mg, 0.257 mmol, 86% yield) as a beige solid. ¹H NMR (400 MHz, DMSO-d₆) δ ppm 9.63 (s, 1H), 8.40 (ddd, J=12.1, 8.1, 2.3 Hz, 1H), 8.24 (s, 1H), 8.19-8.28 (m, 1H), 7.71 (ddd, J=10.5, 8.5 Hz, 1H), 7.54-7.62 (m, 3H), 7.30-7.40 (m, 1H), 3.99 (s, 3H), 3.59 (s, 3H). MS (ES+) m/e 425 [M+H]⁺.

141(d) 2-(3,4-difluorophenyl)-7-(3-fluorophenyl)-6-hydroxy-5-quinoxalinecarboxylic acid. A solution of methyl 2-(3,4-difluorophenyl)-7-(3-fluorophenyl)-6-(methyloxy)-5-quinoxalinecarboxylate (109 mg, 0.257 mmol) in dichloromethane (10 mL) was treated with boron tribromide (1M solution in dichloromethane) (1.027 mL, 1.027 mmol) at room temperature overnight. The reaction mixture was quenched with water and extracted twice with dichloromethane. The combined organic portions were dried over magnesium sulfate, filtered and concentrated to give 2-(3,4-difluorophenyl)-7-(3-fluorophenyl)-6-hydroxy-5-quinoxalinecarboxylic acid (67 mg, 0.169 mmol, 65.8% yield) as a yellow solid. ¹H NMR (400 MHz, DMSO-d₆) δ ppm 14.21 (br. s., 1H), 9.65 (s, 1H), 8.41 (s, 1H), 8.37 (ddd, J=12.1, 7.8, 2.3 Hz, 1H), 8.12-8.25 (m, 1H), 7.73 (ddd, J=10.4, 8.6, 1.8 Hz, 1H), 7.57-7.65 (m, 3H), 7.29-7.42 (m, 1H). MS (ES+) m/e 397 [M+H]⁺.

141(e) ethyl N-{[2-(3,4-difluorophenyl)-7-(3-fluorophenyl)-6-hydroxy-5-quinoxalinyl]carbonyl}glycinate. A solution of 2-(3,4-difluorophenyl)-7-(3-fluorophenyl)-6-hydroxy-5-quinoxalinecarboxylic acid (67 mg, 0.169 mmol) and ethyl glycine hydrochloride (47.2 mg, 0.338 mmol) in N,N-Dimethylformamide (DMF) (3.0 mL) was treated with triethylamine (0.071 mL, 0.507 mmol) and PyBOP (97 mg, 0.186 mmol). The reaction mixture was stirred for 3 h at ambient temperature, quenched by water, filtered and dried in vacuo to obtain ethyl N-{[2-(3,4-difluorophenyl)-7-(3-fluorophenyl)-6-hydroxy-5-quinoxalinyl]carbonyl}glycinate (45 mg, 0.093 mmol, 55.3% yield) as a pale yellow solid. ¹H NMR (400 MHz, DMSO-d₆) δ ppm 16.11 (s, 1H), 11.58 (t, J=5.4 Hz, 1H), 9.59 (s, 1H), 8.40 (ddd, J=12.1, 8.1, 1.8 Hz, 1H), 8.30 (s, 1H), 8.18-8.26 (m, 1H), 7.70 (ddd, J=10.4, 8.6, 1.8 Hz, 1H), 7.52-7.64 (m, 3H), 7.23-7.38 (m, 1H), 4.39 (d, J=5.4 Hz, 2H), 4.20 (q, J=7.1 Hz, 2H), 1.26 (t, J=7.1 Hz, 3H). MS (ES+) m/e 482 [M+H]⁺.

141(f) N-{[2-(3,4-difluorophenyl)-7-(3-fluorophenyl)-6-hydroxy-5-quinoxalinyl]carbonyl}glycine. To a suspension of ethyl N-{[2-(3,4-difluorophenyl)-7-(3-fluorophenyl)-6-hydroxy-5-quinoxalinyl]carbonyl}glycinate (45 mg, 0.093 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[2-(3,4-difluorophenyl)-7-(3-fluorophenyl)-6-hydroxy-5-quinoxalinyl]carbonyl}glycine (34 mg, 0.075 mmol, 80% yield) as a pale yellow solid. ¹H NMR (400 MHz, DMSO-d₆) δ ppm 16.22 (s, 1H), 11.52 (t, J=5.4 Hz, 1H), 9.58 (s, 1H), 8.39 (ddd, J=12.1, 8.1, 2.0 Hz, 1H), 8.28 (s, 1H), 8.18-8.25 (m, 1H), 7.69 (ddd, J=10.4, 8.6, 1.8 Hz, 1H), 7.52-7.63 (m, 3H), 7.26-7.38 (m, 1H), 4.30 (d, J=5.4 Hz, 2H). MS (ES+) m/e 454 [M+H]⁺.

### Example 142

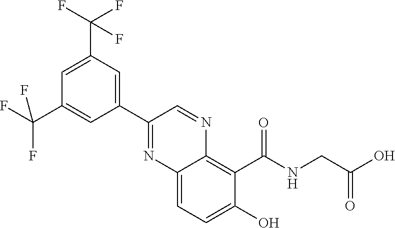

### N-({2-[3,5-bis(trifluoromethyl)phenyl]-6-hydroxy-5-quinoxalinyl}carbonyl)glycine

142(a) Ethyl N-({2-[3,5-bis(trifluoromethyl)phenyl]-6-hydroxy-5-quinoxalinyl}carbonyl)glycinate. To a mixture of the compound from example 5(a) (0.400 g, 1.13 mmol), 3,5-bis(trifluoromethyl)phenylboronic acid (0.350 g, 1.36 mmol) and potassium carbonate (0.312 g, 2.26 mmol) in 1,4-dioxane (2.5 mL) and water (1.0 mL) was added tetrakis(triphenylphosphine)palladium (0.065 g, 0.056 mmol) followed by evacuation of the reaction vessel and purging with nitrogen. The reaction mixture was heated in a Biotage Initiator® microwave synthesizer at 120° C. for 90 min. The reaction mixture was filtered and washed through with tetrahydrofuan. The mixture was concentrated in vacuo to afford the title compound (0.380 g, 73.2% yield) as a yellow solid, MS (ES+) m/e 488 [M+H]⁺, used in the next step without further purification.

142(b) N-({2-[3,5-bis(trifluoromethyl)phenyl]-6-hydroxy-5-quinoxalinyl}carbonyl)glycine. To the above crude ester (0.380 g, 0.78 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.098 g, 27.4% yield) as an off-white solid. ¹H NMR (400 MHz, DMSO-d6) □ ppm 15.39 (s, 1H, br), 13.00 (s, 1H, br), 11.36 (t, 1H, br, J=5.1 Hz), 9.80 (s, 1H), 8.95 (s, 2H), 8.31 (t, 2H, J=6.9 Hz), 7.61 (d, 1H, J=7.2 Hz), 4.29 (d, 2H, J=5.2 Hz). MS (ES+) m/e 460 [M+H]⁺.

### Example 143

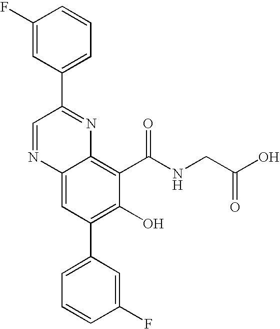

### N-{[3,7-bis(3-fluorophenyl)-6-hydroxy-5-quinoxalinyl]carbonyl}glycine

143(a) Methyl 7-bromo-6-(methyloxy)-3-oxo-3,4-dihydro-5-quinoxalinecarboxylate. To a solution of methyl 2,3-diamino-5-bromo-6-methoxybenzoate (example 18(a), 16.0 g, 51.1 mmol) in methanol (200 mL) was added ethyl glyoxylate (50% w/w solution in toluene, 13.8 mL, 61.3 mmol) and the mixture refluxed at 80° C. for 2 h. Upon cooling, the mixture was concentrated and purified via Silica Gel Column Chromatography (SGC, MeOH in dichloromethane from 0% to 10%) to afford the title compound (7.5 g, 41.2% yield) as an off-white solid. ¹H NMR (400 MHz, DMSO-d6) δ ppm 12.24 (s, 1H, br), 8.21 (s, 1H), 8.18 (s, 1H), 3.92 (s, 3H), 3.86 (s, 3H). MS (ES+) m/e 313/315 [M+H]⁺.

143(b) Methyl 7-(3-fluorophenyl)-6-(methyloxy)-3-oxo-3,4-dihydro-5-quinoxalinecarboxylate. To a mixture of methyl 7-bromo-6-(methyloxy)-3-oxo-3,4-dihydro-5-quinoxalinecarboxylate (3.10 g, 9.9 mmol), 3-fluorophenylboronic acid (1.66 g, 11.9 mmol) and potassium carbonate (2.73 g, 198. mmol) in 1,4-dioxane (35 mL) was added tetrakis(triphenylphosphine)palladium(0) (572 mg, 0.5 mmol) under nitrogen. The reaction mixture was refluxed at 110° C. for 3 h. Upon cooling, following removal of the solvent, the residue was dissolved in tetrahydrofuran, filtered through silica, and concentrated to afford the title compound (2.31 g, 71.1% yield) as a yellow solid. MS (ES+) m/e 329 [M+H]⁺.

143(c) Methyl 3-chloro-7-(3-fluorophenyl)-6-(methyloxy)-5-quinoxalinecarboxylate. A mixture of methyl 7-(3-fluorophenyl)-6-(methyloxy)-3-oxo-3,4-dihydro-5-quinoxalinecarboxylate (2.31 g, 7.0 mmol) and phosphoryl trichloride (6.58 mL, 70.4 mmol) was refluxed at 115° C. for 2 h. Upon cooling, the residual phosphoryl trichloride was evaporated and to the residue was added iced-water. The resulting precipitate was filtered, washed thoroughly with iced-ether to afford the crude title compound (2.10 g, 86.1% yield) as a brown solid. MS (ES+) m/e 347 [M+H]⁺.

143(d) 3-bromo-7-(3-fluorophenyl)-6-hydroxy-5-quinoxalinecarboxylic acid. To methyl 3-chloro-7-(3-fluorophenyl)-6-(methyloxy)-5-quinoxalinecarboxylate (2.11 g, 6.1 mmol) in anhydrous dichloromethane (25 mL) was added boron tribromide (7.63 g, 30.5 mmol) dropwise at 0° C. carefully. The mixture was warmed to ambient temperature and stirred overnight and then evaporated. To the residue was added iced-water, then the solid filtered and washed thoroughly with iced-ether to afford the title compound (1.75 g, 79.5% yield) as a brown solid. Further purification by silica gel chromatography (tetrahydrofuran in hexane from 50% to 100%) affords the title compound. MS (ES+) m/e 363/365 [M+H]⁺.

143(e) Ethyl N-{[3-bromo-7-(3-fluorophenyl)-6-hydroxy-5-quinoxalinyl]carbonyl}glycinate. To a mixture of 3-bromo-7-(3-fluorophenyl)-6-hydroxy-5-quinoxalinecarboxylic acid (0.603 g, 1.66 mmol) and dichloromethane (10 mL) was added 1-[3-(dimethylamino)propyl]-3-ethylcarbodiimide hydrochloride (0.478 g, 2.50 mmol) and triethylamine (0.336 g, 3.32 mmol). After stirring for 10 min, ethyl glycinate hydrochloride (0.305 g, 2.50 mmol) was added and the mixture stirred overnight. The volatiles were removed under reduced pressure and the residue triturated with 1M acetic acid and then 1M sodium bicarbonate (NaHCO₃). The solid was filtered and washed with methanol to afford the title compound (0.450 g, 60.5% yield) as a solid. MS (ES+) m/e 448/450 [M+H]⁺.

143(f) N-{[3,7-bis(3-fluorophenyl)-6-hydroxy-5-quinoxalinyl]carbonyl}glycine. A mixture of ethyl N-{[3-bromo-7-(3-fluorophenyl)-6-hydroxy-5-quinoxalinyl]carbonyl}glycinate (0.400 g, 0.89 mmol), 3-fluorophenylboronic acid (0.150 g, 1.07 mmol) and potassium carbonate (0.271 g, 1.96 mmol) in 1,4-dioxane (2.5 mL) and water (1 mL) was added tetrakis(triphenylphosphine)palladium(0) (0.052 g, 0.05 mmol) under nitrogen. The mixture was heated in a Biotage Initiator microwave synthesizer at 120° C. for 1 h. Upon cooling, the reaction mixture was concentrated in vacuo and purified via reversed-phase HPLC (C18 column, MeCN in water [0.01% TFA] from 20% to 85% in 25 min) to afford the title compound (51 mg, 13.1% yield) as a yellow solid. ¹H NMR (400 MHz, DMSO-d6) δ ppm 11.31 (d, 1H, J=3.6 Hz), 9.41 (s, 1H), 8.16 (m, 3H), 7.55 (m, 4H), 7.39 (t, 1H, J=7.6 Hz), 7.27 (t, 1H, J=8.8 Hz), 4.25 (d, 2H, J=4.4 Hz). MS (ES+) m/e 436 [M+H]⁺.

### Example 144

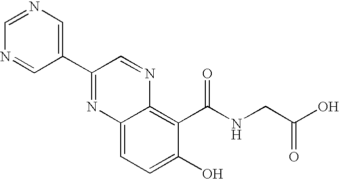

### N-{[6-hydroxy-2-(5-pyrimidinyl)-5-quinoxalinyl]carbonyl}glycine

144(a) Ethyl N-{[6-hydr oxy-2-(5-pyrimidinyl)-5-quinoxalinyl]carbonyl}glycinate. To a mixture of the compound from example 5(a) (0.400 g, 1.13 mmol), pyrimidin-5-ylboronic acid (0.168 g, 1.36 mmol) and potassium carbonate (0.312 g, 2.26 mmol) in 1,4-dioxane (2.5 mL) and water (1.0 mL) was added tetrakis(triphenylphosphine)palladium (0.065 g, 0.056 mmol) followed by evacuation of the reaction vessel and purging with nitrogen. The reaction mixture was heated in a Biotage Initiator® microwave synthesizer at 120° C. for 90 min. The reaction mixture was filtered and washed through with tetrahydrofuran. The organic phase was concentrated in vacuo to afford the title compound (0.413 g, 112.5% yield) as a brown solid, MS (ES+) m/e 354 [M+H]⁺, used in the next step without further purification.

144(b) N-{[6-hydroxy-2-(5-pyrimidinyl)-5-quinoxalinyl]carbonyl}glycine. To the above crude ester (0.413 g, 1.17 mmol) was added aqueous sodium hydroxide (1N, 6.0 mL) and tetrahydrofuran (8.0 mL). The mixture was stirred at ambient temperature for 10 min and tetrahydrofuran was removed in vacuo. 1N hydrochloric acid was added in to adjust pH to 3. The precipitate was collected by filtration to get crude product, which was purified by rp-HPLC (acetonitrile/water+0.1% trifluoroacetic acid) to afford the title compound (0.055 g, 14.5% yield) as an off-white solid. ¹H NMR (400 MHz, DMSO-d6) δ ppm 15.38 (s, 1H, br), 11.30 (s, 1H), 9.63 (s, 3H), 9.35 (s, 1H), 8.24 (d, 1H, J=9.2 Hz), 7.60 (d, 1H, J=9.2 Hz), 4.27 (d, 2H, J=4.8 Hz). MS (ES+) m/e 326 [M+H]⁺.

### Example 145

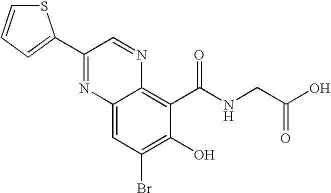

### N-{[7-bromo-6-hydroxy-2-(2-thienyl)-5-quinoxalinyl]carbonyl}glycine

145(a) methyl 7-bromo-6-(methyloxy)-2-{[(trifluoromethyl)sulfonyl]oxy}-5-quinoxalinecarboxylate. To a solution of methyl 7-bromo-6-(methyloxy)-2-oxo-1,2-dihydro-5-quinoxalinecarboxylate (500 mg, 1.597 mmol) and triethylamine (0.623 mL, 4.47 mmol) in dichloromethane (25 mL) at 0° C. was added triflic anhydride (0.378 mL, 2.236 mmol) dropwise. After stirring at 0° C. for 2 h, the reaction mixture was carefully treated with ice water, the layers were separated and the aqueous layer was further extracted twice with dichloromethane. The combined organic portions were dried over magnesium sulfate, filtered and concentrated to obtain methyl 7-bromo-6-(methyloxy)-2-{[(trifluoromethyl)sulfonyl]oxy}-5-quinoxalinecarboxylate (812 mg, 1.605 mmol, 101% yield) as a sticky brown oil. (The material was used right away, without further purification). ¹H NMR (400 MHz, DMSO-d₆) δ ppm 9.25 (s, 1H), 8.63 (s, 1H), 4.00 (s, 3H), 3.99 (s, 3H). MS (ES+) m/e 445/447 [M+H]⁺.

145(b) methyl 7-bromo-6-(methyloxy)-2-(2-thienyl)-5-quinoxalinecarboxylate. To a solution of methyl 7-bromo-6-(methyloxy)-2-{[(trifluoromethyl)sulfonyl]oxy}-5-quinoxalinecarboxylate (300 mg, 0.674 mmol) in 1,4-dioxane (1.5 ml) was added tributyl(2-thienyl)stannane (251 mg, 0.674 mmol) and tetrakis(triphenylphosphine)palladium(0) (779 mg, 0.674 mmol) followed by heating to 120° C. for 30 min. in a Biotage Initiator® microwave synthesizer. Upon cooling, the reaction mixture was concentrated in vacuo and purified via flash column chromatography (0-100% ethyl acetate in hexanes) to obtain methyl 7-bromo-6-(methyloxy)-2-(2-thienyl)-5-quinoxalinecarboxylate (140 mg, 0.369 mmol, 54.8% yield) as a yellow solid. ¹H NMR (400 MHz, DMSO-d₆) δ ppm 9.59 (s, 1H), 8.49 (s, 1H), 8.22 (dd, J=3.8, 1.0 Hz, 1H), 7.90 (dd, J=5.1, 1.0 Hz, 1H), 7.31 (dd, J=5.1, 3.8 Hz, 1H), 3.98 (s, 3H), 3.96 (s, 3H). MS (ES+) m/e 379/381 [M+H]⁺.

145(c) 7-bromo-6-hydroxy-2-(2-thienyl)-5-quinoxalinecarboxylic acid. A solution of methyl 7-bromo-6-(methyloxy)-2-(2-thienyl)-5-quinoxalinecarboxylate (139 mg, 0.367 mmol) in dichloromethane (10 mL) was treated with boron tribromide (1M solution in dichloromethane) (1.100 mL, 1.100 mmol) at room temperature overnight. The reaction mixture was quenched with water and extracted twice with dichloromethane. The combined organic portions were dried over magnesium sulfate, filtered and concentrated to give 7-bromo-6-hydroxy-2-(2-thienyl)-5-quinoxalinecarboxylic acid (35 mg, 0.100 mmol, 27.2% yield) as a bright orange solid. ¹H NMR (400 MHz, DMSO-d₆) δ ppm 9.59 (s, 1H), 8.66 (s, 1H), 8.15 (dd, J=3.8, 1.0 Hz, 1H), 7.88 (dd, J=5.1, 1.0 Hz, 1H), 7.31 (dd, J=5.1, 3.8 Hz, 1H). MS (ES+) m/e 351/353 [M+H]⁺.

145(d) N-{[7-bromo-6-hydroxy-2-(2-thienyl)-5-quinoxalinyl]carbonyl}glycine. A solution of 7-bromo-6-hydroxy-2-(2-thienyl)-5-quinoxalinecarboxylic acid (35 mg, 0.100 mmol) and ethyl glycine hydrochloride (27.8 mg, 0.199 mmol) in N,N-Dimethylformamide (DMF) (3.0 mL) was treated with triethylamine (0.042 mL, 0.299 mmol) and PyBOP (57.1 mg, 0.110 mmol). The reaction mixture was stirred overnight at ambient temperature, quenched by water, filtered, dried in vacuo and purified via flash column chromatography (0-100% ethyl acetate in hexanes) to give to obtain a yellow solid. This solid was dissolved in ethanol (3.00 mL) and treated with sodium hydroxide (1.0 mL, 1.000 mmol). The solution was stirred at room temperature for 30 min., then quenched with water and filtered to obtain N-{[7-bromo-6-hydroxy-2-(2-thienyl)-5-quinoxalinyl]carbonyl}glycine (10.0 mg, 0.024 mmol, 24.58% yield) as an orange solid. ¹H NMR (400 MHz, DMSO-d₆) δ ppm 16.24 (s, 1H), 11.42 (t, J=5.6 Hz, 1H), 9.54 (s, 1H), 8.56 (s, 1H), 8.21 (dd, J=3.8, 1.0 Hz, 1H), 7.85 (dd, J=5.1, 1.0 Hz, 1H), 7.29 (dd, J=5.1, 3.8 Hz, 1H), 4.28 (d, J=5.6 Hz, 2H). MS (ES+) m/e 408/410 [M+H]⁺

### Example 146

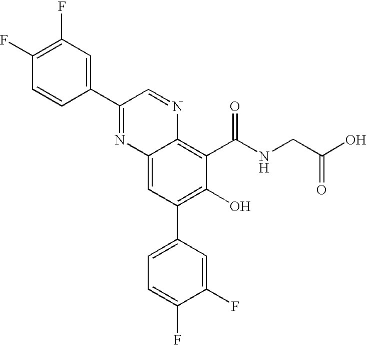

### N-{[2,7-bis(3,4-difluorophenyl)-6-hydroxy-5-quinoxalinyl]carbonyl}glycine

146(a) methyl 2,7-bis(3,4-difluorophenyl)-6-(methyloxy)-5-quinoxalinecarboxylate. A solution of methyl 7-bromo-6-(methyloxy)-2-{[(trifluoromethyl)sulfonyl]oxy}-5-quinoxalinecarboxylate (200 mg, 0.449 mmol), (3,4-difluorophenyl)boronic acid (142 mg, 0.899 mmol), potassium carbonate (186 mg, 1.348 mmol), and tetrakis(triphenylphosphine)palladium(0) (15.57 mg, 0.013 mmol) in 1,4-dioxane (2.0 ml) and water (0.667 ml) was heated to 105° C. overnight in an oil bath. Upon cooling, the reaction mixture was diluted with water and extracted twice with ethyl acetate. The organic portions were dried over magnesium sulfate, filtered and concentrated in vacuo. The resulting residue was purified via flash column chromatography (0-100% ethyl acetate in hexanes) to obtain methyl 2,7-bis(3,4-difluorophenyl)-6-(methyloxy)-5-quinoxalinecarboxylate (71 mg, 0.098 mmol, 21.79% yield) as an amber oil. ¹H NMR (400 MHz, CHLOROFORM-d) δ ppm 8.44 (s, 1H), 8.13 (s, 1H), 7.98-8.10 (m, 2H), 7.80-7.95 (m, 2H), 7.53-7.66 (m, 1H), 7.41-7.50 (m, 1H), 4.13 (s, 3H), 3.67 (s, 3H). MS (ES+) m/e 443 [M+H]⁺.

146(b) 2,7-bis(3,4-difluorophenyl)-6-hydroxy-5-quinoxalinecarboxylic acid. A solution of methyl 2,7-bis(3,4-difluorophenyl)-6-(methyloxy)-5-quinoxalinecarboxylate (71 mg, 0.161 mmol) in dichloromethane (5.0 mL) was treated with boron tribromide (1M solution in dichloromethane) (0.482 mL, 0.482 mmol) at room temperature overnight. The reaction mixture was quenched with water and extracted twice with dichloromethane. The combined organic portions were dried over magnesium sulfate, filtered, concentrated and purified via flash column chromatography (0-100% ethyl acetate in hexanes) to give 2,7-bis(3,4-difluorophenyl)-6-hydroxy-5-quinoxalinecarboxylic acid (29 mg, 0.070 mmol, 43.6% yield) as a yellow solid. MS (ES+) m/e 415 [M+H]⁺.

146(c) ethyl N-{[2,7-bis(3,4-difluorophenyl)-6-hydroxy-5-quinoxalinyl]carbonyl}glycinate. A solution of 2,7-bis(3,4-difluorophenyl)-6-hydroxy-5-quinoxalinecarboxylic acid (29 mg, 0.070 mmol) and ethyl glycine hydrochloride (19.54 mg, 0.140 mmol) in N,N-Dimethylformamide (DMF) (3.0 mL) was treated with triethylamine (0.029 mL, 0.210 mmol) and PyBOP (40.1 mg, 0.077 mmol). The reaction mixture was stirred overnight at ambient temperature, quenched by water, filtered and dried in vacuo to obtain ethyl N-{[2,7-bis(3,4-difluorophenyl)-6-hydroxy-5-quinoxalinyl]carbonyl}glycinate (15 mg, 0.030 mmol, 42.9% yield) as a pale yellow solid. ¹H NMR (400 MHz, DMSO-d₆) δ ppm 16.15 (s, 1H), 11.57 (t, J=5.3 Hz, 1H), 9.60 (s, 1H), 8.36-8.47 (m, 1H), 8.32 (s, 1H), 8.17-8.27 (m, 1H), 7.81-7.90 (m, 1H), 7.66-7.77 (m, 1H), 7.56-7.63 (m, 2H), 4.39 (d, J=5.3 Hz, 1H), 4.20 (q, J=7.1 Hz, 2H), 1.25 (t, J=7.1 Hz, 3H). MS (ES+) m/e 500 [M+H]⁺.

146(d) N-{[2,7-bis(3,4-difluorophenyl)-6-hydroxy-5-quinoxalinyl]carbonyl}glycine. To a suspension of ethyl N-{[2,7-bis(3,4-difluorophenyl)-6-hydroxy-5-quinoxalinyl]carbonyl}glycinate (15 mg, 0.030 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[2,7-bis(3,4-difluorophenyl)-6-hydroxy-5-quinoxalinyl]carbonyl}glycine (3.0 mg, 6.36 μmol, 21.19% yield) as a light yellow solid. MS (ES+) m/e 472 [M+H]⁺.

### Example 147

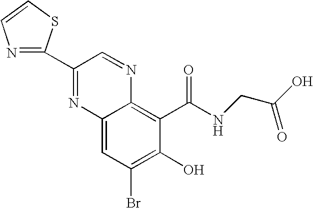

### N-{[7-bromo-6-hydroxy-2-(1,3-thiazol-2-yl)-5-quinoxalinyl]carbonyl}glycine

147(a) methyl 7-bromo-6-(methyloxy)-2-(1,3-thiazol-2-yl)-5-quinoxalinecarboxylate. To a solution of methyl 7-bromo-2-chloro-6-(methyloxy)-5-quinoxalinecarboxylate (example 69(a), 200 mg, 0.603 mmol) in 1,4-dioxane (1.5 ml) was added 2-(tributylstannanyl)-1,3-thiazole (226 mg, 0.603 mmol) and tetrakis(triphenylphosphine)palladium(0) (31.4 mg, 0.027 mmol) followed by heating to 120° C. for 60 min. in a Biotage Initiator® microwave synthesizer. Upon cooling, the reaction mixture was concentrated in vacuo and purified via flash column chromatography (0-100% ethyl acetate in hexanes) to obtain methyl 7-bromo-6-(methyloxy)-2-(1,3-thiazol-2-yl)-5-quinoxalinecarboxylate (164 mg, 0.173 mmol, 28.6% yield) as a pale yellow solid. MS (ES+) m/e 380/382 [M+H]⁺.

147(b) 7-bromo-6-hydroxy-2-(1,3-thiazol-2-yl)-5-quinoxalinecarboxylic acid. A solution of methyl 7-bromo-6-(methyloxy)-2-(1,3-thiazol-2-yl)-5-quinoxalinecarboxylate (164 mg, 0.431 mmol) in dichloromethane (3.00 mL) was treated with boron tribromide (1M solution in dichloromethane) (1.725 mL, 1.725 mmol) at room temperature overnight. The reaction mixture was quenched with water and extracted twice with dichloromethane. The combined organic portions were dried over magnesium sulfate, filtered, concentrated and purified via flash column chromatography (0-10% methanol in dichloromethane) to give 7-bromo-6-hydroxy-2-(1,3-thiazol-2-yl)-5-quinoxalinecarboxylic acid (76 mg, 0.216 mmol, 50.0% yield) as a light orange solid. ¹H NMR (400 MHz, DMSO-d₆) δ ppm 9.59 (s, 1H), 8.75 (s, 1H), 8.16 (d, J=3.0 Hz, 1H), 8.07 (d, J=3.0 Hz, 1H). MS (ES+) m/e 352/354 [M+H]⁺.

147(c) ethyl N-{[7-bromo-6-hydroxy-2-(1,3-thiazol-2-yl)-5-quinoxalinyl]carbonyl}glycinate. A solution of 7-bromo-6-hydroxy-2-(1,3-thiazol-2-yl)-5-quinoxalinecarboxylic acid (38 mg, 0.108 mmol) and ethyl glycine hydrochloride (30.1 mg, 0.216 mmol) in N,N-Dimethylformamide (DMF) (3.0 mL) was treated with triethylamine (0.045 mL, 0.324 mmol) and PyBOP (61.8 mg, 0.119 mmol). The reaction mixture was stirred overnight at ambient temperature, quenched by water, filtered and dried in vacuo to obtain ethyl N-{[7-bromo-6-hydroxy-2-(1,3-thiazol-2-yl)-5-quinoxalinyl]carbonyl}glycinate (20 mg, 0.046 mmol, 42.4% yield) as a yellow solid. ¹H NMR (400 MHz, DMSO-d₆) δ ppm 11.46 (t, J=5.6 Hz, 1H), 9.62 (s, 1H), 8.72 (s, 1H), 8.17 (d, J=3.0 Hz, 1H), 8.08 (d, J=3.0 Hz, 1H), 4.35 (d, J=5.6 Hz, 2H), 4.19 (q, J=7.2 Hz, 2H), 1.24 (t, J=7.2 Hz, 3H). MS (ES+) m/e 437/439 [M+H]⁺.

147(d) N-{[7-bromo-6-hydroxy-2-(1,3-thiazol-2-yl)-5-quinoxalinyl]carbonyl}glycine. To a suspension of ethyl N-{[7-bromo-6-hydroxy-2-(1,3-thiazol-2-yl)-5-quinoxalinyl]carbonyl}glycinate (20 mg, 0.046 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[7-bromo-6-hydroxy-2-(1,3-thiazol-2-yl)-5-quinoxalinyl]carbonyl}glycine (11.0 mg, 0.027 mmol, 58.8% yield) as an orange solid. ¹H NMR (400 MHz, DMSO-d₆) δ ppm 11.43 (t, J=5.1 Hz, 1H), 9.61 (s, 1H), 8.71 (s, 1H), 8.17 (d, J=3.3 Hz, 1H), 8.07 (d, J=3.3 Hz, 1H), 4.27 (d, J=5.1 Hz, 2H). MS (ES+) m/e 409/411 [M+H]⁺.

### Example 148

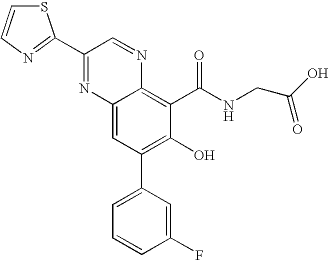

### N-{[7-(3-fluorophenyl)-6-hydroxy-2-(1,3-thiazol-2-yl)-5-quinoxalinyl]carbonyl}glycine

148(a) methyl 7-(3-fluorophenyl)-6-(methyloxy)-2-(1,3-thiazol-2-yl)-5-quinoxalinecarboxylate. To a solution of methyl 7-(3-fluorophenyl)-6-(methyloxy)-2-{[(trifluoromethyl)sulfonyl]oxy}-5-quinoxalinecarboxylate (example 141(b), 0.128 g, 0.278 mmol) in 1,4-dioxane (1.5 ml) was added 2-(tributylstannanyl)-1,3-thiazole (0.104 g, 0.278 mmol) and tetrakis(triphenylphosphine)palladium(0) (0.014 g, 0.013 mmol) followed by heating to 120° C. for 20 min. in a Biotage Initiator® microwave synthesizer, then overnight to 105° C. in an oil bath. Upon cooling, the reaction mixture was concentrated in vacuo and purified via flash column chromatography (0-100% ethyl acetate in hexanes) to obtain a mixture of methyl 2-butyl-7-(3-fluorophenyl)-6-(methyloxy)-5-quinoxalinecarboxylate and methyl 7-(3-fluorophenyl)-6-(methyloxy)-2-(1,3-thiazol-2-yl)-5-quinoxalinecarboxylate (45 mg, 0.024 mmol, 8.60% yield) as a pale orange solid. ¹H NMR (400 MHz, DMSO-d₆) δ ppm 9.66 (s, 1H), 8.25 (s, 1H), 8.18 (d, J=3.3 Hz, 1H), 8.10 (d, J=3.0 Hz, 1H), 7.49-7.66 (m, 2H), 7.24-7.44 (m, 2H), 4.00 (s, 3H), 3.59 (s, 3H). MS (ES+) m/e 396 [M+H]⁺.

148(b) 7-(3-fluorophenyl)-6-hydroxy-2-(1,3-thiazol-2-yl)-5-quinoxalinecarboxylic acid. A mixture of methyl 2-butyl-7-(3-fluorophenyl)-6-(methyloxy)-5-quinoxalinecarboxylate (41.9 mg, 0.114 mmol) and methyl 7-(3-fluorophenyl)-6-(methyloxy)-2-(1,3-thiazol-2-yl)-5-quinoxalinecarboxylate (45 mg, 0.114 mmol) in dichloromethane (10 mL) was treated with boron tribromide (1M solution in dichloromethane) (0.569 mL, 0.569 mmol) at room temperature overnight. The reaction mixture was quenched with water and extracted twice with ethyl acetate. The combined organic portions were dried over magnesium sulfate, filtered and concentrated to give a mixture of 2-butyl-7-(3-fluorophenyl)-6-hydroxy-5-quinoxalinecarboxylic acid MS (ES+) m/e 341 [M+H]⁺ and 7-(3-fluorophenyl)-6-hydroxy-2-(1,3-thiazol-2-yl)-5-quinoxalinecarboxylic acid (30 mg, 0.026 mmol, 22.96% yield) as a green solid. ¹H NMR (400 MHz, DMSO-d₆) δ ppm 9.66 (s, 1H), 8.42 (s, 1H), 8.18 (d, J=3.3 Hz, 1H), 8.09 (d, J=3.3 Hz, 1H), 7.60-7.69 (m, 2H), 7.51-7.60 (m, 2H). MS (ES+) m/e 368 [M+H]⁺

148(c) ethyl N-{[7-(3-fluorophenyl)-6-hydroxy-2-(1,3-thiazol-2-yl)-5-quinoxalinyl]carbonyl}glycinate. A solution of the mixture 7-(3-fluorophenyl)-6-hydroxy-2-(1,3-thiazol-2-yl)-5-quinoxalinecarboxylic acid (30 mg, 0.082 mmol) and 2-butyl-7-(3-fluorophenyl)-6-hydroxy-5-quinoxalinecarboxylic acid (27.8 mg, 0.082 mmol) and ethyl glycine hydrochloride (22.80 mg, 0.163 mmol) in N,N-Dimethylformamide (DMF) (3.0 mL) was treated with triethylamine (0.034 mL, 0.245 mmol) and PyBOP (46.7 mg, 0.090 mmol). The reaction mixture was stirred overnight at ambient temperature, quenched by water, extracted using ethyl acetate, dried over magnesium sulfate, filtered, concentrated and purified via flash column chromatography (0-100% ethyl acetate in hexanes) to obtain ethyl N-{[7-(3-fluorophenyl)-6-hydroxy-2-(1,3-thiazol-2-yl)-5-quinoxalinyl]carbonyl}glycinate (10 mg, 0.022 mmol, 27.1% yield). MS (ES+) m/e 453 [M+H]⁺.

148(d) N-{[7-(3-fluorophenyl)-6-hydroxy-2-(1,3-thiazol-2-yl)-5-quinoxalinyl]carbonyl}glycine. To a suspension of ethyl N-{[7-(3-fluorophenyl)-6-hydroxy-2-(1,3-thiazol-2-yl)-5-quinoxalinyl]carbonyl}glycinate (10 mg, 0.022 mmol) in ethanol (1.0 mL) was added 1N aqueous sodium hydroxide (1.0 ml, 1.000 mmol). After stirring 30 min. at ambient temperature, the reaction was quenched with 1N aqueous hydrochloric acid and the resulting precipitate was filtered, washed with water, and dried in vacuo to obtain N-{[7-(3-fluorophenyl)-6-hydroxy-2-(1,3-thiazol-2-yl)-5-quinoxalinyl]carbonyl}glycine (1.0 mg, 2.356 μmol, 10.66% yield) as a yellow solid. ¹H NMR (400 MHz, DMSO-d₆) δ ppm 11.50 (t, J=6.1 Hz, 1H), 9.61 (s, 1H), 8.30 (s, 1H), 8.17 (d, J=3.3 Hz, 1H), 8.07 (d, J=3.3 Hz, 1H), 7.59-7.63 (m, 2H), 7.48-7.58 (m, 1H), 7.26-7.38 (m, 1H), 4.28 (d, J=6.1 Hz, 2H). MS (ES+) m/e 425 [M+H]⁺.

### Example 149

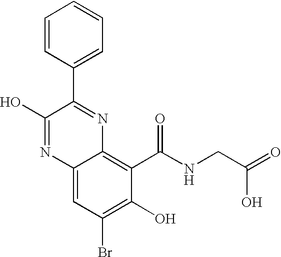

### N-[(7-bromo-6-hydroxy-2-oxo-3-phenyl-1,2-dihydro-5-quinoxalinyl)carbonyl]glycine

149(a) methyl 7-bromo-6-(methyloxy)-2-oxo-3-phenyl-1,2-dihydro-5-quinoxalinecarboxylate. To a solution of methyl 2-amino-5-bromo-6-(methyloxy)-3-nitrobenzoate (2.04 g, 6.69 mmol) in ethanol (100 ml) was added tin(II) chloride hydrate (5.52 g, 24.47 mmol). After stirring at reflux for 3 h, the reaction mixture was allowed to cool to ambient temperature, poured into water, basified with 5% aqueous sodium bicarbonate and extracted thrice with ethyl acetate. The combined organic layers were dried over MgSO₄, filtered, and concentrated in vacuo. The resulting orange oil was diluted in acetonitrile (100 ml) and treated with ethyl oxo(phenyl)acetate (1.311 g, 7.36 mmol) and stirred at room temperature overnight. The reaction mixture was concentrated in vacuo and purified via flash column chromatography (0-100% ethyl acetate in hexanes) to afford a dark yellow solid. This solid was further purified using rp-HPLC (acetonitrile, water, TFA) to obtain methyl 7-bromo-6-(methyloxy)-2-oxo-3-phenyl-1,2-dihydro-5-quinoxalinecarboxylate (0.8616 g, 2.214 mmol, 33.1% yield) as a yellow solid ¹H NMR (400 MHz, DMSO-d₆) δ ppm 12.70 (s, 1H), 8.29 (d, J=1.5 Hz, 1H), 8.27 (d, J=1.8 Hz, 1H), 7.62 (s, 1H), 7.45-7.57 (m, 3H), 3.96 (s, 3H), 3.84 (s, 3H). MS (ES+) m/e 389/391 [M+H]⁺.

149(b) 7-bromo-6-hydroxy-2-oxo-3-phenyl-1,2-dihydro-5-quinoxalinecarboxylic acid. A solution of methyl 7-bromo-6-(methyloxy)-2-oxo-3-phenyl-1,2-dihydro-5-quinoxalinecarboxylate (200 mg, 0.514 mmol) in dichloromethane (10 mL) was treated with boron tribromide (1M solution in dichloromethane) (2.57 mL, 2.57 mmol) at room temperature overnight. The reaction mixture was poured into water and extracted twice with ethyl acetate. The combined organic portions were dried over magnesium sulfate, filtered and concentrated to give 7-bromo-6-hydroxy-2-oxo-3-phenyl-1,2-dihydro-5-quinoxalinecarboxylic acid (127 mg, 0.352 mmol, 68.4% yield) as a bright orange solid. ¹H NMR (400 MHz, DMSO-d₆) δ ppm 15.26 (br. s., 1H), 12.88 (s, 1H), 11.84 (br. s., 1H), 8.25 (d, J=1.5 Hz, 1H), 8.23 (d, J=1.5 Hz, 1H), 7.74 (s, 1H), 7.52-7.62 (m, 3H). MS (ES+) m/e 361/363 [M+H]⁺.

149(c) ethyl N-[(7-bromo-6-hydroxy-2-oxo-3-phenyl-1,2-dihydro-5-quinoxalinyl)carbonyl]glycinate. A solution of 7-bromo-6-hydroxy-2-oxo-3-phenyl-1,2-dihydro-5-quinoxalinecarboxylic acid (127 mg, 0.352 mmol) and ethyl glycine hydrochloride (98 mg, 0.703 mmol) in N,N-Dimethylformamide (DMF) (3.0 mL) was treated with triethylamine (0.147 mL, 1.055 mmol) and PyBOP (201 mg, 0.387 mmol). The reaction mixture was stirred overnight at ambient temperature, quenched by water, filtered and dried in vacuo. The resulting solid was washed with dichloromethane and dried in vacuo to obtain ethyl N-[(7-bromo-6-hydroxy-2-oxo-3-phenyl-1,2-dihydro-5-quinoxalinyl)carbonyl]glycinate (10 mg, 0.022 mmol, 6.37% yield) as a yellow solid. ¹H NMR (400 MHz, DMSO-d₆) δ ppm 14.71 (br. s., 1H), 12.85 (br. s., 1H), 11.17 (t, J=5.6 Hz, 1H), 8.28 (br. s., 1H), 8.27 (d, J=1.3 Hz, 1H), 7.79 (s, 1H), 7.56-7.63 (m, 1H), 7.49-7.56 (m, 2H), 4.36 (d, J=5.6 Hz, 2H), 4.16 (q, J=7.2 Hz, 2H), 1.20 (t, J=7.2 Hz, 3H). MS (ES+) m/e 446/448 [M+H]⁺.

149(d) N-[(7-bromo-6-hydroxy-2-oxo-3-phenyl-1,2-dihydro-5-quinoxalinyl)carbonyl]glycine. To a suspension of ethyl N-[(7-bromo-6-hydroxy-2-oxo-3-phenyl-1,2-dihydro-5-quinoxalinyl)carbonyl]glycinate (10 mg, 0.022 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-[(7-bromo-6-hydroxy-2-oxo-3-phenyl-1,2-dihydro-5-quinoxalinyl)carbonyl]glycine (9.0 mg, 0.022 mmol, 96% yield) as a yellow solid. ¹H NMR (400 MHz, DMSO-d₆) δ ppm 10.99 (t, J=5.4 Hz, 1H), 8.13 (br. s., 1H), 8.11 (d, J=1.5 Hz, 1H), 8.04 (s, 1H), 7.42-7.49 (m, 1H), 7.34-7.42 (m, 2H), 4.16 (d, J=5.4 Hz, 2H). MS (ES+) m/e 418/420 [M+H]⁺.

### Example 150

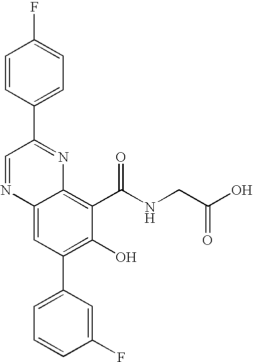

### N-{[7-(3-fluorophenyl)-3-(4-fluorophenyl)-6-hydroxy-5-quinoxalinyl]carbonyl}glycine

To a mixture of ethyl N-{[3-bromo-7-(3-fluorophenyl)-6-hydroxy-5-quinoxalinyl]carbonyl}glycinate (example 143(e), 0.220 g, 0.49 mmol), 4-fluorophenylboronic acid (0.083 g, 0.59 mmol) and potassium carbonate (0.149 g, 1.08 mmol) in 1,4-dioxane (2.5 mL) and water (1 mL) was added tetrakis(triphenylphosphine)palladium (0) (0.025 g, 0.022 mmol) under the protection of nitrogen. The mixture was heated in a Biotage Initiator microwave synthesizer at 120° C. for 1 h. Upon cooling, the reaction mixture was treated with 1N sodium hydroxide aqueous solution (8 mL) in tetrahydrofuran (6 mL) for 10 min and then concentrated in vacuo and purified via rp-HPLC (C18 column, MeCN in water [0.01% TFA] from 20% to 85% in 25 min) to afford the title compound (38 mg, 17.8% yield) as a brown solid. 1H NMR (400 MHz, DMSO-d6) δ ppm 16.30 (s, 1H, br), 11.54 (t, 1H, br), 9.53 (s, 1H), 8.47 (t, 2H, J=3.6 Hz), 8.26 (s, 1H), 7.61 (t, 3H, J=6.4 Hz), 7.47 (t, 2H, J=8.8 Hz), 7.36 (m, 1H), 4.20 (d, 2H, J=5.2 Hz). MS (ES+) m/e 436 [M+H]⁺.

### Example 151

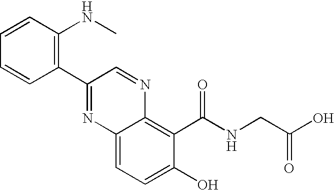

### N-({6-hydroxy-2-[2-(methylamino)phenyl]-5-quinoxalinyl}carbonyl)glycine

To a mixture of the compound from example 5(a) (0.091 g, 0.26 mmol), 2-(methylamino)phenylboronic acid pinacol ester (0.060 g, 0.257 mmol) and potassium carbonate (0.071 g, 0.514 mmol) in 1,4-dioxane (3.5 mL) and was added tetrakis(triphenylphosphine)palladium (0.019 g, 0.016 mmol) followed by evacuation of the reaction vessel and purging with nitrogen. The reaction mixture was heated in a Biotage Initiator® microwave synthesizer at 120° C. for 20 min to get the intermediate ester and upon cooling, tetrahydrofuran (5.0 mL) and 1N aqueous sodium hydroxide (5.0 mL) were added. After stirring for 5 min at ambient temperature, the mixture was quenched with 1N aqueous hydrochloric acid and the resulting precipitate was filtered, purified via rp-HPLC (acetonitrile/water+0.1% trifluoroacetic acid) to afford the title compound (0.038 g, 41.5% yield) as an orange solid. ¹H NMR (400 MHz, DMSO-d6) δ ppm 15.09 (s, 1H), 12.93 (s, 1H), 11.37 (t, 1H, J=6.0 Hz), 9.44 (s, 1H), 8.32 (d, 1H, J=9.2 Hz), 7.972 (m, 1H), 7.57 (d, 1H, J=9.2 Hz), 7.36 (m, 1H), 6.78 (m, 2H), 4.26 (d, 2H, J=5.6 Hz), 2.92 (s, 3H). MS (ES+) m/e 353 [M+H]⁺.

### Example 152

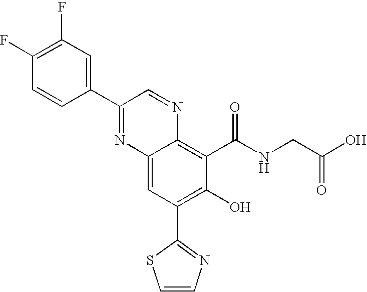

### N-{[2-(3,4-difluorophenyl)-6-hydroxy-7-(1,3-thiazol-2-yl)-5-quinoxalinyl]carbonyl}glycine

152(a) methyl 7-bromo-2-(3,4-difluorophenyl)-6-(methyloxy)-5-quinoxalinecarboxylate. A solution of methyl 7-bromo-2-chloro-6-(methyloxy)-5-quinoxalinecarboxylate (example 69(a), 550 mg, 1.659 mmol), (3,4-difluorophenyl)boronic acid (262 mg, 1.659 mmol), potassium carbonate (688 mg, 4.98 mmol), and tetrakis(triphenylphosphine)palladium(0) (57.5 mg, 0.050 mmol) in 1,4-dioxane (2.0 ml) and water (0.667 ml) was heated to 100° C. in an oil bath for 3 h. Upon cooling, the reaction mixture was diluted with water, extracted using ethyl acetate, dried over magnesium sulfate, filtered and concentrated in vacuo. The resulting residue was purified via flash cromatography (0-10% methanol in dichloromethane) to obtain a mixture of methyl 7-bromo-2-(3,4-difluorophenyl)-6-(methyloxy)-5-quinoxalinecarboxylate (237 mg, 0.578 mmol, 34.8% yield), ¹H NMR (400 MHz, CHLOROFORM-d) δ ppm 9.27 (s, 1H), 8.46 (s, 1H), 8.00-8.10 (m, 1H), 7.83-7.95 (m, 1H), 7.31-7.43 (m, 1H), 4.11 (s, 3H), 4.09 (s, 3H), MS (ES+) m/e 409/411 [M+H]⁺ and methyl 2,7-bis(3,4-difluorophenyl)-6-(methyloxy)-5-quinoxalinecarboxylate (69 mg, 0.156 mmol, 9.40% yield), MS (ES+) m/e 443 [M+H]⁺ as a white solid.

152(b) 7-bromo-2-(3,4-difluorophenyl)-6-hydroxy-5-quinoxalinecarboxylic acid. A solution of the mixture methyl 2,7-bis(3,4-difluorophenyl)-6-(methyloxy)-5-quinoxalinecarboxylate (69 mg, 0.156 mmol) and methyl 7-bromo-2-(3,4-difluorophenyl)-6-(methyloxy)-5-quinoxalinecarboxylate (237 mg, 0.579 mmol) in dichloromethane (25 mL) was treated with boron tribromide (1M solution in dichloromethane) (5.0 mL, 5.00 mmol) at room temperature overnight. The reaction mixture was poured into water and extracted twice with ethyl acetate. The combined organic portions were dried over magnesium sulfate, filtered and concentrated to give a mixture of 7-bromo-2-(3,4-difluorophenyl)-6-hydroxy-5-quinoxalinecarboxylic acid (0.518 g crude), ¹H NMR (400 MHz, DMSO-d₆) δ ppm 9.58 (s, 1H), 8.75 (s, 1H), 8.31 (ddd, J=12.1, 7.8, 2.3 Hz, 1H), 8.09-8.17 (m, 1H), 7.71 (ddd, J=12.1, 7.8, 2.3 Hz, 1H), MS (ES+) m/e 381/383 [M+H]⁺ and 2,7-bis(3,4-difluorophenyl)-6-hydroxy-5-quinoxalinecarboxylic acid, MS (ES+) m/e 415 [M+H]⁺.

152(c) ethyl N-{[7-bromo-2-(3,4-difluorophenyl)-6-hydroxy-5-quinoxalinyl]carbonyl}glycinate. A solution of the mixture 2,7-bis(3,4-difluorophenyl)-6-hydroxy-5-quinoxalinecarboxylic acid (563 mg, 1.359 mmol) and 7-bromo-2-(3,4-difluorophenyl)-6-hydroxy-5-quinoxalinecarboxylic acid (518 mg, 1.359 mmol) and ethyl glycine hydrochloride (379 mg, 2.72 mmol) in N,N-Dimethylformamide (DMF) (3.0 mL) was treated with triethylamine (0.568 mL, 4.08 mmol) and PyBOP (778 mg, 1.495 mmol). The reaction mixture was stirred overnight at ambient temperature, quenched by water, filtered and dried in vacuo to obtain a solid, which was purified via rp-HPLC (0.1% TFA acetonitrile and water) to obtain ethyl N-{[7-bromo-2-(3,4-difluorophenyl)-6-hydroxy-5-quinoxalinyl]carbonyl}glycinate (60 mg, 0.129 mmol, 9.47% yield) as a light yellow solid. ¹H NMR (400 MHz, CHLOROFORM-d) δ ppm 16.17 (br. s., 1H), 11.64 (br. s., 1H), 9.24 (s, 1H), 8.52 (s, 1H), 8.06 (ddd, J=11.4, 7.6, 2.3 Hz, 1H), 7.86-7.94 (m, 1H), 7.36 (q, J=9.6 Hz, 1H), 4.39 (d, J=5.1 Hz, 2H), 4.32 (q, J=7.1 Hz, 2H), 1.36 (t, J=7.2 Hz, 3H). MS (ES+) m/e 466/468 [M+H]⁺.

152(d) ethyl N-{[2-(3,4-difluorophenyl)-6-hydroxy-7-(1,3-thiazol-2-yl)-5-quinoxalinyl]carbonyl}glycinate. A slurry of ethyl N-{[7-bromo-2-(3,4-difluorophenyl)-6-hydroxy-5-quinoxalinyl]carbonyl}glycinate (60 mg, 0.129 mmol) in 1,4-dioxane (1.5 ml) was treated with tetrakis(triphenylphosphine)palladium(0) (6.69 mg, 5.79 μmol) and degassed using argon. 2-(tributylstannanyl)-1,3-thiazole (100 mg, 0.267 mmol) was then added and heating to 150° C. for 20 min. in a Biotage Initiator® microwave synthesizer followed. Upon cooling, the reaction mixture was filtered through silica gel and the residue was concentrated in vacuo. The resulting solid was washed with ether and then filtered to obtain ethyl N-{[2-(3,4-difluorophenyl)-6-hydroxy-7-(1,3-thiazol-2-yl)-5-quinoxalinyl]carbonyl}glycinate (20 mg, 0.043 mmol, 33.0% yield) as a green solid. ¹H NMR (400 MHz, CHLOROFORM-d) δ ppm 11.73 (t, J=5.1 Hz, 1H), 9.30 (s, 1H), 9.23 (s, 1H), 8.08 (d, J=3.3 Hz, 1H), 8.05-8.11 (m, 1H), 7.90-7.99 (m, 1H), 7.62 (d, J=3.3 Hz, 1H), 7.37 (q, J=8.3 Hz, 1H), 4.42 (d, J=5.1 Hz, 2H), 4.33 (q, J=7.2 Hz, 2H), 1.37 (t, J=7.2 Hz, 3H). MS (ES+) m/e 471 [M+H]⁺.

152(e) N-{[2-(3,4-difluorophenyl)-6-hydroxy-7-(1,3-thiazol-2-yl)-5-quinoxalinyl]carbonyl}glycine. To a suspension of ethyl N-{[2-(3,4-difluorophenyl)-6-hydroxy-7-(1,3-thiazol-2-yl)-5-quinoxalinyl]carbonyl}glycinate (20 mg, 0.043 mmol) in ethanol (1.0 mL) was added 1N aqueous sodium hydroxide (2.0 ml, 2.000 mmol). After stirring 30 min. at ambient temperature, the reaction was quenched with 1N aqueous hydrochloric acid and the resulting precipitate was filtered, washed with water, and dried in vacuo to obtain N-{[2-(3,4-difluorophenyl)-6-hydroxy-7-(1,3-thiazol-2-yl)-5-quinoxalinyl]carbonyl}glycine (18 mg, 0.041 mmol, 96% yield) as a green solid. ¹H NMR (400 MHz, DMSO-d₆) □ ppm 10.95 (br. s., 1H), 9.50 (s, 1H), 9.00 (s, 1H), 8.30-8.44 (m, 1H), 8.15-8.23 (m, 1H), 8.12 (d, J=3.3 Hz, 1H), 7.97 (d, J=3.3 Hz, 1H), 7.58-7.73 (m, 1H), 4.12 (d, J=5.1 Hz, 2H). MS (ES+) m/e 443 [M+H]⁺.

### Example 153

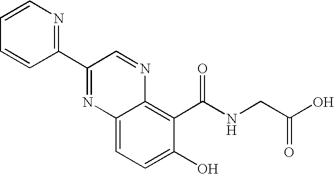

### N-{[6-hydroxy-2-(2-pyridinyl)-5-quinoxalinyl]carbonyl}glycine

A solution of the compound of example 5(a) (354 mg, 1.0 mmol), 2-(tributylstannyl)pyridine (736 mg, 2.0 mmol), and tetrakis(triphenylphosphine)palladium (0) (115 mg, 0.1 mmol0) in N-methylpyrrolidinone (12 mL) was degassed with nitrogen for 10 min, then heated at 150° C. for 30 min in a microwave reactor. After cooling, 30 mL of water was added to the mixture and the solid filtered to get 320 mg of crude product. The crude ester (320 mg) in tetrahydrofuran (10 mL) was added to 2M aqueous sodium hydroxide (10 mL) and the mixture stirred at room temperature for 0.5 h. TLC showed the reaction was complete. 1M aqueous hydrochloric acid was added to adjust pH to 6-7. The crude precipitate (500 mg) was filtered, then purified by rp-HPLC to give the desired product (33.5 mg, 10.3%) as a solid. 1H NMR (400 MHz, DMSO-d6) δ 15.37 (s, 1H), 12.92 (b, 1H), 11.43 (t, 1H, J=5.2 Hz), 9.82 (s, 1H), 8.81 (d, 1H, J=4.4 Hz), 8.50 (d, 1H, J=8.0 Hz), 8.26 (d, 1H, J=89.2 Hz), 8.07 (dt, 1H, J1=8.0 Hz, J2=1.6 Hz0), 7.60 (d, 1H, J=9.2 Hz), 7.58 (m, 1H), 4.26 (d, 2H, J=9.2 Hz). MS (ES+) m/e 325 [M+H]⁺.

### BIOLOGICAL BACKGROUND

The following references set out information about the target enzymes, HIF prolyl hydroxylases, and methods and materials for measuring inhibition of same by small molecules.
M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.
C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regul., 2004, 44, 75-92
M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.
S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1
C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.
   For the expression of EGLN3 see:
R. K. Bruick and S. L. McKnight “A Conserved Family of Prolyl-4-Hydroxylases That Modify HIF” Science, 2001, 294, 1337-1340.
   For the expression of HIF2α-CODD see:
a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.
b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂Sensing” Science, 2001, 292, 464-468.
   For the expression of VHL, elongin b and elongin c see:
A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### BIOLOGICAL ASSAY(S)

### EGLN3 Assay

### Materials:

His-MBP-EGLN3 (6HisMBPAttB1EGLN3(1-239)) was expressed in E. Coli and purified from an amylase affinity column. Biotin-VBC [6HisSumoCysVHL(2-213), 6HisSumoElonginB(1-118), and 6HisSumoElonginC(1-112)] and His-GB1-HIF2α-CODD (6HisGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 mL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 μL EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)̂d), where a is the minimum % activity, b is the maximum % activity, c is the pIC₅₀, and d is the Hill slope.

The IC₅₀ for exemplified compounds in the EGLN3 assay ranged from approximately 1-13000 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37 deg C/5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀ for exemplar compounds in the Hep3B ELISA assay ranged from approximately 0.1-greater than 100 micromolar using the reagents and under the conditions outlined herein above. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

1. A compound of formula (I): 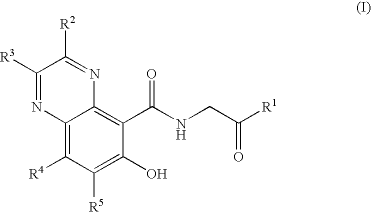
wherein:
R¹ is —NR⁶R⁷ or —OR⁸;
R², R³, R⁴, and R⁵ are each independently selected from the group consisting of hydrogen, nitro, cyano, halogen, —C(O)R¹¹, —C(O)OR¹¹, —OR¹¹, —SR¹¹, —S(O)R¹¹, —S(O)₂R¹¹, —NR⁹R¹⁰, —CONR⁹R¹⁰, —N(R⁹)C(O)R¹¹, —N(R⁹)C(O)OR¹¹, —OC(O)NR⁹R¹⁰, —N(R⁹)C(O)N⁹R¹⁰, —P(O)(OR¹¹)₂, —SO₂NR⁹R¹⁰, —N(R⁹)SO₂R¹¹, C₁-C₁₀ alkyl, C₂-C₁₀ alkenyl, C₂-C₁₀ alkynyl, C₃-C₈ cycloalkyl, C₃-C₈ heterocycloalkyl, C₅-C₈ cycloalkenyl, aryl, and heteroaryl;
R⁶ and R⁷ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;
R⁸ is hydrogen, or a cation, or C₁-C₄ alkyl;
R⁹ and R¹⁰ are each independently selected from the group consisting of hydrogen, C₁-C₁₀ alkyl, C₃-C₈ cycloalkyl, C₁-C₁₀ alkyl-C₃-C₈ cycloalkyl, C₃-C₈ heterocycloalkyl, C₁-C₁₀ alkyl-C₃-C₈ heterocycloalkyl, aryl, C₁-C₁₀ alkyl-aryl, heteroaryl, C₁-C₁₀ alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁹ and R¹⁰ taken together with the nitrogen to which they are attached form a 5- or 6- or 7-membered saturated ring optionally containing one other heteroatom which is oxygen, nitrogen or sulphur;
each R¹¹ is independently selected from the group consisting of hydrogen, C₁-C₁₀ alkyl, C₂-C₁₀ alkenyl, C₂-C₁₀ alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈ heterocycloalkyl, aryl, C₁-C₁₀ alkyl-aryl, heteroaryl, and C₁-C₁₀ alkyl-heteroaryl;
any carbon or heteroatom of R², R³, R⁴, R⁵, R⁶, R⁷, R⁸, R⁹, R¹⁰, or R¹¹ is unsubstituted or, where possible, is substituted with one or more substituents independently selected from C₁-C₆ alkyl, aryl, heteroaryl, halogen, —NR⁹R¹⁰, cyano, nitro, —C(O)R¹¹, —C(O)OR¹¹, —SR¹¹, —S(O)R¹¹, —S(O)₂R¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₁₀ alkenyl, C₂-C₁₀ alkynyl, C₃-C₈ cycloalkyl, C₃-C₈ heterocycloalkyl, C₅-C₈ cycloalkenyl, aryl or heteroaryl, wherein R⁹, R¹⁰, and R¹¹ are the same as defined above;
or a pharmaceutically acceptable salt or solvate thereof.

2. A compound according to claim 1 wherein:
R¹ is —OR⁸;
R², R³, R⁴, and R⁵ are each independently selected from the group consisting of hydrogen, cyano, halogen, —OR¹¹, —NR⁹R¹⁰, —CONR⁹R¹⁰, C₁-C₆ alkyl, C₃-C₆ cycloalkyl, C₃-C₆ heterocycloalkyl, aryl, and heteroaryl;
R⁸ is hydrogen, or a cation;
R⁹ and R¹⁰ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁹ and R¹⁰ taken together with the nitrogen to which they are attached form a 5- or 6- or 7-membered saturated ring optionally containing one other heteroatom which is oxygen, nitrogen or sulphur;
each R¹¹ is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;
any carbon or heteroatom of R², R³, R⁴, R⁵, R⁸, R⁹, R¹⁰, or R¹¹ is unsubstituted or, where possible, is substituted with one or more substituents independently selected from C₁-C₆ alkyl, aryl, heteroaryl, halogen, —OR¹¹, —NR⁹R¹⁰, cyano, —C(O)R¹¹, C(O)OR¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R⁹, R¹⁰, and R¹¹ are the same as defined above;
or a pharmaceutically acceptable salt or solvate thereof.

3. A compound according to claim 1 wherein:
R¹ is —OR⁸;
R⁴ is hydrogen;
R², R³, and R⁵ are each independently selected from the group consisting of hydrogen, cyano, halogen, —OR¹¹, —NR⁹R¹⁰, —CONR⁹R¹⁰, C₁-C₆ alkyl, C₃-C₆ cycloalkyl, C₃-C₆ heterocycloalkyl, aryl, and heteroaryl;
R⁸ is hydrogen, or a cation;
R⁹ and R¹⁰ are each independently selected from the group consisting of hydrogen, C₁-C₆ alkyl, C₃-C₆ cycloalkyl, C₃-C₆ heterocycloalkyl, aryl, and heteroaryl; or R⁹ and R¹⁰ taken together with the nitrogen to which they are attached form a 5- or 6- or 7-membered saturated ring optionally containing one other heteroatom which is oxygen, nitrogen or sulphur;
each R¹¹ is independently selected from the group consisting of hydrogen, C₁-C₆ alkyl, C₃-C₆ cycloalkyl, C₃-C₆ heterocycloalkyl, aryl, and heteroaryl;
any carbon or heteroatom of R², R³, R⁵, R⁸, R⁹, R¹⁰, or R¹¹ is unsubstituted or, where possible, is substituted with one or more substituents independently selected from C₁-C₆ alkyl, aryl, heteroaryl, halogen, —OR¹¹, —NR⁹R¹⁰, cyano, —C(O)R¹¹, —C(O)OR¹¹, —CONR⁹R¹⁰, —N(R⁹)C(O)R¹¹, —N(R⁹)C(O)OR¹¹, —OC(O)NR⁹R¹⁰, —N(R⁹)C(O)NR⁹R¹⁰, —SO₂NR⁹R¹⁰, —N(R⁹)SO₂R¹¹, C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R⁹, R¹⁰, and R¹¹ are the same as defined above;
or a pharmaceutically acceptable salt or solvate thereof.

4. A compound according to claim 1 which is:
N-[(6-hydroxy-3-phenyl-5-quinoxalinyl)carbonyl]glycine;
N-[(6-hydroxy-3-methyl-5-quinoxalinyl)carbonyl]glycine;
N-[(6-hydroxy-5-quinoxalinyl)carbonyl]glycine;
N-[(2-bromo-6-hydroxy-5-quinoxalinyl)carbonyl]glycine;
N-({6-hydroxy-2-[4-(trifluoromethyl)phenyl]-5-quinoxalinyl}carbonyl)glycine;
N-({6-hydroxy-2-[(phenylmethyl)amino]-5-quinoxalinyl}carbonyl)glycine;
N-{[6-hydroxy-2-(phenylamino)-5-quinoxalinyl]carbonyl}glycine;
N-{[6-hydroxy-2-(phenyloxy)-5-quinoxalinyl]carbonyl}glycine;
N-{[6-hydroxy-2-(1-piperidinyl)-5-quinoxalinyl]carbonyl}glycine;
N-{[7-(3,5-difluorophenyl)-6-hydroxy-5-quinoxalinyl]carbonyl}glycine;
N-[(7-bromo-6-hydroxy-5-quinoxalinyl)carbonyl]glycine;
N-[7-(2-chlorophenyl)-6-hydroxy-5-quinoxalinyl)carbonyl]glycine;
N-{[6-hydroxy-7-(1-methylethyl)-5-quinoxalinyl]carbonyl}glycine;
N-[(6-hydroxy-2,3-dimethyl-5-quinoxalinyl)carbonyl]glycine;
N-[(7-bromo-6-hydr oxy-3-phenyl-5-quinoxalinyl)carbonyl]glycine;
N-{[7-bromo-3-(3,4-difluorophenyl)-6-hydroxy-5-quinoxalinyl]carbonyl}glycine;
N-{[7-bromo-3-(2,4-difluorophenyl)-6-hydroxy-5-quinoxalinyl]carbonyl}glycine;
N-{[7-bromo-3-(1,1-dimethylethyl)-6-hydroxy-5-quinoxalinyl]carbonyl}glycine;
N-{[7-bromo-3-(4-cyclohexylphenyl)-6-hydroxy-5-quinoxalinyl]carbonyl}glycine;
N-{[7-bromo-3-(4-fluorophenyl)-6-hydroxy-5-quinoxalinyl]carbonyl}glycine;
N-{[6-hydroxy-7-(2-pyridinyl)-5-quinoxalinyl]carbonyl}glycine;
N-{[6-hydroxy-7-(1,3-thiazol-2-yl)-5-quinoxalinyl]carbonyl}glycine;
N-[(6-hydroxy-7-phenyl-5-quinoxalinyl)carbonyl]glycine;
N-{[6-hydroxy-7-(1-methyl-1H-imidazol-2-yl)-5-quinoxalinyl]carbonyl}glycine;
N-{[6-hydroxy-3-phenyl-7-(2-pyridinyl)-5-quinoxalinyl]carbonyl}glycine;
N-{[6-hydroxy-7-(3-pyridinyl)-5-quinoxalinyl]carbonyl}glycine;
N-{[3-(3,4-difluorophenyl)-6-hydroxy-7-(2-pyridinyl)-5-quinoxalinyl]carbonyl}glycine;
N-{[6-hydroxy-3-phenyl-7-(1,3-thiazol-2-yl)-5-quinoxalinyl]carbonyl}glycine;
N-{[3-(3,4-difluorophenyl)-6-hydroxy-7-(1,3-thiazol-2-yl)-5-quinoxalinyl]carbonyl}glycine;
N-[(7-butyl-6-hydroxy-5-quinoxalinyl)carbonyl]glycine;
N-{[6-hydroxy-7-(4-pyridinyl)-5-quinoxalinyl]carbonyl}glycine;
N-{[6-hydroxy-7-(5-pyrimidinyl)-5-quinoxalinyl]carbonyl}glycine;
N-{[6-hydroxy-7-(1-methyl-1H-pyrazol-4-yl)-5-quinoxalinyl]carbonyl}glycine;
N-{[6-hydroxy-7-(2-pyrazinyl)-5-quinoxalinyl]carbonyl}glycine;
N-{[6-hydroxy-7-(4-methyl-1,3-thiazol-2-yl)-5-quinoxalinyl]carbonyl}glycine;
N-{[7-(2-furanyl)-6-hydroxy-5-quinoxalinyl]carbonyl}glycine;
N-{[6-hydroxy-7-(2-thienyl)-5-quinoxalinyl]carbonyl}glycine;
N-{[6-hydroxy-7-(2-pyrimidinyl)-5-quinoxalinyl]carbonyl}glycine;
N-{[6-hydroxy-7-(5-methyl-1,3-thiazol-2-yl)-5-quinoxalinyl]carbonyl}glycine;
N-{[6-hydroxy-7-(1,3-oxazol-2-yl)-5-quinoxalinyl]carbonyl}glycine;
N-[(6-hydroxy-8-phenyl-5-quinoxalinyl)carbonyl]glycine;
N-{[6-hydroxy-7-(1H-indol-3-yl)-5-quinoxalinyl]carbonyl}glycine;
N-{[6-hydroxy-7-(1H-pyrrol-3-yl)-5-quinoxalinyl]carbonyl}glycine;
N-[(6-hydroxy-2-phenyl-5-quinoxalinyl)carbonyl]glycine;
N-{[6-hydroxy-7-(1H-indol-2-yl)-5-quinoxalinyl]carbonyl}glycine;
N-[(6-hydroxy-2-methyl-5-quinoxalinyl)carbonyl]glycine;
N-{[3-(3,4-difluorophenyl)-6-hydroxy-7-(2-thienyl)-5-quinoxalinyl]carbonyl}glycine;
N-{[6-hydroxy-2-phenyl-7-(2-pyridinyl)-5-quinoxalinyl]carbonyl}glycine;
N-{[7-(1-cyclohexen-1-yl)-3-(3,4-difluorophenyl)-6-hydroxy-5-quinoxalinyl]carbonyl}glycine;
N-{[7-(1,3-benzothiazol-2-yl)-6-hydroxy-5-quinoxalinyl]carbonyl}glycine;
N-{[6-hydroxy-7-(1,3-thiazol-5-yl)-5-quinoxalinyl]carbonyl}glycine;
N-[(7-fluoro-6-hydroxy-5-quinoxalinyl)carbonyl]glycine;
N-{[7-cyclohexyl-3-(3,4-difluorophenyl)-6-hydroxy-5-quinoxalinyl]carbonyl}glycine;
N-{[6-hydroxy-7-(3-thienyl)-5-quinoxalinyl]carbonyl}glycine;
N-{[6-hydroxy-7-(1,3-thiazol-4-yl)-5-quinoxalinyl]carbonyl}glycine;
N-{[7-(1-benzothien-2-yl)-6-hydroxy-5-quinoxalinyl]carbonyl}glycine;
N-{[7-(1-benzothien-3-yl)-6-hydroxy-5-quinoxalinyl]carbonyl}glycine;
N-[(6-hydroxy-3,7-diphenyl-5-quinoxalinyl)carbonyl]glycine;
N-[(8-bromo-6-hydroxy-5-quinoxalinyl)carbonyl]glycine;
N-({3-(3,4-difluorophenyl)-7-[4-(1,1-dimethylethyl)phenyl]-6-hydroxy-5-quinoxalinyl}carbonyl)glycine;
N-{[3-(3,4-difluorophenyl)-6-hydroxy-7-phenyl-5-quinoxalinyl]carbonyl}glycine;
N-{[3-(3,4-difluorophenyl)-7-(4-fluorophenyl)-6-hydroxy-5-quinoxalinyl]carbonyl}glycine;
N-[(3-(3,4-difluorophenyl)-6-hydroxy-7-{3-[(1-methylethyl)oxy]phenyl}-5-quinoxalinyl)carbonyl]glycine;
N-[(3-(3,4-difluorophenyl)-6-hydroxy-7-{4-[(1-methylethyl)oxy]phenyl}-5-quinoxalinyl)carbonyl]glycine;
N-{[3-(3,4-difluorophenyl)-7-(3-fluorophenyl)-6-hydroxy-5-quinoxalinyl]carbonyl}glycine;
N-[(6-hydroxy-2,3-diphenyl-5-quinoxalinyl)carbonyl]glycine;
N-{[2-(3-fluorophenyl)-6-hydroxy-5-quinoxalinyl]carbonyl}glycine;
N-{[6-hydroxy-8-(3-pyridinyl)-5-quinoxalinyl]carbonyl}glycine;
N-[(6-hydroxy-2,7-diphenyl-5-quinoxalinyl)carbonyl]glycine;
N-{[6-hydroxy-2-phenyl-7-(2-thienyl)-5-quinoxalinyl]carbonyl}glycine;
N-{[6-hydroxy-8-(3-thienyl)-5-quinoxalinyl]carbonyl}glycine;
N-[(6-hydroxy-2,7-di-2-thienyl-5-quinoxalinyl)carbonyl]glycine;
N-[{6-hydroxy-8-(2-pyridinyl)-5-quinoxalinyl]carbonyl}glycine;
N-[{6-hydroxy-8-(2-thienyl)-5-quinoxalinyl]carbonyl}glycine;
N-[(6-hydroxy-2,7-di-1,3-thiazol-2-yl-5-quinoxalinyl)carbonyl]glycine;
N-{[8-(2-furanyl)-6-hydroxy-5-quinoxalinyl]carbonyl}glycine;
N-{[6-hydroxy-8-(1,3-thiazol-5-yl)-5-quinoxalinyl]carbonyl}glycine;
N-{[6-hydroxy-8-(1,3-thiazol-4-yl)-5-quinoxalinyl]carbonyl}glycine;
N-{[6-hydroxy-2-(3-pyridinyl)-5-quinoxalinyl]carbonyl}glycine;
N-({6-hydroxy-2-[3-(methyloxy)phenyl]-5-quinoxalinyl}carbonyl)glycine;
N-{[6-hydroxy-2-(2-hydroxyphenyl)-5-quinoxalinyl]carbonyl}glycine;
N-({6-hydroxy-2-[4-(methyloxy)phenyl]-5-quinoxalinyl}carbonyl)glycine;
N-[(6-hydroxy-2-{3-[(1-methylethyl)oxy]phenyl}-5-quinoxalinyl)carbonyl]glycine;
N-{[8-(1-benzothien-2-yl)-6-hydroxy-5-quinoxalinyl]carbonyl}glycine;
N-{[8-(1-cyclohexen-1-yl)-6-hydroxy-5-quinoxalinyl]carbonyl}glycine;
N-({8-[2-fluoro-4-(trifluoromethyl)phenyl]-6-hydroxy-5-quinoxalinyl}carbonyl)glycine;
N-{[8-(3-bromo-5-fluorophenyl)-6-hydroxy-5-quinoxalinyl]carbonyl}glycine;
N-{[8-(4-bromo-2-fluorophenyl)-6-hydroxy-5-quinoxalinyl]carbonyl}glycine;
N-{[2-(3,4-difluorophenyl)-6-hydroxy-7-(2-thienyl)-5-quinoxalinyl]carbonyl}glycine;
N-{[8-(1-benzothien-3-yl)-6-hydroxy-5-quinoxalinyl]carbonyl}glycine;
N-{[2-(3,5-difluorophenyl)-6-hydr oxy-5-quinoxalinyl]carbonyl}glycine;
N-{[6-hydroxy-2-(4-hydroxyphenyl)-5-quinoxalinyl]carbonyl}glycine;
N-({2-[4-(dimethylamino)phenyl]-6-hydroxy-5-quinoxalinyl}carbonyl)glycine;
N-({2-[2,4-bis(methyloxy)phenyl]-6-hydroxy-5-quinoxalinyl}carbonyl)glycine;
N-{[2-(1-benzothien-2-yl)-6-hydroxy-5-quinoxalinyl]carbonyl}glycine;
N-[(6-hydroxy-2-{4-[(1-methylethyl)oxy]phenyl}-5-quinoxalinyl)carbonyl]glycine;
N-[6-hydroxy-2-(4-pyridinyl)-5-quinoxalinyl]carbonyl glycine;
N-{[2-(4-fluorophenyl)-6-hydroxy-5-quinoxalinyl]carbonyl}glycine;
N-{[2-(3,4-difluorophenyl)-6-hydroxy-5-quinoxalinyl]carbonyl}glycine;
N-({6-hydr oxy-2-[3-(trifluoromethyl)phenyl]-5-quinoxalinyl}carbonyl)glycine;
N-({2-[3-(dimethylamino)phenyl]-6-hydroxy-5-quinoxalinyl}carbonyl)glycine;
N-({6-hydr oxy-2-[2-(methyloxy)phenyl]-5-quinoxalinyl}carbonyl)glycine;
N-{[6-hydroxy-2-(2-thienyl)-5-quinoxalinyl]carbonyl}glycine;
N-{[(6-hydroxy-2-{2-[(1-methylethyl)oxy]phenyl}-5-quinoxalinyl]carbonyl}glycine;
N-{[6-hydroxy-8-(1-methyl-1-pyrazol-3-yl)-5-quinoxalinyl]carbonyl}glycine;
N-{[8-(3,4-difluorophenyl)-6-hydroxy-5-quinoxalinyl]carbonyl}glycine;
N-{[6-hydroxy-8-(1,3-thiazol-2-yl)-5-quinoxalinyl]carbonyl}glycine;
N-{[6-hydroxy-2-(3-hydroxyphenyl)-5-quinoxalinyl]carbonyl}glycine;
N-({2-[2,3-bis(methyloxy)phenyl]-6-hydroxy-5-quinoxalinyl}carbonyl)glycine;
N-({2-[3,5-bis(methyloxy)phenyl]-6-hydroxy-5-quinoxalinyl}carbonyl)glycine;
N-{[2-(1-benzothien-3-yl)-6-hydroxy-5-quinoxalinyl]carbonyl}glycine;
N-({6-hydroxy-2-[2-(trifluoromethyl)phenyl]-5-quinoxalinyl}carbonyl)glycine;
N-{[2-(2,4-difluorophenyl)-6-hydroxy-5-quinoxalinyl]carbonyl}glycine;
N-{[8-(3-furanyl)-6-hydroxy-5-quinoxalinyl]carbonyl}glycine;
N-[(6-hydroxy-2-oxo-3-phenyl-1,2-dihydro-5-quinoxalinyl)carbonyl]glycine;
N-{[6-hydroxy-8-(3-nitrophenyl)-5-quinoxalinyl]carbonyl}glycine;
N-{[6-hydroxy-8-(2-nitrophenyl)-5-quinoxalinyl]carbonyl}glycine;
N-{[6-hydroxy-3-phenyl-2-(propylamino)-5-quinoxalinyl]carbonyl}glycine;
N-({7-[2-fluoro-4-(trifluoromethyl)phenyl]-6-hydroxy-5-quinoxalinyl}carbonyl)glycine;
N-{[6-hydroxy-2-(1-methyl-1H-pyrazol-4-yl)-5-quinoxalinyl]carbonyl}glycine;
N-{[2-(2-fluorophenyl)-6-hydroxy-5-quinoxalinyl]carbonyl}glycine;
N-({6-hydr oxy-3-phenyl-2-[(phenylmethyl)amino]-5-quinoxalinyl}carbonyl)glycine;
N-{[6-hydroxy-2-phenyl-3-(1,3-thiazol-2-yl)-5-quinoxalinyl]carbonyl}glycine;
N-({2-[3,4-bis(methyloxy)phenyl]-6-hydroxy-5-quinoxalinyl}carbonyl)glycine;
N-{[6-hydroxy-2-(3-thienyl)-5-quinoxalinyl]carbonyl}glycine;
N-{[6-hydroxy-2-(1,3-thiazol-2-yl)-5-quinoxalinyl]carbonyl}glycine;
N-{[2-(2,3-difluorophenyl)-6-hydroxy-5-quinoxalinyl]carbonyl}glycine;
N-{[2-(1,3-benzothiazol-2-yl)-6-hydroxy-5-quinoxalinyl]carbonyl}glycine;
N-({2-[3-(1,1-dimethylethyl)phenyl]-6-hydroxy-5-quinoxalinyl}carbonyl)glycine;
N-({2-[4-(1,1-dimethylethyl)phenyl]-6-hydroxy-5-quinoxalinyl}carbonyl)glycine;
N-{[7-(4-bromo-2-fluorophenyl)-6-hydroxy-5-quinoxalinyl]carbonyl}glycine;
N-{[7-(3-bromo-5-fluorophenyl)-6-hydroxy-5-quinoxalinyl]carbonyl}glycine;
N-{[6-hydroxy-3-phenyl-2-(1,3-thiazol-2-yl)-5-quinoxalinyl]carbonyl}glycine;
N-[(7-chloro-6-hydroxy-5-quinoxalinyl)carbonyl]glycine;
N-({2-[2-(dimethylamino)phenyl]-6-hydroxy-5-quinoxalinyl}carbonyl)glycine;
N-{[7-(3,4-difluorophenyl)-6-hydroxy-2-(2-thienyl)-5-quinoxalinyl]carbonyl}glycine;
N-({2-[2-(1,1-dimethylethyl)phenyl]-6-hydroxy-5-quinoxalinyl}carbonyl)glycine;
N-({6-hydr oxy-2-[4-(methylamino)phenyl]-5-quinoxalinyl}carbonyl)glycine;
N-({6-hydroxy-2-[3-(methylamino)phenyl]-5-quinoxalinyl}carbonyl)glycine;
N-[(7-ethenyl-6-hydroxy-5-quinoxalinyl)carbonyl]glycine;
N-{[2-(3,4-difluorophenyl)-7-(3-fluorophenyl)-6-hydroxy-5-quinoxalinyl]carbonyl}glycine;
N-({2-[3,5-bis(trifluoromethyl)phenyl]-6-hydroxy-5-quinoxalinyl}carbonyl)glycine;
N-{[3,7-bis(3-fluorophenyl)-6-hydroxy-5-quinoxalinyl]carbonyl}glycine;
N-{[6-hydroxy-2(5-pyrimidinyl)-5-quinoxalinyl]carbonyl}glycine;
N-{[7-bromo-6-hydroxy-2-(2-thienyl)-5-quinoxalinyl]carbonyl}glycine;
N-{[2,7-bis(3,4-difluorophenyl)-6-hydroxy-5-quinoxalinyl]carbonyl}glycine;
N-{[7-bromo-6-hydroxy-2-(1,3-thiazol-2-yl)-5-quinoxalinyl]carbonyl}glycine;
N-{[7-(3-fluorophenyl)-6-hydroxy-2-(1,3-thiazol-2-yl)-5-quinoxalinyl]carbonyl}glycine;
N-[(7-bromo-6-hydroxy-2-oxo-3-phenyl-1,2-dihydro-5-quinoxalinyl)carbonyl]glycine;
N-{[7-(3-fluorophenyl)-3-(4-fluorophenyl)-6-hydroxy-5-quinoxalinyl]carbonyl}glycine;
N-({6-hydroxy-2-[2-(methylamino)phenyl]-5-quinoxalinyl}carbonyl)glycine;
N-{[2-(3,4-difluorophenyl)-6-hydroxy-7-(1,3-thiazol-2-yl)-5-quinoxalinyl]carbonyl}glycine;
N-{[6-hydroxy-2-(2-pyridinyl)-5-quinoxalinyl]carbonyl}glycine;
or a pharmaceutically acceptable salt thereof.

5. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

6. A pharmaceutical composition comprising a compound of formula (I) or a salt, solvate, according to claim 1 and one or more of pharmaceutically acceptable carriers, diluents and excipients.

7. A process for preparing a compound of formula (I) 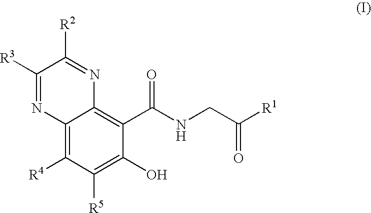
wherein R¹, R², R³, R⁴, and R⁵ are the same as defined above for formula (I), the process comprising treating a compound of formula A: 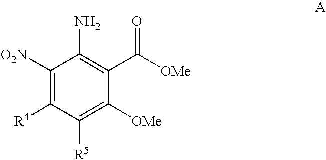
wherein R⁴ and R⁵ are the same as for those groups in formula (I), in a hydrogen atmosphere with an appropriate catalyst, such as palladium on charcoal, in an appropriate solvent, such as ethyl acetate or with an appropriate reducing agent, such as tin(II) chloride dihydrate, in an appropriate solvent, such as ethanol with or without acetonitrile, followed by addition of an appropriately substituted 1,2-dicarbonyl compound or a hydrate thereof, such as phenylglyoxal monohydrate, methyl glyoxal, glyoxal, glyoxylic acid ethyl ester, 2,3-butanedione, 3,4-difluorophenylglyoxal hydrate, 2,4-difluorophenylglyoxal hydrate, t-butylglyoxal, 4-cyclohexylphenylglyoxal hydrate, or 4-fluorophenylglyoxal hydrate, in an appropriate solvent, such as acetonitrile/water or methanol, with heating under either conventional thermal conditions or by microwave irradiation, to form a compound of formula B: 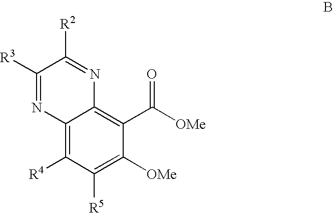
wherein R², R³, R⁴, and R⁵ are the same as for those groups in formula (I), which undergoes ether cleavage/ester hydrolysis with an appropriate reagent, such as boron tribromide, in an appropriate solvent, such as dichloromethane, and is then coupled with an appropriate glycine ester, such as glycine ethyl ester hydrochloride, and an appropriate base, such as triethylamine or diisopropylethylamine, and an appropriate coupling reagent, such as HATU or PyBOP, in an appropriate solvent, such as N,N-dimethylformamide or dichloromethane, followed by ester hydrolysis with an appropriate base, such as sodium hydroxide, in an appropriate solvent, such as ethanol or tetrahydrofuran/methanol, to form a compound of formula (I) where R¹ is —OH.
